(19) United States
(12) Patent Application Publication
(10) Pub. No.: US 2025/0177564 A1
(43) Pub. Date: Jun. 5, 2025

(54) GENE THERAPY FOR THE TREATMENT OF HT1

(71) Applicant: LogicBio Therapeutics, Inc., Lexington, MA (US)

(72) Inventors: Qiang Xiong, Newton, MA (US); Chih-Wei Ko, Brighton, MA (US)

(73) Assignee: LogicBio Therapeutics, Inc., Lexington, MA (US)

(21) Appl. No.: 18/701,866

(22) PCT Filed: Oct. 18, 2022
(86) PCT No.: PCT/US2022/047003

§ 371 Date: Apr. 16, 2024

Related U.S. Application Data
(60) Provisional application No. 63/257,028, filed on Oct. 18, 2021.
(60) Provisional application No. 63/339,783, filed on May 9, 2022.

(51) Int. Cl. A61K 48/00; A61K 38/46; A61P 3/00; C07K 14/005; C12N 15/86; C12N 15/90

(52) U.S. Cl. CPC A61K 48/005; A61K 38/46; A61K 48/0083; A61P 3/00; C07K 14/005; C12N 15/86; C12N 15/907; C12Y 307/01002; C12N 2750/14122; C12N 2750/14143; C12N 2750/14145

(57) ABSTRACT

The present disclosure provides compositions and methods for gene therapy. Further, the present disclosure provides compositions and methods for treatment of HT1 through novel gene therapy mechanisms.

### CROSS-REFERENCE TO RELATED APPLICATIONS

This application claims priority to United States Provisional Application Nos. 63/339,783, filed May 9, 2022, and 63/257,028, filed Oct. 18, 2021, the entirety of each of which is incorporated herein by reference.

### BACKGROUND

There is a subset of human diseases that can be traced to changes in the DNA that are either inherited or acquired early in embryonic development. Of particular interest for developers of genetic therapies are diseases caused by a mutation in a single gene, known as monogenic diseases. There are believed to be over 6,000 monogenic diseases. Typically, any particular genetic disease caused by inherited mutations is relatively rare, but taken together, the toll of genetic-related disease is high. Well-known genetic diseases include cystic fibrosis, Duchenne muscular dystrophy, Huntington's disease and sickle cell disease. Other classes of genetic diseases include metabolic disorders, such as organic acidemias, and lysosomal storage diseases where dysfunctional genes result in defects in metabolic processes and the accumulation of toxic byproducts that can lead to serious morbidity and mortality both in the short-term and long-term.

### SUMMARY

Monogenic diseases have been of particular interest to biomedical innovators due to the perceived simplicity of their disease pathology. However, the vast majority of these diseases and disorders remain substantially untreatable. Thus, there remains a long felt need in the art for the treatment of such diseases.

In some embodiments, the present disclosure provides methods of integrating a transgene into the genome of at least a population of cells in a tissue in a subject. In some embodiments, such methods may include a step of administering to a subject in which cells in the tissue fail to express a functional protein encoded by a gene product, a composition that delivers a transgene encoding the functional protein, wherein the composition includes: a polynucleotide cassette comprising an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell, a third nucleic acid sequence positioned 5′ to the expression cassette and comprising a sequence that is substantially homologous to a genomic sequence 5′ of the target integration site in the genome of the cell, and a fourth nucleic acid sequence positioned 3′ to the expression cassette and comprising a sequence that is substantially homologous to a genomic sequence 3′ of the target integration site in the genome of the cell, wherein, after administering the composition, the transgene is integrated into the genome of the population of cells.

In some embodiments, the present disclosure provides methods of increasing a level of expression of a transgene in a tissue over a period of time, said methods including the step of administering to a subject in need thereof a composition that delivers a transgene that integrates into the genome of at least a population of cells in the tissue of the subject, wherein the composition includes: a polynucleotide cassette comprising an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell, a third nucleic acid sequence positioned 5′ to the expression cassette and comprising a sequence that is substantially homologous to a genomic sequence 5′ of the target integration site in the genome of the cell, and a fourth nucleic acid sequence positioned 3′ to the expression cassette and comprising a sequence that is substantially homologous to a genomic sequence 3′ of the target integration site in the genome of the cell, wherein, after administering the composition, the transgene is integrated into the genome of the population of cells and the level of expression of the transgene in the tissue increases over a period of time. In some embodiments, the increased level of expression comprises an increased percent of cells in the tissue expressing the transgene.

In some embodiments, the present disclosure provides methods including a step of administering to a subject a dose of a composition that delivers to cells in a tissue of the subject a transgene, wherein the transgene (i) encodes fumarylacetoacetate hydrolase (FAH); (ii) integrates at a target integration site in the genome of a plurality of the cells; (iii) functionally expresses FAH once integrated; and (iv) confers a selective advantage to the plurality of cells relative to other cells in the tissue, so that, over time, the tissue achieves a level of functional expression of FAH, wherein the composition comprises: a polynucleotide cassette comprising an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products when the transgene is integrated at the target integration site, a third nucleic acid sequence positioned 5′ to the expression cassette and comprising a sequence that is substantially homologous to a genomic sequence 5′ of the target integration site, and a fourth nucleic acid sequence positioned 3′ to the expression cassette and comprising a sequence that is substantially homologous to a genomic sequence 3′ of the target integration site. In some embodiments, the selective advantage comprises an increased percent of cells in the tissue expressing the transgene.

In some embodiments, the present disclosure provides methods of treatment of a monogenic disease. In some embodiments, the present disclosure provides methods of treating hereditary tyrosinemia type 1 (HT1). In some embodiments, a method of HT1 comprises administering to a subject a dose of a composition comprising a polynucleotide cassette comprising an expression cassette comprising a first nucleic acid sequence and a second nucleic acid sequence, wherein the first nucleic acid sequence encodes an FAH transgene; and the second nucleic acid sequence is positioned 5′ or 3′ to the first nucleic acid sequence and promotes the production of two independent gene products when the transgene is integrated at the target integration site; a third nucleic acid sequence positioned 5′ to the expression cassette and comprising a sequence that is substantially homologous to a genomic sequence 5′ of the target integration site; and a fourth nucleic acid sequence positioned 3′ to the expression cassette and comprising a sequence that is substantially homologous to a genomic sequence 3′ of the target integration site. In some embodiments, the third nucleic acid sequence is selected from SEQ ID NO: 1, SEQ ID NO: 3, and SEQ ID NO: 4. In some embodiments, the fourth nucleic acid sequence is selected from SEQ ID NO: 2 and SEQ ID NO: 5.

In some embodiments, a composition comprises a delivery vehicle. In some embodiments, a delivery vehicle is a particle, e.g., a nanoparticle, e.g., a lipid nanoparticle. In some embodiments, a delivery vehicle is recombinant viral vector. In some embodiments, a recombinant viral vector is a recombinant AAV vector. In some embodiments, a recombinant viral vector is or comprises a capsid protein comprising an amino acid sequence having at least 95% sequence identity with the amino acid sequence of AAV8, AAV-DJ; AAV-LK03; sL65 or AAVNP59. In some embodiments, the composition further comprises AAV2 ITR sequences. In some embodiments, the composition comprises a portion of an AAV2 ITR sequence. In some embodiments, the composition comprises an ITR having at least 80%, 85%, 90%, 95%, 99%, or 100% sequence identity to an AAV2 ITR. In some embodiments, the composition comprises ITR sequences selected from SEQ ID NO: 27, SEQ ID NO: 28, SEQ ID NO: 29, and SEQ ID NO: 30.

In accordance with various embodiments, any of a variety of transgenes may be expressed in accordance with the methods and compositions described herein. For example, in some embodiments, a transgene is or comprises an FAH transgene. In some embodiments, an FAH transgene is a wt human FAH; a codon optimized FAH; a synthetic FAH; an FAH variant; an FAH mutant, or an FAH fragment. In some embodiments, a transgene is or comprises a sequence with at least 80% identity to any one of SEQ ID NO: 18, SEQ ID NO: 19, SEQ ID NO: 20, SEQ ID NO: 21, or SEQ ID NO: 22.

In some embodiments, the present invention provides recombinant viral vectors for integrating a transgene into a target integration site in the genome of a cell, including: a polynucleotide cassette comprising an expression cassette comprising a first nucleic acid sequence and a second nucleic acid sequence, wherein the first nucleic acid sequence comprises an FAH transgene; and the second nucleic acid sequence is positioned 5′ or 3′ to the first nucleic acid sequence and promotes the production of two independent gene products upon integration into the target integration site in the genome of the cell, a third nucleic acid sequence positioned 5′ to the expression cassette and comprising a sequence that is substantially homologous to a genomic sequence 5′ of the target integration site in the genome of the cell, and a fourth nucleic acid sequence positioned 3′ of the expression cassette and comprising a sequence that is substantially homologous to a genomic sequence 3′ of the target integration site in the genome of the cell. In some embodiments, the second nucleic acid sequence is a sequence encoding a P2A peptide. In some embodiments, the second nucleic acid sequence has at least 80% identity to SEQ ID NO: 6. In some embodiments, the second nucleic acid sequence encodes a P2A peptide having at least 90% sequence identity to SEQ ID NO: 7. In some embodiments, provided recombinant viral vectors comprise a sequence of SEQ ID NO: 23, SEQ ID NO: 24, SEQ ID NO: 25, or SEQ ID NO: 26.

As is described herein, the present disclosure encompasses several advantageous recognitions regarding the integration of one or more transgenes into the genome of a cell. For example, in some embodiments, integration does not comprise nuclease activity.

While any application-appropriate tissue may be targeted, in some embodiments, the tissue is the liver.

As is described herein, provided methods and compositions include polynucleotide cassettes with at least four nucleic acid sequences. In some embodiments, the second nucleic acid sequence comprises: a) a nucleic acid sequence encoding a 2A peptide, b) a nucleic acid sequence encoding an internal ribosome entry site (IRES), c) a nucleic acid sequence encoding an N-terminal intein splicing region and C-terminal intein splicing region, or d) a nucleic acid sequence encoding a splice donor and a splice acceptor. In some embodiments, the third and fourth nucleic acid sequences are homology arms that integrate the transgene and the second nucleic acid sequence into a target integration site. In some embodiments a target integration site comprises an endogenous promoter and an endogenous gene. In some embodiments a target integration site is an endogenous albumin gene locus comprising an endogenous albumin promoter and an endogenous albumin gene. In some embodiments, the homology arms direct integration of the expression cassette immediately 3′ of the start codon of the endogenous albumin gene or immediately 5′ of the stop codon of the endogenous albumin gene.

In accordance with various aspects, the third and/or fourth nucleic acids may be of significant length (e.g., at least 800 nucleotides in length). In some embodiments, the third nucleic acid is between 200-3,000 nucleotides. In some embodiments, the fourth nucleic acid is between 200-3,000 nucleotides.

In some embodiments, a polynucleotide cassette does not comprise a promoter sequence. In some embodiments, upon integration of an expression cassette into a target integration site in the genome of the cell, the transgene is expressed under control of an endogenous promoter at the target integration site. In some embodiments, the target integration site is an albumin locus comprising an endogenous albumin promoter and an endogenous albumin gene. In some embodiments, upon integration of an expression cassette into a target integration site in the genome of a cell, the transgene is expressed under control of the endogenous albumin promoter without disruption of the endogenous albumin gene expression.

In some embodiments, provided compositions may be administered to a subject in dosages between 1E12 and 1E14 vg/kg. In some embodiments, provided compositions may be administered to a subject in dosages between 3E12 and 1E13 vg/kg. In some embodiments, provided compositions may be administered to a subject in dosages between 3E12 and 3E13 vg/kg. In some embodiments, provided compositions may be administered to a subject in dosages of no more than 3E13 vg/kg. In some embodiments, provided compositions may be administered to a subject in dosages of no more than 3E12 vg/kg. In some embodiments, provided compositions may be administered to a subject only once. In some embodiments, provided compositions may be administered to a subject more than once.

In some embodiments, the present disclosure provides an insight that provided compositions may be administered to a newborn subject. Further, in some embodiments, provided compositions may be administered to subject between 0 days and 1 month of age, 3 months of age and 1 year of age, 1 year of age and 5 years of age, and 5 years of age and older. In some embodiments, provided compositions may be administered to a subject, wherein the subject is an animal. In some embodiments, provided compositions may be administered to a subject, wherein the subject is a human.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

FIGS. 1A-1I show results of treatment of HT1 mouse models with therapies as described herein. Figure TA shows an exemplary polynucleotide cassette. FIG. 1B shows an exemplary therapy time course. FIG. 1C shows assessment of circulating biomarker. FIG. 1D shows body weight changes of treated mice. FIGS. 1E and 1F show markers of liver function in treated mice. FIG. 1G shows immunohistochemical staining of liver samples with FAH antibody. FIG. 1H shows assessment of integration into host genomic DNA (gDNA INT). Figure TI shows assessment of presence of alfa-fetoprotein (AFP) which is a clinically validated in-life biomarker for hepatocellular carcinoma (HCC).

FIGS. 2A-2E, show results and selective advantage of treatment of HT1 mouse models with therapies as described herein. FIG. 2A shows an exemplary vector and treatment schedule. FIG. 2B shows assessment of GENERIDE™ biomarkers. FIG. 2C shows survival rate of treated mice. FIGS. 2D and 2E show markers of liver function in treated mice.

FIG. 3A-3D shows results of treatment of pediatric HT1 mouse models with therapies as described herein. FIG. 3A shows treatment time-course. FIG. 3B shows assessment of GENERIDE™ biomarkers. FIG. 3C shows body weight changes of treated mice. FIG. 3D shows AFP level in treated mice.

FIGS. 4A-4C, show results of various treatment schedules of HT1 mouse models with therapies as described herein. FIG. 4A shows treatment of mice with various treatment schedule. FIG. 4B shows assessment of circulating biomarker. FIG. 4C shows markers of liver function in treated mice.

FIG. 5 shows a schematic of the tyrosine metabolism pathway.

FIGS. 6A-6F, show results of NTBC cycling. FIG. 6A shows an exemplary study design. FIG. 6B shows the level of a toxic metabolite that accumulates in HT1 (SUAC); FIG. 6C shows a marker of liver injury (e.g., ALT). FIG. 6D shows a marker of liver synthetic function (e.g., activated partial thromboplastin time (aPTT), which shows clotting time). FIG. 6E shows blood glucose levels. FIG. 6F shows body weight changes of mice.

FIGS. 7A-7F, show results of GENERIDE™ treatment during 28 days post treatment, in the absence of NTBC. FIG. 7A shows an assessment of the circulating biomarker (e.g., ALB2A). FIG. 7B shows the accumulation of the HT1-related toxic metabolite (e.g., SUAC). FIG. 7C shows a marker of liver function (e.g., ALT). FIG. 7D shows clotting time. FIG. 7E shows blood glucose levels. FIG. 7F shows body weight changes of treated mice.

FIGS. 8A-8D, show results of GENERIDE™ treatment over 4 months post treatment. FIG. 8A shows an exemplary study design. FIG. 8B shows an assessment of the circulating biomarker (e.g., ALB2A) (left panel) and immunohistochemical staining of liver samples with an anti-FAH antibody (right panel). FIG. 8C shows an assessment of liver function as a measure of various biomarkers (e.g., aspartate aminotransferase (AST), alanine aminotransferase (ALT), alkaline phosphatase (ALP), gamma-glutamyl transferase (GGT), total bilirubin (TBIL), albumin (ALB)). FIG. 8D shows an assessment of kidney function as a measure of various biomarkers (e.g., urea nitrogen (UREAN), creatinine (CREAT)).

FIG. 9A-9E, show results of GENERIDE™ treatment over several months post treatment. FIG. 9A shows an exemplary study design. FIG. 9B shows the toxic metabolite that accumulates in HT1 (e.g., SUAC). FIG. 9C shows an assessment of the circulating biomarker (e.g., ALB2A). FIG. 9D shows an assessment of alfa-fetoprotein (AFP) which is a clinically validated in-life biomarker for hepatocellular carcinoma (HCC). FIG. 9E shows an assessment of tyrosine levels.

FIG. 10A-10B, shows results of pediatric HT1 mouse models administered GENERIDE™ treatment. FIG. 10A shows schematic of treatment time-course. FIG. 3B shows alfa-fetoprotein (AFP) level in mice treated at post-natal day 14 (PND14).

FIG. 11, shows an exemplary immunohistochemical staining of markers of liver function in mice administered GENERIDE™ treatment.

### DEFINITIONS

In order for the present invention to be more readily understood, certain terms are first defined below. Additional definitions for the following terms and other terms are set forth throughout the specification.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Adult: As used herein, the term “adult” refers to a human eighteen years of age or older. In some embodiments, a human adult has a weight within the range of about 90 pounds to about 250 pounds.

Associated: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Biomarker: The term “biomarker” is used herein, consistent with its use in the art, to refer to an entity whose presence, level, or form correlates with a particular biological event or state of interest, so that it is considered to be a “marker” of that event or state. Among other things, the present disclosure provides biomarkers for gene therapy (e.g., that are useful to assess one or more features or characteristics of a gene therapy treatment, such as, for instance, extent, level, and/or persistence of payload expression). In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, etc). In certain embodiments, the present disclosure demonstrates effectiveness of biomarkers that can be detected in a sample obtained from a subject who has received gene therapy for use in assessing one or more features or characteristics of that gene therapy; in some such embodiments, the sample is of cells, tissue, and/or fluid other than that to which the gene therapy was delivered and/or other than that where the payload is active.

Codon optimization: As used herein, the term “codon optimization” refers to a process of changing codons of a given gene in such a manner that the polypeptide sequence encoded by the gene remains the same while the changed codons improve the process of expression of the polypeptide sequence. For example, if the polypeptide is of a human protein sequence and expressed in E. coli, expression will often be improved if codon optimization is performed on the DNA sequence to change the human codons to codons that are more effective for expression in E. coli.

Detectable Moiety: The term “detectable moiety” as used herein refers to any entity (e.g., molecule, complex, or portion or component thereof). In some embodiments, a detectable moiety is provided and/or utilizes as a discrete molecular entity; in some embodiments, it is part of and/or associated with another molecular entity. Examples of detectable moieties include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, antibodies, and/or proteins for which antisera or monoclonal antibodies are available.

Child: As used herein, the term “child” refers to a human between two and 18 years of age. Body weight can vary widely across ages and specific children, with a typical range being 30 pounds to 150 pounds.

Combination therapy: As used herein, the term “combination therapy” refers to those situations in which a subject is simultaneously exposed to two or more therapeutic regimens (e.g., two or more therapeutic agents, for example a gene therapy and a non-gene therapy therapeutic modality). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time).

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Gene: As used herein, the term “gene” refers to a DNA sequence that encodes a gene product (e.g., an RNA product and/or a polypeptide product). In some embodiments, a gene includes a coding sequence (e.g., a sequence that encodes a particular gene product); in some embodiments, a gene includes a non-coding sequence. In some particular embodiments, a gene may include both coding (e.g., exonic) and non-coding (e.g., intronic) sequences. In some embodiments, a gene may include one or more regulatory elements (e.g. promoters, enhancers, silencers, termination signals) that, for example, may control or impact one or more aspects of gene expression (e.g., cell-type-specific expression, inducible expression). In some embodiments, a gene is located or found (or has a nucleotide sequence identical to that located or found) in a genome (e.g., in or on a chromosome or other replicable nucleic acid).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Infant: As used herein, the term “infant” refers to a human under two years of age. Typical body weights for an infant range from 3 pounds up to 20 pounds.

Neonate: As used herein, the term “neonate” refers to a newborn human.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Peptide: As used herein, the term “peptide” or “polypeptide” refers to any polymeric chain of amino acids. In some embodiments, a peptide has an amino acid sequence that occurs in nature. In some embodiments, a peptide has an amino acid sequence that does not occur in nature. In some embodiments, a peptide has an amino acid sequence that is engineered in that it is designed and/or produced through action of the hand of man. In some embodiments, a peptide may comprise or consist of natural amino acids, non-natural amino acids, or both. In some embodiments, a peptide may comprise or consist of only natural amino acids or only non-natural amino acids. In some embodiments, a peptide may comprise D-amino acids, L-amino acids, or both. In some embodiments, a peptide may comprise only D-amino acids. In some embodiments, a peptide may comprise only L-amino acids. In some embodiments, a peptide is linear. In some embodiments, the term “peptide” may be appended to a name of a reference peptide, activity, or structure; in such instances it is used herein to refer to peptides that share the relevant activity or structure and thus can be considered to be members of the same class or family of peptides. For each such class, the present specification provides and/or those skilled in the art will be aware of exemplary peptides within the class whose amino acid sequences and/or functions are known; in some embodiments, such exemplary peptides are reference peptides for the peptide class or family. In some embodiments, a member of a peptide class or family shows significant sequence homology or identity with, shares a common sequence motif (e.g., a characteristic sequence element) with, and/or shares a common activity (in some embodiments at a comparable level or within a designated range) with a reference peptide of the class; in some embodiments with all peptides within the class). For example, in some embodiments, a member peptide shows an overall degree of sequence homology or identity with a reference peptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION OF CERTAIN EMBODIMENTS

### Gene Therapy

Gene therapies alter the gene expression profile of a patient's cells by gene transfer, a process of delivering a therapeutic gene, called a transgene. Various delivery vehicles are known to be used as vectors to transport transgenes into the nucleus of a cell to alter or augment the cell's capabilities (e.g., proteome, functionality, etc.). Developers have made great strides in introducing genes into cells in tissues such as the liver, the retina of the eye and the blood-forming cells of the bone marrow using a variety of vectors. These approaches have in some cases led to approved therapies and, in other cases, have shown very promising results in clinical trials.

There are multiple gene therapy approaches. In conventional AAV gene therapy, the transgene is introduced into the nucleus of the host cell, but is not intended to integrate in chromosomal DNA. The transgene is expressed from a non-integrated genetic element called an episome that exists inside the nucleus. A second type of gene therapy employs the use of a different type of virus, such as lentivirus, that inserts itself, along with the transgene, into the chromosomal DNA but at arbitrary sites.

Episomal expression of a gene must be driven by an exogenous promoter, leading to production of a protein that corrects or ameliorates the disease condition.

### Limitations of Gene Therapy

Dilution effects as cells divide and tissues grow. In the case of gene therapy based on episomal expression, when cells divide during the process of growth or tissue regeneration, the benefits of the therapy typically decline because the transgenes were not intended to integrate into the host chromosome, thus not replicated during cell division. Each new generation of cells thus further reduces the proportion of cells expressing the transgene in the target tissue, leading to the reduction or elimination of the therapeutic benefit over time.

Inability to control site of insertion. While the use of some gene therapy using viral mediated insertion has the potential to provide long-term benefit because the gene is inserted into the host chromosome, there is no ability to control where the gene is inserted, which presents a risk of disrupting an essential gene or inserting into a location that can promote undesired effects such as tumor formation. For this reason, these integrating gene therapy approaches are primarily limited to ex vivo approaches, where the cells are treated outside the body and then re-inserted.

Use of exogenous promoters increases the risk of tumor formation. A common feature of both gene therapy approaches is that the transgene is introduced into cells together with an exogenous promoter. Promoters are required to initiate the transcription and amplification of DNA to messenger RNA, or mRNA, which will ultimately be translated into protein. Expression of high levels of therapeutic proteins from a gene therapy transgene requires strong, engineered promoters. While these promoters are essential for protein expression, previous studies conducted by others in animal models have shown that non-specific integration of gene therapy vectors can result in significant increases in the development of tumors. The strength of the promoters plays a crucial role in the increase of the development of these tumors. Thus, attempts to drive high levels of expression with strong promoters may have long-term deleterious consequences.

### A. Gene Editing

Gene editing is the deletion, alteration or augmentation of aberrant genes by introducing breaks in the DNA of cells using exogenously delivered gene editing mechanisms. Most current gene editing approaches have been limited in their efficacy due to high rates of unwanted on- and off-target modifications and low efficiency of gene correction, resulting in part from the cell trying to rapidly repair the introduced DNA break. The current focus of gene editing is on disabling a dysfunctional gene or correcting or skipping an individual deleterious mutation within a gene. Due to the number of possible mutations, neither of these approaches can address the entire population of mutations within a particular genetic disease, as would be addressed by the insertion of a full corrective gene.

Unlike the gene therapy approach, gene editing allows for the repaired genetic region to propagate to new generations of cells through normal cell division. Furthermore, the desired protein can be expressed using the cell's own regulatory machinery. The traditional approach to gene editing is nuclease-based, and it uses nuclease enzymes derived from bacteria to cut the DNA at a specific place in order to cause a deletion, make an alteration or apply a corrective sequence to the body's DNA.

Once nucleases have cut the DNA, traditional gene editing techniques modify DNA using two routes: homology-directed repair, or HDR and non-homologous end joining, or NHEJ. HDR involves highly precise incorporation of correct DNA sequences complementary to a site of DNA damage. HDR has key advantages in that it can repair DNA with high fidelity and it avoids the introduction of unwanted mutations at the site of correction. NHEJ is a less selective, more error-prone process that rapidly joins the ends of broken DNA, resulting in a high frequency of insertions or deletions at the break site.

### 1. Nuclease-Based Gene Editing

Nuclease-based gene editing uses nucleases, enzymes that were engineered or initially identified in bacteria that cut DNA. Nuclease-based gene editing is a two-step process. First, an exogenous nuclease, which is capable of cutting one or both strands in the double-stranded DNA, is directed to the desired site by a synthetic guide RNA and makes a specific cut. After the nuclease makes the desired cut or cuts, the cell's DNA repair machinery is activated and completes the editing process through either NHEJ or, less commonly, HDR.

NHEJ can occur in the absence of a DNA template for the cell to copy as it repairs a DNA cut. This is the primary or default pathway that the cell uses to repair double-stranded breaks. The NHEJ mechanism can be used to introduce small insertions or deletions, known as indels, resulting in the knocking out of the function of the gene. NHEJ creates insertions and deletions in the DNA due to its mode of repair and can also result in the introduction of off-target, unwanted mutations including chromosomal aberrations.

Nuclease-mediated HDR occurs with the co-delivery of the nuclease, a guide RNA and a DNA template that is similar to the DNA that has been cut. Consequently, the cell can use this template to construct reparative DNA, resulting in the replacement of defective genetic sequences with correct ones. We believe the HDR mechanism is the preferred repair pathway when using a nuclease-based approach to insert a corrective sequence due to its high fidelity. However, a majority of the repair to the genome after being cut with a nuclease continues to use the NHEJ mechanism. The more frequent NHEJ repair pathway has the potential to cause unwanted mutations at the cut site, thus limiting the range of diseases that any nuclease-based gene editing approaches can target at this time.

Traditional gene editing has used one of three nuclease-based approaches: Transcription activator-like effector nucleases, or TALENs; Clustered, Regularly Interspaced Short Palindromic Repeats Associated protein-9, or CRISPR/Cas9; and Zinc Finger Nucleases, or ZFN. While these approaches have already contributed to significant advances in research and product development, they have inherent limitations.

### 2. Limitations of Nuclease-Based Gene Editing

Nuclease-based gene editing approaches are limited by their use of bacterial nuclease enzymes to cut DNA and by their reliance on exogenous promoters for transgene expression. These limitations include:

Nucleases cause on- and off-target mutations. Conventional gene editing technologies can result in genotoxicity, including chromosomal alterations, based on the error-prone NHEJ process and potential off-target nuclease activity.

Delivery of gene editing components to cells is complex. Gene editing requires multiple components to be delivered into the same cell at the same time. This is technically challenging and currently requires the use of multiple vectors.

Bacterially derived nucleases are immunogenic. Because the nucleases used in conventional gene editing approaches are mostly bacterially derived, they have a higher potential for immunogenicity, which in turn limits their utility.

Because of these limitations, gene editing has been primarily restricted to ex vivo applications in cells, such as hematopoietic cells.

### GENERIDE™ Technology Platform

GENERIDE™ is a novel AAV-based, nuclease-free, genome editing technology that precisely inserts a therapeutic transgene into the genome via homologous recombination. GENERIDE™ provides durable transgene expression regardless of cell proliferation and tissue growth, and GENERIDE™-corrected hepatocytes show selective expansion in the presence of intrinsic liver damage due to genetic defects (e.g., HT1 due to faulty FAH). Without wishing to be bound by any particular theory, it is contemplated that GENERIDE™ is a genome editing technology that harnesses homologous recombination, or HR, a naturally occurring DNA repair process that maintains the fidelity of the genome. In some embodiments, by using HR, GENERIDE™ allows insertion of transgenes into specific targeted genomic locations without using exogenous nucleases, which are enzymes engineered to cut DNA. GENERIDE™-directed transgene integration is designed to leverage endogenous promoters at these targeted locations to drive high levels of tissue-specific gene expression, without the detrimental issues that have been associated with the use of exogenous promoters.

GENERIDE™ technology is designed to precisely integrate corrective genes into a patient's genome to provide a stable therapeutic effect. Because GENERIDE™ is designed to have this durable therapeutic effect, it can be applied to targeting rare liver disorders in pediatric patients where it is critical to provide treatment early in a patient's life before irreversible disease pathology can occur. In some embodiments, described herein, compositions comprising GENERIDE™ constructs can be used for the treatment of hereditary tyrosinemia type 1 or HTT, a life-threatening disease that presents at birth.

GENERIDE™ platform technology has the potential to overcome some of the key limitations of both traditional gene therapy and conventional gene editing approaches in a way that is well-positioned to treat genetic diseases, particularly in pediatric patients. In some embodiments, GENERIDE™ uses an AAV vector to deliver a gene into the nucleus of the cell. It then uses HR to stably integrate the corrective gene into the genome of the recipient at a location where it is regulated by an endogenous promoter, leading to the potential for lifelong protein production, even as the body grows and changes over time, which is not feasible with conventional AAV gene therapy.

GENERIDE™ offers several key advantages over gene therapy and gene editing technologies that rely on exogenous promoters and nucleases. By harnessing the naturally occurring process of HR, GENERIDE™ does not face the same challenges associated with gene editing approaches that rely on engineered bacterial nuclease enzymes. The use of these enzymes has been associated with significantly increased risk of unwanted and potentially dangerous modifications in the host cell's DNA, which can lead to an increased risk of tumor formation. Furthermore, in contrast to conventional gene therapy, GENERIDE™ is intended to provide precise, site-specific, stable and durable integration of a corrective gene into the chromosome of a host cell. In preclinical animal studies with GENERIDE™ constructs, integration of the corrective gene in a specific location in the genome is observed. Thus, in some embodiments, methods and compositions of the present disclosure (e.g., those comprising GENERIDE™ constructs) provide a more durable approach than gene therapy technologies that do not integrate into the genome and lose their effect as cells divide. These benefits make GENERIDE™ well-positioned to treat genetic diseases, particularly in pediatric patients.

The modular approach disclosed herein can be applied to allow GENERIDE™ to deliver robust, tissue-specific gene expression that will be reproducible across different therapeutics delivered to the same tissue. In some embodiments, this approach allows leverage of common manufacturing processes and analytics across different GENERIDE™ product candidates and could shorten the development process of treatment programs.

Previous work on non-disruptive gene targeting is described in WO 2013/158309, and is incorporated herein by reference. Previous work on genome editing without nucleases is described in WO 2015/143177, and is incorporated herein by reference.

### B. Genome Editing Using GENERIDE™: Mechanism and Attributes

In some embodiments, genome editing with the GENERIDE™ platform differs from gene editing because it uses HR to deliver the corrective gene to one specific location in the genome. In some embodiments, GENERIDE™ inserts the corrective gene in a precise manner, leading to site-specific integration in the genome. In some embodiments, GENERIDE™ does not require the use of exogenous nucleases or promoters; instead, it leverages the cell's existing machinery to integrate and initiate transcription of therapeutic transgenes.

In some embodiments, GENERIDE™ comprises at least three components, each of which contributes to the potential benefits of the GENERIDE™ approach. In some embodiments, compositions and methods of the present disclosure comprise: homology arms, a transgene, and a nucleic acid that promotes the production of two independent gene products. In some embodiments, compositions and methods of the present disclosure comprise a first nucleic acid sequence encoding a transgene. In some embodiments, compositions and methods of the present disclosure comprise a second nucleic acid that promotes the production of two independent gene products (e.g., a 2A peptide). In some embodiments, the present disclosure provides and expression cassette comprising a first nucleic acid sequence and a second nucleic acid sequence as described herein.

In some embodiments, a second nucleic acid comprises a nucleic acid sequence encoding a 2A peptide; a nucleic acid sequence encoding an internal ribosome entry site (IRES); a nucleic acid sequence encoding an N-terminal intein splicing region and C-terminal intein splicing region; and/or a nucleic acid sequence encoding a splice donor and a splice acceptor. In some embodiments, compositions and methods of the present disclosure comprise a polynucleotide cassette comprising an expression cassette comprising said first nucleic acid and said second nucleic acid. In some embodiments, compositions and methods of the present disclosure comprise a third nucleic acid sequence comprising a sequence that is substantially homologous to a genomic sequence. In some embodiments, compositions and methods of the present disclosure comprise a fourth nucleic acid sequence comprising a sequence that is substantially homologous to a genomic sequence. In some embodiments, said third nucleic acid sequence is positioned 5′ to the expression cassette and comprises a sequence that is substantially homologous to a genomic sequence 5′ of a target integration site in a genome of a cell. In some embodiments, said fourth nucleic acid sequence is positioned 3′ to the expression cassette and comprises a sequence that is substantially homologous to a genomic sequence 3′ of a target integration site in the genome of the cell

### Homology Arms Comprised of Hundreds of Nucleotides.

In some embodiments, methods and compositions of the present disclosure comprise flanking sequences, known as homology arms. In some embodiments, homology arms direct site-specific integration (also referred to herein as promoting integration) and limit off-target insertion of the construct. In some embodiments, said third and fourth nucleic acid sequences comprise homology arms. In some embodiments, each homology arm is hundreds of nucleotides long, in contrast to guide sequences used in CRISPR/Cas9, which are only dozens of base pairs long. In some embodiments, this increased length may promote improved precision and site-specific integration. In some embodiments, GENERIDE™'s homology arms direct integration of the transgene immediately behind a highly expressed gene. In some embodiments, integration of the transgene immediately behind a highly expressed gene results in high levels of expression without the need to introduce an exogenous promoter.

In some embodiments, a third or fourth nucleic acid is between 200-3000; 200-350; 250-400; 300-450; 350-500, 500-750; 600-850; 700-950; 800-1050; 900-1150; 1000-1250; 1100-1350; 1200-1450; 1300-1550; 1400-1650; 1500-1750; 1600-1850; 1700-1950; 1800-2050; 1900-2150; 2000-2250; 2100-2350; 2200-2450; 2300-2550; 2400-2650; 2500-2750; 2600-2850; 2700-2950; or 2800-3000 nucleotides in length. In some embodiments, a third or fourth nucleic acid is about 300, 400, 500, 600, 700, 800, 900; 1000; 1100; 1200; 1300; 1400; 1500; 1600; 1700, 1800, 1900, 2000, 2100, 2200 or 1700 nucleotides in length. In some embodiments, a fourth nucleic acid is 1000 nucleotides in length. In some embodiments, a third nucleic acid is 1600 nucleotides in length.

In some embodiments, homology arms contain at least 70% homology to a target locus. In some embodiments, homology arms contain at least 80% homology to a target locus. In some embodiments, homology arms contain at least 90% homology to a target locus. In some embodiments, homology arms contain at least 95% homology to a target locus. In some embodiments, homology arms contain at least 99% homology to a target locus. In some embodiments, homology arms contain 100% homology to a target locus.

In some embodiments, homology arms are of the same length (also referred to as balanced homology arms or even homology arms). In some embodiments, homology arms are of different lengths (also referred to as unbalanced homology arms or uneven homology arms). In some embodiments, compositions comprising unbalanced homology arms of different lengths provide improved effects (e.g., increased rate of target site integration) as compared to a reference sequence or balanced homology arms. In some embodiments, compositions comprising homology arms of different lengths, wherein each homology arm is at least a certain length, provide improved effects (e.g., increased rate of target site integration) as compared to a reference sequence (e.g., a composition comprising homology arms of the same length).

In some embodiments, each homology arm is greater than 50 nt in length. In some embodiments, each homology arm is greater than 100 nt in length. In some embodiments, each homology arm is greater than 400 nt in length. In some embodiments, each homology arm is at least 750 nt length. In some embodiments, each homology arm is at least 1000 nt in length. In some embodiments, one homology arm is at least 750 nt in length and another homology arm is at least 1000 nt in length. In some embodiments, one homology arm is at least 750 nt in length and another homology arm is at least 1100 nt in length. In some embodiments, one homology arm is at least 750 nt in length and another homology arm is at least 1200 nt in length. In some embodiments, one homology arm is at least 750 nt in length and another homology arm is at least 1300 nt in length. In some embodiments, one homology arm is at least 750 nt in length and another homology arm is at least 1400 nt in length. In some embodiments, one homology arm is at least 750 nt in length and another homology arm is at least 1500 nt in length. In some embodiments, one homology arm is at least 750 nt in length and another homology arm is at least 1600 nt in length. In some embodiments, one homology arm is at least 750 nt in length and another homology arm is at least 1700 nt in length. In some embodiments, one homology arm is at least 750 nt in length and another homology arm is at least 1800 nt in length. In some embodiments, one homology arm is at least 750 nt in length and another homology arm is at least 1900 nt in length. In some embodiments, one homology arm is at least 750 nt in length and another homology arm is at least 2000 nt in length. In some embodiments, one homology arm is at least 1000 nt in length and another homology arm is at least 1100 nt in length. In some embodiments, one homology arm is at least 1000 nt in length and another homology arm is at least 1200 nt in length. In some embodiments, one homology arm is at least 1000 nt in length and another homology arm is at least 1300 nt in length. In some embodiments, one homology arm is at least 1000 nt in length and another homology arm is at least 1400 nt in length. In some embodiments, one homology arm is at least 1000 nt in length and another homology arm is at least 1500 nt in length. In some embodiments, one homology arm is at least 1000 nt in length and another homology arm is at least 1600 nt in length. In some embodiments, one homology arm is at least 1000 nt in length and another homology arm is at least 1700 nt in length. In some embodiments, one homology arm is at least 1000 nt in length and another homology arm is at least 1800 nt in length. In some embodiments, one homology arm is at least 1000 nt in length and another homology arm is at least 1900 nt in length. In some embodiments, one homology arm is at least 1000 nt in length and another homology arm is at least 2000 nt in length. In some embodiments, one homology arm is at least 1300 nt in length and another homology arm is at least 1400 nt in length. In some embodiments, one homology arm is at least 1600 nt in length and another homology arm is at least 1000 nt in length. In some embodiments, one homology arm is at least 1250 nt in length and another homology arm is at least 1250 nt in length. In some embodiments, one homology arm is at least 400 nt in length and another homology arm is at least 800 nt in length. In some embodiments, one homology arm is at least 600 nt in length and another homology arm is at least 600 nt in length.

In some embodiments, a 5′ homology arm is longer than a 3′ homology arm. In some embodiments, a 3′ homology arm is longer than a 5′ homology arm. For example, in some embodiments, a 5′ homology arm is approximately 1600 nt in length and a 3′ homology arm is approximately 1000 nt in length. In some embodiments, a 5′ homology arm is approximately 1000 nt in length and a 3′ homology arm is approximately 1600 nt in length. In some embodiments, viral vectors comprising homology arms provide improved effects (e.g., increased rate of target site integration) as compared to an appropriate reference sequence (e.g., viral vectors lacking homology arms). In some embodiments, viral vectors comprising homology arms provide rates of target site integration of 0.01% or more (e.g., 0.05% or more, 0.1% or more, 0.2% or more, 0.3% or more, 0.4% or more, 0.5% or more, 0.6% or more, 0.7% or more, 0.8% or more, 0.9% or more, 1% or more, 1.5% or more, 2% or more, 5% or more, 10% or more, 20% or more, 30% or more). In some embodiments, viral vectors comprising homology arms provide increasing rates of target site integration over time. In some embodiments, rates of target site integration increase over time relative to an initial measurement of target site integration. In some embodiments, rates of target site integration over time are at least 1.5× higher than an initial measurement of target site integration (e.g., 1.5×, 2×, 3×, 4×, 5×, 10×, 20×, 30×, 40×, 50×, 60×, 70×, 80×, 90×, 100×, 200×). In some embodiments, rates of target site integration are measured after one or more days. In some embodiments, rates of target site integration are measured after one or more weeks. In some embodiments, rates of target site integration are measured after one or more months. In some embodiments, rates of target site integration are measured after one or more years.

In some embodiments, viral vectors comprising homology arms of different lengths provide improved effects (e.g., increased rate of target site integration) relative to a reference sequence (e.g., viral vectors with homology arms of the same length, viral vectors with at least one homology arm below 500 nt). In some embodiments, viral vectors comprising homology arms of different lengths provide at least 1.1×, at least 1.2×, at least 1.3×, at least 1.4×, at least 1.5×, at least 1.6×, at least 1.7×, at least 1.8×, at least 1.9×, at least 2.0×, at least 2.5×, at least 3.0×, at least 3.5×, or at least 4.0× improved editing activity relative to a reference composition (e.g., viral vectors with homology arms of the same length, viral vectors with at least one homology arm below 500 nt).

In some embodiments, viral vectors comprising homology arms of different lengths provide rates of target site integration of 0.01% or more (e.g., 0.05% or more, 0.1% or more, 0.2% or more, 0.3% or more, 0.4% or more, 0.5% or more, 0.6% or more, 0.7% or more, 0.8% or more, 0.9% or more, 1% or more, 1.5% or more, 2% or more, 5% or more, 10% or more, 20% or more, 30% or more). In some embodiments, viral vectors comprising homology arms of different lengths provide increasing rates of target site integration over time. In some embodiments, rates of target site integration increase over time relative to an initial measurement of target site integration. In some embodiments, rates of target site integration over time are at least 1.5× higher than an initial measurement of target site integration (e.g., 1.5×, 2×, 3×, 4×, 5×, 10×, 20×, 30×, 40×, 50×, 60×, 70×, 80×, 90×, 100×, 200×).

In some embodiments, viral vectors comprising homology arms of different lengths may provide improved gene editing in a species or a model system for a species (e.g., mouse, human, or models thereof). In some embodiments, viral vectors may comprise different combinations of homology arm lengths when optimized for expression in a particular species or a model system for a particular species (e.g., mouse, human, or models thereof). In some embodiments, viral vectors comprising specific combinations of homology arm lengths may provide improved gene editing in one species or a model system of one species (e.g., human, humanized mouse model) as compared to a second species or a model system of a second species (e.g., mouse, pure mouse model). In some embodiments, viral vectors comprising specific combinations of homology arm lengths may be optimized for high levels of gene editing in one species or a model of one species (e.g., human, humanized mouse model) as compared to a second species or a model system of a second species (e.g., mouse, pure mouse model).

In some embodiments, homology arms direct integration of a transgene immediately behind a highly expressed endogenous gene. In some embodiments, homology arms direct integration of a transgene without disrupting endogenous gene expression (non-disruptive integration).

In some embodiments, one or more homology arm sequences may have at least 80%, 85%, 90%, 95%, 99%, or 100% identity to a corresponding wild-type reference nucleotide sequence (e.g., a wild-type genomic sequence). In some embodiments, one or more homology arm sequences may be or comprise a sequence having a least 80%, 85%, 90%, 95%, 99%, or 100% identity to a portion of a corresponding wild-type reference nucleotide sequence (e.g., a wild-type genomic sequence).

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 1 and a 3′ homology arm sequence that has at least 70%, at least 75%, at least 80%, at least 85%, at least 90%, at least 95%, at least 99%, or 100% sequence identity with SEQ ID NO: 2. In some embodiments, a viral vector comprises a 5′ homology arm comprising SEQ ID NO: 1 and a 3′ homology arm comprising SEQ ID NO: 2. In some embodiments, a viral vector comprises a 5′ homology arm consisting of SEQ ID NO: 1 and a 3′ homology arm consisting of SEQ ID NO: 2.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 3 and a 3′ homology arm sequence that has at least 70%, at least 75%, at least 80%, at least 85%, at least 90%, at least 95%, at least 99%, or 100% sequence identity with SEQ ID NO: 2. In some embodiments, a viral vector comprises a 5′ homology arm comprising SEQ ID NO: 3 and a 3′ homology arm comprising SEQ ID NO: 2. In some embodiments, a viral vector comprises a 5′ homology arm consisting of SEQ ID NO: 3 and a 3′ homology arm consisting of SEQ ID NO: 2.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 4 and a 3′ homology arm sequence that has at least 70%, at least 75%, at least 80%, at least 85%, at least 90%, at least 95%, at least 99%, or 100% sequence identity with SEQ ID NO: 5. In some embodiments, a viral vector comprises a 5′ homology arm comprising SEQ ID NO: 4 and a 3′ homology arm comprising SEQ ID NO: 5. In some embodiments, a viral vector comprises a 5′ homology arm consisting of SEQ ID NO: 4 and a 3′ homology arm consisting of SEQ ID NO: 5.

Exemplary homology arm sequences are provided below:

```
Human albumin 1.6 kb 5′ homology arm
  (SEQ ID NO: 1)
tttctagatgtaaataattattttaagtttgccctatggtggccccacacatgagacaaacccccaagatgtga
cttttgagaatgagacttggataaaaaacatgtagaaatgcaagccctgaagctcaactccctattgctatcac
aggggttataattgcataaaatttagctatagaaagttgctgtcatctcttgtgggctgtaatcatcgtctagg
cttaagagtaatattgcaaaacctgtcatgcccacacaaatctctccctggcattgttgtctttgcagatgtca
gtgaaagagaaccagcagctcccatgagtttggatagccttattttctatagcctccccactattagctttgaa
gggagcaaagtttaagaaccaaatataaagtttctcatctttatagatgagaaaaattttaaataaagtccaag
ataattaaatttttaaggatcatttttagctctttaatagcaataaaactcaatatgacataatatggcacttc
caaaatctgaataatatataattgcaatgacatacttcttttcagagatttactgaaaagaaatttgttgacac
tacataacgtgatgagtggtttatactgattgtttcagttggtcttcccaccaactccatgaaagtggatttta
ttatcctcatcatgcagatgagaatattgagacttatagcggtatgcctgagccccaaagtactcagagttgcc
tggctccaagatttataatcttaaatgatgggactaccatccttactctctccatttttctatacgtgagtaat
gttttttctgttttttttttttctttttccattcaaactcagtgcacttgttgagctcgtgaaacacaagccca
aggcaacaaaagagcaactgaaagctgttatggatgatttcgcagcttttgtagagaagtgctgcaaggctgac
gataaggagacctgctttgccgaggaggtactacagttctcttcattttaatatgtccagtattcatttttgca
tgtttggttaggctagggcttagggatttatatatcaaaggaggctttgtacatgtgggacagggatottattt
tacaaacaattgtcttacaaaatgaataaaacagcacttttttttatctcctgctctattgtgccatactgtt
aaatgtttataatgcctgttctgtttccaaatttgtgatgcttatgaatattaataggaatatttgtaaggcct
gaaatattttgatcatgaaatcaaaacattaatttatttaaacatttacttgaaatgtggtggtttgtgattta
gttgattttataggctagtgggagaatttacattcaaatgtctaaatcacttaaaattgccctttatggcctga
cagtaacttttttttattcatttggggacaactatgtccgtgagcttccgtccagagattatagtagtaaattg
taattaaaggatatgatgcacgtgaaatcactttgcaatcatcaatagcttcataaatgttaattttgtatcct
aatagtaatgctaatattttcctaacatctgtcatgtctttgtgttcagggtaaaaaacttgttgctgcaagtc
aagctgccttaggctta
Human albumin 1 kb 3′ homology arm
  (SEQ ID NO: 2)
Taacatcacatttaaaagcatctcaggtaactatattttgaattttttaaaaaagtaactataatagttattat
taaaatagcaaagattgaccatttccaagagccatatagaccagcaccgaccactattctaaactatttatgta
tgtaaatattagcttttaaaattctcaaaatagttgctgagttgggaaccactattatttctattttgtagatg
agaaaatgaagataaacatcaaagcatagattaagtaattttccaaagggtcaaaattcaaaattgaaaccaaa
gtttcagtgttgcccattgtcctgttctgacttatatgatgcggtacacagagccatccaagtaagtgatggct
cagcagtggaatactctgggaattaggctgaaccacatgaaagagtgctttatagggcaaaaacagttgaatat
cagtgatttcacatggttcaacctaatagttcaactcatcctttccattggagaatatgatggatctaccttct
gtgaactttatagtgaagaatctgctattacatttccaatttgtcaacatgctgagctttaataggacttatct
tcttatgacaacatttattggtgtgtccccttgcctagcccaacagaagaattcagcagccgtaagtctaggac
aggcttaaattgttttcactggtgtaaattgcagaaagatgatctaagtaatttggcatttattttaataggtt
tgaaaaacacatgccattttacaaataagacttatatttgtcctttttttttcagcctaccatgagaataaga
gaaagaaaatgaagatcaaaagcttattcatctgtttttctttttcgttggtgtaaagccaacaccctgtctaa
aaaacataaatttctttaatcattttgcctcttttctctgtgcttcaattaataaaaaatggaaagaatctaat
agagtggtacagcactgttatttttcaaagatgtgttg
Human albumin 1 kb 5′ homology arm
  (SEQ ID NO: 3)
Actccatgaaagtggattttattatcctcatcatgcagatgagaatattgagacttatagcggtatgcctgagc
cccaaagtactcagagttgcctggctccaagatttataatcttaaatgatgggactaccatccttactctctcc
atttttctatacgtgagtaatgttttttctgttttttttttttctttttccattcaaactcagtgcacttgttg
agctcgtgaaacacaagcccaaggcaacaaaagagcaactgaaagctgttatggatgatttcgcagcttttgta
gagaagtgctgcaaggctgacgataaggagacctgctttgccgaggaggtactacagttctcttcattttaata
tgtccagtattcatttttgcatgtttggttaggctagggcttagggatttatatatcaaaggaggctttgtaca
tgtgggacagggatcttattttacaaacaattgtcttacaaaatgaataaaacagcactttgtttttatctcct
gctctattgtgccatactgttaaatgtttataatgcctgttctgtttccaaatttgtgatgcttatgaatatta
ataggaatatttgtaaggcctgaaatattttgatcatgaaatcaaaacattaatttatttaaacatttacttga
aatgtggtggtttgtgatttagttgattttataggctagtgggagaatttacattcaaatgtctaaatcactta
aaattgccctttatggcctgacagtaacttttttttattcatttggggacaactatgtccgtgagcttccgtcc
agagattatagtagtaaattgtaattaaaggatatgatgcacgtgaaatcactttgcaatcatcaatagcttca
taaatgttaattttgtatcctaatagtaatgctaatattttcctaacatctgtcatgtctttgtgttcagggta
aaaaacttgttgctgcaagtcaagctgccttaggctta
Mouse albumin 1 kb 5′ homology arm
  (SEQ ID NO: 4)
Ctgaaactagacaaaacccgtgtgactggcatcgattattctatttgatctagctagtcctagcaaagtgacaa
ctgctactcccctcctacacagccaagattcctaagttggcagtggcatgcttaatcctcaaagccaaagttac
ttggctccaagatttatagccttaaactgtggcctcacattccttcctatcttactttcctgcactggggtaaa
tgtctccttgctcttcttgctttctgtcctactgcagggctcttgctgagctggtgaagcacaagcccaaggct
acagcggagcaactgaagactgtcatggatgactttgcacagttcctggatacatgttgcaaggctgctgacaa
ggacacctgcttctcgactgaggtcagaaacgtttttgcattttgacgatgttcagtttccattttctgtgcac
gtggtcaggtgtagctctctggaactcacacactgaataactccaccaatctagatgttgttctctacgtaact
gtaatagaaactgacttacgtagcttttaatttttattttctgccacactgctgcctattaaatacctattatc
actatttggtttcaaatttgtgacacagaagagcatagttagaaatacttgcaaagcctagaatcatgaactca
tttaaaccttgccctgaaatgtttctttttgaattgagttattttacacatgaatggacagttaccattatata
tctgaatcatttcacattccctcccatggcctaacaacagtttatcttcttattttgggcacaacagatgtcag
agagcctgctttaggaattctaagtagaactgtaattaagcaatgcaaggcacgtacgtttactatgtcattgc
ctatggctatgaagtgcaaatcctaacagtcctgctaatacttttctaacatccatcatttctttgttttcagg
gtccaaaccttgtcactagatgcaaagacgccttagcc
Mouse albumin 1.6 kb 3′ homology arm
  (SEQ ID NO: 5)
taaacacatcacaaccacaaccttctcaggtaactatacttgggacttaaaaaacataatcataatcatttttc
ctaaaacgatcaagactgataaccatttgacaagagccatacagacaagcaccagctggcactcttaggtcttc
acgtatggtcatcagtttgggttccatttgtagataagaaactgaacatataaaggtctaggttaatgcaattt
acacaaaaggagaccaaaccagggagagaaggaaccaaaattaaaaattcaaaccagagcaaaggagttagccc
tggttttgctctgacttacatgaaccactatgtggagtcctccatgttagcctagtcaagcttatcctctggat
gaagttgaaaccatatgaaggaatatttgggggggggtcaaaacagttgtgtatcaatgattccatgtggttt
gacccaatcattctgtgaatccatttcaacagaagatacaacgggttctgtttcataataagtgatccacttcc
aaatttctgatgtgccccatgctaagctttaacagaatttatcttcttatgacaaagcagcctcctttgaaaat
atagccaactgcacacagctatgttgatcaattttgtttataatcttgcagaagagaattttttaaaatagggc
aataatggaaggctttggcaaaaaaattgtttctccatatgaaaacaaaaaacttatttttttattcaagcaaa
gaacctatagacataaggctatttcaaaattatttcagttttagaaagaattgaaagttttgtagcattctgag
aagacagctttcatttgtaatcataggtaatatgtaggtcctcagaaatggtgagacccctgactttgacactt
ggggactctgagggaccagtgatgaagagggcacaacttatatcacacatgcacgagttggggtgagagggtgt
cacaacatctatcagtgtgtcatctgcccaccaagtaacagatgtcagctaagactaggtcatgtgtaggctgt
ctacaccagtgaaaatcgcaaaaagaatctaagaaattccacatttctagaaaataggtttggaaaccgtattc
cattttacaaaggacacttacatttctctttttgttttccaggctaccctgagaaaaaaagacatgaagactca
ggactcatcttttctgttggtgtaaaatcaacaccctaaggaacacaaatttctttaaacatttgacttcttgt
ctctgtgctgcaattaataaaaaatggaaagaatctactctgtggttcagaactctatcttccaaaggcgcgct
tcaccctagcagcctctttggctcagaggaatccctgcctttcctcccttcatctcagcagagaatgtagttcc
acatgggcaacacaatgaaaataaacgttaatactctcccatcttatgggtggtgaccctagaaaccaatactt
caacattacgagaattctgaatgagagactaaaagcttatgaactgtggctttcctttgtcagtgggactctaa
gaatgagttggggacaaaagagataggaatggctttaaaggtgactagttgaactgataaagtaaatgaactga
ggaaaaaaaatatcactcaa
```

### Measurement of Target Site Integration

As described in the present application, one of the problems with traditional use of nucleases to introduce nucleic acid material into cells is a significant chance of off target integration (e.g., of a transgene). Accordingly, it is important to verify correct integration through one or more specifically targeted assays, as described below.

In accordance with various embodiments, rate of integration may be measured at any of a variety of points in time. In some embodiments, rates of target site integration are measured after one or more days. In some embodiments, rates of target site integration are measured after one or more weeks. In some embodiments, rates of target site integration are measured after one or more months. In some embodiments, rates of target site integration are measured after one or more years. In some embodiments, rates of target site integration are measured through assessment of one or more biomarkers (e.g., biomarkers comprising a 2A peptide). In some embodiments, rates of target site integration are measured through assessment of one or more isolated nucleic acids (e.g., mRNA, gDNA). In some embodiments, rates of target site integration are measured through assessment of gene expression (e.g., through immunohistochemical staining).

TABLE 1
Exemplary methods for assessment of target site integration

| | Sample | Exemplary | |
|---|---|---|---|
| Assay type | analyzed | method | Exemplary protocol |
| Genomic DNA | Liver (frozen) | qPCR | Liver biopsy subjected to genomic DNA |
| integration rate | | | extraction. qPCR method run to detect |
| (gDNA Int %) | | | percentage of allele (e.g. albumin) containing |
| | | | on-target insertion. |
| Fused mRNA | Liver (frozen) | ddPCR | Liver biopsy subjected to RNA extraction. |
| | | | ddPCR method run to quantify the copy |
| | | | number of fused mRNA (unique chimeric |
| | | | mRNA transcribed from edited allele). This |
| | | | assay measures the transcriptional activity |
| | | | after target insertion. |
| ALB-2A | Plasma | ELISA | Blood collected and processed for plasma. |
| | | | Proprietary ELISA used to measure 2A- |
| | | | tagged albumin (universal circulating |
| | | | biomarker for targeted integration) This |
| | | | assay measures total protein expression after |
| | | | target insertion. |
| Hepatocyte | Fixed liver | IHC | Fixed liver sectioned and stained against |
| editing % | section | | transgene. Transgene-positive cells counted |
| | | | and used to calculate percentage of |
| | | | hepatocyte editing. For targeted integration |
| | | | into a target integration site in the albumin |
| | | | locus, transgene expression should be |
| | | | hepatocyte-specific. This assay focuses on |
| | | | per-cell target integration and is orthogonal |
| | | | to gDNA Int %, which focuses on per allele |
| | | | target integration. |
| GFP | Fixed cells (e.g, | ICC/IHC | Fixed cells counterstained with the nuclear |
| expression | HepG2) and/or | | dye. GFP+ cells imaged directly or stained |
| | fixed tissue | | using anti-HA tag antibody. This assay |
| | (e.g., liver) | | measures the percentage of cells that express |
| | section | | the GFP transgene and is an indicator of viral |
| | | | vector editing efficiency. |
| FAH | Fixed cells (e.g, | ICC/IHC | Fixed cells counterstained with the nuclear |
| expression | HepG2) and/or | | dye. Cells stained using anti-FAH antibody. |
| | fixed tissue | | This assay measures the percentage of cells |
| | (e.g., liver) | | that express the FAH and is an indicator of |
| | section | | viral vector editing efficiency. |

### Transgene

In some embodiments, methods and compositions of the present disclosure provide one or more transgenes (e.g., FAH). In some embodiments transgenes, are chosen to integrate into a genome. In some embodiments, transgenes are functional versions of a disease associated gene found in a subject's cells. In some embodiments, combined size of the transgenes and the homology arms can be optimized to increase the likelihood that these transgenes are of a suitable sequence length to be efficiently packaged in a delivery vehicle, which can increase the likelihood that the transgenes will ultimately be delivered appropriately in the patient.

In some embodiments, a nucleotide sequence encoding a transgene is codon-optimized. In some embodiments, a nucleotide sequence encoding a transgene is codon-optimized for a certain cell type (e.g., mammalian, insect, bacterial, fungal, etc.). In some embodiments, a nucleotide sequence encoding a transgene is codon-optimized for a human cell. In some embodiments, a nucleotide sequence encoding a transgene is codon-optimized for a human cell of a particular tissue type (e.g., liver, muscle, CNS, lung).

In certain embodiments, a nucleotide sequence encoding a transgene may be codon optimized to have a nucleotide homology with a reference nucleotide sequence (e.g., a wild-type gene sequence) of less than 100%. In certain embodiments, nucleotide homology between a codon-optimized nucleotide sequence encoding a transgene and a reference nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In some embodiments, transgene expression in a subject results substantially from integration at a target integration site. In some embodiments, 75% or more (e.g., 80% or more, 85% or more, 90% or more, 95% or more, 99% or more, 99.5% or more) of total transgene expression in a subject is from transgene integration at a target integration site. In some embodiments, 25% or less (e.g., 20% or less, 15% or less, 10% or less, 5% or less, 1% or less, 0.5% or less, 0.1% or less) of total transgene expression in a subject is from a source other than transgene integration at a target integration site (e.g., episomal expression, integration at a non-target integration site).

In some embodiments, transgenes are transiently expressed in a subject (e.g., episomal expression from plasmids, minicircle DNAs, viruses, etc.). In some embodiments, 75% or more (e.g., 80% or more, 85% or more, 90% or more, 95% or more, 99% or more, 99.5% or more) of total transgene expression in a subject is from transient expression. In some embodiments, 25% or less (e.g., 20% or less, 15% or less, 10% or less, 5% or less, 1% or less, 0.5% or less, 0.1% or less) of total transgene expression in a subject is from a source other than transient expression (e.g., integration at a non-target integration site). In some embodiments, transgenes are transiently expressed in a subject (e.g., episomal expression from plasmids, minicircle DNAs, viruses, etc.) for one or more weeks after treatment. In some embodiments, transgenes are transiently expressed in a subject (e.g., episomal expression from plasmids, minicircle DNAs, viruses, etc.) for one or more months after treatment.

In some embodiments, transgenes are transiently expressed in a subject (e.g., episomal expression from plasmids, minicircle DNAs, viruses, etc.) one or more weeks after treatment at a level comparable to that observed within one or more days after treatment. In some embodiments, transgenes are transiently expressed in a subject (e.g., episomal expression from plasmids, minicircle DNAs, viruses, etc.) one or more months after treatment at a level comparable to that observed within one or more days after treatment.

In some embodiments, transgenes are transiently expressed in a subject (e.g., episomal expression from plasmids, minicircle DNAs, viruses, etc.) one or more weeks after treatment at a level that is reduced relative to that observed within one or more days after treatment. In some embodiments, transgenes are transiently expressed in a subject (e.g., episomal expression from plasmids, minicircle DNAs, viruses, etc.) one or more months after treatment at a level that is reduced relative to that observed within one or more days after treatment.

In some embodiments, transgenes are transiently expressed in a subject (e.g., episomal expression from plasmids, minicircle DNAs, viruses, etc.) for no more than one month after treatment. In some embodiments, transgenes are transiently expressed in a subject (e.g., episomal expression from plasmids, minicircle DNAs, viruses, etc.) for no more than two months after treatment. In some embodiments, transgenes are transiently expressed in a subject (e.g., episomal expression from plasmids, minicircle DNAs, viruses, etc.) for no more than three months after treatment. In some embodiments, transgenes are transiently expressed in a subject (e.g., episomal expression from plasmids, minicircle DNAs, viruses, etc.) for no more than four months after treatment. In some embodiments, transgenes are transiently expressed in a subject (e.g., episomal expression from plasmids, minicircle DNAs, viruses, etc.) for no more than five months after treatment. In some embodiments, transgenes are transiently expressed in a subject (e.g., episomal expression from plasmids, minicircle DNAs, viruses, etc.) for no more than six months after treatment.

In some embodiments, combined size of transgenes and homology arms can be optimized to increase the likelihood that these transgenes are of a suitable sequence length to be efficiently packaged in a delivery vehicle, which can increase the likelihood that the transgenes will ultimately be delivered appropriately in the patient.

In some embodiments, a nucleotide sequence encoding a transgene is codon-optimized. In some embodiments, a nucleotide sequence encoding a transgene is codon-optimized for a certain cell type (e.g., mammalian, insect, bacterial, fungal, etc.). In some embodiments, a nucleotide sequence encoding a transgene is codon-optimized for a human cell. In some embodiments, a nucleotide sequence encoding a transgene is codon-optimized for a human cell of a particular tissue type (e.g., liver, muscle, CNS, lung).

In some embodiments, a transgene may be or comprise a sequence having at least 80%, 85%, 90%, 95%, 99%, or 100% identity to a corresponding wild-type reference nucleotide sequence (e.g., a wild-type gene sequence). In some embodiments, a transgene may be or comprise a sequence having a least 80%, 85%, 90%, 95%, 99%, or 100% identity to a portion of a corresponding wild-type reference nucleotide sequence (e.g., a wild-type gene sequence).

Nucleic Acids that Promote the Production of Two Independent Gene Products

2A peptide for polycistronic expression. In some embodiments, methods and compositions of the present disclosure comprise a nucleic acid encoding a 2A peptide. Without wishing to be bound by any particular theory a nucleic acid sequence encoding a 2A peptide can play a number of important roles. In some embodiments, a 2A peptide facilitates polycistronic expression, which is the production of two distinct proteins from the same mRNA. This, in turn, allows integration of a transgene in a non-disruptive way by coupling transcription of the transgene to a highly expressed target gene in the tissue of interest, driven by a strong endogenous promoter. In some embodiments, liver-directed therapeutic programs the albumin locus can function as the site of integration. In some embodiments, through a process known as ribosomal skipping, the 2A peptide facilitates production of the therapeutic protein at the same level as the endogenous target gene (e.g., albumin) in each modified cell. In some embodiments, a subject's endogenous target gene (e.g., albumin) is produced normally, except for the addition of a C-terminal tag that serves as a circulating biomarker to indicate successful integration and expression of the transgene. In some embodiments, modification to the endogenous target gene (e.g., albumin) has minimal effect on its function. The 2A peptide has been incorporated into other potential therapeutics such as T cell receptor chimeric antigen receptors, or CAR-Ts (Qasim et al. Sci Transl Med 2017).

Exemplary sequences encoding one or more 2A peptides are provided below:

```
P2A Nucleotide Sequence
  (SEQ ID NO: 6)
GGAAGCGGCGCCACCAATTTCAGCCTGCTGAAACAGGCCGGCGACGTGG
AAGAGAACCCTGGCCCT
P2A Peptide Sequence
  (SEQ ID NO: 7)
GSGATNFSLLKQAGDVEENPGP
```

In some embodiments, targeting a particular locus allows leverage of a strong endogenous promotor that drives a high level of production to maximize the expression of a transgene. In some embodiments, linking expression of the transgene to a highly expressed endogenous protein (e.g., albumin) can allow expression of the transgene at therapeutic levels without requiring the addition of exogenous promoters or the integration of the transgene in a majority of target cells.

This is supported by animal models of MMA, hemophilia B and Crigler-Najjar syndrome. In these models, integration of the transgene into approximately 1% of cells resulted in therapeutic benefit. In some embodiments, the strength of the albumin promoter overcomes the modest levels of integration to yield potentially therapeutic levels of transgene expression.

Without wishing to be bound by any particular theory, potential advantages of the GENERIDE™ approach include the following:

Targeted Integration of Transgene into the Genome.

Conventional gene therapy approaches deliver therapeutic transgenes to target cells. A major shortcoming with most of these approaches is that once the genes are inside the cell, they do not integrate into the host cell's chromosomes and do not benefit from the natural processes that lead to replication and segregation of DNA during cell division. This is particularly problematic when conventional gene therapies are introduced early in the patient's life, because the rapid growth of tissues during the child's normal development will result in dilution and eventual loss of the therapeutic benefit associated with the transgene. Non-integrated genes expressed outside the genome on a separate strand of DNA are called episomes. This episomal expression can be effective in the initial cells that are transduced, some of which may last for a long time or for the life of a patient. However, episomal expression is typically transient in target tissues such as the liver, in which there is high turnover of cells and which tends to grow considerably in size during the course of a pediatric patient's life. With GENERIDE™ technology, the transgene is integrated into the genome, which has the potential to provide stable and durable transgene expression as the cells divide and the tissue of the patient grows, and may result in a durable therapeutic benefit.

Transgene Expression without Exogenous Promoters.

In some embodiments, with GENERIDE™ technology, the transgene is expressed at a location where it is regulated by a potent endogenous promoter. In some embodiments homology arms can be used to insert the transgene at a precise site in the genome that is expressed under the control of a potent endogenous promoter (e.g., the albumin promoter). By not using exogenous promoters to drive expression of a transgene, this technology avoids the potential for off-target integration of promoters, which has been associated with an increased risk of cancer. In some embodiments, the choice of strong endogenous promoters will allow reaching therapeutic levels of protein expression from the transgene with the modest integration rates typical of the highly accurate and reliable process of HR.

### Nuclease-Free Genome Editing.

By harnessing the naturally occurring process of HR, GENERIDE™ is designed to avoid undesired side effects associated with exogenous nucleases used in conventional gene editing technologies. The use of these engineered enzymes has been associated with genotoxicity, including chromosomal alterations, resulting from the error-prone DNA repair of double-stranded DNA cuts. Avoiding the use of nucleases also reduces the number of exogenous components needed to be delivered to the cell.

### Payloads

In some embodiments, one or more vectors or constructs described herein may comprise a polynucleotide sequence encoding one or more payloads. In accordance with various aspects, any of a variety of payloads may be used (e.g., those with a diagnostic and/or therapeutic purpose), alone or in combination. In some embodiments, a payload may be or comprise a polynucleotide sequence encoding a peptide or polypeptide. In some embodiments, a payload is a peptide that has cell-intrinsic or cell-extrinsic activity that promotes a biological process to treat a medical condition. In some embodiments, a payload may be or comprise a transgene (also referred to herein as a gene of interest (GOI)). In some embodiments, a payload may be or comprise one or more inverted terminal repeat (ITR) sequences (e.g., one or more AAV ITRs). In some embodiments, a payload may be or comprise one or more transgenes with flanking ITR sequences. In some embodiments, a payload may be or comprise one or more heterologous nucleic acid sequences encoding a reporter gene (e.g., a fluorescent or luminescent reporter). In some embodiments, a payload may be or comprise one or more biomarkers (e.g., proxy for payload expression). In some embodiments, a payload may comprise a sequence for polycistronic expression (including, e.g., a 2A peptide, or intronic sequence, internal ribosomal entry site). In some embodiments, 2A peptides are small (e.g., approximately 18-22 amino acids) peptide sequences enabling co-expression of two or more discrete protein products within a single coding sequence. In some embodiments, 2A peptides allows co-expression of two or more discrete protein products regardless of arrangement of protein coding sequences. In some embodiments, 2A peptides are or comprise a consensus motif (e.g., DVEXNPGP). In some embodiments, 2A peptides promote protein cleavage. In some embodiments, 2A peptides are or comprise viral sequences (e.g., foot-and-mouth diseases virus (F2A), equine Rhinitis A virus, porcine teschovirus-1 (P2A), or Thosea asigna virus (T2A)).

In some embodiments, a payload may be or comprise a polynucleotide sequence, which comprises an expression cassette. In some embodiments, an expression cassette comprises a first nucleic acid sequence and a second nucleic acid sequence, wherein the first nucleic acid sequence encodes a transgene and the second nucleic acid sequence is positioned 5′ or 3′ to the first nucleic acid sequence and promotes the production of two independent gene products (e.g., a sequence encoding a 2A peptide).

### Methods of Monitoring

In some embodiments, the present disclosure provides methods of and/or otherwise assessing gene therapy. In some embodiments, the present disclosure provides for detection of products (e.g., polypeptides or nucleic acids) and/or biomarkers generated or encoded by compositions described herein. In some embodiments, presence of a product or biomarker is assessed in a biological sample taken from a subject who has received an integrating gene therapy treatment as described herein. In some embodiments, a biological sample is or comprises hair, skin, feces, blood, plasma, serum, cerebrospinal fluid, urine, saliva, tears, vitreous humor, liver biopsy or mucus.

In some embodiments, a product or biomarker is expressed intracellularly. In some embodiments, a product or biomarker is secreted extracellularly. In some embodiments, a product or biomarker comprises a 2A peptide. In some embodiments, a product or biomarker comprises albumin (e.g., a modified albumin, e.g., with a C-terminal tag). Methods of detecting various products or biomarkers are known in the art. In some embodiments, a product or biomarker is detected by an immunological assay or a nucleic acid amplification assay. In some embodiments, methods of detecting products or biomarkers are described in WO/2020/214582, the entire contents of which are incorporated herein by reference. In some embodiments, detection of products or biomarkers is performed 1, 2, 3, 4, 5, 6, 7, 8 or more weeks after the subject has received the gene therapy treatment or gene-integrating composition.

### Delivery Vehicles

There are multiple gene therapy approaches understood in the art. As such there are multiple mechanisms of delivery understood in the art. In some embodiments, a transgene is provided using a delivery vehicle. In some embodiment, compositions of the present disclosure comprise a delivery vehicle. In some embodiments, a delivery vehicle is or comprises a non-viral particle. In some embodiments, a delivery vehicle is a lipid particle (e.g., a lipid nanoparticle). Various lipid nanoparticles for delivery of nucleic acids are known in the art, for example, those described in WO2015184256; WO2013149140; WO2014089486A1; WO2009127060; WO2011071860; WO2020219941 the contents of each of which is incorporated herein by reference.

In some embodiments, a delivery vehicle is or comprises an exosome. One of skill in the art will recognize various methods of exosome production and use. Examples of such methods and uses are described in Luan et al., Acta Pharmacologica Sinica volume 38, pages 754-763 (2017).

In some embodiments, a delivery vehicle is or comprises a closed circular cDNA integrating gene therapy construct. In some embodiments, a delivery vehicle is or comprises a recombinant viral vector. In some embodiments, a recombinant viral vector is an adeno associated viral (AAV) vector. In some embodiments, a recombinant AAV vector comprises a capsid of, AAV8, AAV-DJ; AAV-LK03; sL65; or AAVNP59. In some embodiments, a recombinant viral vector is or comprises a capsid protein comprising an amino acid sequence having at least 90%, 95%, 99%, or 100% sequence identity with the amino acid sequence of, AAV8, AAV-DJ; AAV-LK03; sL65 or AAVNP59. In some embodiments, a recombinant viral vector is or comprises a variant (e.g., codon-optimized variant) of AAV8, AAV-DJ; AAV-LK03; sL65; or AAVNP59.

| | SEQ | |
|---|---|---|
| AAV | ID | |
| Capsid | NO. | SEQUENCE |
| AAV | 8 | atggctgctgacggttatcttccagattggctcgaggacaacctttctgaa |
| LK03 | | ggcattcgagagtggtgggcgctgcaacctggagcccctaaacccaaggca |
| (nucleo- | | aatcaacaacatcaggacaacgctcggggtcttgtgcttccgggttacaaa |
| tide) | | tacctcggacccggcaacggactcgacaagggggaacccgtcaacgcagcg |
| | | gacgcggcagccctcgagcacgacaaggcctacgaccagcagctcaaggcc |
| | | ggtgacaacccctacctcaagtacaaccacgccgacgccgagttccaggag |
| | | cggctcaaagaagatacgtcttttgggggcaacctcgggcgagcagtcttc |
| | | caggccaaaaagaggcttcttgaacctcttggtctggttgaggaagcggct |
| | | aagacggctcctggaaagaagaggcctgtagatcagtctcctcaggaaccg |
| | | gactcatcatctggtgttggcaaatcgggcaaacagcctgccagaaaaaga |
| | | ctaaatttcggtcagactggcgactcagagtcagtcccagaccctcaacct |
| | | ctcggagaaccaccagcagcccccacaagtttgggatctaatacaatggct |
| | | tcaggcggtggcgcaccaatggcagacaataacgagggtgccgatggagtg |
| | | ggtaattcctcaggaaattggcattgcgattcccaatggctgggcgacaga |
| | | gtcatcaccaccagcaccagaacctgggccctgcccacttacaacaaccat |
| | | ctctacaagcaaatctccagccaatcaggagcttcaaacgacaaccactac |
| | | tttggctacagcaccccttgggggtattttgactttaacagattccactgc |
| | | cacttctcaccacgtgactggcagcgactcattaacaacaactggggattc |
| | | cggcccaagaaactcagcttcaagctcttcaacatccaagttaaagaggtc |
| | | acgcagaacgatggcacgacgactattgccaataaccttaccagcacggtt |
| | | caagtgtttacggactcggagtatcagctcccgtacgtgctcgggtcggcg |
| | | caccaaggctgtctcccgccgtttccagcggacgtcttcatggtccctcag |
| | | tatggatacctcaccctgaacaacggaagtcaagcggtgggacgctcatcc |
| | | ttttactgcctggagtacttcccttcgcagatgctaaggactggaaataac |
| | | ttccaattcagctataccttcgaggatgtaccttttcacagcagctacgct |
| | | cacagccagagtttggatcgcttgatgaatcctcttattgatcagtatctg |
| | | tactacctgaacagaacgcaaggaacaacctctggaacaaccaaccaatca |
| | | cggctgctttttagccaggctgggcctcagtctatgtctttgcaggccaga |
| | | aattggctacctgggccctgctaccggcaacagagactttcaaagactgct |
| | | aacgacaacaacaacagtaactttccttggacagcggccagcaaatatcat |
| | | ctcaatggccgcgactcgctggtgaatccaggaccagctatggccagtcac |
| | | aaggacgatgaagaaaaatttttccctatgcacggcaatctaatatttggc |
| | | aaagaagggacaacggcaagtaacgcagaattagataatgtaatgattacg |
| | | gatgaagaagagattcgtaccaccaatcctgtggcaacagagcagtatgga |
| | | actgtggcaaataacttgcagagctcaaatacagctcccacgactagaact |
| | | gtcaatgatcagggggccttacctggcatggtgtggcaagatcgtgacgtg |
| | | taccttcaaggacctatctgggcaaagattcctcacacggatggacacttt |
| | | catccttctcctctgatgggaggctttggactgaaacatccgcctcctcaa |
| | | atcatgatcaaaaatactccggtaccggcaaatcctccgacgactttcagc |
| | | ccggccaagtttgcttcatttatcactcagtactccactggacaggtcagc |
| | | gtggaaattgagtgggagctacagaaagaaaacagcaaacgttggaatcca |
| | | gagattcagtacacttccaactacaacaagtctgttaatgtggactttact |
| | | gtagacactaatggtgtttatagtgaacctcgccccattggcacccgttac |
| | | cttacccgtcccctgtaa |
| AAV- | 9 | MAADGYLPDWLEDNLSEGIREWWALQPGAPKPKANQQHQDNARGLVLPGYK |
| LK03 | | YLGPGNGLDKGEPVNAADAAALEHDKAYDQQLKAGDNPYLKYNHADAEFQE |
| (protein) | | RLKEDTSFGGNLGRAVFQAKKRLLEPLGLVEEAAKTAPGKKRPVDQSPQEP |
| | | DSSSGVGKSGKQPARKRLNFGQTGDSESVPDPQPLGEPPAAPTSLGSNTMA |
| | | SGGGAPMADNNEGADGVGNSSGNWHCDSQWLGDRVITTSTRTWALPTYNNH |
| | | LYKQISSQSGASNDNHYFGYSTPWGYFDFNRFHCHFSPRDWQRLINNNWGF |
| | | RPKKLSFKLFNIQVKEVTQNDGTTTIANNLTSTVQVFTDSEYQLPYVLGSA |
| | | HQGCLPPFPADVFMVPQYGYLTLNNGSQAVGRSSFYCLEYFPSQMLRTGNN |
| | | FQFSYTFEDVPFHSSYAHSQSLDRLMNPLIDQYLYYLNRTQGTTSGTTNQS |
| | | RLLFSQAGPQSMSLQARNWLPGPCYRQQRLSKTANDNNNSNFPWTAASKYH |
| | | LNGRDSLVNPGPAMASHKDDEEKFFPMHGNLIFGKEGTTASNAELDNVMIT |
| | | DEEEIRTTNPVATEQYGTVANNLQSSNTAPTTRTVNDQGALPGMVWQDRDV |
| | | YLQGPIWAKIPHTDGHFHPSPLMGGFGLKHPPPQIMIKNTPVPANPPTTES |
| | | PAKFASFITQYSTGQVSVEIEWELQKENSKRWNPEIQYTSNYNKSVNVDFT |
| | | VDTNGVYSEPRPIGTRYLTRPL |
| AAV8 | 10 | atggctgccgatggttatcttccagattggctcgaggacaacctctctgag |
| (nucleo- | | ggcattcgcgagtggtgggcgctgaaacctggagccccgaagcccaaagcc |
| tide) | | aaccagcaaaagcaggacgacggccggggtctagtgcttcctggctacaag |
| | | tacctcggacccttcaacggactcgacaagggggagcccgtcaacgcggcg |
| | | gacgcagcggccctcgagcacgacaaggcctacgaccagcagctgcaggcg |
| | | ggtgacaatccgtacctgcggtataaccacgccgacgccgagtttcaggag |
| | | cgtctgcaagaagatacgtcttttgggggcaacctcgggcgagcagtcttc |
| | | caggccaagaagcgggttctcgaacctctcggtctggttgaggaaggcgct |
| | | aagacggctcctggaaagaagagaccggtagagccatcaccccagcgttct |
| | | ccagactcctctacgggcatcggcaagaaaggccaacagcccgccagaaaa |
| | | agactcaattttggtcagactggcgactcagagtcagttccagaccctcaa |
| | | cctctcggagaacctccagcagcgccctctggtgtgggacctaatacaatg |
| | | gctgcaggcggtggcgcaccaatggcagacaataacgaaggcgccgacgga |
| | | gtgggtagttcctcgggaaattggcattgcgattccacatggctgggcgac |
| | | agagtcatcaccaccagcacccgaacctgggccctgcccacctacaacaac |
| | | cacctctacaagcaaatctccaacgggacatcgggaggagccaccaacgac |
| | | aacacctacttcggctacagcaccccctgggggtattttgactttaacaga |
| | | ttccactgccacttttcaccacgtgactggcagcgactcatcaacaacaac |
| | | tggggattccggcccaagagactcagcttcaagctcttcaacatccaggtc |
| | | aaggaggtcacgcagaatgaaggcaccaagaccatcgccaataacctcacc |
| | | agcaccatccaggtgtttacggactcggagtaccagctgccgtacgttctc |
| | | ggctctgcccaccagggctgcctgcctccgttcccggcggacgtgttcatg |
| | | attccccagtacggctacctaacactcaacaacggtagtcaggccgtggga |
| | | cgctcctccttctactgcctggaatactttccttcgcagatgctgagaacc |
| | | ggcaacaacttccagtttacttacaccttcgaggacgtgcctttccacagc |
| | | agctacgcccacagccagagcttggaccggctgatgaatcctctgattgac |
| | | cagtacctgtactacttgtctcggactcaaacaacaggaggcacggcaaat |
| | | acgcagactctgggcttcagccaaggtgggcctaatacaatggccaatcag |
| | | gcaaagaactggctgccaggaccctgttaccgccaacaacgcgtctcaacg |
| | | acaaccgggcaaaacaacaatagcaactttgcctggactgctgggaccaaa |
| | | taccatctgaatggaagaaattcattggctaatcctggcatcgctatggca |
| | | acacacaaagacgacgaggagcgtttttttcccagtaacgggatcctgatt |
| | | tttggcaaacaaaatgctgccagagacaatgcggattacagcgatgtcatg |
| | | ctcaccagcgaggaagaaatcaaaaccactaaccctgtggctacagaggaa |
| | | tacggtatcgtggcagataacttgcagcagcaaaacacggctcctcaaatt |
| | | ggaactgtcaacagccagggggccttacccggtatggtctggcagaaccgg |
| | | gacgtgtacctgcagggtcccatctgggccaagattcctcacacggacggc |
| | | aacttccacccgtctccgctgatgggggctttggcctgaaacatcctccg |
| | | cctcagatcctgatcaagaacacgcctgtacctgcggatcctccgaccacc |
| | | ttcaaccagtcaaagctgaactctttcatcacgcaatacagcaccggacag |
| | | gtcagcgtggaaattgaatgggagctgcagaaggaaaacagcaagcgctgg |
| | | aaccccgagatccagtacacctccaactactacaaatctacaagtgtggac |
| | | tttgctgttaatacagaaggcgtgtactctgaaccccgccccattggcacc |
| | | cgttacctcacccgtaatctgtaa |
| AAV8 | 11 | MAADGYLPDWLEDNLSEGIREWWALKPGAPKPKANQQKQDDGRGLVLPGYK |
| (protein) | | YLGPFNGLDKGEPVNAADAAALEHDKAYDQQLQAGDNPYLRYNHADAEFQE |
| | | RLQEDTSFGGNLGRAVFQAKKRVLEPLGLVEEGAKTAPGKKRPVEPSPQRS |
| | | PDSSTGIGKKGQQPARKRLNFGQTGDSESVPDPQPLGEPPAAPSGVGPNTM |
| | | AAGGGAPMADNNEGADGVGSSSGNWHCDSTWLGDRVITTSTRTWALPTYNN |
| | | HLYKQISNGTSGGATNDNTYFGYSTPWGYFDFNRFHCHFSPRDWQRLINNN |
| | | WGFRPKRLSFKLFNIQVKEVTQNEGTKTIANNLTSTIQVFTDSEYQLPYVL |
| | | GSAHQGCLPPFPADVFMIPQYGYLTLNNGSQAVGRSSFYCLEYFPSQMLRT |
| | | GNNFQFTYTFEDVPFHSSYAHSQSLDRLMNPLIDQYLYYLSRTQTTGGTAN |
| | | TQTLGFSQGGPNTMANQAKNWLPGPCYRQQRVSTTTGQNNNSNFAWTAGTK |
| | | YHLNGRNSLANPGIAMATHKDDEERFFPSNGILIFGKQNAARDNADYSDVM |
| | | LTSEEEIKTTNPVATEEYGIVADNLQQQNTAPQIGTVNSQGALPGMVWQNR |
| | | DVYLQGPIWAKIPHTDGNFHPSPLMGGFGLKHPPPQILIKNTPVPADPPTT |
| | | FNQSKLNSFITQYSTGQVSVEIEWELQKENSKRWNPEIQYTSNYYKSTSVD |
| | | FAVNTEGVYSEPRPIGTRYLTRNL |
| AAV-DJ | 12 | ggaataagacagtggtggaagctcaaacctggcccaccaccaccaaagccc |
| (nucleo- | | atggctgccgatggttatcttccagattggctcgaggacactctctctgaa |
| tide) | | gcagagcggcataaggacgacagcaggggtcttgtgcttcctgggtacaag |
| | | tacctcggacccttcaacggactcgacaagggagagccggtcaacgaggca |
| | | gacgccgcggccctcgagcacgacaaagcctacgaccggcagctcgacagc |
| | | ggagacaacccgtacctcaagtacaaccacgccgacgccgagttccaggag |
| | | cggctcaaagaagatacgtcttttgggggcaacctcgggcgagcagtcttc |
| | | caggccaaaaagaggcttcttgaacctcttggtctggttgaggaagcggct |
| | | aagacggctcctggaaagaagaggcctgtagagcactctcctgtggagcca |
| | | gactcctcctcgggaaccggaaaggcgggccagcagcctgcaagaaaaaga |
| | | ttgaattttggtcagactggagacgcagactcagtcccagaccctcaacca |
| | | atcggagaacctcccgcagccccctcaggtgtgggatctcttacaatggct |
| | | gcaggcggtggcgcaccaatggcagacaataacgagggcgccgacggagtg |
| | | ggtaattcctcgggaaattggcattgcgattccacatggatgggcgacaga |
| | | gtcatcaccaccagcacccgaacctgggccctgcccacctacaacaaccac |
| | | ctctacaagcaaatctccaacagcacatctggaggatcttcaaatgacaac |
| | | gcctacttcggctacagcaccccctgggggtattttgactttaacagattc |
| | | cactgccacttttcaccacgtgactggcagcgactcatcaacaacaactgg |
| | | ggattccggcccaagagactcagcttcaagctcttcaacatccaggtcaag |
| | | gaggtcacgcagaatgaaggcaccaagaccatcgccaataacctcaccagc |
| | | accatccaggtgtttacggactcggagtaccagctgccgtacgttctcggc |
| | | tctgcccaccagggctgcctgcctccgttcccggcggacgtgttcatgatt |
| | | ccccagtacggctacctaacactcaacaacggtagtcaggccgtgggacgc |
| | | tcctccttctactgcctggaatactttccttcgcagatgctgagaaccggc |
| | | aacaacttccagtttacttacaccttcgaggacgtgcctttccacagcagc |
| | | tacgcccacagccagagcttggaccggctgatgaatcctctgattgaccag |
| | | tacctgtactacttgtctcggactcaaacaacaggaggcacgacaaatacg |
| | | cagactctgggcttcagccaaggtgggcctaatacaatggccaatcaggca |
| | | aagaactggctgccaggaccctgttaccgccagcagcgagtatcaaagaca |
| | | tctgcggataacaacaacagtgaatactcgtggactggagctaccaagtac |
| | | cacctcaatggcagagactctctggtgaatccgggcccggccatggcaagc |
| | | cacaaggacgatgaagaaaagttttttcctcagagcggggttctcatcttt |
| | | gggaagcaaggctcagagaaaacaaatgtggacattgaaaaggtcatgatt |
| | | acagacgaagaggaaatcaggacaaccaatcccgtggctacggagcagtat |
| | | ggttctgtatctaccaacctccagagaggcaacagacaagcagctaccgca |
| | | gatgtcaacacacaaggcgttcttccaggcatggtctggcaggacagagat |
| | | gtgtaccttcaggggcccatctgggcaaagattccacacacggacggacat |
| | | tttcacccctctcccctcatgggtggattcggacttaaacaccctccgcct |
| | | cagatcctgatcaagaacacgcctgtacctgcggatcctccgaccaccttc |
| | | aaccagtcaaagctgaactctttcatcacccagtattctactggccaagtc |
| | | agcgtggagatcgagtgggagctgcagaaggaaaacagcaagcgctggaac |
| | | cccgagatccagtacacctccaactactacaaatctacaagtgtggacttt |
| | | gctgttaatacagaaggcgtgtactctgaaccccgccccattggcacccgt |
| | | tacctcacccgtaatctgtaa |
| AAV-DJ | 13 | MAADGYLPDWLEDTLSEGIRQWWKLKPGPPPPKPAERHKDDSRGLVLPGYK |
| (protein) | | YLGPFNGLDKGEPVNEADAAALEHDKAYDRQLDSGDNPYLKYNHADAEFQE |
| | | RLKEDTSFGGNLGRAVFQAKKRLLEPLGLVEEAAKTAPGKKRPVEHSPVEP |
| | | DSSSGTGKAGQQPARKRLNFGQTGDADSVPDPQPIGEPPAAPSGVGSLTMA |
| | | AGGGAPMADNNEGADGVGNSSGNWHCDSTWMGDRVITTSTRTWALPTYNNH |
| | | LYKQISNSTSGGSSNDNAYFGYSTPWGYFDFNRFHCHFSPRDWQRLINNNW |
| | | GFRPKRLSFKLFNIQVKEVTQNEGTKTIANNLTSTIQVFTDSEYQLPYVLG |
| | | SAHQGCLPPFPADVFMIPQYGYLTLNNGSQAVGRSSFYCLEYFPSQMLRTG |
| | | NNFQFTYTFEDVPFHSSYAHSQSLDRLMNPLIDQYLYYLSRTQTTGGTTNT |
| | | QTLGFSQGGPNTMANQAKNWLPGPCYRQQRVSKTSADNNNSEYSWTGATKY |
| | | HLNGRDSLVNPGPAMASHKDDEEKFFPQSGVLIFGKQGSEKTNVDIEKVMI |
| | | TDEEEIRTTNPVATEQYGSVSTNLQRGNRQAATADVNTQGVLPGMVWQDRD |
| | | VYLQGPIWAKIPHTDGHFHPSPLMGGFGLKHPPPQILIKNTPVPADPPTTF |
| | | NQSKLNSFITQYSTGQVSVEIEWELQKENSKRWNPEIQYTSNYYKSTSVDF |
| | | AVNTEGVYSEPRPIGTRYLTRNL |
| SL65 | 14 | atggctgccgatggttatcttccagattggctcgaggacactctctctgaa |
| (nucleo- | | ggcattcgcgagtggtgggcgctgaaacctggagctccacaacccaaggcc |
| tide) | | aaccaacagcatcaggacaacggcaggggtcttgtgcttcctgggtacaag |
| | | tacctcggacccttcaacggactcgacaagggagagccggtcaacgaggca |
| | | gacgccgcggccctcgagcacgacaaggcctacgacaagcagctcgagcag |
| | | ggggacaacccgtacctcaagtacaaccacgccgacgccgagtttcaggag |
| | | cgtctgcaagaagatacgtcttttgggggcaacctcgggcgagcagtcttc |
| | | caggccaagaagcgggttctcgaacctctcggtctggttgaggaaggcgct |
| | | aagacggctcctggaaagaagagaccggtagagccgtcacctcagcgttcc |
| | | cccgactcctccacgggcatcggcaagaaaggccagcagcccgccagaaag |
| | | agactcaatttcggtcagactggcgactcagagtcagtccccgaccctcaa |
| | | cctctcggagaacctccagcagcgccctctagtgtgggatctggtacagtg |
| | | gctgcaggcggtggcgcaccaatggcagacaataacgaaggtgccgacgga |
| | | gtgggtaatgcctcaggaaattggcattgcgattccacatggctgggcgac |
| | | agagtcattaccaccagcacccgaacctgggccctgcccacctacaacaac |
| | | cacctctacaagcaaatctccagccaatcaggagcttcaaacgacaaccac |
| | | tactttggctacagcaccccttgggggtattttgactttaacagattccac |
| | | tgccacttctcaccacgtgactggcagcgactcattaacaacaactgggga |
| | | ttccggcccaagagactcaacttcaagctcttcaacatccaagtcaaggag |
| | | gtcacgacgaatgatggcgtcacgaccatcgctaataaccttaccagcacg |
| | | gttcaagtcttctcggactcggagtaccagttgccgtacgtcctcggctct |
| | | gcgcaccagggctgcctccctccgttcccggcggacgtgttcatgattccc |
| | | cagtacggctacctaacactcaacaacggtagtcaggccgtgggacgctcc |
| | | tccttttactgcctggaatatttcccatcgcagatgctgagaacgggcaat |
| | | aactttgagttcagctacagcttcgaggacgtgcctttccacagcagctac |
| | | gcacacagccagagcttggaccgactgatgaatcctctcattgaccagtac |
| | | ctgtactacttatccagaactcagtccacaggaggaactcaaggtacccag |
| | | caattgttattttctcaagctgggcctgcaaacatgtcggctcaggccaag |
| | | aactggctgcctggaccttgctaccggcagcagcgagtctccacgacactg |
| | | tcgcaaaacaacaacagcaactttgcttggactggtgccaccaaatatcac |
| | | ctgaacggcagaaactcgttggttaatcccggcgtcgccatggcaactcac |
| | | aaggacgacgaggaccgctttttcccatccagcggagtcctgatttttgga |
| | | aaaactggagcaactaacaaaactacattggaaaatgtgttaatgacaaat |
| | | gaagaagaaattcgtcctactaatcctgtagccacggaagaatacgggata |
| | | gtcagcagcaacttacaagcggctaatactgcagcccagacacaagttgtc |
| | | aacaaccagggagccttacctggcatggtctggcagaaccgggacgtgtac |
| | | ctgcagggtcccatttgggccaaaattcctcacacagatggacactttcac |
| | | ccgtctcctcttatgggcggctttggactcaagaacccgcctcctcagatc |
| | | ctcatcaaaaacacgcctgttcctgcgaatcctccggcggagttttcagct |
| | | acaaagtttgcttcattcatcacccagtattccacaggacaagtgagcgtg |
| | | gagattgaatgggagctgcagaaagaaaacagcaaacgctggaatcccgaa |
| | | gtgcagtatacatctaactatgcaaaatctgccaacgttgatttcactgtg |
| | | gacaacaatggactttatactgagcctcgccccattggcacccgttacctt |
| | | acccgtcccctgtaa |
| SL65 | 15 | MAADGYLPDWLEDTLSEGIREWWALKPGAPQPKANQQHQDNGRGLVLPGYK |
| (protein) | | YLGPFNGLDKGEPVNEADAAALEHDKAYDKQLEQGDNPYLKYNHADAEFQE |
| | | RLQEDTSFGGNLGRAVFQAKKRVLEPLGLVEEGAKTAPGKKRPVEPSPQRS |
| | | PDSSTGIGKKGQQPARKRLNFGQTGDSESVPDPQPLGEPPAAPSSVGSGTV |
| | | AAGGGAPMADNNEGADGVGNASGNWHCDSTWLGDRVITTSTRTWALPTYNN |
| | | HLYKQISSQSGASNDNHYFGYSTPWGYFDFNRFHCHFSPRDWQRLINNNWG |
| | | FRPKRLNFKLFNIQVKEVTTNDGVTTIANNLTSTVQVFSDSEYQLPYVLGS |
| | | AHQGCLPPFPADVFMIPQYGYLTLNNGSQAVGRSSFYCLEYFPSQMLRTGN |
| | | NFEFSYSFEDVPFHSSYAHSQSLDRLMNPLIDQYLYYLSRTQSTGGTQGTQ |
| | | QLLFSQAGPANMSAQAKNWLPGPCYRQQRVSTTLSQNNNSNFAWTGATKYH |
| | | LNGRNSLVNPGVAMATHKDDEDRFFPSSGVLIFGKTGATNKTTLENVLMTN |
| | | EEEIRPTNPVATEEYGIVSSNLQAANTAAQTQVVNNQGALPGMVWQNRDVY |
| | | LQGPIWAKIPHTDGHFHPSPLMGGFGLKNPPPQILIKNTPVPANPPAEFSA |
| | | TKFASFITQYSTGQVSVEIEWELQKENSKRWNPEVQYTSNYAKSANVDFTV |
| | | DNNGLYTEPRPIGTRYLTRPL |
| AAV- | 16 | atggctgccgatggttatcttccagattggctcgaggacactctctctgaa |
| NP59 | | ggaataagacagtggtggaagctcaaacctggcccaccaccaccaaagccc |
| (nucleo- | | gcagagcggcataaggacgacagcaggggtcttgtgcttcctgggtacaag |
| tide) | | tacctcggacccttcaacggactcgacaagggagagccggtcaacgaggca |
| | | gacgccgcggccctcgagcacgacaaagcctacgaccggcagctcgacagc |
| | | ggagacaacccgtacctcaagtacaaccacgccgacgcggagtttcaggag |
| | | cgccttaaagaagatacgtcttttgggggcaacctcggacgagcagtcttc |
| | | caggcgaaaaagagggttcttgaacctctgggcctggttgaggaacctgtt |
| | | aagacggctccgggaaaaaagaggccggtagagcactctcctgtggagcca |
| | | gactcctcctcgggaaccggcaagacaggccagcagcccgctaaaaagaga |
| | | ctcaattttggtcagactggcgactcagagtcagtcccagaccctcaacct |
| | | ctcggagaaccaccagcagccccctctggtctgggaactaatacgatggct |
| | | acaggcagtggcgcaccaatggcagacaataacgagggtgccgatggagtg |
| | | ggtaattcctcaggaaattggcattgcgattcccaatggctgggcgacaga |
| | | gtcatcaccaccagcacccgaacctgggccctgcccacctacaacaaccat |
| | | ctctacaagcaaatctccagccaatcaggagcttcaaacgacaaccactac |
| | | tttggctacagcaccccttgggggtattttgactttaacagattccactgc |
| | | cacttctcaccacgtgactggcagcgactcattaacaacaactggggattc |
| | | cggcccaagaaactcagcttcaagctcttcaacatccaagttaaagaggtc |
| | | acgcagaacgatggcacgacgactattgccaataaccttaccagcacggtt |
| | | caagtgtttactgactcggagtaccagctcccgtacgtcctcggctcggcg |
| | | catcaaggatgcctcccgccgttcccagcagacgtcttcatggtgccacag |
| | | tatggatacctcaccctgaacaacgggagtcaggcagtaggacgctcttca |
| | | ttttactgcctggagtactttccttctcagatgctgcgtaccggaaacaac |
| | | tttaccttcagctacacttttgaggacgttcctttccacagcagctacgct |
| | | cacagccagagtctggaccgtctcatgaatcctctcatcgaccagtacctg |
| | | tattacttgagcagaacaaacactccaagtggaaccaccacgcagtcaagg |
| | | cttcagttttctcaggccggagcgagtgacattcgggaccagtctaggaac |
| | | tggcttcctggaccctgttaccgccagcagcgagtatcaaagacatctgcg |
| | | gataacaacaacagtgaatactcgtgggctggagctaccaagtaccacctc |
| | | aatggcagagactctctggtgaatccgggcccggccatggcaagccacaag |
| | | gacgatgaagaaaagttttttcctcagagcggggttctcatctttgggaag |
| | | caaggctcagagaaaacaaatgtggacattgaaaaggtcatgattacagac |
| | | gaagaggaaatcaggacaaccaatcccgtggctacggagcagtatggttct |
| | | gtatctaccaacctccagagaggcaacagacaagcagctaccgcagatgtc |
| | | gacacacaaggcgttcttccaggcatggtatggcaggacagagatgtgtac |
| | | cttcagggacccatctgggcaaagattccacacacggacggacattttcac |
| | | ccctctcccctcatgggtggattcggacttaaacaccctcctccacagatt |
| | | ctcatcaagaacaccccggtacctgcgaatccttcgaccaccttcagtgcg |
| | | gcaaagtttgcttccttcatcacacagtactcaacgggacaggtcagcgtg |
| | | gagatcgagtgggagctgcagaaggaaaacagcaaacgctggaatcccgaa |
| | | attcagtacacttccaactacaacaagtctgttaatgtggactttactgtg |
| | | gacactaatggcgtgtattcagagcctcgccccattggcaccagatacctg |
| | | actcgtaatctgtaa |
| AAV- | 17 | MAADGYLPDWLEDTLSEGIRQWWKLKPGPPPPKPAERHKDDSRGLVLPGYK |
| NP59 | | YLGPFNGLDKGEPVNEADAAALEHDKAYDRQLDSGDNPYLKYNHADAEFQE |
| (protein) | | RLKEDTSFGGNLGRAVFQAKKRVLEPLGLVEEPVKTAPGKKRPVEHSPVEP |
| | | DSSSGTGKTGQQPAKKRLNFGQTGDSESVPDPQPLGEPPAAPSGLGTNTMA |
| | | TGSGAPMADNNEGADGVGNSSGNWHCDSQWLGDRVITTSTRTWALPTYNNH |
| | | LYKQISSQSGASNDNHYFGYSTPWGYFDFNRFHCHFSPRDWQRLINNNWGF |
| | | RPKKLSFKLFNIQVKEVTQNDGTTTIANNLTSTVQVFTDSEYQLPYVLGSA |
| | | HQGCLPPFPADVFMVPQYGYLTLNNGSQAVGRSSFYCLEYFPSQMLRTGNN |
| | | FTFSYTFEDVPFHSSYAHSQSLDRLMNPLIDQYLYYLSRTNTPSGTTTQSR |
| | | LQFSQAGASDIRDQSRNWLPGPCYRQQRVSKTSADNNNSEYSWAGATKYHL |
| | | NGRDSLVNPGPAMASHKDDEEKFFPQSGVLIFGKQGSEKTNVDIEKVMITD |
| | | EEEIRTTNPVATEQYGSVSTNLQRGNRQAATADVDTQGVLPGMVWQDRDVY |
| | | LQGPIWAKIPHTDGHFHPSPLMGGFGLKHPPPQILIKNTPVPANPSTTFSA |
| | | AKFASFITQYSTGQVSVEIEWELQKENSKRWNPEIQYTSNYNKSVNVDFTV |
| | | DTNGVYSEPRPIGTRYLTRNL |

In some embodiments, a recombinant AAV vector comprises at least one ITR. In some embodiments, a recombinant AAV vector comprises two ITRs. In some embodiments, a recombinant AAV vector comprises a 5′ ITR. In some embodiments, a recombinant AAV vector comprises a 3′ ITR. In some embodiments, a recombinant AAV vector comprises an AAV2 ITR. In some embodiments, a recombinant AAV vector comprises a portion of an AAV2 ITR. In some embodiments, a recombinant AAV vector comprises an ITR having at least 80%, 85%, 90%, 95%, 99%, or 10000 sequence identity to an AAV2 ITR. In some embodiments, a recombinant AAV vector comprises an ITR having 90%, 95%, 99%, 100% sequence identity to one of SEQ ID Nos. 27-30.

```
145 bp ITR:
  (SEQ ID NO. 27)
aggaacccctagtgatggagttggccactccctctctgegcgctcgctcg
ctcactgaggccgggcgaccaaaggtcgcccgacgcccgggctttgcccg
ggcggcctcagtgagcgagcgagcgcgcagagagggagtggccaa
130 bp ITR:
(SEQ ID NO. 28)
aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcag
B loop deletion ITR:
  (SEQ ID NO. 29)
aggaacccctagtgatggagttggccactccctctctgcgcgctcgctcg
ctcactgaggccgcccgggctttgcccgggcggcctcagtgagcgagcga
gcgcgcagagagggagtggccaa
C loop deletion ITR:
  (SEQ ID NO. 30)
aggaacccctagtgatggagttggccactccctctctgcgcgctcgctcg
ctcactgaggccgggcgaccaaaggtcgcccggcctcagtgagcgagcga
gcgcgcagagagggagtggccaa
```

### Methods of Treatment

Compositions and constructs disclosed herein may be used in any in vitro or in vivo application wherein expression of a payload (e.g. transgene) from a particular target locus in a cell, while maintaining expression of endogenous genes at and surrounding the target locus, is desired. For example, compositions and constructs disclosed herein may be used to treat a disorder, disease, or medical condition in a subject (e.g., through gene therapy).

In some embodiments, treatment comprises obtaining or maintaining a desired pharmacologic and/or physiologic effect. In some embodiments, a desired pharmacologic and/or physiologic effect may comprise completely or partially preventing a disease (e.g., preventing symptoms of disease). In some embodiments, a desired pharmacologic and/or physiologic effect may comprise completely or partially curing a disease (e.g., curing adverse effects associated with a disease). In some embodiments, a desired pharmacologic and/or physiologic effect may comprise preventing recurrence of a disease. In some embodiments, a desired pharmacologic and/or physiologic effect may comprise slowing progression of a disease. In some embodiments, a desired pharmacologic and/or physiologic effect may comprise relieving symptoms of a disease. In some embodiments, a desired pharmacologic and/or physiologic effect may comprise preventing regression of a disease. In some embodiments, a desired pharmacologic and/or physiologic effect may comprise stabilizing and/or reducing symptoms associated with a disease.

In some embodiments, treatment comprises administering a composition before, during, or after onset of a disease (e.g., before, during, or after appearance of symptoms associated with a disease). In some embodiments, treatment comprises combination therapy (e.g., with one or more therapies, including different types of therapies).

### Targeted Integration

In some embodiments, compositions and constructs provided herein direct integration of a payload (e.g., a transgene and/or functional nucleic acid) at a target locus (also referred to herein as a target integration site) (e.g., an endogenous gene). In some embodiments, compositions and constructs provided herein direct integration of a payload at a target locus in a specific cell type (e.g., tissue-specific loci). In some embodiments, integration of a payload occurs in a specific tissue (e.g., liver, central nervous system (CNS), muscle, kidney, vascular, lung). In some embodiments, integration of a payload occurs in multiple tissues (e.g., liver, central nervous system (CNS), muscle, kidney, vascular, lung).

In some embodiments, compositions and constructs provided herein direct integration of a payload at a target locus that is considered a safe-harbor site (e.g., albumin, Apolipoprotein A2 (ApoA2), Haptoglobin). In some embodiments, a target locus may be selected from any genomic site appropriate for use with methods and compositions provided herein. In some embodiments, a target locus encodes a polypeptide. In some embodiments, a target locus encodes a polypeptide that is highly expressed in a subject (e.g., a subject not suffering from a disease, disorder, or condition, or a subject suffering from a disease, disorder, or condition). In some embodiments, integration of a payload occurs at a 5′ or 3′ end of one or more endogenous genes (e.g., genes encoding polypeptides). In some embodiments, integration of a payload occurs between a 5′ or 3′ end of one or more endogenous genes (e.g., genes encoding polypeptides).

In some embodiments, compositions and constructs provided herein direct integration of a payload at a target locus with minimal or no off-target integration (e.g., integration at a non-target locus). In some embodiments, compositions and constructs provided herein direct integration of a payload at a target locus with reduced off-target integration compared to a reference composition or construct (e.g., relative to a composition or construct without flanking homology sequences).

In some embodiments, integration of a transgene at a target locus allows expression of a payload without disrupting endogenous gene expression. In some embodiments, integration of a transgene at a target locus allows expression of a payload from an endogenous promoter. In some embodiments, integration of a transgene at a target locus disrupts endogenous gene expression. In some embodiments, integration of a transgene at a target locus disrupts endogenous gene expression without adversely affecting a target cell and/or subject (e.g., by targeting a safe-harbor site). In some embodiments, integration of a transgene at a target locus does not require use of a nuclease (e.g., Cas proteins, endonucleases, TALENs, ZFNs). In some embodiments, integration of a transgene at a target locus is assisted by use of a nuclease (e.g., Cas proteins, endonucleases, TALENs, ZFNs).

In some embodiments, integration of a transgene at a target locus confers a selective advantage (e.g., increased survival rate in a plurality of cells relative to other cells in a tissue). In some embodiments, a selective advantage may produce an increased percentage of cells in one or more tissues expressing a transgene.

### Compositions

In some embodiments, compositions can be produced using methods and constructs provided herein (e.g., viral vectors). In some embodiments, compositions include liquid, solid, and gaseous compositions. In some embodiments, compositions comprise additional ingredients (e.g., diluents, stabilizer, excipients, adjuvants). In some embodiments, additional ingredients can comprise buffers (e.g., phosphate, citrate, organic acid buffers), antioxidants (e.g., ascorbic acid), low molecular weight polypeptides (e.g., less than 10 residues), various proteins (e.g., serum albumin, gelatin, immunoglobulins), hydrophilic polymers (e.g., polyvinylpyrrolidone), amino acids (e.g., glycine, glutamine, asparagine, arginine, lysine), carbohydrates (e.g., monosaccharides, disaccharides, glucose, mannose, dextrins), chelating agents (e.g., EDTA), sugar alcohols (e.g., mannitol, sorbitol), salt-forming counterions (e.g., sodium, potassium), and/or nonionic surfactants (e.g. Tween™, Pluronics™, polyethylene glycol (PEG)), among other things. In some embodiments, an aqueous carrier is an aqueous pH buffered solution.

In some embodiments, compositions provided herein may be provided in a range of dosages. In some embodiments, compositions provided herein may be provided in a single dose. In some embodiments, compositions provided herein may be provided in multiple dosages. In some embodiments, compositions are provided over a period of time. In some embodiments, compositions are provided at specific intervals (e.g., varying intervals, set intervals). In some embodiments, dosages may vary depending upon dosage form and route of administration. In some embodiments, compositions provided herein may be provided in dosages between 1E12 and 1E14 vg/kg. In some embodiments, compositions provided herein may be provided in dosages between 3E12 and 1E13 vg/kg. In some embodiments, compositions provided herein may be provided in dosages between 1E13 and 3E13 vg/kg. In some embodiments, compositions provided herein may be provided in dosages between 3E12 and 3E13 vg/kg. In some embodiments, compositions provided herein may be provided in dosages of no more than 3E13 vg/kg. In some embodiments, compositions provided herein may be provided in dosages of no more than 1E13 vg/kg. In some embodiments, compositions provided herein may be provided in dosages of no more than 3E12 vg/kg.

In some embodiments, compositions provided herein may be administered to a subject at a particular timepoint (e.g., age of a subject). In some embodiments, compositions provided herein may be administered to a newborn subject. In some embodiments, compositions provided herein may be administered to a neonatal subject. In some embodiments, a neonatal mouse subject is between 0 and 14 days of age. In some embodiments, a neonatal human subject is between 0 days and 1 month of age. In some embodiments compositions provided herein may be administered to a subject between 7 days of age and 30 days of age. In some embodiments, compositions provided herein may be administered to a subject between 3 months of age and 1 year of age. In some embodiments, compositions provided herein may be administered to a subject between 1 year of age and 5 years of age. In some embodiments, compositions provided herein may be administered to a subject between 4 years of age and 7 years of age. In some embodiments, compositions provided herein may be administered to a subject at 5 years of age or older.

In some embodiments, compositions provided herein may be administered to a subject at a particular timepoint based upon growth stage (e.g., percentage of estimated/average adult size or weight) of a particular tissue or organ. In some embodiments, compositions provided herein may be administered to a subject wherein a tissue or organ (e.g., liver, muscle, CNS, lung, etc.) is at least 10%, at least 20%, at least 30%, at least 40%, at least 50%, at least 60%, at least 70%, at least 80%, at least 90%, or at least 99% of estimated/average adult size or weight. In some embodiments, compositions provided herein may be administered to a subject wherein a tissue or organ is approximately 20% (+/−5%) of estimated/average adult size or weight. In some embodiments, compositions provided herein may be administered to a subject wherein a tissue or organ is approximately 50% (+/−5%) of estimated/average adult size or weight. In some embodiments, compositions provided herein may be administered to a subject wherein a tissue or organ is approximately 60% (+/−5%) of estimated/average adult size or weight. In some embodiments, estimated/average adult size or weight of a particular tissue or organ may be determined as described in the art (See, Noda et al. Pediatric radiology, 1997; Johnson et al. Liver transplantation, 2005; and Szpinda et al. Biomed research international, 2015, each of which is incorporated herein by reference in its entirety.

### Routes of Administration

In some embodiments, compositions provided herein may be administered to a subject via any one (or more) of a variety of routes known in the art (e.g., parenteral, subcutaneous, intravenous, intracranial, intraspinal, intraocular, intramuscular, intravaginal, intraperitoneal, epicutaneous, intradermal, rectal, pulmonary, intraosseous, oral, buccal, intraportal, intra-arterial, intratracheal, or nasal). In some embodiments, compositions provided herein may be introduced into cells, which are then introduced into a subject (e.g., liver, muscle, central nervous system (CNS), lung, hematologic cells). In some embodiments, compositions provided herein may be introduced via delivery methods known in the art (e.g., injection, catheter).

In some embodiments, genome editing with the GENERIDE™ platform differs from conventional gene therapy because it uses homologous recombination to deliver a corrective gene to one specific location in the genome. In some embodiments, GENERIDE™ inserts a corrective gene in a precise manner, leading to site-specific integration in the genome. In some embodiments, GENERIDE™ does not require the use of exogenous nucleases or promoters. In some embodiments, GENERIDE™ may be combined with one or more exogenous nucleases and/or promoters.

In some embodiments, provided compositions comprise one or more homology arms, a transgene, and a nucleic acid that promotes the production of two independent gene products. In some embodiments, compositions and methods of the present disclosure comprise a first nucleic acid sequence encoding a transgene. In some embodiments, compositions and methods of the present disclosure comprise a second nucleic acid that promotes the production of two independent gene products (e.g., a 2A peptide). In some embodiments, the present disclosure provides and expression cassette comprising a first nucleic acid sequence and a second nucleic acid sequence as described herein.

In some embodiments, a second nucleic acid comprises a nucleic acid sequence encoding a 2A peptide; a nucleic acid sequence encoding an internal ribosome entry site (IRES); a nucleic acid sequence encoding an N-terminal intein splicing region and C-terminal intein splicing region; and/or a nucleic acid sequence encoding a splice donor and a splice acceptor. In some embodiments, compositions and methods of the present disclosure comprise a polynucleotide cassette comprising an expression cassette comprising said first nucleic acid and said second nucleic acid. In some embodiments, compositions and methods of the present disclosure comprise a third nucleic acid sequence comprising a sequence that is substantially homologous to a genomic sequence. In some embodiments, compositions and methods of the present disclosure comprise a fourth nucleic acid sequence comprising a sequence that is substantially homologous to a genomic sequence. In some embodiments, said third nucleic acid sequence is positioned 5′ to the expression cassette and comprises a sequence that is substantially homologous to a genomic sequence 5′ of a target integration site in a genome of a cell. In some embodiments, said fourth nucleic acid sequence is positioned 3′ to the expression cassette and comprises a sequence that is substantially homologous to a genomic sequence 3′ of a target integration site in the genome of the cell.

In some embodiments, one or more compositions described herein are administered without any additional treatment. In some embodiments, one or more compositions described herein are administered in combination. In some embodiments, a first composition may be administered simultaneously with a second composition. In some embodiments, a first composition and second composition may be administered sequentially (e.g., within minutes, hours, days, weeks, or months of one another). In some embodiments, one or more compositions may be administered via the same route (e.g., parenteral, subcutaneous, intravenous, intracranial, intraspinal, intraocular, intramuscular, intravaginal, intraperitoneal, epicutaneous, intradermal, rectal, pulmonary, intraosseous, oral, buccal, intraportal, intra-arterial, intratracheal, or nasal). In some embodiments, one or more compositions may be administered via different routes (e.g., parenteral, subcutaneous, intravenous, intracranial, intraspinal, intraocular, intramuscular, intravaginal, intraperitoneal, epicutaneous, intradermal, rectal, pulmonary, intraosseous, oral, buccal, intraportal, intra-arterial, intratracheal, or nasal).

In some embodiments, the first and/or second compositions are administered at a particular dose (e.g., a fixed dose or a weight based dose) only once. In some embodiments, the first and/or second compositions are administered at a particular dose (e.g., a fixed dose or a weight based dose) more than once. In some embodiments, where more than one dose is administered (e.g., a fixed dose or a weight based dose) the first and/or second compositions may be administered simultaneously, substantially simultaneously, or consecutively. In some embodiments, multiple doses (e.g., a fixed dose or a weight based dose) are administered within a specified period of time (e.g., within minutes, hours, days, weeks, or months).

In some embodiments, the first and/or second compositions are administered in response to a biomarker (e.g., a circulating biomarker as described in WO2020214582A1). For example, the first and/or second compositions are administered at a particular dose (e.g., a fixed dose or a weight based dose) and within a specified period of time (e.g., within minutes, hours, days, weeks, or months) levels of a biomarker (e.g., as described in WO2020214582A1) are monitored. If levels of a biomarker (e.g., as described in WO2020214582A1) are low (e.g., as compared to an appropriate reference (e.g., levels of a biomarker prior to administration)), then the first and/or second compositions are administered at a particular dose (e.g., a fixed dose or a weight based dose). If levels of a biomarker (e.g., as described in WO2020214582A1) are high (e.g., as compared to an appropriate reference (e.g., levels of a biomarker after an initial administration)), then subsequent dosing (e.g., a fixed dose or a weight based dose) of the first and/or second compositions may be reevaluated (e.g., treatment suspension, reduced fixed dose or weight based dose).

### Methods of Producing Viral Vectors

### Production of Viral Vectors

In some embodiments, production of viral vectors (e.g., AAV viral vectors) may include both upstream steps to generate viral vectors (e.g. cell-based culturing) and downstream steps to process viral vectors (e.g., purification, formulation, etc.). In some embodiments, upstream steps may comprise one or more of cell expansion, cell culture, cell transfection, cell lysis, viral vector production, and/or viral vector harvest.

In some embodiments, downstream steps may comprise one or more of separation, filtration, concentration, clarification, purification, chromatography (e.g., affinity, ion exchange, hydrophobic, mixed-mode), centrifugation (e.g., ultracentrifugation), and/or formulation.

In some embodiments, constructs and methods described herein are designed to increase viral vector yields (e.g., AAV vector yields), reduce levels of replication-competent viral vectors (e.g., replication competent AAV (rcAAV)), improve viral vectors packaging efficiency (e.g., AAV vector capsid packaging), and/or any combinations thereof, relative to a reference construct or method, for example those in Xiao et al. 1998 and Grieger et al. 2015, each of which is incorporated herein by reference in its entirety.

### Cell Lines and Transfection Reagents

In some embodiments, production of viral vectors comprises use of cells (e.g., cell culture). In some embodiments, production of viral vectors comprises use of cell culture comprising one or more cell lines (e.g., mammalian cell lines). In some embodiments, production of viral vectors comprises use of HEK293 cell lines or variants thereof (e.g., HEK293T, HEK293F cell lines). In some embodiments, cells are capable of being grown in suspension. In some embodiments, cells are comprised of adherent cells. In some embodiments, cells are capable of being grown in media that does not comprise animal components (e.g. animal serum). In some embodiments, cells are capable of being grown in serum-free media (e.g., F17 media, Expi293 media). In some embodiments, production of viral vectors comprises transfection of cells with expression constructs (e.g., plasmids). In some embodiments, cells are selected for high expression of viral vectors (e.g. AAV vectors). In some embodiments, cells are selected for high packaging efficiency of viral vectors (e.g., capsid packaging of AAV vectors). In some embodiments, cells are selected for improved transfection efficiency (e.g., with chemical transfection reagents, including cationic molecules). In some embodiments, cells are engineered for high expression of viral vectors (e.g. AAV vectors). In some embodiments, cells are engineered for high packaging efficiency of viral vectors (e.g., capsid packaging of AAV vectors). In some embodiments, cells are engineered for improved transfection efficiency (e.g., with chemical transfection reagents, including cationic molecules). In some embodiments, cells may be engineered or selected for two or more of the above attributes. In some embodiments, cells are contacted with one or more expression constructs (e.g. plasmids). In some embodiments, cells are contacted with one or more transfection reagents (e.g., chemical transfection reagents, including lipids, polymers, and cationic molecules) and one or more expression constructs. In some embodiments, cells are contacted with one or more cationic molecules (e.g., cationic lipid, PEI reagent) and one or more expression constructs. In some embodiments, cells are contacted with a PEIMAX reagent and one or more expression constructs. In some embodiments, cells are contacted with a FectoVir-AAV reagent and one or more expression constructs. In some embodiments, cells are contacted with one or more transfection reagents and one or more expression constructs at particular ratios. In some embodiments, ratios of transfection reagents to expression constructs improves production of viral vectors (e.g., improved vector yield, improved packaging efficiency, and/or improved transfection efficiency).

### Expression Constructs

In some embodiments, expression constructs are or comprise one or more polynucleotide sequences (e.g., plasmids). In some embodiments, expression constructs comprise particular polynucleotide sequence elements (e.g., payloads, promoters, viral genes, etc.). In some embodiments, expression constructs comprise polynucleotide sequences encoding viral genes (e.g., a rep or cap gene or gene variant, one or more helper virus genes or gene variants). In some embodiments, expression constructs of a particular type comprise specific combinations of polynucleotide sequence elements. In some embodiments, expression constructs of a particular type do not comprise specific combinations of polynucleotide sequence elements. In some embodiments, a particular expression construct does not comprise polynucleotide sequence elements encoding both rep and cap genes and/or gene variants.

In some embodiments, expression constructs comprise polynucleotide sequences encoding wild-type viral genes (e.g., wild-type rep genes, cap genes, viral helper genes, or combinations thereof). In some embodiments, expression constructs comprise polynucleotide sequences encoding viral helper genes or gene variants (e.g., herpesvirus genes or gene variants, adenovirus genes or gene variants). In some embodiments, expression constructs comprise polynucleotide sequences encoding one or more gene copies that express one or more wild-type Rep proteins (e.g., 1 copy, 2 copies, 3 copies, 4 copies, 5 copies, etc.). In some embodiments, expression constructs comprise polynucleotide sequences encoding a single gene copy that expresses one or more wild-type Rep proteins (e.g., Rep68, Rep40, Rep52, Rep78, or combinations thereof). In some embodiments, expression constructs comprise polynucleotide sequences encoding one or more wild-type Rep proteins (e.g., Rep68, Rep40, Rep52, Rep78, or combinations thereof). In some embodiments, expression constructs comprise polynucleotide sequences encoding at least four wild-type Rep proteins (e.g., Rep68, Rep40, Rep52, Rep78). In some embodiments, expression constructs comprise polynucleotide sequences encoding each of Rep68, Rep40, Rep52, and Rep78. In some embodiments, expression constructs comprise polynucleotide sequences encoding one or more wild-type adenoviral helper proteins (e.g., E2 and E4).

In some embodiments, expression constructs comprise wild-type polynucleotide sequences encoding wild-type viral genes (e.g., rep genes, cap genes, helper genes). In some embodiments, expression constructs comprise modified polynucleotide sequences (e.g., codon-optimized) encoding wild-type viral genes (e.g., rep genes, cap genes, helper genes). In some embodiments, expression constructs comprise modified polynucleotide sequences encoding modified viral genes (e.g., rep genes, cap genes, helper genes). In some embodiments, modified viral genes are designed and/or engineered for certain improvements (e.g., improved transduction, tissue specificity, reduced size, reduced immune response, improved packaging, reduced rcAAV levels, etc.).

In accordance with various embodiments, expression constructs disclosed herein may offer increased flexibility and modularity as compared to previous technologies. In some embodiments, expression constructs disclosed herein may allow swapping of various polynucleotide sequences (e.g., different rep genes, cap genes, payloads, helper genes, promoters, etc.) while providing certain improvements (e.g., increased viral vector yield, increased packaging, reduced rcAAV levels, etc.). In some embodiments, expression constructs disclosed herein are compatible with various upstream production processes (e.g., different cell culture conditions, different transfection reagents, etc.) while providing certain improvements (e.g., increased viral vector yield, increased packaging, reduced rcAAV levels, etc.)

In some embodiments, expression constructs of different types comprise different combinations of polynucleotide sequences. In some embodiments, an expression construct of one type comprises one or more polynucleotide sequence elements (e.g., payloads, promoters, viral genes, etc.) that is not present in an expression construct of a different type. In some embodiments, an expression construct of one type comprises polynucleotide sequence elements encoding a viral gene (e.g., a rep or cap gene or gene variant) and polynucleotide sequence elements encoding a payload (e.g., a transgene and/or functional nucleic acid). In some embodiments, an expression construct of one type comprises polynucleotide sequence elements encoding one or more viral genes (e.g., a rep or cap gene or gene variant and/or one or more helper virus genes). In some embodiments, an expression construct of one type comprises polynucleotide sequence elements encoding one or more viral genes, wherein the viral genes are from one or more virus types (e.g., genes or gene variants from AAV and adenovirus). In some embodiments, viral genes from adenovirus are genes and/or gene variants. In some embodiments, viral genes from adenovirus are one or more of E2A (e.g., E2A DNA Binding Protein (DBP), E4 (e.g., E4 Open Reading Frame (ORF) 2, ORF3, ORF4, ORF6/7), VA, and/or variants thereof. In some embodiments, expression constructs are used for production of viral vectors (e.g. through cell culture). In some embodiments, expression constructs are contacted with cells in combination with one or more transfection reagents (e.g., chemical transfection reagents). In some embodiments, expression constructs are contacted with cells at particular ratios in combination with one or more transfection reagents. In some embodiments, expression constructs of different types are contacted with cells at particular ratios (e.g., weight ratios) in combination with one or more transfection reagents. In some embodiments, expression constructs of different types are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio). In some embodiments, a first expression construct comprising one or more viral helper genes and a second expression construct comprising one or more payloads are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio) of the first expression construct to the second expression construct. In some embodiments, a first expression construct comprising one or more payloads and a second expression construct comprising one or more viral helper genes are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio) of the first expression construct to the second expression construct. In some embodiments, particular ratios of expression constructs improve production of AAV (e.g., increased viral vector yields, increased packaging efficiency, and/or increased transfection efficiency. In some embodiments, cells are contacted with two or more expression constructs (e.g., sequentially or substantially simultaneously). In some embodiments, three or more expression constructs are contacted with cells. In some embodiments, expression constructs comprise one or more promoters (e.g., one or more exogenous promoters). In some embodiments, promoters are or comprise CMV, RSV, CAG, EFlalpha, PGK, A1AT, C5-12, MCK, desmin, p5, p40, or combinations thereof. In some embodiments, expression constructs comprise one or more promoters upstream of a particular polynucleotide sequence element (e.g., a rep or cap gene or gene variant). In some embodiments, expression constructs comprise one or more promoters downstream of a particular polynucleotide sequence element (e.g., a rep or cap gene or gene variant).

In some embodiments, a first expression construct comprising one or more viral helper genes and a second expression construct comprising one or more payloads are contacted with cells at a ratio greater than or equal to 1:1 up to 3:1, wherein viral titer yields are at at least 1.5× greater than those obtained through administration of a reference system (e.g., a three-plasmid comprising separate plasmids, each encoding one of: 1) an AAV rep and AAV cap sequence, 2) relevant sequence from a helper virus, and 3) a payload). In some embodiments, a first expression construct comprising one or more viral helper genes and a second expression construct comprising one or more payloads are contacted with cells at a ratio greater than or equal to 1:1 up to 5:1, wherein viral titer yields are at at least 1.5× greater than those obtained through administration of a reference system (e.g., a three-plasmid comprising separate plasmids, each encoding one of: 1) an AAV rep and AAV cap sequence, 2) relevant sequence from a helper virus, and 3) a payload). In some embodiments, a first expression construct comprising one or more viral helper genes and a second expression construct comprising one or more payloads are contacted with cells at a ratio greater than or equal to 1:1 up to 6:1, wherein viral titer yields are at at least 1.5× greater than those obtained through administration of a reference system (e.g., a three-plasmid comprising separate plasmids, each encoding one of: 1) an AAV rep and AAV cap sequence, 2) relevant sequence from a helper virus, and 3) a payload). In some embodiments, a first expression construct comprising one or more viral helper genes and a second expression construct comprising one or more payloads are contacted with cells at a ratio greater than or equal to 1:1 up to 8:1, wherein viral titer yields are at at least 1.5× greater than those obtained through administration of a reference system (e.g., a three-plasmid comprising separate plasmids, each encoding one of: 1) an AAV rep and AAV cap sequence, 2) relevant sequence from a helper virus, and 3) a payload). In some embodiments, a first expression construct comprising one or more viral helper genes and a second expression construct comprising one or more payloads are contacted with cells at a ratio greater than or equal to 1:1 up to 10:1, wherein viral titer yields are at at least 1.5× greater than those obtained through administration of a reference system (e.g., a three-plasmid comprising separate plasmids, each encoding one of: 1) an AAV rep and AAV cap sequence, 2) relevant sequence from a helper virus, and 3) a payload).

In some embodiments, a first expression construct comprising one or more viral helper genes and a second expression construct comprising one or more payloads are contacted with cells at a ratio between 10:1 and 1:1. In some embodiments, a first expression construct comprising one or more viral helper genes and a second expression construct comprising one or more payloads are contacted with cells at a ratio between 9:1 and 1:1. In some embodiments, a first expression construct comprising one or more viral helper genes and a second expression construct comprising one or more payloads are contacted with cells at a ratio between 8:1 and 1:1. In some embodiments, a first expression construct comprising one or more viral helper genes and a second expression construct comprising one or more payloads are contacted with cells at a ratio between 7:1 and 1:1. In some embodiments, a first expression construct comprising one or more viral helper genes and a second expression construct comprising one or more payloads are contacted with cells at a ratio between 6:1 and 1:1. In some embodiments, a first expression construct comprising one or more viral helper genes and a second expression construct comprising one or more payloads are contacted with cells at a ratio between 5:1 and 1:1. In some embodiments, a first expression construct comprising one or more viral helper genes and a second expression construct comprising one or more payloads are contacted with cells at a ratio between 4:1 and 1:1. In some embodiments, a first expression construct comprising one or more viral helper genes and a second expression construct comprising one or more payloads are contacted with cells at a ratio between 3:1 and 1:1. In some embodiments, a first expression construct comprising one or more viral helper genes and a second expression construct comprising one or more payloads are contacted with cells at a ratio between 2:1 and 1:1.

In some embodiments, a first expression construct comprising one or more viral helper genes and a second expression construct comprising one or more payloads are contacted with cells at a ratio between 1:1 and 2:1. In some embodiments, a first expression construct comprising one or more viral helper genes and a second expression construct comprising one or more payloads are contacted with cells at a ratio between 1:1 and 3:1. In some embodiments, a first expression construct comprising one or more viral helper genes and a second expression construct comprising one or more payloads are contacted with cells at a ratio between 1:1 and 4:1. In some embodiments, a first expression construct comprising one or more viral helper genes and a second expression construct comprising one or more payloads are contacted with cells at a ratio between 1:1 and 5:1. In some embodiments, a first expression construct comprising one or more viral helper genes and a second expression construct comprising one or more payloads are contacted with cells at a ratio between 1:1 and 6:1. In some embodiments, a first expression construct comprising one or more viral helper genes and a second expression construct comprising one or more payloads are contacted with cells at a ratio between 1:1 and 7:1. In some embodiments, a first expression construct comprising one or more viral helper genes and a second expression construct comprising one or more payloads are contacted with cells at a ratio between 1:1 and 8:1. In some embodiments, a first expression construct comprising one or more viral helper genes and a second expression construct comprising one or more payloads are contacted with cells at a ratio between 1:1 and 9:1. In some embodiments, a first expression construct comprising one or more viral helper genes and a second expression construct comprising one or more payloads are contacted with cells at a ratio between 1:1 and 10:1. In some embodiments, a first expression construct comprising one or more viral helper genes and a second expression construct comprising one or more payloads are contacted with cells at a ratio of 1.5:1.

In some embodiments, expression constructs comprise one or more polynucleotide sequences encoding elements (e.g., selection markers, origins of replication) necessary for cell culture (e.g., bacterial cell culture, mammalian cell culture). In some embodiments, expression constructs comprise one or more polynucleotide sequences encoding antibiotic resistance genes (e.g., kanamycin resistance genes, ampicillin resistance genes). In some embodiments, expression constructs comprise one or more polynucleotide sequences encoding a bacterial original of replication (e.g., colE1 origin of replication).

In some embodiments, expression constructs comprise one or more transcription termination sequences (e.g., a polyA sequence). In some embodiments, expression constructs comprise one or more of BGH polyA, FIX polyA, SV40 polyA, synthetic polyA, or combinations thereof. In some embodiments, expression constructs comprise one or more transcription termination sequences downstream of a particular sequence element (e.g., a rep or cap gene or gene variant). In some embodiments, expression constructs comprise one or more transcription termination sequences upstream of a particular sequence element (e.g., a rep or cap gene or gene variant).

In some embodiments, expression constructs comprise one or more intron sequences. In some embodiments, expression constructs comprise one or more of introns of different origins (e.g., known genes), including but not limited to FIX intron, Albumin intron, or combinations thereof. In some embodiments, expression constructs comprise one or more introns of different lengths (e.g., 133 bp to 4 kb). In some embodiments, expression constructs comprise one or more intron sequences upstream of a particular sequence element (e.g., a rep or cap gene or gene variant). In some embodiments, expression constructs comprise one or more intron sequences within a particular sequence element (e.g., a rep or cap gene or gene variant). In some embodiments, expression constructs comprise one or more intron sequences downstream of particular sequence element (e.g., a rep or cap gene or gene variant). In some embodiments, expression constructs comprise one or more intron sequences after a promoter (e.g., a p5 promoter). In some embodiments, expression constructs comprise one or more intron sequences before a rep gene or gene variant. In some embodiments, expression constructs comprise one or more intron sequences between a promoter and a rep gene or gene variant. In some embodiments, compositions provided herein comprise expression constructs. In some embodiments, compositions comprise: (i) a first expression construct comprising a polynucleotide sequence encoding one or more rep genes and a polynucleotide sequence encoding one or more wild-type adenoviral helper proteins; and (ii) a second expression construct comprising a polynucleotide sequence encoding one or more cap genes and one or more payloads.

In some embodiments, expression constructs will comprise a three-plasmid (e.g., triple transfection) system for production of viral vectors. In some embodiments, a three-plasmid system will comprise: 1) a first plasmid comprising one or more sequences encoding a rep and cap gene, or variant thereof; 2) a second sequence encoding one or more payloads; and 3) a third sequence encoding one or more helper proteins. In some embodiments, a three-plasmid system may be used to produce one or more viral vectors disclosed herein.

### Methods of Characterizing AAV Viral Vectors

In accordance with various embodiments, viral vectors may be characterized through assessment of various characteristics and/or features. In some embodiments, assessment of viral vectors can be conducted at various points in a production process. In some embodiments, assessment of viral vectors can be conducted after completion of upstream production steps. In some embodiments, assessment of viral vectors can be conducted after completion of downstream production steps.

### Viral Yields

In some embodiments, characterization of viral vectors comprises assessment of viral yields (e.g., viral titer). In some embodiments, characterization of viral vectors comprises assessment of viral yields prior to purification and/or filtration. In some embodiments, characterization of viral vectors comprises assessment of viral yields after purification and/or filtration. In some embodiments, characterization of viral vectors comprises assessing whether viral yield is greater than or equal to 1e10 vg/mL.

In some embodiments, characterization of viral vectors comprises assessing whether viral yield in crude cell lysates is greater than or equal to ell vg/mL. In some embodiments, characterization of viral vectors comprises assessing whether viral yield in crude cell lysates is greater than or equal to 5e11 vg/mL. In some embodiments, characterization of viral vectors comprises assessing whether viral yield in crude cell lysates is greater than or equal to 1e12 vg/mL. In some embodiments, characterization of viral vectors comprises assessing whether viral yield in crude lysates is between 5e9 vg/mL and 5e11 vg/mL. In some embodiments, characterization of viral vectors comprises assessing whether viral yield in crude lysates is between 5e9 vg/mL and 1e10 vg/mL. In some embodiments, characterization of viral vectors comprises assessing whether viral yield in crude lysates is between 1e10 vg/mL and 1e11 vg/mL. In some embodiments, characterization of viral vectors comprises assessing whether viral yield in crude lysates is between 1e11 vg/mL and 1e12 vg/mL. In some embodiments, characterization of viral vectors comprises assessing whether viral yield in crude lysates is between 1e12 vg/mL and 1e13 vg/mL.

In some embodiments, characterization of viral vectors comprises assessing whether viral yield in purified drug product is greater than or equal to 1e1 vg/mL. In some embodiments, characterization of viral vectors comprises assessing whether viral yield in purified drug product is greater than or equal to 1e12 vg/mL. In some embodiments, characterization of viral vectors comprises assessing whether viral yield in purified drug product is between 1e10 vg/mL and 1e15 vg/mL. In some embodiments, characterization of viral vectors comprises assessing whether viral yield in purified drug product is between 1e11 vg/mL and 1e15 vg/mL. In some embodiments, characterization of viral vectors comprises assessing whether viral yield in purified drug product is between 1e12 vg/mL and 1e14 vg/mL. In some embodiments, characterization of viral vectors comprises assessing whether viral yield in purified drug product is between 1e13 and 1e14 vg/mL.

In some embodiments, methods and compositions provided herein can provide comparable or increased viral vector yields as compared to previous methods known in the art. For example, in some embodiments, provided methods for producing and/or manufacturing viral vectors comprising use of a two-plasmid transfection system provide comparable or increased viral vector yields as compared to a three-plasmid system. In some embodiments, provided methods for producing and/or manufacturing viral vectors comprising use of a two-plasmid transfection system with particular combinations of sequence elements provide comparable or increased viral vector yields as compared to a two-plasmid system with a different combination of sequence elements. In some embodiments, provided methods for producing and/or manufacturing viral vectors comprising use of a two-plasmid transfection system with particular plasmid ratios provide comparable or increased viral vector yields as compared to a two-plasmid system with different plasmid ratios. In some embodiments, provided methods for producing and/or manufacturing viral vectors comprising use of a two-plasmid transfection system with particular plasmid ratios provide comparable or increased viral vector yields as compared to a reference (e.g., two-plasmid system with different plasmid ratios, three-plasmid system) under particular culture conditions. In some embodiments, provided methods for producing and/or manufacturing viral vectors comprising use of a two-plasmid transfection system with particular plasmid ratios provide comparable or increased viral vector yields as compared to a reference (e.g., two-plasmid system with different plasmid ratios, three-plasmid system) under large-scale culture conditions (e.g., greater than 100 mL, greater than 250 mL, greater than 1 L, greater than 10 L, greater than 20 L, greater than 30 L, greater than 40 L, greater than 50 L, etc.).

### Viral Packaging

In some embodiments, characterization of viral vectors comprises assessment of viral packaging efficiency (e.g., percent of full versus empty capsids). In some embodiments, characterization of viral vectors comprises assessment of viral packaging efficiency prior to purification and/or full capsid enrichment (e.g., cesium chloride-based density gradient, iodixanol-based density gradient or ion exchange chromatography). In some embodiments, characterization of viral vectors comprises assessing whether viral packaging efficiency is greater than or equal to 20% prior to purification and/or filtration (e.g., 20%, 25%, 30%, 35%, 40%, 45%, 50%, 55%, 60%, 65%, 70%, 75%, 80%, 85%, 90%, 95%, 99%, 100%). In some embodiments, characterization of viral vectors comprises assessment of viral packaging efficiency after purification and/or full capsid enrichment. In some embodiments, characterization of viral vectors comprises assessing whether viral packaging efficiency is greater than or equal to 50% after purification and/or filtration (e.g., 50%, 55%, 60%, 65%, 70%, 75%, 80%, 85%, 90%, 95%, 99%, 100%).

In some embodiments, methods and compositions provided herein can provide comparable or increased packaging efficiency as compared to previous methods known in the art. For example, in some embodiments, provided methods for producing and/or manufacturing viral vectors comprising use of a two-plasmid transfection system provide comparable or increased packaging efficiency as compared to a three-plasmid system. In some embodiments, provided methods for producing and/or manufacturing viral vectors comprising use of a two-plasmid transfection system with particular combinations of sequence elements provide comparable or increased packaging efficiency as compared to a two-plasmid system with a different combination of sequence elements. In some embodiments, provided methods for producing and/or manufacturing viral vectors comprising use of a two-plasmid transfection system with particular plasmid ratios provide comparable or increased packaging efficiency as compared to a two-plasmid system with different plasmid ratios.

### Replication Competent Vector Levels

In some embodiments, characterization of viral vectors comprises assessment of levels of replication competent vectors. In some embodiments, characterization of viral vectors comprises assessment of levels of replication competent vectors prior to purification and/or filtration. In some embodiments, characterization of viral vectors comprises assessment of levels of replication competent vectors after purification and/or filtration. In some embodiments, characterization of viral vectors comprises assessing whether replication competent vector levels are less than or equal to 1 rcAAV in 1E10 vg.

In some embodiments, methods and compositions provided herein can provide comparable or reduced replication competent vector levels as compared to previous methods known in the art. For example, in some embodiments, provided methods for producing viral vectors comprising use of a two-plasmid transfection system provide comparable or reduced replication competent vector levels as compared to a three-plasmid system. In some embodiments, provided methods for producing viral vectors comprising use of a two-plasmid transfection system with particular combinations of sequence elements provide comparable or reduced replication competent vector levels as compared to a two-plasmid system with a different combination of sequence elements. In some embodiments, provided methods for producing viral vectors comprise use of a two-plasmid transfection system with one or more intronic sequences inserted in the rep gene provide comparable or reduced replication competent vector levels as compared to a two-plasmid system without said intronic sequence(s).

### Hereditary Tyrosinemia 1 (HT-1)

Hereditary Tyrosinemia Type 1 (HT1) is an ultra-rare neonatal-onset metabolic disorder caused by loss-of-function mutations in furmarylacetoacetate hydrolase (FAH). If untreated, HT1 patients show acute liver failure, renal damage and often develop hepatocellular carcinoma early in childhood. Standard of care for HT1 consists of the lifelong medication Nitisinone (NTBC) and diet restriction. While this treatment is effective in preventing organ failure, there remains a strong unmet medical need due to non-compliance of medication and diet adherence.

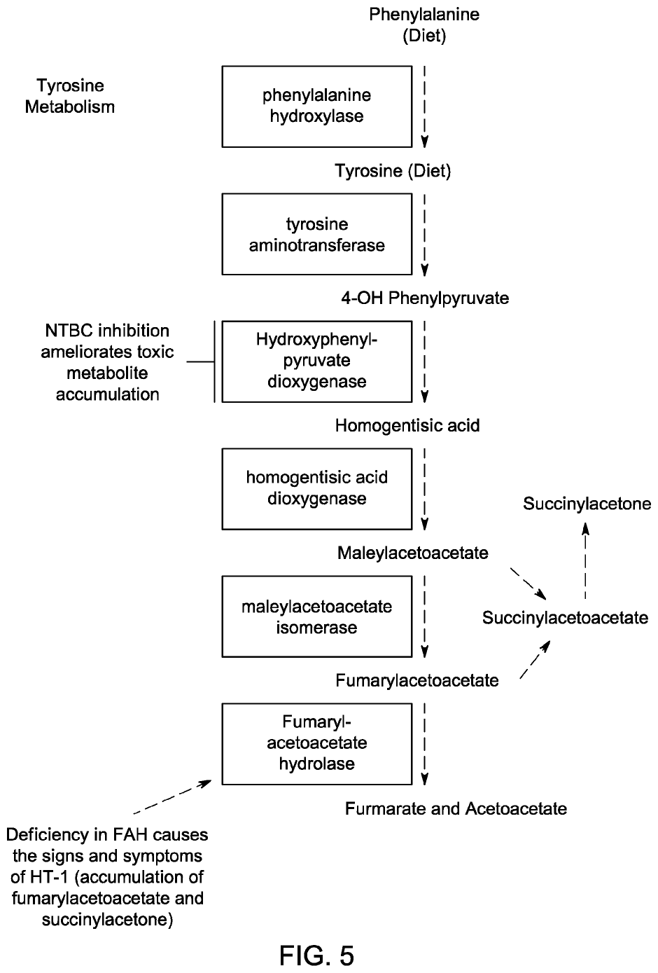

HT1 is caused by defects in furmarylacetoacetate hydrolase (FAH), the last enzyme in the tyrosine catabolic pathway (see FIG. 5). As a result, deficiency in FAH leads to an abnormal accumulation of toxic metabolites (e.g., fumarylacetoacetate (FAA) and succinylacetone (SUAC) causing the signs and symptoms of HT1 (See, LindBlad et al. PNAS, 1977 and Grompe Semin Liver Dis, 2001).

HT1 is a severe autosomal recessive metabolic disorder that presents in early infancy (within 2 years of life). It is estimated that HT-1 affects 1 per 100,000-120,000 newborns globally, but incidence can be more common in some regions, such as Norway or Quebec, Canada (See, Russo et al., Pediatric and Developmental Pathology, 2001). Patients with HT-1 experience hepatic dysfunction (hepatomegaly, cirrhosis, and hepatocellular carcinoma) and can also have associated comorbidities involving the renal and neurological system, and show failure to thrive. Without treatment, HT-1 is fatal whereby hepatic failure is a major cause of early death and all patients with HT-1 are at a high risk of developing hepatocellular carcinoma (HCC) (See, Russo et al, Pediatric and Developmental Pathology, 2001; Morrow et al. Hereditary Tyrosinemia pages 9-21, 2017; Chinsky et al. Genetics in Medicine, 2017; and Ginkel et al. Pediatric Drugs, 2019).

To date the most curative treatment for HT1 involves orthotopic liver transplantation and is only performed in severe HT-1 cases (See, Morrow et al. Hereditary Tyrosinemia pages 9-21, 2017). After transplantation, patients generally show decreased, not suppressed, urine and plasma levels of toxic metabolites (See, Paradis et al. American Journal of Human Genetics, 1990; Forget et al. Pediatric Radiology, 1999). Presumably, because of continued production in the kidneys. Most patients are instead treated via diet restriction (low phenylalanine and tyrosine intake) and 0.5-2.0 mg/kg/day oral administration of NTBC (2-nitro-4-trifluoromethylbenzoyl)-1,3cyclohexanedione, Nitisinone) (See, Chinsky et al. Genetics in Medicine, 2017). NTBC is a reversible inhibitor of 4-hydroxyphenylpyruvate dioxygenase, thereby, blocking the second step in tyrosine metabolism and preventing formation of toxic metabolites (See, Holme et al. Journal of Inherited Metabolic Disease, 1998). Common treatment for HT1 includes initiation of treatment with NTBC and diet restriction to begin within the first month of life, and continued without interruption to prevent the development of hepatic failure, renal failure, and HCC (See, Chinsky et al. Genetics in Medicine, 2017). Therefore, eliminating the urgent need for liver transplantation. However, early diagnosis and treatment is critical, as effectiveness of this treatment depends on how early the disease is recognized. It has been reported that patients who received NTBC treatment after the neonatal period have a 2-12-fold higher risk of developing HCC compared to patients treated as neonates (See, Mayorandan et al. Orphanet Journal of Rare Diseases, 2014).

In some embodiments, a subject of the present disclosure is a neonate, infant, child, or adult. In some embodiments, a subject of the present disclosure is one week old, two weeks old, three weeks old, four weeks old, five weeks old, six weeks old, seven weeks old, eight weeks, nine weeks, ten weeks, or 12 weeks old. In some embodiments, a subject of the present disclosure is between one to three; two to four; three to five; four to six; five to seven; six to eight; six to nine; eight to ten; nine to eleven; or ten to twelve weeks old. In some embodiments, a subject of the present disclosure is less than one month old. In some embodiments, a subject of the present disclosure is one month; two months; three months; four months; five months; six months old. In some embodiments, a subject of the present disclosure is between one to three; two to four; three to five; or four to six months old.

In some embodiments, a subject of the present disclosure is between 1 and 5; 3 and 7; 5 and 9; 7 and 11; 9 and 13; 11 and 15; 13 and 17; 15 and 19; 17 and 21; 19 and 23; 21 and 25; 23 and 27; 25 and 29; 27 and 31; 29 and 33; 31 and 35 years old. In some embodiments, a subject of the present disclosure is 30 and 40; 40 and 50; 50 and 60; 60 and 70; 70 and 80; or 80 and 90 years old.

In some embodiments, a subject has received or is receiving treatment for HT1. In some embodiments, a method of treatment for HT1 comprises standard of care treatment (i.e. NTBC and diet restriction). In some embodiments, a treatment for HT1 comprises NTBC.

In some embodiments, methods of the present disclosure comprise administering a composition comprising a polynucleotide cassette to a subject that has received or is receiving treatment for HT1. In some embodiments, methods of the present disclosure comprise administering a composition comprising a polynucleotide cassette to a subject that has received or is receiving NTBC. In some embodiments, a composition comprising a polynucleotide cassette and a treatment for HT1 (e.g., NTBC) are administered to a subject simultaneously or sequentially.

In some embodiments, administration of a composition of the present disclosure can result in modification of standard of care or prior or concurrent treatment. In some embodiments, a subject receives a lower or reduced dose of the treatment a subject is receiving. In some embodiments, a subject stops or no longer receives the treatment a subject has received or is receiving.

As with most chronic diseases, patients and healthcare providers must consider the risks associated with non-compliance to long-term care. Quality of life for patients, their families, and caregivers is significantly impacted by the disease due to the constraints it places on life and social functioning. Reinforcing medical recommendations for constant lifelong pharmacological and dietary therapy can be challenging as periods of dietary or medical noncompliance may be asymptomatic (See, Chinsky et al. Genetics in Medicine, 2017). Importantly, periods of noncompliance or poor adherence to pharmacological and dietary treatment can directly or indirectly influence patient outcomes as increased levels of toxic metabolites may promote the development of HCC. NTBC treatment can result in higher levels of tyrosine in blood, as it is not being catabolized, and without strict dietary adherence patients can develop corneal disease (comeal crystals) (See, Chinsky et al. Genetics in Medicine, 2017).

NTBC (Brand name Orfadin) is an expensive drug and the effect of long-term NTBC treatment is not known yet. Introduction of a functional copy of the FAH gene into the genome of HT-1 patients would represent a much better approach, potentially providing lifelong therapeutic benefit from a single administration.

In some embodiments, a transgene of the present disclosure comprises a sequence encoding FAH. In some embodiments, a sequence encoding FAH has 80%, 85%, 90%, 95%, 99%, sequence identity to one of SEQ. ID NOs.: 18-22

```
Mouse FAH
  (mFAH, SEQ ID NO: 18)
tcctttattccagtggccgaggactccgactttcccatccaaaacctgccctatggtgttttctccactcaaagcaacccaa
agccacggattggtgtagccatcggtgaccagatcttggacctgagtgtcattaaacacctctttaccggacctgccctttccaaa
catcaacatgtcttcgatgagacaactctcaataacttcatgggtctgggtcaagctgcatggaaggaggcaagagcatccttaca
gaacttactgtctgccagccaagcccggctcagagatgacaaggagcttcggcagcgtgcattcacctcccaggcttctgcgacaa
tgcaccttcctgctaccataggagactacacggacttctactcttctcggcagcatgccaccaatgttggcattatgttcagaggc
aaggagaatgcgctgttgccaaattggctccacttacctgtgggataccatggccgagcttcctccattgtggtatctggaacccc
gattcgaagacccatggggcagatgagacctgataactcaaagcctcctgtgtatggtgcctgcagactcttagacatggagttgg
aaatggctttcttcgtaggccctgggaacagattcggagagccaatccccatttccaaagcccatgaacacattttcgggatggtc
ctcatgaacgactggagcgcacgagacatccagcaatgggagtacgtcccacttgggccattcctggggaaaagctttggaaccac
aatctccccgtgggtggtgcctatggatgccctcatgccctttgtggtgccaaacccaaagcaggaccccaagcccttgccatatc
tctgccacagccagccctacacatttgatatcaacctgtctgtctctttgaaaggagaaggaatgagccaggcggctaccatctgc
aggtctaactttaagcacatgtactggaccatgctgcagcaactcacacaccactctgttaatggatgcaacctgagacctgggga
cctcttggcttctggaaccatcagtggatcagaccctgaaagctttggctccatgctggaactgtcctggaagggaacaaaggcca
tcgatgtggggcaggggcagaccaggaccttcctgctggacggcgatgaagtcatcataacaggtcactgccagggggacggctac
cgtgttggctttggccagtgtgctgggaaagtgctgcctgccctttcaccagcc
Human FAH
  (hFAH, SEQ ID NO: 19)
tccttcatcccggtggccgaggattccgacttccccatccacaacctgccctacggcgtcttctcgaccagaggcga
cccaagaccgaggataggtgtggccattggcgaccagatcctggacctcagcatcatcaagcacctctttactggtcctgtcctct
ccaaacaccaggatgtcttcaatcagcctacactcaacagcttcatgggcctgggtcaggctgcctggaaggaggcgagagtgttc
ttgcagaacttgctgtctgtgagccaagccaggctcagagatgacaccgaacttcggaagtgtgcattcatctcccaggcttctgc
cacgatgcaccttccagccaccataggagactacacagacttctattcctctcggcagcatgctaccaacgtcggaatcatgttca
gggacaaggagaatgcgttgatgccaaattggctgcacttaccagtgggctaccatggccgtgcctcctctgtcgtggtgtctggc
accccaatccgaaggcccatgggacagatgaaacctgatgactctaagcctcccgtatatggtgcctgcaagctcttggacatgga
gctggaaatggctttttttgtaggccctggaaacagattgggagagccgatccccatttccaaggcccatgagcacatttttggaa
tggtccttatgaacgactggagtgcacgagacattcagaagtgggagtatgtccctctcgggccattccttgggaagagttttggg
accactgtctctccgtgggtggtgcccatggatgctctcatgccctttgctgtgcccaacccgaagcaggaccccaggcccctgcc
gtatctgtgccatgacgagccctacacatttgacatcaacctctctgttaacctgaaaggagaaggaatgagccaggcggctacca
tatgcaagtccaattttaagtacatgtactggacgatgctgcagcagctcactcaccactctgtcaacggctgcaacctgcggccg
ggggacctcctggcttctgggaccatcagcgggccggagccagaaaacttcggctccatgttggaactgtcgtggaagggaacgaa
gcccatagacctggggaatggtcagaccaggaagtttctgctggacggggatgaagtcatcataacagggtactgccagggggatg
gttaccgcatcggctttggccagtgtgctggaaaagtgctgcctgctctcctgccatca
Human FAH-Codon Optimized Variant 3
  (hFAH-co3, SEQ ID NO: 20)
tccttcattcctgtggccgaggattctgactttcccattcacaacctgccctatggcgtcttttcaaccaggggaga
tcccaggcccagaatcggggtggccattggagatcagatcctggacctgtcaatcatcaagcacctgtttacaggccctgtgctga
gcaagcaccaggatgtgttcaaccagccaactctgaacagcttcatggggctggggcaggctgcctggaaggaggcaagggtgttt
ctgcagaatctgctgagcgtgtcacaggctaggctgagggatgataccgagctgaggaagtgtgcatttatctcacaggcatcagc
cacaatgcatctgccagctacaatcggcgactataccgacttttactcaagcaggcagcatgccaccaatgtgggcatcatgttca
gggacaaagagaatgccctgatgccaaattggctgcacctgcctgtgggctatcatggcagagccagctcagtggtggtgtcaggg
acaccaattaggaggcctatgggccagatgaagcctgatgacagcaaaccacctgtctacggcgcctgcaagctcctggatatgga
actggagatggctttttttgtgggcccaggcaatagactgggagagcccattccaatctcaaaggcccatgaacatatctttggca
tggtcctgatgaatgactggtcagccagagacattcagaagtgggagtacgtgccactgggaccatttctgggaaagagctttggc
accacagtgtcaccatgggtggtgcccatggatgccctgatgccctttgctgtgccaaatccaaaacaagaccccaggcccctccc
ctatctctgtcatgatgaaccatatacttttgacattaacctgagcgtgaacctgaaaggagaaggcatgagccaagccgccacta
tctgtaagagcaacttcaaatatatgtactggacaatgctgcagcagctcacccaccatagcgtgaatgggtgtaacctgaggcca
ggcgacctgctggcatcaggcactatttcagggcctgagcctgaaaattttggatcaatgctggagctgtcatggaagggaactaa
acccatcgacctggggaatggccagaccagaaagtttctcctggatggcgatgaggtgatcattacaggctactgtcagggggatg
gctatagaattggatttggccaatgcgccggcaaagtcctccctgccctgctgcccagc
Human FAH-Codon Optimized Variant 2
(hFAH-co2, SEQ ID NO: 21)
tccttcatcccagtggctgaagattctgacttccccattcacaacctcccttatggagtctttagcacaagaggagacc
caagacctagaattggcgtggctattggggatcagatcctggacctgagcattatcaaacatctctttacaggcccagtcctcagc
aagcaccaagatgtgttcaatcaacccacactgaactcatttatggggctgggccaagctgcctggaaggaggcaagagtgtttct
ccagaacctcctgtcagtgagccaggccaggctgagagatgacacagagctgaggaagtgtgcctttatctcacaagccagcgcca
caatgcacctgccagcaaccatcggggactacacagacttttacagcagcaggcagcacgccactaatgtgggcatcatgtttaga
gacaaagagaatgccctcatgcctaactggctgcatctgccagtgggctaccatggcagagccagcagcgtggtggtgtctggcac
ccctatcaggaggcccatgggccagatgaagcctgatgacagcaagccacctgtgtatggggcatgtaagctgctggacatggagc
tggaaatggctttctttgtggggcctgggaacaggctgggcgaaccaattcccatcagcaaagctcatgaacacatttttgggatg
gtgctcatgaatgactggtctgccagagacatccagaagtgggagtatgtccccctgggaccattcctgggcaagagctttgggac
cactgtgtcaccatgggtggtgcccatggatgccctgatgccatttgctgtgcctaatcccaaacaagatccaaggccactgccct
acctctgccacgatgagccctacacatttgatatcaacctctctgtgaatctgaagggagaaggaatgagccaagctgctaccatt
tgcaaaagcaactttaagtatatgtattggaccatgctgcagcaactcacccaccacagcgtgaatggctgtaacctgaggcctgg
cgacctgctggcctctggcaccatcagcggccctgaacctgagaactttggcagcatgctggagctgagctggaagggaaccaagc
ccattgacctgggcaatggacagaccaggaaattcctcctggacggggacgaggtgatcatcacaggctattgccagggggatggg
tataggattggctttggccaatgtgccggcaaagtgctgcctgccctgctccctagc
Human FAH-Codon Optimized Variant 1
(hFAH-col, SEQ ID NO: 22)
tcctttattcctgtggccgaagatagcgacttccctatccataacctcccatatggcgtcttctcaaccaggggcg
accccaggcccaggattggggggcaattggagaccagatcctggacctcagcatcattaagcacctctttacaggacctgtgctga
gcaagcaccaggatgtgttcaaccagcctaccctgaatagctttatgggactgggccaggcagcatggaaagaagccagagtgttc
ctccagaatctgctgagcgtgagccaggccaggctcagggatgacacagaactgaggaaatgtgccttcatctcacaggcatcagc
cacaatgcacctccctgccacaattggggactacactgacttctacagcagcagacagcatgccactaatgtggggattatgttta
gagacaaggaaaatgccctcatgccaaattggctgcacctgcctgtgggctaccacggcagagcctcatcagtggtggtgtcaggc
acaccaattaggaggccaatgggacagatgaagcctgacgactcaaagccaccagtctatggcgcctgcaaactgctggacatgga
gctggagatggctttctttgtggggcctggcaacaggctgggagaaccaatccctatttcaaaggcccacgagcacatttttggca
tggtgctcatgaatgactggtctgccagagatatccagaagtgggaatacgtgcccctgggcccttttctgggcaagagctttggc
accacagtgtcaccttgggtggtcccaatggatgccctgatgccctttgccgtgcccaaccccaagcaggatcccagaccactgcc
ctacctgtgccacgatgagccctatacctttgacatcaacctgtcagtcaacctgaagggggagggcatgagccaggccgccacta
tttgcaagagcaactttaaatatatgtactggactatgctccaacagctcactcaccactcagtgaatggctgcaatctgaggcct
ggcgacctcctggctagcggcactatctctggccctgaacctgagaactttgggagcatgctggagctgtcatggaaagggacaaa
gcctattgacctggggaatggccagacaaggaagtttctcctggatggcgatgaggtcatcatcactgggtactgccagggcgatg
gctacaggattggatttggacagtgtgctggcaaagtgctcccagctctgctgccctca
```

Because GENERIDE™ is designed to deliver therapeutic durability, it may provide lifelong benefit to patients with HT-1 by intervening early in their lives with a treatment that restores the function of aberrant genes before declines in function can occur. In some embodiments, therapeutic transgenes are delivered using a GENERIDE™ construct designed to integrate immediately behind the gene coding for albumin, the most highly expressed gene in the liver. In some embodiments, expression of the transgenes “piggybacks” on the expression of albumin, which may provide sufficient therapeutic levels of desirable proteins given the high level of albumin expression in the liver.

In some embodiments, compositions of the present disclosure comprise a viral vector capsid and a polynucleotide cassette as described herein. In some embodiments, a composition of the present disclosure may have 85%, 90%, 95%, 90%, 95%, 99% or 100% sequence identity to a sequence provided below:

GR-FAH-co3_1.6/1.0_kb
(SEQ ID NO. 23)
cgattcattaatgcagctgcgcgctcgctcgctcactgaggccgcccgggcaaagcccgg
gcgtcgggcgacctttggtcgcccggcctcagtgagcgagcgagcgcgcagagagggagt
ggccaactccatcactaggggttccttgtagttaatgattaacccgccatgctacttatc
tacgtaTTTCTAGATGTAAATAATTATTTTAAGTTTGCCCTATGGTGGCCCCACACATGA
GACAAACCCCCAAGATGTGACTTTTGAGAATGAGACTTGGATAAAAAACATGTAGAAATG
CAAGCCCTGAAGCTCAACTCCCTATTGCTATCACAGGGGTTATAATTGCATAAAATTTAG
CTATAGAAAGTTGCTGTCATCTCTTGTGGGCTGTAATCATCGTCTAGGCTTAAGAGTAAT
ATTGCAAAACCTGTCATGCCCACACAAATCTCTCCCTGGCATTGTTGTCTTTGCAGATGT
CAGTGAAAGAGAACCAGCAGCTCCCATGAGTTTGGATAGCCTTATTTTCTATAGCCTCCC
CACTATTAGCTTTGAAGGGAGCAAAGTTTAAGAACCAAATATAAAGTTTCTCATCTTTAT
AGATGAGAAAAATTTTAAATAAAGTCCAAGATAATTAAATTTTTAAGGATCATTTTTAGC
TCTTTAATAGCAATAAAACTCAATATGACATAATATGGCACTTCCAAAATCTGAATAATA
TATAATTGCAATGACATACTTCTTTTCAGAGATTTACTGAAAAGAAATTTGTTGACACTA
CATAACGTGATGAGTGGTTTATACTGATTGTTTCAGTTGGTCTTCCCACCAACTCCATGA
AAGTGGATTTTATTATCCTCATCATGCAGATGAGAATATTGAGACTTATAGCGGTATGCC
TGAGCCCCAAAGTACTCAGAGTTGCCTGGCTCCAAGATTTATAATCTTAAATGATGGGAC
TACCATCCTTACTCTCTCCATTTTTCTATACGTGAGTAATGTTTTTTCTGTTTTTTTTTT
TTCTTTTTCCATTCAAACTCAGTGCACTTGTTGAGCTCGTGAAACACAAGCCCAAGGCAA
CAAAAGAGCAACTGAAAGCTGTTATGGATGATTTCGCAGCTTTTGTAGAGAAGTGCTGCA
AGGCTGACGATAAGGAGACCTGCTTTGCCGAGGAGGTACTACAGTTCTCTTCATTTTAAT
ATGTCCAGTATTCATTTTTGCATGTTTGGTTAGGCTAGGGCTTAGGGATTTATATATCAA
AGGAGGCTTTGTACATGTGGGACAGGGATCTTATTTTACAAACAATTGTCTTACAAAATG
AATAAAACAGCACTTTGTTTTTATCTCCTGCTCTATTGTGCCATACTGTTAAATGTTTAT
AATGCCTGTTCTGTTTCCAAATTTGTGATGCTTATGAATATTAATAGGAATATTTGTAAG
GCCTGAAATATTTTGATCATGAAATCAAAACATTAATTTATTTAAACATTTACTTGAAAT
GTGGTGGTTTGTGATTTAGTTGATTTTATAGGCTAGTGGGAGAATTTACATTCAAATGTC
TAAATCACTTAAAATTGCCCTTTATGGCCTGACAGTAACTTTTTTTTATTCATTTGGGGA
CAACTATGTCCGTGAGCTTCCGTCCAGAGATTATAGTAGTAAATTGTAATTAAAGGATAT
GATGCACGTGAAATCACTTTGCAATCATCAATAGCTTCATAAATGTTAATTTTGTATCCT
AATAGTAATGCTAATATTTTCCTAACATCTGTCATGTCTTTGTGTTCAGGGTAAAAAACT
TGTTGCTGCAAGTCAAGCTGCCTTAGGCTTAGGAAGCGGCGCCACCAATTTCAGCCTGCT
GAAACAGGCCGGCGACGTGGAAGAGAACCCTGGCCCTTCCTTCATTCCTGTGGCCGAGGA
TTCTGACTTTCCCATTCACAACCTGCCCTATGGCGTCTTTTCAACCAGGGGAGATCCCAG
GCCCAGAATCGGGGTGGCCATTGGAGATCAGATCCTGGACCTGTCAATCATCAAGCACCT
GTTTACAGGCCCTGTGCTGAGCAAGCACCAGGATGTGTTCAACCAGCCAACTCTGAACAG
CTTCATGGGGCTGGGGCAGGCTGCCTGGAAGGAGGCAAGGGTGTTTCTGCAGAATCTGCT
GAGCGTGTCACAGGCTAGGCTGAGGGATGATACCGAGCTGAGGAAGTGTGCATTTATCTC
ACAGGCATCAGCCACAATGCATCTGCCAGCTACAATCGGCGACTATACCGACTTTTACTC
AAGCAGGCAGCATGCCACCAATGTGGGCATCATGTTCAGGGACAAAGAGAATGCCCTGAT
GCCAAATTGGCTGCACCTGCCTGTGGGCTATCATGGCAGAGCCAGCTCAGTGGTGGTGTC
AGGGACACCAATTAGGAGGCCTATGGGCCAGATGAAGCCTGATGACAGCAAACCACCTGT
CTACGGCGCCTGCAAGCTCCTGGATATGGAACTGGAGATGGCTTTTTTTGTGGGCCCAGG
CAATAGACTGGGAGAGCCCATTCCAATCTCAAAGGCCCATGAACATATCTTTGGCATGGT
CCTGATGAATGACTGGTCAGCCAGAGACATTCAGAAGTGGGAGTACGTGCCACTGGGACC
ATTTCTGGGAAAgAGCTTTGGCACCACAGTGTCACCATGGGTGGTGCCCATGGATGCCCT
GATGCCCTTTGCTGTGCCAAATCCAAAACAAGACCCCAGGCCCCTCCCCTATCTCTGTCA
TGATGAACCATATACTTTTGACATTAACCTGAGCGTGAACCTGAAAGGAGAAGGCATGAG
CCAAGCCGCCACTATCTGTAAGAGCAACTTCAAATATATGTACTGGACAATGCTGCAGCA
GCTCACCCACCATAGCGTGAATGGGTGTAACCTGAGGCCAGGCGACCTGCTGGCATCAGG
CACTATTTCAGGGCCTGAGCCTGAAAATTTTGGATCAATGCTGGAGCTGTCATGGAAGGG
AACTAAACCCATCGACCTGGGGAATGGCCAGACCAGAAAGTTTCTCCTGGATGGCGATGA
GGTGATCATTACAGGCTACTGTCAGGGGGATGGCTATAGAATTGGATTTGGCCAATGCGC
CGGCAAAGTCCTCCCTGCCCTGCTGCCCAGCTAACATCACATTTAAAAGCATCTCAGGTA
ACTATATTTTGAATTTTTTAAAAAAGTAACTATAATAGTTATTATTAAAATAGCAAAGAT
TGACCATTTCCAAGAGCCATATAGACCAGCACCGACCACTATTCTAAACTATTTATGTAT
GTAAATATTAGCTTTTAAAATTCTCAAAATAGTTGCTGAGTTGGGAACCACTATTATTTC
TATTTTGTAGATGAGAAAATGAAGATAAACATCAAAGCATAGATTAAGTAATTTTCCAAA
GGGTCAAAATTCAAAATTGAAACCAAAGTTTCAGTGTTGCCCATTGTCCTGTTCTGACTT
ATATGATGCGGTACACAGAGCCATCCAAGTAAGTGATGGCTCAGCAGTGGAATACTCTGG
GAATTAGGCTGAACCACATGAAAGAGTGCTTTATAGGGCAAAAACAGTTGAATATCAGTG
ATTTCACATGGTTCAACCTAATAGTTCAACTCATCCTTTCCATTGGAGAATATGATGGAT
CTACCTTCTGTGAACTTTATAGTGAAGAATCTGCTATTACATTTCCAATTTGTCAACATG
CTGAGCTTTAATAGGACTTATCTTCTTATGACAACATTTATTGGTGTGTCCCCTTGCCTA
GCCCAACAGAAGAATTCAGCAGCCGTAAGTCTAGGACAGGCTTAAATTGTTTTCACTGGT
GTAAATTGCAGAAAGATGATCTAAGTAATTTGGCATTTATTTTAATAGGTTTGAAAAACA
CATGCCATTTTACAAATAAGACTTATATTTGTCCTTTTGTTTTTCAGCCTACCATGAGAA
TAAGAGAAAGAAAATGAAGATCAAAAGCTTATTCATCTGTTTTTCTTTTTCGTTGGTGTA
AAGCCAACACCCTGTCTAAAAAACATAAATTTCTTTAATCATTTTGCCTCTTTTCTCTGT
GCTTCAATTAATAAAAAATGGAAAGAATCTAATAGAGTGGTACAGCACTGTTATTTTTCA
AAGATGTGTTGtacgtagataagtagcatggcgggttaatcattaactacaaggaacccc
tagtgatggagttggccactccctctctgcgcgctcgctcgctcactgaggccgggcgac
caaaggtcgcccgacgcccgggctttgcccgggcggcctcagtgagcgagcgagcgcgca
gctggcgtaatagcgaagaggcccgcaccgatcgggtcaccaagcaggaagtcaaagact
ttttccggtgggcaaaggatcacgtggttgaggtggagcatgaattctacgtcaaaaagg
gtggagccaagaaaagacccgcccccagtgacgcagatataagtgagcccaaacgggtgc
gcgagtcagttgcgcagccatcgacgtcagacgcggaagcttcgatcaactacgcagaca
ggtaccaaaacaaatgttctcgtcacgtgggcatgaatctgatgctgtttccctgcagac
aatgcgagagaatgaatcagaattcaaatatctgcttcactcacggacagaaagactgtt
tagagtgctttcccgtgtcagaatctcaacccgtttctgtcgtcaaaaaggcgtatcaga
aactgtgctacattcatcatatcatgggaaaggtgccagacgcttgcactgcctgcgatc
tggtcaatgtggatttggatgactgcatctttgaacaataaatgatttaaatcaggtatg
gctgctgacggttatcttccagattggctcgaggacaacctttctgaaggcattcgagag
tggtgggcgctgcaacctggagcccctaaacccaaggcaaatcaacaacatcaggacaac
gctcggggtcttgtgcttccgggttacaaatacctcggacccggcaacggactcgacaag
ggggaacccgtcaacgcagcggacgcggcagccctcgagcacgacaaggcctacgaccag
cagctcaaggccggtgacaacccctacctcaagtacaaccacgccgacgccgagttccag
gagcggctcaaagaagatacgtcttttgggggcaacctcgggcgagcagtcttccaggcc
aaaaagaggcttcttgaacctcttggtctggttgaggaagcggctaagacggctcctgga
aagaagaggcctgtagatcagtctcctcaggaaccggactcatcatctggtgttggcaaa
tcgggcaaacagcctgccagaaaaagactaaatttcggtcagactggcgactcagagtca
gtcccagaccctcaacctctcggagaaccaccagcagcccccacaagtttgggatctaat
acaatggcttcaggcggtggcgcaccaatggcagacaataacgagggtgccgatggagtg
ggtaattcctcaggaaattggcattgcgattcccaatggctgggcgacagagtcatcacc
accagcaccagaacctgggccctgcccacttacaacaaccatctctacaagcaaatctcc
agccaatcaggagcttcaaacgacaaccactactttggctacagcaccccttgggggtat
tttgactttaacagattccactgccacttctcaccacgtgactggcagcgactcattaac
aacaactggggattccggcccaagaaactcagcttcaagctcttcaacatccaagttaaa
gaggtcacgcagaacgatggcacgacgactattgccaataaccttaccagcacggttcaa
gtgtttacggactcggagtatcagctcccgtacgtgctcgggtcggcgcaccaaggctgt
ctcccgccgtttccagcggacgtcttcatggtccctcagtatggatacctcaccctgaac
aacggaagtcaagcggtgggacgctcatccttttactgcctggagtacttcccttcgcag
atgctaaggactggaaataacttccaattcagctataccttcgaggatgtaccttttcac
agcagctacgctcacagccagagtttggatcgcttgatgaatcctcttattgatcagtat
ctgtactacctgaacagaacgcaaggaacaacctctggaacaaccaaccaatcacggctg
ctttttagccaggctgggcctcagtctatgtctttgcaggccagaaattggctacctggg
ccctgctaccggcaacagagactttcaaagactgctaacgacaacaacaacagtaacttt
ccttggacagcggccagcaaatatcatctcaatggccgcgactcgctggtgaatccagga
ccagctatggccagtcacaaggacgatgaagaaaaatttttccctatgcacggcaatcta
atatttggcaaagaagggacaacggcaagtaacgcagaattagataatgtaatgattacg
gatgaagaagagattcgtaccaccaatcctgtggcaacagagcagtatggaactgtggca
aataacttgcagagctcaaatacagctcccacgactagaactgtcaatgatcagggggcc
ttacctggcatggtgtggcaagatcgtgacgtgtaccttcaaggacctatctgggcaaag
attcctcacacggatggacactttcatccttctcctctgatgggaggctttggactgaaa
catccgcctcctcaaatcatgatcaaaaatactccggtaccggcaaatcctccgacgact
ttcagcccggccaagtttgcttcatttatcactcagtactccactggacaggtcagcgtg
gaaattgagtgggagctacagaaagaaaacagcaaacgttggaatccagagattcagtac
acttccaactacaacaagtctgttaatgtggactttactgtagacactaatggtgtttat
agtgaacctcgccccattggcacccgttaccttacccgtcccctgtaattgcttgttaat
caataaaccgtttaattcgtttcagttgaactttggtctctgcgtatttctttcttatct
agtttccatatgcatgtagataagtagcatggcgggttaatcattaactaaccggtacct
ctagaactatagctagcatgcgcaaatttaaagcgctgatatcgatcgcgcgcagatctg
tcatgatgatcattgcaattggatccatatatagggcccgggttataattacctcaggtc
gacgtcccatggccattcgaattcgtaatcatggtcatagctgtttcctgtgtgaaattg
ttatccgctcacaattccacacaacatacgagccggaagcataaagtgtaaagcctgggg
tgcctaatgagtgagctaactcacattaattgcgttgcgctcactgcccgctttccagtc
gggaaacctgtcgtgccagctgcattaatgaatcggccaacgcgcggggagaggcggttt
gcgtattgggcgctcttccgcttcctcgctcactgactcgctgcgctcggtcgttcggct
gcggcgagcggtatcagctcactcaaaggcggtaatacggttatccacagaatcagggga
taacgcaggaaagaacatgtgagcaaaaggccagcaaaaggccaggaaccgtaaaaaggc
cgcgttgctggcgtttttccataggctccgcccccctgacgagcatcacaaaaatcgacg
ctcaagtcagaggtggcgaaacccgacaggactataaagataccaggcgtttccccctgg
aagctccctcgtgcgctctcctgttccgaccctgccgcttaccggatacctgtccgcctt
tctcccttcgggaagcgtggcgctttctcatagctcacgctgtaggtatctcagttcggt
gtaggtcgttcgctccaagctgggctgtgtgcacgaaccccccgttcagcccgaccgctg
cgccttatccggtaactatcgtcttgagtccaacccggtaagacacgacttatcgccact
ggcagcagccactggtaacaggattagcagagcgaggtatgtaggcggtgctacagagtt
cttgaagtggtggcctaactacggctacactagaagaacagtatttggtatctgcgctct
gctgaagccagttaccttcggaaaaagagttggtagctcttgatccggcaaacaaaccac
cgctggtagcggtggtttttttgtttgcaagcagcagattacgcgcagaaaaaaaggatc
tcaagaagatcctttgatcttttctacggggtctgacgctcagtggaacgaaaactcacg
ttaagggattttggtcatgagattatcaaaaaggatcttcacctagatccttttaaatta
aaaatgaagttttaaatcaatctaaagtatatatgagtaaacttggtctgacagttagaa
aaactcatcgagcatcaaatgaaactgcaatttattcatatcaggattatcaataccata
tttttgaaaaagccgtttctgtaatgaaggagaaaactcaccgaggcagttccataggat
ggcaagatcctggtatcggtctgcgattccgactcgtccaacatcaatacaacctattaa
tttcccctcgtcaaaaataaggttatcaagtgagaaatcaccatgagtgacgactgaatc
cggtgagaatggcaaaagtttatgcatttctttccagacttgttcaacaggccagccatt
acgctcgtcatcaaaatcactcgcatcaaccaaaccgttattcattcgtgattgcgcctg
agcgagacgaaatacgcgatcgctgttaaaaggacaattacaaacaggaatcgaatgcaa
ccggcgcaggaacactgccagcgcatcaacaatattttcacctgaatcaggatattcttc
taatacctggaatgctgtttttccggggatcgcagtggtgagtaaccatgcatcatcagg
agtacggataaaatgcttgatggtcggaagaggcataaattccgtcagccagtttagtct
gaccatctcatctgtaacatcattggcaacgctacctttgccatgtttcagaaacaactc
tggcgcatcgggcttcccatacaagcgatagattgtcgcacctgattgcccgacattatc
gcgagcccatttatacccatataaatcagcatccatgttggaatttaatcgcggcctcga
cgtttcccgttgaatatggctcatactcttcctttttcaatattattgaagcatttatca
gggttattgtctcatgagcggatacatatttgaatgtatttagaaaaataaacaaatagg
ggttccgcgcacatttccccgaaaagtgccacctgacgtctaagaaaccattattatcat
gacattaacctataaaaataggcgtatcacgaggccctttcgtctcgcgcgtttcggtga
tgacggtgaaaacctctgacacatgcagctcccggagacggtcacagcttgtctgtaagc
ggatgccgggagcagacaagcccgtcagggcgcgtcagcgggtgttggcgggtgtcgggg
ctggcttaactatgcggcatcagagcagattgtactgagagtgcaccataaaattgtaaa
cgttaatattttgttaaaattcgcgttaaatttttgttaaatcagctcattttttaacca
ataggccgaaatcggcaaaatcccttataaatcaaaagaatagcccgagatagggttgag
tgttgttccagtttggaacaagagtccactattaaagaacgtggactccaacgtcaaagg
gcgaaaaaccgtctatcagggcgatggcccactacgtgaaccatcacccaaatcaagttt
tttggggtcgaggtgccgtaaagcactaaatcggaaccctaaagggagcccccgatttag
agcttgacggggaaagccggcgaacgtggcgagaaaggaagggaagaaagcgaaaggagc
gggcgctagggcgctggcaagtgtagcggtcacgctgcgcgtaaccaccacacccgccgc
gcttaatgcgccgctacagggcgcgtactatggttgctttgacgtatgcggtgtgaaata
ccgcacagatgcgtaaggagaaaataccgcatcaggcgccattcgccattcaggctgcgc
aactgttgggaagggcgatcggtgcgggcctcttcgctattacgccagctggcgaaaggg
ggatgtgctgcaaggcgattaagttgggtaacgccagggttttcccagtcacgacgttgt
aaaacgacggccaacatgttctttcctgcgttatcccctgattctgtggataaccgtatt
accgcctttgagtgagctgataccgctcgccgcagccgaacgaccgagcgcagcgagtca
gtgagcgaggaagcggaagagcgcccaatacgcaaaccgcctctccccgcgcgttggc
GR-FAH-co2_1.6/1.0_kb
(SEQ ID NO. 24)
cgattcattaatgcagctgcgcgctcgctcgctcactgaggccgcccgggcaaagcccgg
gcgtcgggcgacctttggtcgcccggcctcagtgagcgagcgagcgcgcagagagggagt
ggccaactccatcactaggggttccttgtagttaatgattaacccgccatgctacttatc
tacgtaTTTCTAGATGTAAATAATTATTTTAAGTTTGCCCTATGGTGGCCCCACACATGA
GACAAACCCCCAAGATGTGACTTTTGAGAATGAGACTTGGATAAAAAACATGTAGAAATG
CAAGCCCTGAAGCTCAACTCCCTATTGCTATCACAGGGGTTATAATTGCATAAAATTTAG
CTATAGAAAGTTGCTGTCATCTCTTGTGGGCTGTAATCATCGTCTAGGCTTAAGAGTAAT
ATTGCAAAACCTGTCATGCCCACACAAATCTCTCCCTGGCATTGTTGTCTTTGCAGATGT
CAGTGAAAGAGAACCAGCAGCTCCCATGAGTTTGGATAGCCTTATTTTCTATAGCCTCCC
CACTATTAGCTTTGAAGGGAGCAAAGTTTAAGAACCAAATATAAAGTTTCTCATCTTTAT
AGATGAGAAAAATTTTAAATAAAGTCCAAGATAATTAAATTTTTAAGGATCATTTTTAGC
TCTTTAATAGCAATAAAACTCAATATGACATAATATGGCACTTCCAAAATCTGAATAATA
TATAATTGCAATGACATACTTCTTTTCAGAGATTTACTGAAAAGAAATTTGTTGACACTA
CATAACGTGATGAGTGGTTTATACTGATTGTTTCAGTTGGTCTTCCCACCAACTCCATGA
AAGTGGATTTTATTATCCTCATCATGCAGATGAGAATATTGAGACTTATAGCGGTATGCC
TGAGCCCCAAAGTACTCAGAGTTGCCTGGCTCCAAGATTTATAATCTTAAATGATGGGAC
TACCATCCTTACTCTCTCCATTTTTCTATACGTGAGTAATGTTTTTTCTGTTTTTTTTTT
TTCTTTTTCCATTCAAACTCAGTGCACTTGTTGAGCTCGTGAAACACAAGCCCAAGGCAA
CAAAAGAGCAACTGAAAGCTGTTATGGATGATTTCGCAGCTTTTGTAGAGAAGTGCTGCA
AGGCTGACGATAAGGAGACCTGCTTTGCCGAGGAGGTACTACAGTTCTCTTCATTTTAAT
ATGTCCAGTATTCATTTTTGCATGTTTGGTTAGGCTAGGGCTTAGGGATTTATATATCAA
AGGAGGCTTTGTACATGTGGGACAGGGATCTTATTTTACAAACAATTGTCTTACAAAATG
AATAAAACAGCACTTTGTTTTTATCTCCTGCTCTATTGTGCCATACTGTTAAATGTTTAT
AATGCCTGTTCTGTTTCCAAATTTGTGATGCTTATGAATATTAATAGGAATATTTGTAAG
GCCTGAAATATTTTGATCATGAAATCAAAACATTAATTTATTTAAACATTTACTTGAAAT
GTGGTGGTTTGTGATTTAGTTGATTTTATAGGCTAGTGGGAGAATTTACATTCAAATGTC
TAAATCACTTAAAATTGCCCTTTATGGCCTGACAGTAACTTTTTTTTATTCATTTGGGGA
CAACTATGTCCGTGAGCTTCCGTCCAGAGATTATAGTAGTAAATTGTAATTAAAGGATAT
GATGCACGTGAAATCACTTTGCAATCATCAATAGCTTCATAAATGTTAATTTTGTATCCT
AATAGTAATGCTAATATTTTCCTAACATCTGTCATGTCTTTGTGTTCAGGGTAAAAAACT
TGTTGCTGCAAGTCAAGCTGCCTTAGGCTTAGGAAGCGGCGCCACCAATTTCAGCCTGCT
GAAACAGGCCGGCGACGTGGAAGAGAACCCTGGCCCTTCCTTCATCCCAGTGGCTGAAGA
TTCTGACTTCCCCATTCACAACCTCCCTTATGGAGTCTTTAGCACAAGAGGAGACCCAAG
ACCTAGAATTGGCGTGGCTATTGGGGATCAGATCCTGGACCTGAGCATTATCAAACATCT
CTTTACAGGCCCAGTCCTCAGCAAGCACCAAGATGTGTTCAATCAACCCACACTGAACTC
ATTTATGGGGCTGGGCCAAGCTGCCTGGAAGGAGGCAAGAGTGTTTCTCCAGAACCTCCT
GTCAGTGAGCCAGGCCAGGCTGAGAGATGACACAGAGCTGAGGAAGTGTGCCTTTATCTC
ACAAGCCAGCGCCACAATGCACCTGCCAGCAACCATCGGGGACTACACAGACTTTTACAG
CAGCAGGCAGCACGCCACTAATGTGGGCATCATGTTTAGAGACAAAGAGAATGCCCTCAT
GCCTAACTGGCTGCATCTGCCAGTGGGCTACCATGGCAGAGCCAGCAGCGTGGTGGTGTC
TGGCACCCCTATCAGGAGGCCCATGGGCCAGATGAAGCCTGATGACAGCAAGCCACCTGT
GTATGGGGCATGTAAGCTGCTGGACATGGAGCTGGAAATGGCTTTCTTTGTGGGGCCTGG
GAACAGGCTGGGCGAACCAATTCCCATCAGCAAAGCTCATGAACACATTTTTGGGATGGT
GCTCATGAATGACTGGTCTGCCAGAGACATCCAGAAGTGGGAGTATGTCCCCCTGGGACC
ATTCCTGGGCAAGAGCTTTGGGACCACTGTGTCACCATGGGTGGTGCCCATGGATGCCCT
GATGCCATTTGCTGTGCCTAATCCCAAACAAGATCCAAGGCCACTGCCCTACCTCTGCCA
CGATGAGCCCTACACATTTGATATCAACCTCTCTGTGAATCTGAAGGGAGAAGGAATGAG
CCAAGCTGCTACCATTTGCAAAAGCAACTTTAAGTATATGTATTGGACCATGCTGCAGCA
ACTCACCCACCACAGCGTGAATGGCTGTAACCTGAGGCCTGGCGACCTGCTGGCCTCTGG
CACCATCAGCGGCCCTGAACCTGAGAACTTTGGCAGCATGCTGGAGCTGAGCTGGAAGGG
AACCAAGCCCATTGACCTGGGCAATGGACAGACCAGGAAATTCCTCCTGGACGGGGACGA
GGTGATCATCACAGGCTATTGCCAGGGGGATGGGTATAGGATTGGCTTTGGCCAATGTGC
CGGCAAAGTGCTGCCTGCCCTGCTCCCTAGCTAACATCACATTTAAAAGCATCTCAGGTA
ACTATATTTTGAATTTTTTAAAAAAGTAACTATAATAGTTATTATTAAAATAGCAAAGAT
TGACCATTTCCAAGAGCCATATAGACCAGCACCGACCACTATTCTAAACTATTTATGTAT
GTAAATATTAGCTTTTAAAATTCTCAAAATAGTTGCTGAGTTGGGAACCACTATTATTTC
TATTTTGTAGATGAGAAAATGAAGATAAACATCAAAGCATAGATTAAGTAATTTTCCAAA
GGGTCAAAATTCAAAATTGAAACCAAAGTTTCAGTGTTGCCCATTGTCCTGTTCTGACTT
ATATGATGCGGTACACAGAGCCATCCAAGTAAGTGATGGCTCAGCAGTGGAATACTCTGG
GAATTAGGCTGAACCACATGAAAGAGTGCTTTATAGGGCAAAAACAGTTGAATATCAGTG
ATTTCACATGGTTCAACCTAATAGTTCAACTCATCCTTTCCATTGGAGAATATGATGGAT
CTACCTTCTGTGAACTTTATAGTGAAGAATCTGCTATTACATTTCCAATTTGTCAACATG
CTGAGCTTTAATAGGACTTATCTTCTTATGACAACATTTATTGGTGTGTCCCCTTGCCTA
GCCCAACAGAAGAATTCAGCAGCCGTAAGTCTAGGACAGGCTTAAATTGTTTTCACTGGT
GTAAATTGCAGAAAGATGATCTAAGTAATTTGGCATTTATTTTAATAGGTTTGAAAAACA
CATGCCATTTTACAAATAAGACTTATATTTGTCCTTTTGTTTTTCAGCCTACCATGAGAA
TAAGAGAAAGAAAATGAAGATCAAAAGCTTATTCATCTGTTTTTCTTTTTCGTTGGTGTA
AAGCCAACACCCTGTCTAAAAAACATAAATTTCTTTAATCATTTTGCCTCTTTTCTCTGT
GCTTCAATTAATAAAAAATGGAAAGAATCTAATAGAGTGGTACAGCACTGTTATTTTTCA
AAGATGTGTTGtacgtagataagtagcatggcgggttaatcattaactacaaggaacccc
tagtgatggagttggccactccctctctgcgcgctcgctcgctcactgaggccgggcgac
caaaggtcgcccgacgcccgggctttgcccgggcggcctcagtgagcgagcgagcgcgca
gctggcgtaatagcgaagaggcccgcaccgatcgggtcaccaagcaggaagtcaaagact
ttttccggtgggcaaaggatcacgtggttgaggtggagcatgaattctacgtcaaaaagg
gtggagccaagaaaagacccgcccccagtgacgcagatataagtgagcccaaacgggtgc
gcgagtcagttgcgcagccatcgacgtcagacgcggaagcttcgatcaactacgcagaca
ggtaccaaaacaaatgttctcgtcacgtgggcatgaatctgatgctgtttccctgcagac
aatgcgagagaatgaatcagaattcaaatatctgcttcactcacggacagaaagactgtt
tagagtgctttcccgtgtcagaatctcaacccgtttctgtcgtcaaaaaggcgtatcaga
aactgtgctacattcatcatatcatgggaaaggtgccagacgcttgcactgcctgcgatc
tggtcaatgtggatttggatgactgcatctttgaacaataaatgatttaaatcaggtatg
gctgctgacggttatcttccagattggctcgaggacaacctttctgaaggcattcgagag
tggtgggcgctgcaacctggagcccctaaacccaaggcaaatcaacaacatcaggacaac
gctcggggtcttgtgcttccgggttacaaatacctcggacccggcaacggactcgacaag
ggggaacccgtcaacgcagcggacgcggcagccctcgagcacgacaaggcctacgaccag
cagctcaaggccggtgacaacccctacctcaagtacaaccacgccgacgccgagttccag
gagcggctcaaagaagatacgtcttttgggggcaacctcgggcgagcagtcttccaggcc
aaaaagaggcttcttgaacctcttggtctggttgaggaagcggctaagacggctcctgga
aagaagaggcctgtagatcagtctcctcaggaaccggactcatcatctggtgttggcaaa
tcgggcaaacagcctgccagaaaaagactaaatttcggtcagactggcgactcagagtca
gtcccagaccctcaacctctcggagaaccaccagcagcccccacaagtttgggatctaat
acaatggcttcaggcggtggcgcaccaatggcagacaataacgagggtgccgatggagtg
ggtaattcctcaggaaattggcattgcgattcccaatggctgggcgacagagtcatcacc
accagcaccagaacctgggccctgcccacttacaacaaccatctctacaagcaaatctcc
agccaatcaggagcttcaaacgacaaccactactttggctacagcaccccttgggggtat
tttgactttaacagattccactgccacttctcaccacgtgactggcagcgactcattaac
aacaactggggattccggcccaagaaactcagcttcaagctcttcaacatccaagttaaa
gaggtcacgcagaacgatggcacgacgactattgccaataaccttaccagcacggttcaa
gtgtttacggactcggagtatcagctcccgtacgtgctcgggtcggcgcaccaaggctgt
ctcccgccgtttccagcggacgtcttcatggtccctcagtatggatacctcaccctgaac
aacggaagtcaagcggtgggacgctcatccttttactgcctggagtacttcccttcgcag
atgctaaggactggaaataacttccaattcagctataccttcgaggatgtaccttttcac
agcagctacgctcacagccagagtttggatcgcttgatgaatcctcttattgatcagtat
ctgtactacctgaacagaacgcaaggaacaacctctggaacaaccaaccaatcacggctg
ctttttagccaggctgggcctcagtctatgtctttgcaggccagaaattggctacctggg
ccctgctaccggcaacagagactttcaaagactgctaacgacaacaacaacagtaacttt
ccttggacagcggccagcaaatatcatctcaatggccgcgactcgctggtgaatccagga
ccagctatggccagtcacaaggacgatgaagaaaaatttttccctatgcacggcaatcta
atatttggcaaagaagggacaacggcaagtaacgcagaattagataatgtaatgattacg
gatgaagaagagattcgtaccaccaatcctgtggcaacagagcagtatggaactgtggca
aataacttgcagagctcaaatacagctcccacgactagaactgtcaatgatcagggggcc
ttacctggcatggtgtggcaagatcgtgacgtgtaccttcaaggacctatctgggcaaag
attcctcacacggatggacactttcatccttctcctctgatgggaggctttggactgaaa
catccgcctcctcaaatcatgatcaaaaatactccggtaccggcaaatcctccgacgact
ttcagcccggccaagtttgcttcatttatcactcagtactccactggacaggtcagcgtg
gaaattgagtgggagctacagaaagaaaacagcaaacgttggaatccagagattcagtac
acttccaactacaacaagtctgttaatgtggactttactgtagacactaatggtgtttat
agtgaacctcgccccattggcacccgttaccttacccgtcccctgtaattgcttgttaat
caataaaccgtttaattcgtttcagttgaactttggtctctgcgtatttctttcttatct
agtttccatatgcatgtagataagtagcatggcgggttaatcattaactaaccggtacct
ctagaactatagctagcatgcgcaaatttaaagcgctgatatcgatcgcgcgcagatctg
tcatgatgatcattgcaattggatccatatatagggcccgggttataattacctcaggtc
gacgtcccatggccattcgaattcgtaatcatggtcatagctgtttcctgtgtgaaattg
ttatccgctcacaattccacacaacatacgagccggaagcataaagtgtaaagcctgggg
tgcctaatgagtgagctaactcacattaattgcgttgcgctcactgcccgctttccagtc
gggaaacctgtcgtgccagctgcattaatgaatcggccaacgcgcggggagaggcggttt
gcgtattgggcgctcttccgcttcctcgctcactgactcgctgcgctcggtcgttcggct
gcggcgagcggtatcagctcactcaaaggcggtaatacggttatccacagaatcagggga
taacgcaggaaagaacatgtgagcaaaaggccagcaaaaggccaggaaccgtaaaaaggc
cgcgttgctggcgtttttccataggctccgcccccctgacgagcatcacaaaaatcgacg
ctcaagtcagaggtggcgaaacccgacaggactataaagataccaggcgtttccccctgg
aagctccctcgtgcgctctcctgttccgaccctgccgcttaccggatacctgtccgcctt
tctcccttcgggaagcgtggcgctttctcatagctcacgctgtaggtatctcagttcggt
gtaggtcgttcgctccaagctgggctgtgtgcacgaaccccccgttcagcccgaccgctg
cgccttatccggtaactatcgtcttgagtccaacccggtaagacacgacttatcgccact
ggcagcagccactggtaacaggattagcagagcgaggtatgtaggcggtgctacagagtt
cttgaagtggtggcctaactacggctacactagaagaacagtatttggtatctgcgctct
gctgaagccagttaccttcggaaaaagagttggtagctcttgatccggcaaacaaaccac
cgctggtagcggtggtttttttgtttgcaagcagcagattacgcgcagaaaaaaaggatc
tcaagaagatcctttgatcttttctacggggtctgacgctcagtggaacgaaaactcacg
ttaagggattttggtcatgagattatcaaaaaggatcttcacctagatccttttaaatta
aaaatgaagttttaaatcaatctaaagtatatatgagtaaacttggtctgacagttagaa
aaactcatcgagcatcaaatgaaactgcaatttattcatatcaggattatcaataccata
tttttgaaaaagccgtttctgtaatgaaggagaaaactcaccgaggcagttccataggat
ggcaagatcctggtatcggtctgcgattccgactcgtccaacatcaatacaacctattaa
tttcccctcgtcaaaaataaggttatcaagtgagaaatcaccatgagtgacgactgaatc
cggtgagaatggcaaaagtttatgcatttctttccagacttgttcaacaggccagccatt
acgctcgtcatcaaaatcactcgcatcaaccaaaccgttattcattcgtgattgcgcctg
agcgagacgaaatacgcgatcgctgttaaaaggacaattacaaacaggaatcgaatgcaa
ccggcgcaggaacactgccagcgcatcaacaatattttcacctgaatcaggatattcttc
taatacctggaatgctgtttttccggggatcgcagtggtgagtaaccatgcatcatcagg
agtacggataaaatgcttgatggtcggaagaggcataaattccgtcagccagtttagtct
gaccatctcatctgtaacatcattggcaacgctacctttgccatgtttcagaaacaactc
tggcgcatcgggcttcccatacaagcgatagattgtcgcacctgattgcccgacattatc
gcgagcccatttatacccatataaatcagcatccatgttggaatttaatcgcggcctcga
cgtttcccgttgaatatggctcatactcttcctttttcaatattattgaagcatttatca
gggttattgtctcatgagcggatacatatttgaatgtatttagaaaaataaacaaatagg
ggttccgcgcacatttccccgaaaagtgccacctgacgtctaagaaaccattattatcat
gacattaacctataaaaataggcgtatcacgaggccctttcgtctcgcgcgtttcggtga
tgacggtgaaaacctctgacacatgcagctcccggagacggtcacagcttgtctgtaagc
ggatgccgggagcagacaagcccgtcagggcgcgtcagcgggtgttggcgggtgtcgggg
ctggcttaactatgcggcatcagagcagattgtactgagagtgcaccataaaattgtaaa
cgttaatattttgttaaaattcgcgttaaatttttgttaaatcagctcattttttaacca
ataggccgaaatcggcaaaatcccttataaatcaaaagaatagcccgagatagggttgag
tgttgttccagtttggaacaagagtccactattaaagaacgtggactccaacgtcaaagg
gcgaaaaaccgtctatcagggcgatggcccactacgtgaaccatcacccaaatcaagttt
tttggggtcgaggtgccgtaaagcactaaatcggaaccctaaagggagcccccgatttag
agcttgacggggaaagccggcgaacgtggcgagaaaggaagggaagaaagcgaaaggagc
gggcgctagggcgctggcaagtgtagcggtcacgctgcgcgtaaccaccacacccgccgc
gcttaatgcgccgctacagggcgcgtactatggttgctttgacgtatgcggtgtgaaata
ccgcacagatgcgtaaggagaaaataccgcatcaggcgccattcgccattcaggctgcgc
aactgttgggaagggcgatcggtgcgggcctcttcgctattacgccagctggcgaaaggg
ggatgtgctgcaaggcgattaagttgggtaacgccagggttttcccagtcacgacgttgt
aaaacgacggccaacatgttctttcctgcgttatcccctgattctgtggataaccgtatt
accgcctttgagtgagctgataccgctcgccgcagccgaacgaccgagcgcagcgagtca
gtgagcgaggaagcggaagagcgcccaatacgcaaaccgcctctccccgcgcgttggc
GR-FAH-co1_1.6/1.0_kb
(SEQ ID NO. 25)
cgattcattaatgcagctgcgcgctcgctcgctcactgaggccgcccgggcaaagcccgg
gcgtcgggcgacctttggtcgcccggcctcagtgagcgagcgagcgcgcagagagggagt
ggccaactccatcactaggggttccttgtagttaatgattaacccgccatgctacttatc
tacgtaTTTCTAGATGTAAATAATTATTTTAAGTTTGCCCTATGGTGGCCCCACACATGA
GACAAACCCCCAAGATGTGACTTTTGAGAATGAGACTTGGATAAAAAACATGTAGAAATG
CAAGCCCTGAAGCTCAACTCCCTATTGCTATCACAGGGGTTATAATTGCATAAAATTTAG
CTATAGAAAGTTGCTGTCATCTCTTGTGGGCTGTAATCATCGTCTAGGCTTAAGAGTAAT
ATTGCAAAACCTGTCATGCCCACACAAATCTCTCCCTGGCATTGTTGTCTTTGCAGATGT
CAGTGAAAGAGAACCAGCAGCTCCCATGAGTTTGGATAGCCTTATTTTCTATAGCCTCCC
CACTATTAGCTTTGAAGGGAGCAAAGTTTAAGAACCAAATATAAAGTTTCTCATCTTTAT
AGATGAGAAAAATTTTAAATAAAGTCCAAGATAATTAAATTTTTAAGGATCATTTTTAGC
TCTTTAATAGCAATAAAACTCAATATGACATAATATGGCACTTCCAAAATCTGAATAATA
TATAATTGCAATGACATACTTCTTTTCAGAGATTTACTGAAAAGAAATTTGTTGACACTA
CATAACGTGATGAGTGGTTTATACTGATTGTTTCAGTTGGTCTTCCCACCAACTCCATGA
AAGTGGATTTTATTATCCTCATCATGCAGATGAGAATATTGAGACTTATAGCGGTATGCC
TGAGCCCCAAAGTACTCAGAGTTGCCTGGCTCCAAGATTTATAATCTTAAATGATGGGAC
TACCATCCTTACTCTCTCCATTTTTCTATACGTGAGTAATGTTTTTTCTGTTTTTTTTTT
TTCTTTTTCCATTCAAACTCAGTGCACTTGTTGAGCTCGTGAAACACAAGCCCAAGGCAA
CAAAAGAGCAACTGAAAGCTGTTATGGATGATTTCGCAGCTTTTGTAGAGAAGTGCTGCA
AGGCTGACGATAAGGAGACCTGCTTTGCCGAGGAGGTACTACAGTTCTCTTCATTTTAAT
ATGTCCAGTATTCATTTTTGCATGTTTGGTTAGGCTAGGGCTTAGGGATTTATATATCAA
AGGAGGCTTTGTACATGTGGGACAGGGATCTTATTTTACAAACAATTGTCTTACAAAATG
AATAAAACAGCACTTTGTTTTTATCTCCTGCTCTATTGTGCCATACTGTTAAATGTTTAT
AATGCCTGTTCTGTTTCCAAATTTGTGATGCTTATGAATATTAATAGGAATATTTGTAAG
GCCTGAAATATTTTGATCATGAAATCAAAACATTAATTTATTTAAACATTTACTTGAAAT
GTGGTGGTTTGTGATTTAGTTGATTTTATAGGCTAGTGGGAGAATTTACATTCAAATGTC
TAAATCACTTAAAATTGCCCTTTATGGCCTGACAGTAACTTTTTTTTATTCATTTGGGGA
CAACTATGTCCGTGAGCTTCCGTCCAGAGATTATAGTAGTAAATTGTAATTAAAGGATAT
GATGCACGTGAAATCACTTTGCAATCATCAATAGCTTCATAAATGTTAATTTTGTATCCT
AATAGTAATGCTAATATTTTCCTAACATCTGTCATGTCTTTGTGTTCAGGGTAAAAAACT
TGTTGCTGCAAGTCAAGCTGCCTTAGGCTTAGGAAGCGGCGCCACCAATTTCAGCCTGCT
GAAACAGGCCGGCGACGTGGAAGAGAACCCTGGCCCTTCCTTTATTCCTGTGGCCGAAGA
TAGCGACTTCCCTATCCATAACCTCCCATATGGCGTCTTCTCAACCAGGGGCGACCCCAG
GCCCAGGATTGGGGTGGCAATTGGAGACCAGATCCTGGACCTCAGCATCATTAAGCACCT
CTTTACAGGACCTGTGCTGAGCAAGCACCAGGATGTGTTCAACCAGCCTACCCTGAATAG
CTTTATGGGACTGGGCCAGGCAGCATGGAAAGAAGCCAGAGTGTTCCTCCAGAATCTGCT
GAGCGTGAGCCAGGCCAGGCTCAGGGATGACACAGAACTGAGGAAATGTGCCTTCATCTC
ACAGGCATCAGCCACAATGCACCTCCCTGCCACAATTGGGGACTACACTGACTTCTACAG
CAGCAGACAGCATGCCACTAATGTGGGGATTATGTTTAGAGACAAGGAAAATGCCCTCAT
GCCAAATTGGCTGCACCTGCCTGTGGGCTACCACGGCAGAGCCTCATCAGTGGTGGTGTC
AGGCACACCAATTAGGAGGCCAATGGGACAGATGAAGCCTGACGACTCAAAGCCACCAGT
CTATGGCGCCTGCAAACTGCTGGACATGGAGCTGGAGATGGCTTTCTTTGTGGGGCCTGG
CAACAGGCTGGGAGAACCAATCCCTATTTCAAAGGCCCACGAGCACATTTTTGGCATGGT
GCTCATGAATGACTGGTCTGCCAGAGATATCCAGAAGTGGGAATACGTGCCCCTGGGCCC
TTTTCTGGGCAAGAGCTTTGGCACCACAGTGTCACCTTGGGTGGTCCCAATGGATGCCCT
GATGCCCTTTGCCGTGCCCAACCCCAAGCAGGATCCCAGACCACTGCCCTACCTGTGCCA
CGATGAGCCCTATACCTTTGACATCAACCTGTCAGTCAACCTGAAGGGGGAGGGCATGAG
CCAGGCCGCCACTATTTGCAAGAGCAACTTTAAATATATGTACTGGACTATGCTCCAACA
GCTCACTCACCACTCAGTGAATGGCTGCAATCTGAGGCCTGGCGACCTCCTGGCTAGCGG
CACTATCTCTGGCCCTGAACCTGAGAACTTTGGGAGCATGCTGGAGCTGTCATGGAAAGG
GACAAAGCCTATTGACCTGGGGAATGGCCAGACAAGGAAGTTTCTCCTGGATGGCGATGA
GGTCATCATCACTGGGTACTGCCAGGGCGATGGCTACAGGATTGGATTTGGACAGTGTGC
TGGCAAAGTGCTCCCAGCTCTGCTGCCCTCATAACATCACATTTAAAAGCATCTCAGGTA
ACTATATTTTGAATTTTTTAAAAAAGTAACTATAATAGTTATTATTAAAATAGCAAAGAT
TGACCATTTCCAAGAGCCATATAGACCAGCACCGACCACTATTCTAAACTATTTATGTAT
GTAAATATTAGCTTTTAAAATTCTCAAAATAGTTGCTGAGTTGGGAACCACTATTATTTC
TATTTTGTAGATGAGAAAATGAAGATAAACATCAAAGCATAGATTAAGTAATTTTCCAAA
GGGTCAAAATTCAAAATTGAAACCAAAGTTTCAGTGTTGCCCATTGTCCTGTTCTGACTT
ATATGATGCGGTACACAGAGCCATCCAAGTAAGTGATGGCTCAGCAGTGGAATACTCTGG
GAATTAGGCTGAACCACATGAAAGAGTGCTTTATAGGGCAAAAACAGTTGAATATCAGTG
ATTTCACATGGTTCAACCTAATAGTTCAACTCATCCTTTCCATTGGAGAATATGATGGAT
CTACCTTCTGTGAACTTTATAGTGAAGAATCTGCTATTACATTTCCAATTTGTCAACATG
CTGAGCTTTAATAGGACTTATCTTCTTATGACAACATTTATTGGTGTGTCCCCTTGCCTA
GCCCAACAGAAGAATTCAGCAGCCGTAAGTCTAGGACAGGCTTAAATTGTTTTCACTGGT
GTAAATTGCAGAAAGATGATCTAAGTAATTTGGCATTTATTTTAATAGGTTTGAAAAACA
CATGCCATTTTACAAATAAGACTTATATTTGTCCTTTTGTTTTTCAGCCTACCATGAGAA
TAAGAGAAAGAAAATGAAGATCAAAAGCTTATTCATCTGTTTTTCTTTTTCGTTGGTGTA
AAGCCAACACCCTGTCTAAAAAACATAAATTTCTTTAATCATTTTGCCTCTTTTCTCTGT
GCTTCAATTAATAAAAAATGGAAAGAATCTAATAGAGTGGTACAGCACTGTTATTTTTCA
AAGATGTGTTGtacgtagataagtagcatggcgggttaatcattaactacaaggaacccc
tagtgatggagttggccactccctctctgcgcgctcgctcgctcactgaggccgggcgac
caaaggtcgcccgacgcccgggctttgcccgggcggcctcagtgagcgagcgagcgcgca
gctggcgtaatagcgaagaggcccgcaccgatcgggtcaccaagcaggaagtcaaagact
ttttccggtgggcaaaggatcacgtggttgaggtggagcatgaattctacgtcaaaaagg
gtggagccaagaaaagacccgcccccagtgacgcagatataagtgagcccaaacgggtgc
gcgagtcagttgcgcagccatcgacgtcagacgcggaagcttcgatcaactacgcagaca
ggtaccaaaacaaatgttctcgtcacgtgggcatgaatctgatgctgtttccctgcagac
aatgcgagagaatgaatcagaattcaaatatctgcttcactcacggacagaaagactgtt
tagagtgctttcccgtgtcagaatctcaacccgtttctgtcgtcaaaaaggcgtatcaga
aactgtgctacattcatcatatcatgggaaaggtgccagacgcttgcactgcctgcgatc
tggtcaatgtggatttggatgactgcatctttgaacaataaatgatttaaatcaggtatg
gctgctgacggttatcttccagattggctcgaggacaacctttctgaaggcattcgagag
tggtgggcgctgcaacctggagcccctaaacccaaggcaaatcaacaacatcaggacaac
gctcggggtcttgtgcttccgggttacaaatacctcggacccggcaacggactcgacaag
ggggaacccgtcaacgcagcggacgcggcagccctcgagcacgacaaggcctacgaccag
cagctcaaggccggtgacaacccctacctcaagtacaaccacgccgacgccgagttccag
gagcggctcaaagaagatacgtcttttgggggcaacctcgggcgagcagtcttccaggcc
aaaaagaggcttcttgaacctcttggtctggttgaggaagcggctaagacggctcctgga
aagaagaggcctgtagatcagtctcctcaggaaccggactcatcatctggtgttggcaaa
tcgggcaaacagcctgccagaaaaagactaaatttcggtcagactggcgactcagagtca
gtcccagaccctcaacctctcggagaaccaccagcagcccccacaagtttgggatctaat
acaatggcttcaggcggtggcgcaccaatggcagacaataacgagggtgccgatggagtg
ggtaattcctcaggaaattggcattgcgattcccaatggctgggcgacagagtcatcacc
accagcaccagaacctgggccctgcccacttacaacaaccatctctacaagcaaatctcc
agccaatcaggagcttcaaacgacaaccactactttggctacagcaccccttgggggtat
tttgactttaacagattccactgccacttctcaccacgtgactggcagcgactcattaac
aacaactggggattccggcccaagaaactcagcttcaagctcttcaacatccaagttaaa
gaggtcacgcagaacgatggcacgacgactattgccaataaccttaccagcacggttcaa
gtgtttacggactcggagtatcagctcccgtacgtgctcgggtcggcgcaccaaggctgt
ctcccgccgtttccagcggacgtcttcatggtccctcagtatggatacctcaccctgaac
aacggaagtcaagcggtgggacgctcatccttttactgcctggagtacttcccttcgcag
atgctaaggactggaaataacttccaattcagctataccttcgaggatgtaccttttcac
agcagctacgctcacagccagagtttggatcgcttgatgaatcctcttattgatcagtat
ctgtactacctgaacagaacgcaaggaacaacctctggaacaaccaaccaatcacggctg
ctttttagccaggctgggcctcagtctatgtctttgcaggccagaaattggctacctggg
ccctgctaccggcaacagagactttcaaagactgctaacgacaacaacaacagtaacttt
ccttggacagcggccagcaaatatcatctcaatggccgcgactcgctggtgaatccagga
ccagctatggccagtcacaaggacgatgaagaaaaatttttccctatgcacggcaatcta
atatttggcaaagaagggacaacggcaagtaacgcagaattagataatgtaatgattacg
gatgaagaagagattcgtaccaccaatcctgtggcaacagagcagtatggaactgtggca
aataacttgcagagctcaaatacagctcccacgactagaactgtcaatgatcagggggcc
ttacctggcatggtgtggcaagatcgtgacgtgtaccttcaaggacctatctgggcaaag
attcctcacacggatggacactttcatccttctcctctgatgggaggctttggactgaaa
catccgcctcctcaaatcatgatcaaaaatactccggtaccggcaaatcctccgacgact
ttcagcccggccaagtttgcttcatttatcactcagtactccactggacaggtcagcgtg
gaaattgagtgggagctacagaaagaaaacagcaaacgttggaatccagagattcagtac
acttccaactacaacaagtctgttaatgtggactttactgtagacactaatggtgtttat
agtgaacctcgccccattggcacccgttaccttacccgtcccctgtaattgcttgttaat
caataaaccgtttaattcgtttcagttgaactttggtctctgcgtatttctttcttatct
agtttccatatgcatgtagataagtagcatggcgggttaatcattaactaaccggtacct
ctagaactatagctagcatgcgcaaatttaaagcgctgatatcgatcgcgcgcagatctg
tcatgatgatcattgcaattggatccatatatagggcccgggttataattacctcaggtc
gacgtcccatggccattcgaattcgtaatcatggtcatagctgtttcctgtgtgaaattg
ttatccgctcacaattccacacaacatacgagccggaagcataaagtgtaaagcctgggg
tgcctaatgagtgagctaactcacattaattgcgttgcgctcactgcccgctttccagtc
gggaaacctgtcgtgccagctgcattaatgaatcggccaacgcgcggggagaggcggttt
gcgtattgggcgctcttccgcttcctcgctcactgactcgctgcgctcggtcgttcggct
gcggcgagcggtatcagctcactcaaaggcggtaatacggttatccacagaatcagggga
taacgcaggaaagaacatgtgagcaaaaggccagcaaaaggccaggaaccgtaaaaaggc
cgcgttgctggcgtttttccataggctccgcccccctgacgagcatcacaaaaatcgacg
ctcaagtcagaggtggcgaaacccgacaggactataaagataccaggcgtttccccctgg
aagctccctcgtgcgctctcctgttccgaccctgccgcttaccggatacctgtccgcctt
tctcccttcgggaagcgtggcgctttctcatagctcacgctgtaggtatctcagttcggt
gtaggtcgttcgctccaagctgggctgtgtgcacgaaccccccgttcagcccgaccgctg
cgccttatccggtaactatcgtcttgagtccaacccggtaagacacgacttatcgccact
ggcagcagccactggtaacaggattagcagagcgaggtatgtaggcggtgctacagagtt
cttgaagtggtggcctaactacggctacactagaagaacagtatttggtatctgcgctct
gctgaagccagttaccttcggaaaaagagttggtagctcttgatccggcaaacaaaccac
cgctggtagcggtggtttttttgtttgcaagcagcagattacgcgcagaaaaaaaggatc
tcaagaagatcctttgatcttttctacggggtctgacgctcagtggaacgaaaactcacg
ttaagggattttggtcatgagattatcaaaaaggatcttcacctagatccttttaaatta
aaaatgaagttttaaatcaatctaaagtatatatgagtaaacttggtctgacagttagaa
aaactcatcgagcatcaaatgaaactgcaatttattcatatcaggattatcaataccata
tttttgaaaaagccgtttctgtaatgaaggagaaaactcaccgaggcagttccataggat
ggcaagatcctggtatcggtctgcgattccgactcgtccaacatcaatacaacctattaa
tttcccctcgtcaaaaataaggttatcaagtgagaaatcaccatgagtgacgactgaatc
cggtgagaatggcaaaagtttatgcatttctttccagacttgttcaacaggccagccatt
acgctcgtcatcaaaatcactcgcatcaaccaaaccgttattcattcgtgattgcgcctg
agcgagacgaaatacgcgatcgctgttaaaaggacaattacaaacaggaatcgaatgcaa
ccggcgcaggaacactgccagcgcatcaacaatattttcacctgaatcaggatattcttc
taatacctggaatgctgtttttccggggatcgcagtggtgagtaaccatgcatcatcagg
agtacggataaaatgcttgatggtcggaagaggcataaattccgtcagccagtttagtct
gaccatctcatctgtaacatcattggcaacgctacctttgccatgtttcagaaacaactc
tggcgcatcgggcttcccatacaagcgatagattgtcgcacctgattgcccgacattatc
gcgagcccatttatacccatataaatcagcatccatgttggaatttaatcgcggcctcga
cgtttcccgttgaatatggctcatactcttcctttttcaatattattgaagcatttatca
gggttattgtctcatgagcggatacatatttgaatgtatttagaaaaataaacaaatagg
ggttccgcgcacatttccccgaaaagtgccacctgacgtctaagaaaccattattatcat
gacattaacctataaaaataggcgtatcacgaggccctttcgtctcgcgcgtttcggtga
tgacggtgaaaacctctgacacatgcagctcccggagacggtcacagcttgtctgtaagc
ggatgccgggagcagacaagcccgtcagggcgcgtcagcgggtgttggcgggtgtcgggg
ctggcttaactatgcggcatcagagcagattgtactgagagtgcaccataaaattgtaaa
cgttaatattttgttaaaattcgcgttaaatttttgttaaatcagctcattttttaacca
ataggccgaaatcggcaaaatcccttataaatcaaaagaatagcccgagatagggttgag
tgttgttccagtttggaacaagagtccactattaaagaacgtggactccaacgtcaaagg
gcgaaaaaccgtctatcagggcgatggcccactacgtgaaccatcacccaaatcaagttt
tttggggtcgaggtgccgtaaagcactaaatcggaaccctaaagggagcccccgatttag
agcttgacggggaaagccggcgaacgtggcgagaaaggaagggaagaaagcgaaaggagc
gggcgctagggcgctggcaagtgtagcggtcacgctgcgcgtaaccaccacacccgccgc
gcttaatgcgccgctacagggcgcgtactatggttgctttgacgtatgcggtgtgaaata
ccgcacagatgcgtaaggagaaaataccgcatcaggcgccattcgccattcaggctgcgc
aactgttgggaagggcgatcggtgcgggcctcttcgctattacgccagctggcgaaaggg
ggatgtgctgcaaggcgattaagttgggtaacgccagggttttcccagtcacgacgttgt
aaaacgacggccaacatgttctttcctgcgttatcccctgattctgtggataaccgtatt
accgcctttgagtgagctgataccgctcgccgcagccgaacgaccgagcgcagcgagtca
gtgagcgaggaagcggaagagcgcccaatacgcaaaccgcctctccccgcgcgttggc
GR-FAH-co1_1 kb
(SEQ ID NO. 26)
cgattcattaatgcagctgcgcgctcgctcgctcactgaggccgcccgggcaaagcccgg
gcgtcgggcgacctttggtcgcccggcctcagtgagcgagcgagcgcgcagagagggagt
ggccaactccatcactaggggttccttgtagttaatgattaacccgccatgctacttatc
tacgtaACTCCATGAAAGTGGATTTTATTATCCTCATCATGCAGATGAGAATATTGAGAC
TTATAGCGGTATGCCTGAGCCCCAAAGTACTCAGAGTTGCCTGGCTCCAAGATTTATAAT
CTTAAATGATGGGACTACCATCCTTACTCTCTCCATTTTTCTATACGTGAGTAATGTTTT
TTCTGTTTTTTTTTTTTCTTTTTCCATTCAAACTCAGTGCACTTGTTGAGCTCGTGAAAC
ACAAGCCCAAGGCAACAAAAGAGCAACTGAAAGCTGTTATGGATGATTTCGCAGCTTTTG
TAGAGAAGTGCTGCAAGGCTGACGATAAGGAGACCTGCTTTGCCGAGGAGGTACTACAGT
TCTCTTCATTTTAATATGTCCAGTATTCATTTTTGCATGTTTGGTTAGGCTAGGGCTTAG
GGATTTATATATCAAAGGAGGCTTTGTACATGTGGGACAGGGATCTTATTTTACAAACAA
TTGTCTTACAAAATGAATAAAACAGCACTTTGTTTTTATCTCCTGCTCTATTGTGCCATA
CTGTTAAATGTTTATAATGCCTGTTCTGTTTCCAAATTTGTGATGCTTATGAATATTAAT
AGGAATATTTGTAAGGCCTGAAATATTTTGATCATGAAATCAAAACATTAATTTATTTAA
ACATTTACTTGAAATGTGGTGGTTTGTGATTTAGTTGATTTTATAGGCTAGTGGGAGAAT
TTACATTCAAATGTCTAAATCACTTAAAATTGCCCTTTATGGCCTGACAGTAACTTTTTT
TTATTCATTTGGGGACAACTATGTCCGTGAGCTTCCGTCCAGAGATTATAGTAGTAAATT
GTAATTAAAGGATATGATGCACGTGAAATCACTTTGCAATCATCAATAGCTTCATAAATG
TTAATTTTGTATCCTAATAGTAATGCTAATATTTTCCTAACATCTGTCATGTCTTTGTGT
TCAGGGTAAAAAACTTGTTGCTGCAAGTCAAGCTGCCTTAGGCTTAGGAAGCGGCGCCAC
CAATTTCAGCCTGCTGAAACAGGCCGGCGACGTGGAAGAGAACCCTGGCCCTTCCTTTAT
TCCTGTGGCCGAAGATAGCGACTTCCCTATCCATAACCTCCCATATGGCGTCTTCTCAAC
CAGGGGCGACCCCAGGCCCAGGATTGGGGTGGCAATTGGAGACCAGATCCTGGACCTCAG
CATCATTAAGCACCTCTTTACAGGACCTGTGCTGAGCAAGCACCAGGATGTGTTCAACCA
GCCTACCCTGAATAGCTTTATGGGACTGGGCCAGGCAGCATGGAAAGAAGCCAGAGTGTT
CCTCCAGAATCTGCTGAGCGTGAGCCAGGCCAGGCTCAGGGATGACACAGAACTGAGGAA
ATGTGCCTTCATCTCACAGGCATCAGCCACAATGCACCTCCCTGCCACAATTGGGGACTA
CACTGACTTCTACAGCAGCAGACAGCATGCCACTAATGTGGGGATTATGTTTAGAGACAA
GGAAAATGCCCTCATGCCAAATTGGCTGCACCTGCCTGTGGGCTACCACGGCAGAGCCTC
ATCAGTGGTGGTGTCAGGCACACCAATTAGGAGGCCAATGGGACAGATGAAGCCTGACGA
CTCAAAGCCACCAGTCTATGGCGCCTGCAAACTGCTGGACATGGAGCTGGAGATGGCTTT
CTTTGTGGGGCCTGGCAACAGGCTGGGAGAACCAATCCCTATTTCAAAGGCCCACGAGCA
CATTTTTGGCATGGTGCTCATGAATGACTGGTCTGCCAGAGATATCCAGAAGTGGGAATA
CGTGCCCCTGGGCCCTTTTCTGGGCAAGAGCTTTGGCACCACAGTGTCACCTTGGGTGGT
CCCAATGGATGCCCTGATGCCCTTTGCCGTGCCCAACCCCAAGCAGGATCCCAGACCACT
GCCCTACCTGTGCCACGATGAGCCCTATACCTTTGACATCAACCTGTCAGTCAACCTGAA
GGGGGAGGGCATGAGCCAGGCCGCCACTATTTGCAAGAGCAACTTTAAATATATGTACTG
GACTATGCTCCAACAGCTCACTCACCACTCAGTGAATGGCTGCAATCTGAGGCCTGGCGA
CCTCCTGGCTAGCGGCACTATCTCTGGCCCTGAACCTGAGAACTTTGGGAGCATGCTGGA
GCTGTCATGGAAAGGGACAAAGCCTATTGACCTGGGGAATGGCCAGACAAGGAAGTTTCT
CCTGGATGGCGATGAGGTCATCATCACTGGGTACTGCCAGGGCGATGGCTACAGGATTGG
ATTTGGACAGTGTGCTGGCAAAGTGCTCCCAGCTCTGCTGCCCTCATAACATCACATTTA
AAAGCATCTCAGGTAACTATATTTTGAATTTTTTAAAAAAGTAACTATAATAGTTATTAT
TAAAATAGCAAAGATTGACCATTTCCAAGAGCCATATAGACCAGCACCGACCACTATTCT
AAACTATTTATGTATGTAAATATTAGCTTTTAAAATTCTCAAAATAGTTGCTGAGTTGGG
AACCACTATTATTTCTATTTTGTAGATGAGAAAATGAAGATAAACATCAAAGCATAGATT
AAGTAATTTTCCAAAGGGTCAAAATTCAAAATTGAAACCAAAGTTTCAGTGTTGCCCATT
GTCCTGTTCTGACTTATATGATGCGGTACACAGAGCCATCCAAGTAAGTGATGGCTCAGC
AGTGGAATACTCTGGGAATTAGGCTGAACCACATGAAAGAGTGCTTTATAGGGCAAAAAC
AGTTGAATATCAGTGATTTCACATGGTTCAACCTAATAGTTCAACTCATCCTTTCCATTG
GAGAATATGATGGATCTACCTTCTGTGAACTTTATAGTGAAGAATCTGCTATTACATTTC
CAATTTGTCAACATGCTGAGCTTTAATAGGACTTATCTTCTTATGACAACATTTATTGGT
GTGTCCCCTTGCCTAGCCCAACAGAAGAATTCAGCAGCCGTAAGTCTAGGACAGGCTTAA
ATTGTTTTCACTGGTGTAAATTGCAGAAAGATGATCTAAGTAATTTGGCATTTATTTTAA
TAGGTTTGAAAAACACATGCCATTTTACAAATAAGACTTATATTTGTCCTTTTGTTTTTC
AGCCTACCATGAGAATAAGAGAAAGAAAATGAAGATCAAAAGCTTATTCATCTGTTTTTC
TTTTTCGTTGGTGTAAAGCCAACACCCTGTCTAAAAAACATAAATTTCTTTAATCATTTT
GCCTCTTTTCTCTGTGCTTCAATTAATAAAAAATGGAAAGAATCTAATAGAGTGGTACAG
CACTGTTATTTTTCAAAGATGTGTTGtacgtagataagtagcatggcgggttaatcatta
actacaaggaacccctagtgatggagttggccactccctctctgcgcgctcgctcgctca
ctgaggccgggcgaccaaaggtcgcccgacgcccgggctttgcccgggcggcctcagtga
gcgagcgagcgcgcagctggcgtaatagcgaagaggcccgcaccgatcgggtcaccaagc
aggaagtcaaagactttttccggtgggcaaaggatcacgtggttgaggtggagcatgaat
tctacgtcaaaaagggtggagccaagaaaagacccgcccccagtgacgcagatataagtg
agcccaaacgggtgcgcgagtcagttgcgcagccatcgacgtcagacgcggaagcttcga
tcaactacgcagacaggtaccaaaacaaatgttctcgtcacgtgggcatgaatctgatgc
tgtttccctgcagacaatgcgagagaatgaatcagaattcaaatatctgcttcactcacg
gacagaaagactgtttagagtgctttcccgtgtcagaatctcaacccgtttctgtcgtca
aaaaggcgtatcagaaactgtgctacattcatcatatcatgggaaaggtgccagacgctt
gcactgcctgcgatctggtcaatgtggatttggatgactgcatctttgaacaataaatga
tttaaatcaggtatggctgctgacggttatcttccagattggctcgaggacaacctttct
gaaggcattcgagagtggtgggcgctgcaacctggagcccctaaacccaaggcaaatcaa
caacatcaggacaacgctcggggtcttgtgcttccgggttacaaatacctcggacccggc
aacggactcgacaagggggaacccgtcaacgcagcggacgcggcagccctcgagcacgac
aaggcctacgaccagcagctcaaggccggtgacaacccctacctcaagtacaaccacgcc
gacgccgagttccaggagcggctcaaagaagatacgtcttttgggggcaacctcgggcga
gcagtcttccaggccaaaaagaggcttcttgaacctcttggtctggttgaggaagcggct
aagacggctcctggaaagaagaggcctgtagatcagtctcctcaggaaccggactcatca
tctggtgttggcaaatcgggcaaacagcctgccagaaaaagactaaatttcggtcagact
ggcgactcagagtcagtcccagaccctcaacctctcggagaaccaccagcagcccccaca
agtttgggatctaatacaatggcttcaggcggtggcgcaccaatggcagacaataacgag
ggtgccgatggagtgggtaattcctcaggaaattggcattgcgattcccaatggctgggc
gacagagtcatcaccaccagcaccagaacctgggccctgcccacttacaacaaccatctc
tacaagcaaatctccagccaatcaggagcttcaaacgacaaccactactttggctacagc
accccttgggggtattttgactttaacagattccactgccacttctcaccacgtgactgg
cagcgactcattaacaacaactggggattccggcccaagaaactcagcttcaagctcttc
aacatccaagttaaagaggtcacgcagaacgatggcacgacgactattgccaataacctt
accagcacggttcaagtgtttacggactcggagtatcagctcccgtacgtgctcgggtcg
gcgcaccaaggctgtctcccgccgtttccagcggacgtcttcatggtccctcagtatgga
tacctcaccctgaacaacggaagtcaagcggtgggacgctcatccttttactgcctggag
tacttcccttcgcagatgctaaggactggaaataacttccaattcagctataccttcgag
gatgtaccttttcacagcagctacgctcacagccagagtttggatcgcttgatgaatcct
cttattgatcagtatctgtactacctgaacagaacgcaaggaacaacctctggaacaacc
aaccaatcacggctgctttttagccaggctgggcctcagtctatgtctttgcaggccaga
aattggctacctgggccctgctaccggcaacagagactttcaaagactgctaacgacaac
aacaacagtaactttccttggacagcggccagcaaatatcatctcaatggccgcgactcg
ctggtgaatccaggaccagctatggccagtcacaaggacgatgaagaaaaatttttccct
atgcacggcaatctaatatttggcaaagaagggacaacggcaagtaacgcagaattagat
aatgtaatgattacggatgaagaagagattcgtaccaccaatcctgtggcaacagagcag
tatggaactgtggcaaataacttgcagagctcaaatacagctcccacgactagaactgtc
aatgatcagggggccttacctggcatggtgtggcaagatcgtgacgtgtaccttcaagga
cctatctgggcaaagattcctcacacggatggacactttcatccttctcctctgatggga
ggctttggactgaaacatccgcctcctcaaatcatgatcaaaaatactccggtaccggca
aatcctccgacgactttcagcccggccaagtttgcttcatttatcactcagtactccact
ggacaggtcagcgtggaaattgagtgggagctacagaaagaaaacagcaaacgttggaat
ccagagattcagtacacttccaactacaacaagtctgttaatgtggactttactgtagac
actaatggtgtttatagtgaacctcgccccattggcacccgttaccttacccgtcccctg
taattgcttgttaatcaataaaccgtttaattcgtttcagttgaactttggtctctgcgt
atttctttcttatctagtttccatatgcatgtagataagtagcatggcgggttaatcatt
aactaaccggtacctctagaactatagctagcatgcgcaaatttaaagcgctgatatcga
tcgcgcgcagatctgtcatgatgatcattgcaattggatccatatatagggcccgggtta
taattacctcaggtcgacgtcccatggccattcgaattcgtaatcatggtcatagctgtt
tcctgtgtgaaattgttatccgctcacaattccacacaacatacgagccggaagcataaa
gtgtaaagcctggggtgcctaatgagtgagctaactcacattaattgcgttgcgctcact
gcccgctttccagtcgggaaacctgtcgtgccagctgcattaatgaatcggccaacgcgc
ggggagaggcggtttgcgtattgggcgctcttccgcttcctcgctcactgactcgctgcg
ctcggtcgttcggctgcggcgagcggtatcagctcactcaaaggcggtaatacggttatc
cacagaatcaggggataacgcaggaaagaacatgtgagcaaaaggccagcaaaaggccag
gaaccgtaaaaaggccgcgttgctggcgtttttccataggctccgcccccctgacgagca
tcacaaaaatcgacgctcaagtcagaggtggcgaaacccgacaggactataaagatacca
ggcgtttccccctggaagctccctcgtgcgctctcctgttccgaccctgccgcttaccgg
atacctgtccgcctttctcccttcgggaagcgtggcgctttctcatagctcacgctgtag
gtatctcagttcggtgtaggtcgttcgctccaagctgggctgtgtgcacgaaccccccgt
tcagcccgaccgctgcgccttatccggtaactatcgtcttgagtccaacccggtaagaca
cgacttatcgccactggcagcagccactggtaacaggattagcagagcgaggtatgtagg
cggtgctacagagttcttgaagtggtggcctaactacggctacactagaagaacagtatt
tggtatctgcgctctgctgaagccagttaccttcggaaaaagagttggtagctcttgatc
cggcaaacaaaccaccgctggtagcggtggtttttttgtttgcaagcagcagattacgcg
cagaaaaaaaggatctcaagaagatcctttgatcttttctacggggtctgacgctcagtg
gaacgaaaactcacgttaagggattttggtcatgagattatcaaaaaggatcttcaccta
gatccttttaaattaaaaatgaagttttaaatcaatctaaagtatatatgagtaaacttg
gtctgacagttagaaaaactcatcgagcatcaaatgaaactgcaatttattcatatcagg
attatcaataccatatttttgaaaaagccgtttctgtaatgaaggagaaaactcaccgag
gcagttccataggatggcaagatcctggtatcggtctgcgattccgactcgtccaacatc
aatacaacctattaatttcccctcgtcaaaaataaggttatcaagtgagaaatcaccatg
agtgacgactgaatccggtgagaatggcaaaagtttatgcatttctttccagacttgttc
aacaggccagccattacgctcgtcatcaaaatcactcgcatcaaccaaaccgttattcat
tcgtgattgcgcctgagcgagacgaaatacgcgatcgctgttaaaaggacaattacaaac
aggaatcgaatgcaaccggcgcaggaacactgccagcgcatcaacaatattttcacctga
atcaggatattcttctaatacctggaatgctgtttttccggggatcgcagtggtgagtaa
ccatgcatcatcaggagtacggataaaatgcttgatggtcggaagaggcataaattccgt
cagccagtttagtctgaccatctcatctgtaacatcattggcaacgctacctttgccatg
tttcagaaacaactctggcgcatcgggcttcccatacaagcgatagattgtcgcacctga
ttgcccgacattatcgcgagcccatttatacccatataaatcagcatccatgttggaatt
taatcgcggcctcgacgtttcccgttgaatatggctcatactcttcctttttcaatatta
ttgaagcatttatcagggttattgtctcatgagcggatacatatttgaatgtatttagaa
aaataaacaaataggggttccgcgcacatttccccgaaaagtgccacctgacgtctaaga
aaccattattatcatgacattaacctataaaaataggcgtatcacgaggccctttcgtct
cgcgcgtttcggtgatgacggtgaaaacctctgacacatgcagctcccggagacggtcac
agcttgtctgtaagcggatgccgggagcagacaagcccgtcagggcgcgtcagcgggtgt
tggcgggtgtcggggctggcttaactatgcggcatcagagcagattgtactgagagtgca
ccataaaattgtaaacgttaatattttgttaaaattcgcgttaaatttttgttaaatcag
ctcattttttaaccaataggccgaaatcggcaaaatcccttataaatcaaaagaatagcc
cgagatagggttgagtgttgttccagtttggaacaagagtccactattaaagaacgtgga
ctccaacgtcaaagggcgaaaaaccgtctatcagggcgatggcccactacgtgaaccatc
acccaaatcaagttttttggggtcgaggtgccgtaaagcactaaatcggaaccctaaagg
gagcccccgatttagagcttgacggggaaagccggcgaacgtggcgagaaaggaagggaa
gaaagcgaaaggagcgggcgctagggcgctggcaagtgtagcggtcacgctgcgcgtaac
caccacacccgccgcgcttaatgcgccgctacagggcgcgtactatggttgctttgacgt
atgcggtgtgaaataccgcacagatgcgtaaggagaaaataccgcatcaggcgccattcg
ccattcaggctgcgcaactgttgggaagggcgatcggtgcgggcctcttcgctattacgc
cagctggcgaaagggggatgtgctgcaaggcgattaagttgggtaacgccagggttttcc
cagtcacgacgttgtaaaacgacggccaacatgttctttcctgcgttatcccctgattct
gtggataaccgtattaccgcctttgagtgagctgataccgctcgccgcagccgaacgacc
gagcgcagcgagtcagtgagcgaggaagcggaagagcgcccaatacgcaaaccgcctctc
cccgcgcgttggc

In some embodiments, the present disclosure provides a composition comprising a recombinant AAV construct comprising: a polynucleotide cassette comprising: an expression cassette comprising a first nucleic acid sequence and a second nucleic acid sequence, wherein the first nucleic acid sequence has 80% sequence identity to SEQ ID NO. 18, 19, 20, 21, or 22; and the second nucleic acid sequence (i) is positioned 5′ or 3′ to the first nucleic acid sequence; and (ii) promotes the production of two independent gene products upon integration into a target integration site in the genome of a cell; a third nucleic acid sequence positioned 5′ to the expression cassette and comprising a sequence that is substantially homologous to a genomic sequence 5′ of a target integration site in a genome of a cell; and a fourth nucleic acid sequence positioned 3′ to expression cassette and comprising a sequence that is substantially homologous to a genomic sequence 3′ of a target integration site in the genome of the cell. In some embodiments, the AAV construct comprises a capsid protein comprising an amino acid sequence having at least 95% sequence identity with the amino acid sequence of, AAV8, AAV-DJ; AAV-LK03; sL65; or AAVNP59. In some embodiments, the composition further comprises AAV2 ITR sequences. In some embodiments, the second nucleic acid sequence has 80% sequence identity to SEQ ID NO. 6. In some embodiments, the second nucleic acid sequence encodes a P2A peptide having 90% sequence identity to SEQ ID NO. 7. In some embodiments, the third nucleic acid sequence has 80% sequence identity to SEQ ID NO. 1, 3, or 4. In some embodiments, the fourth nucleic acid sequence has 80% sequence identity to SEQ ID NO. 2 or 5.

### EXEMPLIFICATION

### Example 1. Materials and Methods

### Animal Study

FAH knock out (Fah−/−, KO) and heterozygous Fah+/− littermates (HET) animals are purchased from Jackson Laboratories. FRG mice were purchased from Yecuris corporation.

### GENERIDE™ PoC in Tyrosinemia FRG Mice

Four-week-old FRG male animals were treated with either vehicle or rAAV.DJ-GR-hFAH at 1e14 vg/kg via retro-orbital sinus under anesthesia. All mice were kept on 8 mg/L of Nitisinone (NTBC) prior to the initiation of the study and one week post dosing, and then NTBC were cycled up to 5 weeks post dosing based on body weight loss and then NTBC was discontinued. During the study, animals were sampled periodically by submandibular bleed and plasma was collected and stored at −80° C. until further analysis. Terminal harvest was conducted at week 9 and 16 post dosing. At sacrifices, blood was collected for plasma via cardiac puncture. Animal were either dissected the whole liver or underwent liver perfusion to collect hepatocyte. For animals which the whole liver was dissected, one lobe of liver was fixed 10% formalin, and the remaining was snap-frozen and stored at −80° C. Next day, formalin-fixed liver was transferred to 70% ethanol for paraffin embedding. Following the liver perfusion, isolated hepatocytes were centrifuged at 300×g for 5 min at 4° C. and stored at −80° C.

### Dose-Response PoC of GENERIDE™ in FAH Mice

Pediatric Fah−/−(KO) and Fah+/−(HET) animals at 14 days old were treated with rAAV.DJ-GR-mFAH at dosages of 1e13, 3e13, or 1e14 vg/kg via retro-orbital sinus under anesthesia. All mice were kept on 8 mg/L of NTBC prior to the initiation of the study and one week post dosing, and then NTBC were cycled up to 2 weeks post dosing based on body weight loss. During the study, animals were sampled periodically by submandibular bleed and plasma was collected and stored at −80° C. until further analysis. Terminal harvest was conducted at 16 weeks post dosing. At sacrifices, blood was collected for plasma via cardiac puncture. Animal underwent liver perfusion to collect hepatocyte. One lobe of liver was sutured and dissected for formalin fixation prior perfusion began. Following the liver perfusion, isolated hepatocytes were centrifuged at 300×g for 5 min at 4° C. and stored at −80° C. Next day, formalin-fixed liver was transferred to 70% ethanol for paraffin embedding.

### Assessment of Hepatocellular Carcinoma (HCC) Risk Between NTBC Vs GeneRide

Fah−/− animals were maintained on 8 mg/L NTBC since birth. At Four-week-old age, a group of Fah−/− animals were randomly selected and treated with rAAV.DJ-GR-mFAH at dosages of 1e14 vg/kg via retro-orbital sinus under anesthesia and then withdrawn from NTBC (GENERIDE™ treatment group). Another group of Fah−/− animals were maintained on 8 mg/L NTBC (Standard of Care group). A third group of Fah+/− littermates were enrolled in the study but not receiving any treatment (no NTBC or GeneRide). All animals were followed up till one year of age and HCC biomarker (AFP level) were assessed periodically.

### Assessment of Compatibility Between NTBC (Standard of Care) and GENERIDE™

Four-week-old Fah−/− animals were treated with rAAV.DJ-GR-mFAH at dosages of 1e14 vg/kg via retro-orbital sinus under anesthesia. All mice were kept on 8 mg/L of NTBC prior to the initiation of the study until 4 weeks post dosing, and then NTBC were either maintained at 8 mg/L (control) or titrated down to 3 mg/L, 0.8 mg/L, or 0.3 mg/L for 8 weeks. During the study, animals were sampled periodically by submandibular bleed and plasma was collected and stored at −80° C. until further analysis. At sacrifices, blood was collected for plasma via cardiac puncture. For liver dissection, one lobe of liver was fixed by 10% formalin, and the remaining was snap-frozen and stored at −80° C. Next day, formalin-fixed liver was transferred to 70% ethanol for paraffin embedding.

### Targeted Genomic DNA Integration in Liver

Genomic DNA was extracted from frozen liver tissues and targeted genomic DNA integration was analyzed by long-range polymerase chain reaction (PCR) amplification, followed by quantitative polymerase chain reaction (qPCR) quantification using a qualified method (see below figure). Long Range PCR was performed using a forward primer (F1) and a reverse primer (R1). The PCR product was cleaned by solid phase reversible immobilization beads (ABM, G950) and used as template for qPCR using the forward primer (F1), a reverse primer (R2) and a probe (P1). The primers and probes are (F1) 5′-ATGTTCCACGAAGAAGCCA-3′, (RI) 5′-TCAGCAGGCTGAAATTGGT-3, (R2) 5′-AGCTGTTTCTTACTCCATTCTCA-3′, (P1) 5′-AGGCAACGTCATGGGTGTGACTTT-3′. The mouse transferrin receptor (Tfrc) was used as an internal control in qPCR.

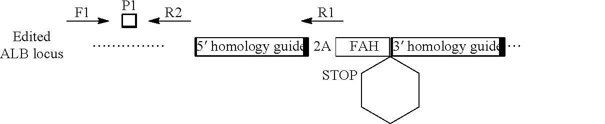

### Plasma Albumin-2A Fusion Protein Quantification

Mouse Albumin-2A in plasma was measured by chemoluminescence ELISA, using a proprietary rabbit polyclonal anti-2A antibody for capture and an HRP-labeled polyclonal goat anti-mouse Albumin antibody (abcam ab19195) for detection. Recombinant mouse Albumin-2A expressed in mammalian cells and affinity-purified was used to build the standard curve in 1% control mouse plasma to account for matrix effects. Milk at 1% (Cell Signaling 9999S) in PBS was used for blocking and BSA at 1% for sample dilution in PBST.

### Plasma Liver Injury Biomarkers Quantification

Alanine aminotransferase (ALT) activity and total bilirubin level in mouse plasma were quantified as biomarkers for liver injury. Plasma ALT activity was quantified using an alanine aminotransferase activity colorimetric assay kit (BioVision) following the vendor's instructions. Total bilirubin in plasma was measured using a certified clinical analyzer, Advanced® BR2 Bilirubin Stat-Analyzer™ (Advanced Instruments, LLC) according to the manufacturer's protocol.

### Plasma Alpha-Fetoprotein Quantification

Plasma alpha-fetoprotein was quantified using chemoluminescence ELISA kit (R&D Systems) according to the manufacturer's protocol.

### Immunohistochemistry

Immunohistochemistry was performed on a robotic platform (Ventana discover Ultra Staining Module, Ventana Co., Tucson, AZ). Tissue sections (4 μm) were deparaffinized and underwent heat-induced antigen retrieval for 64 min. Endogenous peroxidases were blocked with peroxidase inhibitor (CM1) for 8 min before incubating the section with anti-FAH antibody (Yecuris, Portland, OR) at 1:400 dilution for 60 min at room temperature. Antigen-antibody complex was then detected using DISC. OmniMap anti-rabbit multimer RUO detection system and DISCOVERY ChromoMap DAB Kit Ventana Co., Tucson, AZ). All the slides were counterstained with hematoxylin (Fisher Sci, Waltham, MA) subsequently, dehydrated, cleared and mounted for image scanning using digital slide scanner (Hamamatsu, Bridgewater, NJ). Scanned images were evaluated in a blinded fashion using ImageJ software to quantify the area of positive staining. Exemplary sequences used in the present Examples are provided below:

AAV-DJ-mha-mFAH
(PM-0550; SEQ ID NO. 27)
cagatcctctacgccggacgcatcgtggccggcatcaccggcgccacaggtgcggttgct
ggcgcctatatcgccgacatcaccgatggggaagatcgggctcgccacttcgggctcatg
agcgcttgtttcggcgtgggtatggtggcaggccccgtggccgggggactgttgggcgcc
atctccttgcatgcaccattccttgcggcggcggtgctcaacggcctcaacctactactg
ggctgcttcctaatgcaggagtcgcataagggagagcgtcgaatggtgcactctcagtac
aatctgctctgatgccgcatagttaagccagccccgacacccgccaacacccgctgacgc
gccctgacgggcttgtctgctcccggcatccgcttacagacaagctgtgaccgtctccgg
gagctgcatgtgtcagaggttttcaccgtcatcaccgaaacgcgcgagacgaaagggcct
cgtgatacgcctatttttataggttaatgtcatgataataatggtttcttagacgtcagg
tggcacttttcggggaaatgtgcgcggaacccctatttgtttatttttctaaatacattc
aaatatgtatccgctcatgagacaataaccctgataaatgcttcaataatattgaaaaag
gaagagtatgagccatattcaacgggaaacgtcgaggccgcgattaaattccaacatgga
tgctgatttatatgggtataaatgggctcgcgataatgtcgggcaatcaggtgcgacaat
ctatcgcttgtatgggaagcccgatgcgccagagttgtttctgaaacatggcaaaggtag
cgttgccaatgatgttacagatgagatggtcagactaaactggctgacggaatttatgcc
tcttccgaccatcaagcattttatccgtactcctgatgatgcatggttactcaccactgc
gatccccggaaaaacagcattccaggtattagaagaatatcctgattcaggtgaaaatat
tgttgatgcgctggcagtgttcctgcgccggttgcattcgattcctgtttgtaattgtcc
ttttaacagcgatcgcgtatttcgtctcgctcaggcgcaatcacgaatgaataacggttt
ggttgatgcgagtgattttgatgacgagcgtaatggctggcctgttgaacaagtctggaa
agaaatgcataaacttttgccattctcaccggattcagtcgtcactcatggtgatttctc
acttgataaccttatttttgacgaggggaaattaataggttgtattgatgttggacgagt
cggaatcgcagaccgataccaggatcttgccatcctatggaactgcctcggtgagttttc
tccttcattacagaaacggctttttcaaaaatatggtattgataatcctgatatgaataa
attgcagtttcatttgatgctcgatgagtttttctaactgtcagaccaagtttactcata
tatactttagattgatttaaaacttcatttttaatttaaaaggatctaggtgaagatcct
ttttgataatctcatgaccaaaatcccttaacgtgagttttcgttccactgagcgtcaga
ccccgtagaaaagatcaaaggatcttcttgagatcctttttttctgcgcgtaatctgctg
cttgcaaacaaaaaaaccaccgctaccagcggtggtttgtttgccggatcaagagctacc
aactctttttccgaaggtaactggcttcagcagagcgcagataccaaatactgttcttct
agtgtagccgtagttaggccaccacttcaagaactctgtagcaccgcctacatacctcgc
tctgctaatcctgttaccagtggctgctgccagtggcgataagtcgtgtcttaccgggtt
ggactcaagacgatagttaccggataaggcgcagcggtcgggctgaacggggggttcgtg
cacacagcccagcttggagcgaacgacctacaccgaactgagatacctacagcgtgagct
atgagaaagcgccacgcttcccgaagggagaaaggcggacaggtatccggtaagcggcag
ggtcggaacaggagagcgcacgagggagcttccagggggaaacgcctggtatctttatag
tcctgtcgggtttcgccacctctgacttgagcgtcgatttttgtgatgctcgtcaggggg
gcggagcctatggaaaaacgccagcaacgcggcctttttacggttcctggccttttgctg
gccttttgctcacatgttctttcctgcgttatcccctgattctgtggataaccgtattac
cgcctttgagtgagctgataccgctcgccgcagccgaacgaccgagcgcagcgagtcagt
gagcgaggaagcggaagagcgcccaatacgcaaaccgcctctccccgcgcgttggccgat
tcattaatgcagctgtggaatgtgtgtcagttagggtgtggaaagtccccaggctcccca
gcaggcagaagtatgcaaagcatgcatctcaattagtcagcaaccaggtgtggaaagtcc
ccaggctccccagcaggcagaagtatgcaaagcatgcatctcaattagtcagcaaccata
gtcccgcccctaactccgcccatcccgcccctaactccgcccagttccgcccattctccg
ccccatggctgactaattttttttatttatgcagaggccgaggccgcctcggcctctgag
ctattccagaagtagtgaggaggcttttttggaggcctaggcttttgcaaaaagttggcc
actccctctctgcgcgctcgctcgctcactgaggccgcccgggcaaagcccgggcgtcgg
gcgacctttggtcgcccggcctcagtgagcgagcgagcgcgcagagagggagtggccaac
tccatcactaggggttcctTACGTAATGCATGGATCCCCTAGGgcggccgcCTGAAACTA
GACAAAACCCGTGTGACTGGCATCGATTATTCTATTTGATCTAGCTAGTCCTAGCAAAGT
GACAACTGCTACTCCCCTCCTACACAGCCAAGATTCCTAAGTTGGCAGTGGCATGCTTAA
TCCTCAAAGCCAAAGTTACTTGGCTCCAAGATTTATAGCCTTAAACTGTGGCCTCACATT
CCTTCCTATCTTACTTTCCTGCACTGGGGTAAATGTCTCCTTGCTCTTCTTGCTTTCTGT
CCTACTGCAGGGCTCTTGCTGAGCTGGTGAAGCACAAGCCCAAGGCTACAGCGGAGCAAC
TGAAGACTGTCATGGATGACTTTGCACAGTTCCTGGATACATGTTGCAAGGCTGCTGACA
AGGACACCTGCTTCTCGACTGAGGTCAGAAACGTTTTTGCATTTTGACGATGTTCAGTTT
CCATTTTCTGTGCACGTGGTCAGGTGTAGCTCTCTGGAACTCACACACTGAATAACTCCA
CCAATCTAGATGTTGTTCTCTACGTAACTGTAATAGAAACTGACTTACGTAGCTTTTAAT
TTTTATTTTCTGCCACACTGCTGCCTATTAAATACCTATTATCACTATTTGGTTTCAAAT
TTGTGACACAGAAGAGCATAGTTAGAAATACTTGCAAAGCCTAGAATCATGAACTCATTT
AAACCTTGCCCTGAAATGTTTCTTTTTGAATTGAGTTATTTTACACATGAATGGACAGTT
ACCATTATATATCTGAATCATTTCACATTCCCTCCCATGGCCTAACAACAGTTTATCTTC
TTATTTTGGGCACAACAGATGTCAGAGAGCCTGCTTTAGGAATTCTAAGTAGAACTGTAA
TTAAGCAATGCAAGGCACGTACGTTTACTATGTCATTGCCTATGGCTATGAAGTGCAAAT
CCTAACAGTCCTGCTAATACTTTTCTAACATCCATCATTTCTTTGTTTTCAGGGTCCAAA
CCTTGTCACTAGATGCAAAGACGCCTTAGCCggaagcggcgccaccaatttcagcctgct
gaaacaggccggcgacgtggaagagaaccctggccctTCCTTTATTCCAGTGGCCGAGGA
CTCCGACTTTCCCATCCAAAACCTGCCCTATGGTGTTTTCTCCACTCAAAGCAACCCAAA
GCCACGGATTGGTGTAGCCATCGGTGACCAGATCTTGGACCTGAGTGTCATTAAACACCT
CTTTACCGGACCTGCCCTTTCCAAACATCAACATGTCTTCGATGAGACAACTCTCAATAA
CTTCATGGGTCTGGGTCAAGCTGCATGGAAGGAGGCAAGAGCATCCTTACAGAACTTACT
GTCTGCCAGCCAAGCCCGGCTCAGAGATGACAAGGAGCTTCGGCAGCGTGCATTCACCTC
CCAGGCTTCTGCGACAATGCACCTTCCTGCTACCATAGGAGACTACACGGACTTCTACTC
TTCTCGGCAGCATGCCACCAATGTTGGCATTATGTTCAGAGGCAAGGAGAATGCGCTGTT
GCCAAATTGGCTCCACTTACCTGTGGGATACCATGGCCGAGCTTCCTCCATTGTGGTATC
TGGAACCCCGATTCGAAGACCCATGGGGCAGATGAGACCTGATAACTCAAAGCCTCCTGT
GTATGGTGCCTGCAGACTCTTAGACATGGAGTTGGAAATGGCTTTCTTCGTAGGCCCTGG
GAACAGATTCGGAGAGCCAATCCCCATTTCCAAAGCCCATGAACACATTTTCGGGATGGT
CCTCATGAACGACTGGAGCGCACGAGACATCCAGCAATGGGAGTACGTCCCACTTGGGCC
ATTCCTGGGGAAAAGCTTTGGAACCACAATCTCCCCGTGGGTGGTGCCTATGGATGCCCT
CATGCCCTTTGTGGTGCCAAACCCAAAGCAGGACCCCAAGCCCTTGCCATATCTCTGCCA
CAGCCAGCCCTACACATTTGATATCAACCTGTCTGTCTCTTTGAAAGGAGAAGGAATGAG
CCAGGCGGCTACCATCTGCAGGTCTAACTTTAAGCACATGTACTGGACCATGCTGCAGCA
ACTCACACACCACTCTGTTAATGGATGCAACCTGAGACCTGGGGACCTCTTGGCTTCTGG
AACCATCAGTGGATCAGACCCTGAAAGCTTTGGCTCCATGCTGGAACTGTCCTGGAAGGG
AACAAAGGCCATCGATGTGGGGCAGGGGCAGACCAGGACCTTCCTGCTGGACGGCGATGA
AGTCATCATAACAGGTCACTGCCAGGGGGACGGCTACCGTGTTGGCTTTGGCCAGTGTGC
TGGGAAAGTGCTGCCTGCCCTTTCACCAGCCTAAACACATCACAACCACAACCTTCTCAG
GTAACTATACTTGGGACTTAAAAAACATAATCATAATCATTTTTCCTAAAACGATCAAGA
CTGATAACCATTTGACAAGAGCCATACAGACAAGCACCAGCTGGCACTCTTAGGTCTTCA
CGTATGGTCATCAGTTTGGGTTCCATTTGTAGATAAGAAACTGAACATATAAAGGTCTAG
GTTAATGCAATTTACACAAAAGGAGACCAAACCAGGGAGAGAAGGAACCAAAATTAAAAA
TTCAAACCAGAGCAAAGGAGTTAGCCCTGGTTTTGCTCTGACTTACATGAACCACTATGT
GGAGTCCTCCATGTTAGCCTAGTCAAGCTTATCCTCTGGATGAAGTTGAAACCATATGAA
GGAATATTTGGGGGGTGGGTCAAAACAGTTGTGTATCAATGATTCCATGTGGTTTGACCC
AATCATTCTGTGAATCCATTTCAACAGAAGATACAACGGGTTCTGTTTCATAATAAGTGA
TCCACTTCCAAATTTCTGATGTGCCCCATGCTAAGCTTTAACAGAATTTATCTTCTTATG
ACAAAGCAGCCTCCTTTGAAAATATAGCCAACTGCACACAGCTATGTTGATCAATTTTGT
TTATAATCTTGCAGAAGAGAATTTTTTAAAATAGGGCAATAATGGAAGGCTTTGGCAAAA
AAATTGTTTCTCCATATGAAAACAAAAAACTTATTTTTTTATTCAAGCAAAGAACCTATA
GACATAAGGCTATTTCAAAATTATTTCAGTTTTAGAAAGAATTGAAAGTTTTGTAGCATT
CTGAGAAGACAGCTTTCATTTGTAATCATAGGTAATATGTAGGTCCTCAGAAATGGTGAG
ACCCCTGACTTTGACACTTGGGGACTCTGAGGGACCAGTGATGAAGAGGGCACAACTTAT
ATCACACATGCACGAGTTGGGGTGAGAGGGTGTCACAACATCTATCAGTGTGTCATCTGC
CCACCAAGTAACAGATGTCAGCTAAGACTAGGTCATGTGTAGGCTGTCTACACCAGTGAA
AATCGCAAAAAGAATCTAAGAAATTCCACATTTCTAGAAAATAGGTTTGGAAACCGTATT
CCATTTTACAAAGGACACTTACATTTCTCTTTTTGTTTTCCAGGCTACCCTGAGAAAAAA
AGACATGAAGACTCAGGACTCATCTTTTCTGTTGGTGTAAAATCAACACCCTAAGGAACA
CAAATTTCTTTAAACATTTGACTTCTTGTCTCTGTGCTGCAATTAATAAAAAATGGAAAG
AATCTACTCTGTGGTTCAGAACTCTATCTTCCAAAGGCGCGCTTCACCCTAGCAGCCTCT
TTGGCTCAGAGGAATCCCTGCCTTTCCTCCCTTCATCTCAGCAGAGAATGTAGTTCCACA
TGGGCAACACAATGAAAATAAACGTTAATACTCTCCCATCTTATGGGTGGTGACCCTAGA
AACCAATACTTCAACATTACGAGAATTCTGAATGAGAGACTAAAAGCTTATGAACTGTGG
CTTTCCTTTGTCAGTGGGACTCTAAGAATGAGTTGGGGACAAAAGAGATAGGAATGGCTT
TAAAGGTGACTAGTTGAACTGATAAAGTAAATGAACTGAGGAAAAAAAATATCACTCAAc
ctgcaggGGACGTCCTACGTAATGCATaggaacccctagtgatggagttggccactccct
ctctgcgcgctcgctcgctcactgaggccgggcgaccaaaggtcgcccgacgcccgggct
ttgcccgggcggcctcagtgagcgagcgagcgcgcagagagggagtggccaagaattaat
tccgtgtattctatagtgtcacctaaatcgtatgtgtatgatacataaggttatgtatta
attgtagccgcgttctaacgacaatatgtacaagcctaattgtgtagcatctggcttact
gaagcagaccctatcatctctctcgtaaactgccgtcagagtcggtttggttggacgaac
cttctgagtttctggtaacgccgtcccgcacccggaaatggtcagcgaaccaatcagcag
ggtcatcgctagc
AAV-DJ-mha-hFAH
(PM-0549; SEQ ID NO. 28)
cagatcctctacgccggacgcatcgtggccggcatcaccggcgccacaggtgcggttgct
ggcgcctatatcgccgacatcaccgatggggaagatcgggctcgccacttcgggctcatg
agcgcttgtttcggcgtgggtatggtggcaggccccgtggccgggggactgttgggcgcc
atctccttgcatgcaccattccttgcggcggcggtgctcaacggcctcaacctactactg
ggctgcttcctaatgcaggagtcgcataagggagagcgtcgaatggtgcactctcagtac
aatctgctctgatgccgcatagttaagccagccccgacacccgccaacacccgctgacgc
gccctgacgggcttgtctgctcccggcatccgcttacagacaagctgtgaccgtctccgg
gagctgcatgtgtcagaggttttcaccgtcatcaccgaaacgcgcgagacgaaagggcct
cgtgatacgcctatttttataggttaatgtcatgataataatggtttcttagacgtcagg
tggcacttttcggggaaatgtgcgcggaacccctatttgtttatttttctaaatacattc
aaatatgtatccgctcatgagacaataaccctgataaatgcttcaataatattgaaaaag
gaagagtatgagccatattcaacgggaaacgtcgaggccgcgattaaattccaacatgga
tgctgatttatatgggtataaatgggctcgcgataatgtcgggcaatcaggtgcgacaat
ctatcgcttgtatgggaagcccgatgcgccagagttgtttctgaaacatggcaaaggtag
cgttgccaatgatgttacagatgagatggtcagactaaactggctgacggaatttatgcc
tcttccgaccatcaagcattttatccgtactcctgatgatgcatggttactcaccactgc
gatccccggaaaaacagcattccaggtattagaagaatatcctgattcaggtgaaaatat
tgttgatgcgctggcagtgttcctgcgccggttgcattcgattcctgtttgtaattgtcc
ttttaacagcgatcgcgtatttcgtctcgctcaggcgcaatcacgaatgaataacggttt
ggttgatgcgagtgattttgatgacgagcgtaatggctggcctgttgaacaagtctggaa
agaaatgcataaacttttgccattctcaccggattcagtcgtcactcatggtgatttctc
acttgataaccttatttttgacgaggggaaattaataggttgtattgatgttggacgagt
cggaatcgcagaccgataccaggatcttgccatcctatggaactgcctcggtgagttttc
tccttcattacagaaacggctttttcaaaaatatggtattgataatcctgatatgaataa
attgcagtttcatttgatgctcgatgagtttttctaactgtcagaccaagtttactcata
tatactttagattgatttaaaacttcatttttaatttaaaaggatctaggtgaagatcct
ttttgataatctcatgaccaaaatcccttaacgtgagttttcgttccactgagcgtcaga
ccccgtagaaaagatcaaaggatcttcttgagatcctttttttctgcgcgtaatctgctg
cttgcaaacaaaaaaaccaccgctaccagcggtggtttgtttgccggatcaagagctacc
aactctttttccgaaggtaactggcttcagcagagcgcagataccaaatactgttcttct
agtgtagccgtagttaggccaccacttcaagaactctgtagcaccgcctacatacctcgc
tctgctaatcctgttaccagtggctgctgccagtggcgataagtcgtgtcttaccgggtt
ggactcaagacgatagttaccggataaggcgcagcggtcgggctgaacggggggttcgtg
cacacagcccagcttggagcgaacgacctacaccgaactgagatacctacagcgtgagct
atgagaaagcgccacgcttcccgaagggagaaaggcggacaggtatccggtaagcggcag
ggtcggaacaggagagcgcacgagggagcttccagggggaaacgcctggtatctttatag
tcctgtcgggtttcgccacctctgacttgagcgtcgatttttgtgatgctcgtcaggggg
gcggagcctatggaaaaacgccagcaacgcggcctttttacggttcctggccttttgctg
gccttttgctcacatgttctttcctgcgttatcccctgattctgtggataaccgtattac
cgcctttgagtgagctgataccgctcgccgcagccgaacgaccgagcgcagcgagtcagt
gagcgaggaagcggaagagcgcccaatacgcaaaccgcctctccccgcgcgttggccgat
tcattaatgcagctgtggaatgtgtgtcagttagggtgtggaaagtccccaggctcccca
gcaggcagaagtatgcaaagcatgcatctcaattagtcagcaaccaggtgtggaaagtcc
ccaggctccccagcaggcagaagtatgcaaagcatgcatctcaattagtcagcaaccata
gtcccgcccctaactccgcccatcccgcccctaactccgcccagttccgcccattctccg
ccccatggctgactaattttttttatttatgcagaggccgaggccgcctcggcctctgag
ctattccagaagtagtgaggaggcttttttggaggcctaggcttttgcaaaaagttggcc
actccctctctgcgcgctcgctcgctcactgaggccgcccgggcaaagcccgggcgtcgg
gcgacctttggtcgcccggcctcagtgagcgagcgagcgcgcagagagggagtggccaac
tccatcactaggggttcctTACGTAATGCATGGATCCCCTAGGgcggccgcCTGAAACTA
GACAAAACCCGTGTGACTGGCATCGATTATTCTATTTGATCTAGCTAGTCCTAGCAAAGT
GACAACTGCTACTCCCCTCCTACACAGCCAAGATTCCTAAGTTGGCAGTGGCATGCTTAA
TCCTCAAAGCCAAAGTTACTTGGCTCCAAGATTTATAGCCTTAAACTGTGGCCTCACATT
CCTTCCTATCTTACTTTCCTGCACTGGGGTAAATGTCTCCTTGCTCTTCTTGCTTTCTGT
CCTACTGCAGGGCTCTTGCTGAGCTGGTGAAGCACAAGCCCAAGGCTACAGCGGAGCAAC
TGAAGACTGTCATGGATGACTTTGCACAGTTCCTGGATACATGTTGCAAGGCTGCTGACA
AGGACACCTGCTTCTCGACTGAGGTCAGAAACGTTTTTGCATTTTGACGATGTTCAGTTT
CCATTTTCTGTGCACGTGGTCAGGTGTAGCTCTCTGGAACTCACACACTGAATAACTCCA
CCAATCTAGATGTTGTTCTCTACGTAACTGTAATAGAAACTGACTTACGTAGCTTTTAAT
TTTTATTTTCTGCCACACTGCTGCCTATTAAATACCTATTATCACTATTTGGTTTCAAAT
TTGTGACACAGAAGAGCATAGTTAGAAATACTTGCAAAGCCTAGAATCATGAACTCATTT
AAACCTTGCCCTGAAATGTTTCTTTTTGAATTGAGTTATTTTACACATGAATGGACAGTT
ACCATTATATATCTGAATCATTTCACATTCCCTCCCATGGCCTAACAACAGTTTATCTTC
TTATTTTGGGCACAACAGATGTCAGAGAGCCTGCTTTAGGAATTCTAAGTAGAACTGTAA
TTAAGCAATGCAAGGCACGTACGTTTACTATGTCATTGCCTATGGCTATGAAGTGCAAAT
CCTAACAGTCCTGCTAATACTTTTCTAACATCCATCATTTCTTTGTTTTCAGGGTCCAAA
CCTTGTCACTAGATGCAAAGACGCCTTAGCCggaagcggcgccaccaatttcagcctgct
gaaacaggccggcgacgtggaagagaaccctggcccttccttcatcccggtggccgagga
ttccgacttccccatccacaacctgccctacggcgtcttctcgaccagaggcgacccaag
accgaggataggtgtggccattggcgaccagatcctggacctcagcatcatcaagcacct
ctttactggtcctgtcctctccaaacaccaggatgtcttcaatcagcctacactcaacag
cttcatgggcctgggtcaggctgcctggaaggaggcgagagtgttcttgcagaacttgct
gtctgtgagccaagccaggctcagagatgacaccgaacttcggaagtgtgcattcatctc
ccaggcttctgccacgatgcaccttccagccaccataggagactacacagacttctattc
ctctcggcagcatgctaccaacgtcggaatcatgttcagggacaaggagaatgcgttgat
gccaaattggctgcacttaccagtgggctaccatggccgtgcctcctctgtcgtggtgtc
tggcaccccaatccgaaggcccatgggacagatgaaacctgatgactctaagcctcccgt
atatggtgcctgcaagctcttggacatggagctggaaatggctttttttgtaggccctgg
aaacagattgggagagccgatccccatttccaaggcccatgagcacatttttggaatggt
ccttatgaacgactggagtgcacgagacattcagaagtgggagtatgtccctctcgggcc
attccttgggaagagttttgggaccactgtctctccgtgggtggtgcccatggatgctct
catgccctttgctgtgcccaacccgaagcaggaccccaggcccctgccgtatctgtgcca
tgacgagccctacacatttgacatcaacctctctgttaacctgaaaggagaaggaatgag
ccaggcggctaccatatgcaagtccaattttaagtacatgtactggacgatgctgcagca
gctcactcaccactctgtcaacggctgcaacctgcggccgggggacctcctggcttctgg
gaccatcagcgggccggagccagaaaacttcggctccatgttggaactgtcgtggaaggg
aacgaagcccatagacctggggaatggtcagaccaggaagtttctgctggacggggatga
agtcatcataacagggtactgccagggggatggttaccgcatcggctttggccagtgtgc
tggaaaagtgctgcctgctctcctgccatcaTAAACACATCACAACCACAACCTTCTCAG
GTAACTATACTTGGGACTTAAAAAACATAATCATAATCATTTTTCCTAAAACGATCAAGA
CTGATAACCATTTGACAAGAGCCATACAGACAAGCACCAGCTGGCACTCTTAGGTCTTCA
CGTATGGTCATCAGTTTGGGTTCCATTTGTAGATAAGAAACTGAACATATAAAGGTCTAG
GTTAATGCAATTTACACAAAAGGAGACCAAACCAGGGAGAGAAGGAACCAAAATTAAAAA
TTCAAACCAGAGCAAAGGAGTTAGCCCTGGTTTTGCTCTGACTTACATGAACCACTATGT
GGAGTCCTCCATGTTAGCCTAGTCAAGCTTATCCTCTGGATGAAGTTGAAACCATATGAA
GGAATATTTGGGGGGTGGGTCAAAACAGTTGTGTATCAATGATTCCATGTGGTTTGACCC
AATCATTCTGTGAATCCATTTCAACAGAAGATACAACGGGTTCTGTTTCATAATAAGTGA
TCCACTTCCAAATTTCTGATGTGCCCCATGCTAAGCTTTAACAGAATTTATCTTCTTATG
ACAAAGCAGCCTCCTTTGAAAATATAGCCAACTGCACACAGCTATGTTGATCAATTTTGT
TTATAATCTTGCAGAAGAGAATTTTTTAAAATAGGGCAATAATGGAAGGCTTTGGCAAAA
AAATTGTTTCTCCATATGAAAACAAAAAACTTATTTTTTTATTCAAGCAAAGAACCTATA
GACATAAGGCTATTTCAAAATTATTTCAGTTTTAGAAAGAATTGAAAGTTTTGTAGCATT
CTGAGAAGACAGCTTTCATTTGTAATCATAGGTAATATGTAGGTCCTCAGAAATGGTGAG
ACCCCTGACTTTGACACTTGGGGACTCTGAGGGACCAGTGATGAAGAGGGCACAACTTAT
ATCACACATGCACGAGTTGGGGTGAGAGGGTGTCACAACATCTATCAGTGTGTCATCTGC
CCACCAAGTAACAGATGTCAGCTAAGACTAGGTCATGTGTAGGCTGTCTACACCAGTGAA
AATCGCAAAAAGAATCTAAGAAATTCCACATTTCTAGAAAATAGGTTTGGAAACCGTATT
CCATTTTACAAAGGACACTTACATTTCTCTTTTTGTTTTCCAGGCTACCCTGAGAAAAAA
AGACATGAAGACTCAGGACTCATCTTTTCTGTTGGTGTAAAATCAACACCCTAAGGAACA
CAAATTTCTTTAAACATTTGACTTCTTGTCTCTGTGCTGCAATTAATAAAAAATGGAAAG
AATCTACTCTGTGGTTCAGAACTCTATCTTCCAAAGGCGCGCTTCACCCTAGCAGCCTCT
TTGGCTCAGAGGAATCCCTGCCTTTCCTCCCTTCATCTCAGCAGAGAATGTAGTTCCACA
TGGGCAACACAATGAAAATAAACGTTAATACTCTCCCATCTTATGGGTGGTGACCCTAGA
AACCAATACTTCAACATTACGAGAATTCTGAATGAGAGACTAAAAGCTTATGAACTGTGG
CTTTCCTTTGTCAGTGGGACTCTAAGAATGAGTTGGGGACAAAAGAGATAGGAATGGCTT
TAAAGGTGACTAGTTGAACTGATAAAGTAAATGAACTGAGGAAAAAAAATATCACTCAAC
ctgcaggGGACGTCCTACGTAATGCATaggaacccctagtgatggagttggccactccct
ctctgcgcgctcgctcgctcactgaggccgggcgaccaaaggtcgcccgacgcccgggct
ttgcccgggcggcctcagtgagcgagcgagcgcgcagagagggagtggccaagaattaat
tccgtgtattctatagtgtcacctaaatcgtatgtgtatgatacataaggttatgtatta
attgtagccgcgttctaacgacaatatgtacaagcctaattgtgtagcatctggcttact
gaagcagaccctatcatctctctcgtaaactgccgtcagagtcggtttggttggacgaac
cttctgagtttctggtaacgccgtcccgcacccggaaatggtcagcgaaccaatcagcag
ggtcatcgctagc
AAV-DJ-mha-mFAH
(PM-0549; SEQ ID NO. 29)
gtatatatgagtaaacttggtctgacagttaccaatgcttaatcagtgaggcacctatct
cagcgatctgtctatttcgttcatccatagttgcctgactccccgtcgtgtagataacta
cgatacgggagggcttaccatctggccccagtgctgcaatgataccgcgagacccacgct
caccggctccagatttatcagcaataaaccagccagccggaagggccgagcgcagaagtg
gtcctgcaactttatccgcctccatccagtctattaattgttgccgggaagctagagtaa
gtagttcgccagttaatagtttgcgcaacgttgttgccattgctacaggcatcgtggtgt
cacgctcgtcgtttggtatggcttcattcagctccggttcccaacgatcaaggcgagtta
catgatcccccatgttgtgcaaaaaagcggttagctccttcggtcctccgatcgttgtca
gaagtaagttggccgcagtgttatcactcatggttatggcagcactgcataattctctta
ctgtcatgccatccgtaagatgcttttctgtgactggtgagtactcaaccaagtcattct
gagaatagtgtatgcggcgaccgagttgctcttgcccggcgtcaatacgggataataccg
cgccacatagcagaactttaaaagtgctcatcattggaaaacgttcttcggggcgaaaac
tctcaaggatcttaccgctgttgagatccagttcgatgtaacccactcgtgcacccaact
gatcttcagcatcttttactttcaccagcgtttctgggtgagcaaaaacaggaaggcaaa
atgccgcaaaaaagggaataagggcgacacggaaatgttgaatactcatactcttccttt
ttcaatattattgaagcatttatcagggttattgtctcatgagcggatacatatttgaat
gtatttagaaaaataaacaaataggggttccgcgcacatttccccgaaaagtgccacctg
acgtctaagaaaccattattatcatgacattaacctataaaaataggcgtatcacgaggc
cctttcgtctcgcgcgtttcggtgatgacggtgaaaacctctgacacatgcagctcccgg
agacggtcacagcttgtctgtaagcggatgccgggagcagacaagcccgtcagggcgcgt
cagcgggtgttggcgggtgtcggggctggcttaactatgcggcatcagagcagattgtac
tgagagtgcaccattcgacgctctcccttatgcgactcctgcattaggaagcagcccagt
agtaggttgaggccgttgagcaccgccgccgcaaggaatggtgcatgcaaggagatggcg
cccaacagtcccccggccacggggcctgccaccatacccacgccgaaacaagcgctcatg
agcccgaagtggcgagcccgatcttccccatcggtgatgtcggcgatataggcgccagca
accgcacctgtggcgccggtgggtcaccaagcaggaagtcaaagactttttccggtgggc
aaaggatcacgtggttgaggtggagcatgaattctacgtcaaaaagggtggagccaagaa
aagacccgcccccagtgacgcagatataagtgagcccaaacgggtgcgcgagtcagttgc
gcagccatcgacgtcagacgcggaagcttcgatcaactacgcagacaggtaccaaaacaa
atgttctcgtcacgtgggcatgaatctgatgctgtttccctgcagacaatgcgagagaat
gaatcagaattcaaatatctgcttcactcacggacagaaagactgtttagagtgctttcc
cgtgtcagaatctcaacccgtttctgtcgtcaaaaaggcgtatcagaaactgtgctacat
tcatcatatcatgggaaaggtgccagacgcttgcactgcctgcgatctggtcaatgtgga
tttggatgactgcatctttgaacaataaatgATTTAAATCAGGTatggctgccgatggtt
atcttccagattggctcgaggacactctctctgaaggaataagacagtggtggaagctca
aacctggcccaccaccaccaaagcccgcagagcggcataaggacgacagcaggggtcttg
tgcttcctgggtacaagtacctcggacccttcaacggactcgacaagggagagccggtca
acgaggcagacgccgcggccctcgagcacgacaaagcctacgaccggcagctcgacagcg
gagacaacccgtacctcaagtacaaccacgccgacgccgagttccaggagcggctcaaag
aagatacgtcttttgggggcaacctcgggcgagcagtcttccaggccaaaaagaggcttc
ttgaacctcttggtctggttgaggaagcggctaagacggctcctggaaagaagaggcctg
tagagcactctcctgtggagccagactcctcctcgggaaccggaaaggcgggccagcagc
ctgcaagaaaaagattgaattttggtcagactggagacgcagactcagtcccagaccctc
aaccaatcggagaacctcccgcagccccctcaggtgtgggatctcttacaatggctgcag
gcggtggcgcaccaatggcagacaataacgagggcgccgacggagtgggtaattcctcgg
gaaattggcattgcgattccacatggatgggcgacagagtcatcaccaccagcacccgaa
cctgggccctgcccacctacaacaaccacctctacaagcaaatctccaacagcacatctg
gaggatcttcaaatgacaacgcctacttcggctacagcaccccctgggggtattttgact
ttaacagattccactgccacttttcaccacgtgactggcagcgactcatcaacaacaact
ggggattccggcccaagagactcagcttcaagctcttcaacatccaggtcaaggaggtca
cgcagaatgaaggcaccaagaccatcgccaataacctcaccagcaccatccaggtgttta
cggactcggagtaccagctgccgtacgttctcggctctgcccaccagggctgcctgcctc
cgttcccggcggacgtgttcatgattccccagtacggctacctaacactcaacaacggta
gtcaggccgtgggacgctcctccttctactgcctggaatactttccttcgcagatgctga
gaaccggcaacaacttccagtttacttacaccttcgaggacgtgcctttccacagcagct
acgcccacagccagagcttggaccggctgatgaatcctctgattgaccagtacctgtact
acttgtctcggactcaaacaacaggaggcacgacaaatacgcagactctgggcttcagcc
aaggtgggcctaatacaatggccaatcaggcaaagaactggctgccaggaccctgttacc
gccagcagcgagtatcaaagacatctgcggataacaacaacagtgaatactcgtggactg
gagctaccaagtaccacctcaatggcagagactctctggtgaatccgggcccggccatgg
caagccacaaggacgatgaagaaaagttttttcctcagagcggggttctcatctttggga
agcaaggctcagagaaaacaaatgtggacattgaaaaggtcatgattacagacgaagagg
aaatcaggacaaccaatcccgtggctacggagcagtatggttctgtatctaccaacctcc
agagaggcaacagacaagcagctaccgcagatgtcaacacacaaggcgttcttccaggca
tggtctggcaggacagagatgtgtaccttcaggggcccatctgggcaaagattccacaca
cggacggacattttcacccctctcccctcatgggggattcggacttaaacaccctccgc
ctcagatcctgatcaagaacacgcctgtacctgcggatcctccgaccaccttcaaccagt
caaagctgaactctttcatcacccagtattctactggccaagtcagcgtggagatcgagt
gggagctgcagaaggaaaacagcaagcgctggaaccccgagatccagtacacctccaact
actacaaatctacaagtgtggactttgctgttaatacagaaggcgtgtactctgaacccc
gccccattggcacccgttacctcacccgtaatctgtaaTTGCTTGTTAATCAATAAACCG
TTTAATTCGTTTCAGTTGAACTTTGGTCTCTGCGTATTTCTTTCTTATCTAGTTTCCATA
TGCATGTAGATAAGTAGCATGGCGGGTTAATCATTAACTAAccggtacctctagaactat
agctagcgatgaccctgctgattggttcgctgaccatttccgggtgcgggacggcgttac
cagaaactcagaaggttcgtccaaccaaaccgactctgacggcagtttacgagagagatg
atagggtctgcttcagtaagccagatgctacacaattaggcttgtacatattgtcgttag
aacgcggctacaattaatacataaccttatgtatcatacacatacgatttaggtgacact
atagaatacacggaattaattcttggccactccctctctgcgcgctcgctcgctcactga
ggccgcccgggcaaagcccgggcgtcgggcgacctttggtcgcccggcctcagtgagcga
gcgagcgcgcagagagggagtggccaactccatcactaggggttccttaccgaCTGAAAC
TAGACAAAACCCGTGTGACTGGCATCGATTATTCTATTTGATCTAGCTAGTCCTAGCAAA
GTGACAACTGCTACTCCCCTCCTACACAGCCAAGATTCCTAAGTTGGCAGTGGCATGCTT
AATCCTCAAAGCCAAAGTTACTTGGCTCCAAGATTTATAGCCTTAAACTGTGGCCTCACA
TTCCTTCCTATCTTACTTTCCTGCACTGGGGTAAATGTCTCCTTGCTCTTCTTGCTTTCT
GTCCTACTGCAGGGCTCTTGCTGAGCTGGTGAAGCACAAGCCCAAGGCTACAGCGGAGCA
ACTGAAGACTGTCATGGATGACTTTGCACAGTTCCTGGATACATGTTGCAAGGCTGCTGA
CAAGGACACCTGCTTCTCGACTGAGGTCAGAAACGTTTTTGCATTTTGACGATGTTCAGT
TTCCATTTTCTGTGCACGTGGTCAGGTGTAGCTCTCTGGAACTCACACACTGAATAACTC
CACCAATCTAGATGTTGTTCTCTACGTAACTGTAATAGAAACTGACTTACGTAGCTTTTA
ATTTTTATTTTCTGCCACACTGCTGCCTATTAAATACCTATTATCACTATTTGGTTTCAA
ATTTGTGACACAGAAGAGCATAGTTAGAAATACTTGCAAAGCCTAGAATCATGAACTCAT
TTAAACCTTGCCCTGAAATGTTTCTTTTTGAATTGAGTTATTTTACACATGAATGGACAG
TTACCATTATATATCTGAATCATTTCACATTCCCTCCCATGGCCTAACAACAGTTTATCT
TCTTATTTTGGGCACAACAGATGTCAGAGAGCCTGCTTTAGGAATTCTAAGTAGAACTGT
AATTAAGCAATGCAAGGCACGTACGTTTACTATGTCATTGCCTATGGCTATGAAGTGCAA
ATCCTAACAGTCCTGCTAATACTTTTCTAACATCCATCATTTCTTTGTTTTCAGGGTCCA
AACCTTGTCACTAGATGCAAAGACGCCTTAGCCggaagcggcgccaccaatttcagcctg
ctgaaacaggccggcgacgtggaagagaaccctggccctTCCTTTATTCCAGTGGCCGAG
GACTCCGACTTTCCCATCCAAAACCTGCCCTATGGTGTTTTCTCCACTCAAAGCAACCCA
AAGCCACGGATTGGTGTAGCCATCGGTGACCAGATCTTGGACCTGAGTGTCATTAAACAC
CTCTTTACCGGACCTGCCCTTTCCAAACATCAACATGTCTTCGATGAGACAACTCTCAAT
AACTTCATGGGTCTGGGTCAAGCTGCATGGAAGGAGGCAAGAGCATCCTTACAGAACTTA
CTGTCTGCCAGCCAAGCCCGGCTCAGAGATGACAAGGAGCTTCGGCAGCGTGCATTCACC
TCCCAGGCTTCTGCGACAATGCACCTTCCTGCTACCATAGGAGACTACACGGACTTCTAC
TCTTCTCGGCAGCATGCCACCAATGTTGGCATTATGTTCAGAGGCAAGGAGAATGCGCTG
TTGCCAAATTGGCTCCACTTACCTGTGGGATACCATGGCCGAGCTTCCTCCATTGTGGTA
TCTGGAACCCCGATTCGAAGACCCATGGGGCAGATGAGACCTGATAACTCAAAGCCTCCT
GTGTATGGTGCCTGCAGACTCTTAGACATGGAGTTGGAAATGGCTTTCTTCGTAGGCCCT
GGGAACAGATTCGGAGAGCCAATCCCCATTTCCAAAGCCCATGAACACATTTTCGGGATG
GTCCTCATGAACGACTGGAGCGCACGAGACATCCAGCAATGGGAGTACGTCCCACTTGGG
CCATTCCTGGGGAAAAGCTTTGGAACCACAATCTCCCCGTGGGTGGTGCCTATGGATGCC
CTCATGCCCTTTGTGGTGCCAAACCCAAAGCAGGACCCCAAGCCCTTGCCATATCTCTGC
CACAGCCAGCCCTACACATTTGATATCAACCTGTCTGTCTCTTTGAAAGGAGAAGGAATG
AGCCAGGCGGCTACCATCTGCAGGTCTAACTTTAAGCACATGTACTGGACCATGCTGCAG
CAACTCACACACCACTCTGTTAATGGATGCAACCTGAGACCTGGGGACCTCTTGGCTTCT
GGAACCATCAGTGGATCAGACCCTGAAAGCTTTGGCTCCATGCTGGAACTGTCCTGGAAG
GGAACAAAGGCCATCGATGTGGGGCAGGGGCAGACCAGGACCTTCCTGCTGGACGGCGAT
GAAGTCATCATAACAGGTCACTGCCAGGGGGACGGCTACCGTGTTGGCTTTGGCCAGTGT
GCTGGGAAAGTGCTGCCTGCCCTTTCACCAGCCTAAACACATCACAACCACAACCTTCTC
AGGTAACTATACTTGGGACTTAAAAAACATAATCATAATCATTTTTCCTAAAACGATCAA
GACTGATAACCATTTGACAAGAGCCATACAGACAAGCACCAGCTGGCACTCTTAGGTCTT
CACGTATGGTCATCAGTTTGGGTTCCATTTGTAGATAAGAAACTGAACATATAAAGGTCT
AGGTTAATGCAATTTACACAAAAGGAGACCAAACCAGGGAGAGAAGGAACCAAAATTAAA
AATTCAAACCAGAGCAAAGGAGTTAGCCCTGGTTTTGCTCTGACTTACATGAACCACTAT
GTGGAGTCCTCCATGTTAGCCTAGTCAAGCTTATCCTCTGGATGAAGTTGAAACCATATG
AAGGAATATTTGGGGGGTGGGTCAAAACAGTTGTGTATCAATGATTCCATGTGGTTTGAC
CCAATCATTCTGTGAATCCATTTCAACAGAAGATACAACGGGTTCTGTTTCATAATAAGT
GATCCACTTCCAAATTTCTGATGTGCCCCATGCTAAGCTTTAACAGAATTTATCTTCTTA
TGACAAAGCAGCCTCCTTTGAAAATATAGCCAACTGCACACAGCTATGTTGATCAATTTT
GTTTATAATCTTGCAGAAGAGAATTTTTTAAAATAGGGCAATAATGGAAGGCTTTGGCAA
AAAAATTGTTTCTCCATATGAAAACAAAAAACTTATTTTTTTATTCAAGCAAAGAACCTA
TAGACATAAGGCTATTTCAAAATTATTTCAGTTTTAGAAAGAATTGAAAGTTTTGTAGCA
TTCTGAGAAGACAGCTTTCATTTGTAATCATAGGTAATATGTAGGTCCTCAGAAATGGTG
AGACCCCTGACTTTGACACTTGGGGACTCTGAGGGACCAGTGATGAAGAGGGCACAACTT
ATATCACACATGCACGAGTTGGGGTGAGAGGGTGTCACAACATCTATCAGTGTGTCATCT
GCCCACCAAGTAACAGATGTCAGCTAAGACTAGGTCATGTGTAGGCTGTCTACACCAGTG
AAAATCGCAAAAAGAATCTAAGAAATTCCACATTTCTAGAAAATAGGTTTGGAAACCGTA
TTCCATTTTACAAAGGACACTTACATTTCTCTTTTTGTTTTCCAGGCTACCCTGAGAAAA
AAAGACATGAAGACTCAGGACTCATCTTTTCTGTTGGTGTAAAATCAACACCCTAAGGAA
CACAAATTTCTTTAAACATTTGACTTCTTGTCTCTGTGCTGCAATTAATAAAAAATGGAA
AGAATCTACTCTGTGGTTCAGAACTCTATCTTCCAAAGGCGCGCTTCACCCTAGCAGCCT
CTTTGGCTCAGAGGAATCCCTGCCTTTCCTCCCTTCATCTCAGCAGAGAATGTAGTTCCA
CATGGGCAACACAATGAAAATAAACGTTAATACTCTCCCATCTTATGGGTGGTGACCCTA
GAAACCAATACTTCAACATTACGAGAATTCTGAATGAGAGACTAAAAGCTTATGAACTGT
GGCTTTCCTTTGTCAGTGGGACTCTAAGAATGAGTTGGGGACAAAAGAGATAGGAATGGC
TTTAAAGGTGACTAGTTGAACTGATAAAGTAAATGAACTGAGGAAAAAAAATATCACTCA
Atcggtaaggaacccctagtgatggagttggccactccctctctgcgcgctcgctcgctc
actgaggccgggcgaccaaaggtcgcccgacgcccgggctttgcccgggcggcctcagtg
agcgagcgagcgcgcagagagggagtggccaactttttgcaaaagcctaggcctccaaaa
aagcctcctcactacttctggaatagctcagaggccgaggcggcctcggcctctgcataa
ataaaaaaaattagtcagccatggggcggagaatgggcggaactgggcggagttaggggc
gggatgggcggagttaggggcgggactatggttgctgactaattgagatgcatgctttgc
atacttctgcctgctggggagcctggggactttccacacctggttgctgactaattgaga
tgcatgctttgcatacttctgcctgctggggagcctggggactttccacaccctaactga
cacacattccacagctgcattaatgaatcggccaacgcgcggggagaggcggtttgcgta
ttgggcgctcttccgcttcctcgctcactgactcgctgcgctcggtcgttcggctgcggc
gagcggtatcagctcactcaaaggcggtaatacggttatccacagaatcaggggataacg
caggaaagaacatgtgagcaaaaggccagcaaaaggccaggaaccgtaaaaaggccgcgt
tgctggcgtttttccataggctccgcccccctgacgagcatcacaaaaatcgacgctcaa
gtcagaggtggcgaaacccgacaggactataaagataccaggcgtttccccctggaagct
ccctcgtgcgctctcctgttccgaccctgccgcttaccggatacctgtccgcctttctcc
cttcgggaagcgtggcgctttctcatagctcacgctgtaggtatctcagttcggtgtagg
tcgttcgctccaagctgggctgtgtgcacgaaccccccgttcagcccgaccgctgcgcct
tatccggtaactatcgtcttgagtccaacccggtaagacacgacttatcgccactggcag
cagccactggtaacaggattagcagagcgaggtatgtaggcggtgctacagagttcttga
agtggtggcctaactacggctacactagaagaacagtatttggtatctgcgctctgctga
agccagttaccttcggaaaaagagttggtagctcttgatccggcaaacaaaccaccgctg
gtagcggtggtttttttgtttgcaagcagcagattacgcgcagaaaaaaaggatctcaag
aagatcctttgatcttttctacggggtctgacgctcagtggaacgaaaactcacgttaag
ggattttggtcatgagattatcaaaaaggatcttcacctagatccttttaaattaaaaat
gaagttttaaatcaatctaaa

Characterization of Disease Progression and Resolution with and without NTBC Treatment in Fah−/− Mice

Fah−/− mice were kept on 8 mg/L of NTBC-drinking water prior to the initiation of the study at 7 weeks of age. Animals were then supplied with regular water (NTBC off) for 7 days, followed by NTBC-containing water at 8 mg/L for another 7 days. The above cycling was repeated for another 14 days. During the study, animals were monitored for daily body weight. Submandibular blood and urine were sampled periodically, and serum and plasma were collected and stored at −80° C. until further analysis. At sacrifice, blood was collected for plasma via cardiac puncture. For liver dissection, one lobe of liver was fixed by 10% formalin, and the remaining was snap-frozen and stored at −80° C. The following day, the formalin-fixed liver was transferred to 70% ethanol for paraffin embedding.

### Measuring the Severity of Disease Phenotypes in GeneRide-Treated Fah−/− Mice During the Selective Expansion of Edited Hepatocytes.

Four-week-old Fah^{-/-} animals were treated with rAAV.DJ-GR-mFah at a dose of 1e14 vg/kg via retro-orbital sinus under anesthesia. All mice were kept on 8 mg/L of NTBC-drinking water prior to the initiation of the study and 3 weeks post dosing. Thereafter, animals were supplied regular water (NTBC off) until the conclusion of study. During the study, animals were monitored for daily body weight. Submandibular blood and urine were sampled periodically, and serum and plasma were collected and stored at −80° C. until further analysis. At sacrifice, blood was collected for plasma via cardiac puncture. For liver dissection, one lobe of liver was fixed by 10% formalin, and the remaining was snap-frozen and stored at −80° C. The following day, the formalin-fixed liver was transferred to 70% ethanol for paraffin embedding.

### Evaluation of Preventive Effect of GENERIDE™ and Nitisinone Therapy (NTBC) on Hepatocellular Carcinoma Development.

Four-week-old Fah^{-/-} animals were treated with rAAV.DJ-GR-mFah at a dose of 1e14 vg/kg via retro-orbital sinus under anesthesia. GeneRide-treated mice were kept on 8 mg/L of NTBC-drinking water prior to the initiation of the study and 1 week post dosing. Thereafter, animals were supplied regular water (NTBC off) until the conclusion of study. Fah^{-/-} animals receiving NTBC treatment at 8 mg/L and Fah^{+/-} animals were the control of the study. During the 1-year study, animals were monitored for body weight. For animals receiving sub-optimal NTBC dosage (0.8 mg/L of NTBC), body weights were monitored 2 times per week since the beginning of 0.8 mg/L NTBC treatment. When any animal was observed with a 10% body weight drop from the previous week, the animal was subjected to 8 mg/L NTBC for 7 days. Thereafter, the animal was returned to receive 0.8 mg/L NTBC until or if the next event of body weight drop. For all animals, submandibular blood and urine were sampled periodically, and serum and plasma were collected and stored at −80° C. until further analysis. At sacrifice, blood was collected for plasma via cardiac puncture. For liver dissection, one lobe of liver was fixed by 10% formalin, and the remaining was snap-frozen and stored at −80° C. The following day, the formalin-fixed liver was transferred to 70% ethanol for paraffin embedding.

Serum Activated Partial Thromboplastin Time (aPTT) Assay

Serum aPTT quantification was performed using STart® Stago blood coagulation analyzer with provided kit (#00595) according to the manufacturer's protocol (Diagnostica Stago, Parsippany, NJ).

### Serum Clinical Chemistry Evaluation

Analysis of serum samples was conducted at Charles River Laboratory (Ashland, OH) using a Liquid Chromatography-Tandem Mass Spectrometry (LC-MS/MS). Clinical chemistry evaluation includes aspartate aminotransferase (AST), alanine aminotransferase (ALT), alkaline phosphatase (ALP), gamma-glutamyl transferase (GGT), total bilirubin (TBIL), albumin (ALB), total protein (TPROT), Globulin (GLOB), creatine kinase (CK), urea nitrogen (UREAN), creatinine (CREAT), electrocytes (PHOS, NA, K, CL), glucose (GLUC), cholesterol (CHOL), and triglyceride (TRIG).

### Example 2. GENERIDE™ Treatment May Improve Liver Function In Vivo

The present example demonstrates that, among other things, viral vectors comprising a sequence encoding fumarylacetoacetate hydrolase (FAH) may be used to treat or prevent tyrosinemia in vivo (e.g., in one or more mouse models).

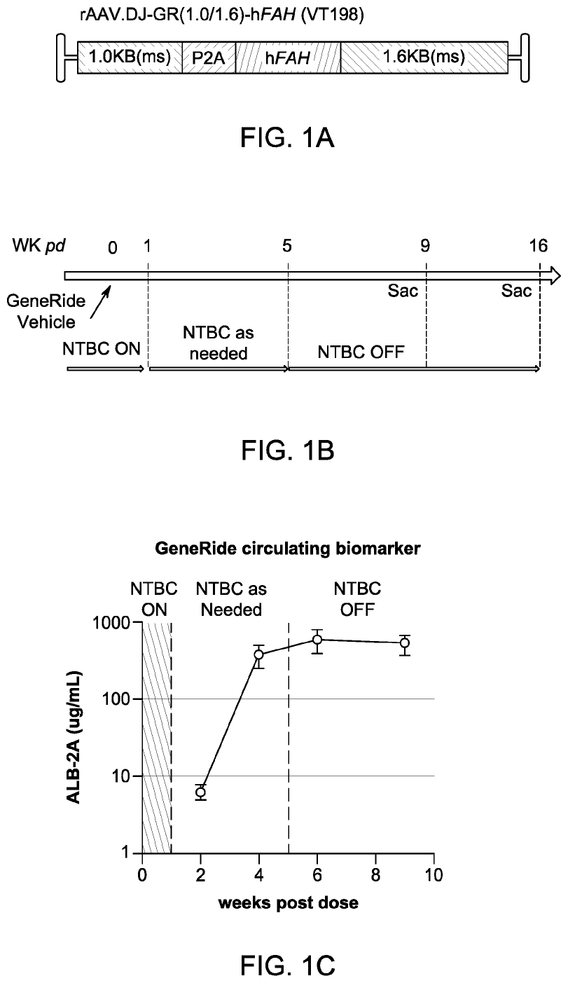

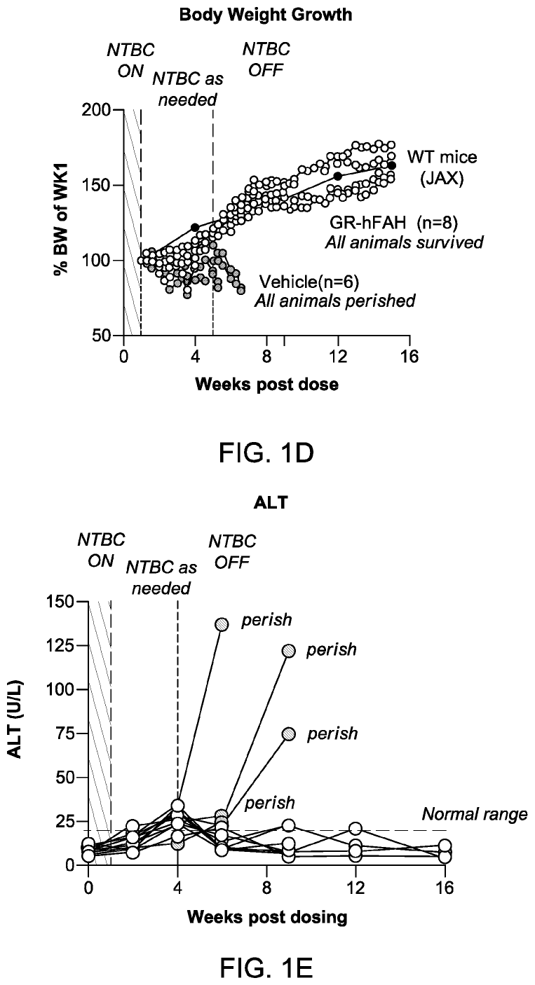

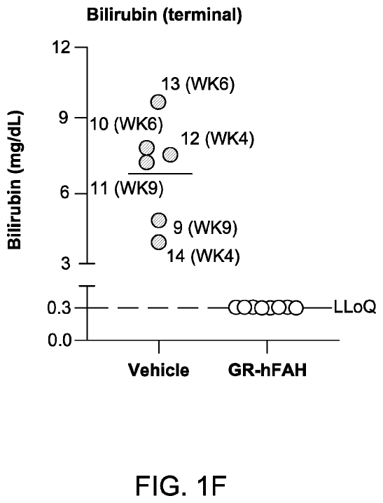

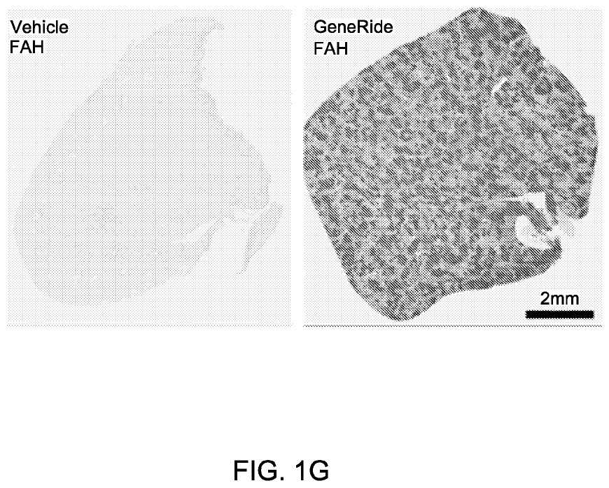

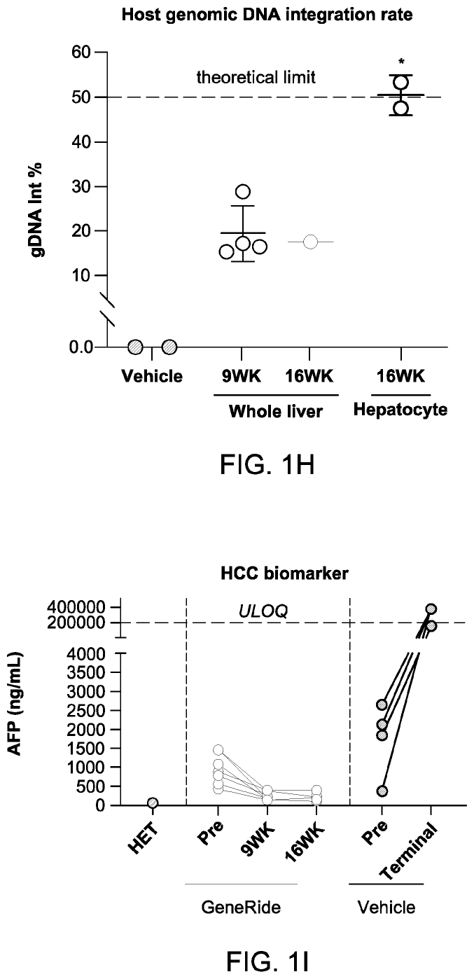

Viral vectors comprising an AAV-DJ viral capsid, human FAH (hFAH) transgene, P2A sequence, a flanking 5′ homology arm 1000 nucleotides (nt) in length, and a 3′ homology arm 1600 nt in length were constructed (FIG. 1A). Homology arms were designed for complementarity to a mouse genomic albumin target integration site. Viral vectors were administered to FRG mice, a mouse model of tyrosinemia, at 4 weeks of age via intravenous injection and maintained on NTBC drinking water (8 mg/L) since birth until 1 week post dosing (FIG. 1B). Between 1 and 4 weeks post dosing, NTBC dosages were adjusted as needed (e.g., if animal body weight dropped 10% below the previous week measurement, animal was cycled back to 8 mg/L NTBC drinking water for one week). From 4 weeks post dosing until sacrifice (at 9 or 16 weeks post dosing), NTBC administration was discontinued.

Mice were assessed for circulating GENERIDE™ biomarkers (e.g., levels of ALB-2A) for up to 9 weeks post-treatment (FIG. 1C) and for body growth (through percent body weight change) for up to 16 weeks post-treatment (FIG. 1D). Markers of liver function (e.g., alanine aminotransferase (ALT), bilirubin) were also assessed (FIG. 1E and FIG. 1F). Mice were sacrificed at 9 weeks (9 WK; 4 animals harvested) and 16 weeks (16 WK; 4 animals harvested) post-treatment and mouse livers were isolated. A subset of treated mice were sacrificed and liver samples were harvested and analyzed through immunohistochemical staining with anti-FAH antibodies (FIG. 1G). Hepatocytes were isolated from a separate subset of treated mice whose livers had been perfused (n=2). Isolated hepatocytes were assessed for gDNA integration (FIG. 1H). Plasma samples from mice were also assessed for presence of alfa-fetoprotein (AFP), a marker for hepatocellular carcinoma (HCC) (FIG. 1I), as compared to healthy Fah+/− heterozygous (HET) mice and vehicle only treated mice.

Among other things, the present disclosure demonstrates that treatment with viral vectors comprising a sequence encoding human FAH (hFAH) may provide improved liver function in a subject suffering from hereditary tyrosinemia 1 (HT1) (e.g., in a FRG mouse model system) as compared to a reference (e.g., untreated or vehicle). In some embodiments, treatment of a subject with viral vectors of the present disclosure may provide reduced levels of biomarkers associated with reduced liver function (e.g., ALT, bilirubin) as compared to a reference (e.g., untreated or vehicle). In some embodiments, treatment of a subject (e.g., a subject suffering from hereditary tyrosinemia) with viral vectors of the present disclosure may allow or restore normal growth (e.g., measured through percentage body weight changes over time) relative to a reference (e.g., untreated or vehicle). In some embodiments, treatment of a subject (e.g., a subject suffering from hereditary tyrosinemia) with viral vectors of the present disclosure may provide reduced levels of a biomarker (e.g., AFP) associated with a disease (e.g., cancer, including HCC). In some embodiments, treatment of a subject (e.g., a subject suffering from hereditary tyrosinemia) with viral vectors of the present disclosure may provide one or more of improved liver function (e.g., measured through assessment of markers of liver function), normal growth (e.g., measured through percentage body weight changes over time) relative to a reference (e.g., untreated or vehicle), and reduced levels of a biomarker (e.g., AFP) associated with a disease (e.g., cancer, including HCC).

Among other things, as shown in FIG. 1H, the present disclosure demonstrates that viral vectors of the present disclosure are capable of integrating a transgene sequence (e.g., FAH) into a genomic target site in a subject. In some embodiments, integration of a transgene sequence (e.g., FAH) may provide a selective advantage for cells (e.g. liver cells) in a subject (e.g., a subject suffering from hereditary tyrosinemia). In some embodiments, treatment of a subject (e.g., a subject suffering from hereditary tyrosinemia) with viral vectors of the present disclosure may enable more than 10% (e.g., 20%, 30%, 40%, 50%) genomic DNA integration of a delivered transgene (e.g., FAH) in cells (e.g., liver cells) within a particular tissue type (e.g. liver). In some embodiments, treatment of a subject (e.g., a subject suffering from hereditary tyrosinemia) with viral vectors of the present disclosure may enable more than 50% (e.g., 60%, 70%, 80%, 90%, 95%, 99%, 100%, etc.) of cells (e.g., liver cells) within a particular tissue type (e.g. liver) to consist of cells that have successfully integrated a delivered transgene (e.g., FAH)

### Example 3. GENERIDE™ Treatment Allows Rapid Selective Expansion of Edited Cells In Vivo

The present example demonstrates that, among other things, viral vectors comprising a sequence encoding fumarylacetoacetate hydrolase (FAH) may be administered to a subject (e.g., a subject suffering from hereditary tyrosinemia type 1) at certain dosages and provide a selective advantage for cells that have successfully integrated a FAH-encoding sequence.

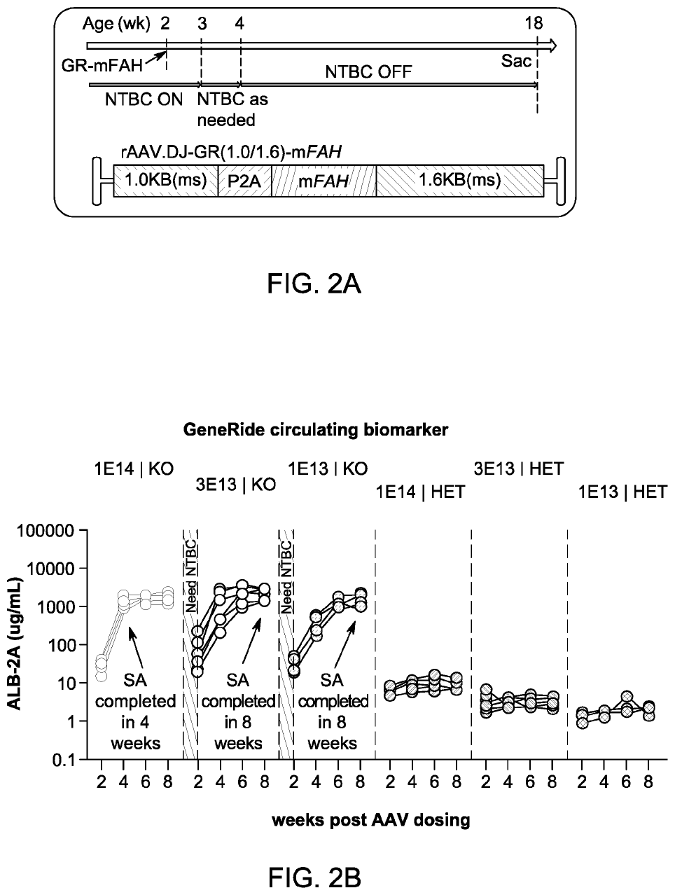

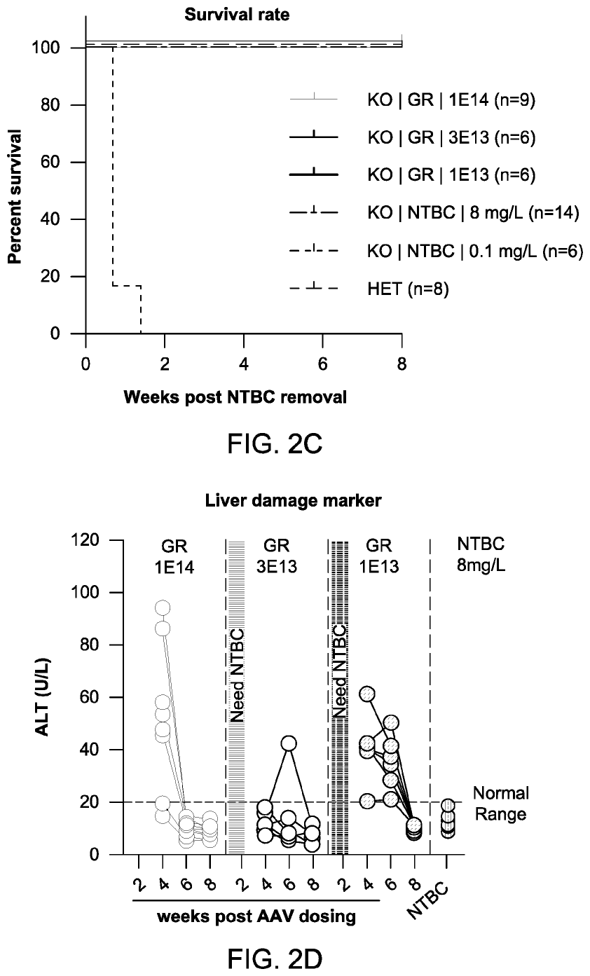

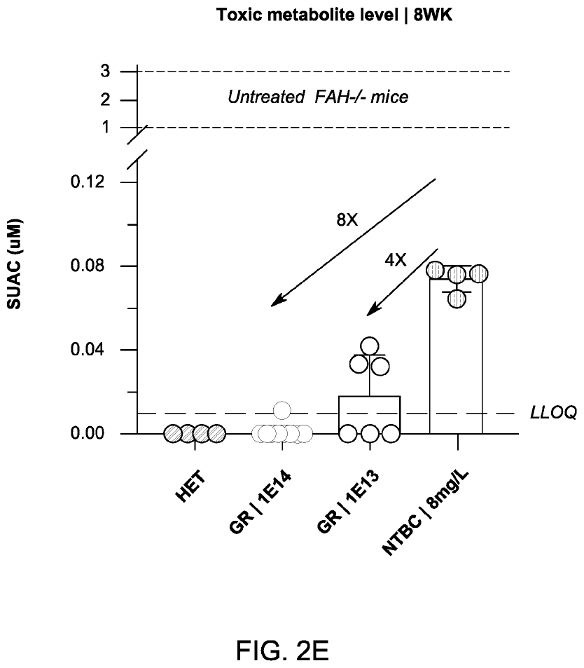

Viral vectors comprising an AAV-DJ viral capsid, mouse FAH (mFAH) transgene, P2A sequence, a flanking 5′ homology arm 1000 nucleotides (nt) in length, and a 3′ homology arm 1600 nt in length were constructed (FIG. 2A). Homology arms were designed for complementarity to a mouse genomic albumin target integration site. Vectors herein described were administered to Fah−/− mice via intravenous injection at two weeks of age (FIG. 2A). Mice were supplied with 8 mg/L of NTBC from birth until 1 week post administration of GENERIDE™ vectors (3 weeks of age). Between 3 and 4 weeks of age, 8 mg/L NTBC drinking water was only supplied to animals observed with 10% body weight drop from the previous week. From 4 weeks of age until sacrifice (at least 18 weeks of age), NTBC administration was discontinued. Another group of Fah−/− mice was always maintained with 8 mg/L NTBC drinking water and served as standard of care control group. Mice were assessed for circulating biomarkers (e.g., levels of ALB-2A) for up to 8 weeks post-treatment (FIG. 2B) and survival rate post NTBC removal (FIG. 2C). Markers of liver function (e.g., ALT) and presence of toxic metabolites (e.g., succinylacetone (SUAC)) were assessed (FIG. 2D and FIG. 2E).

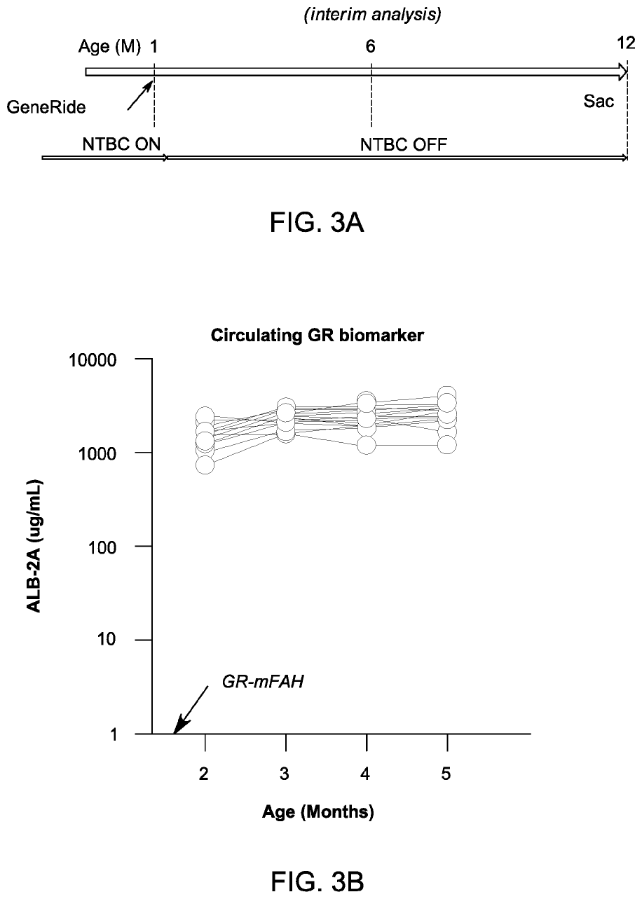

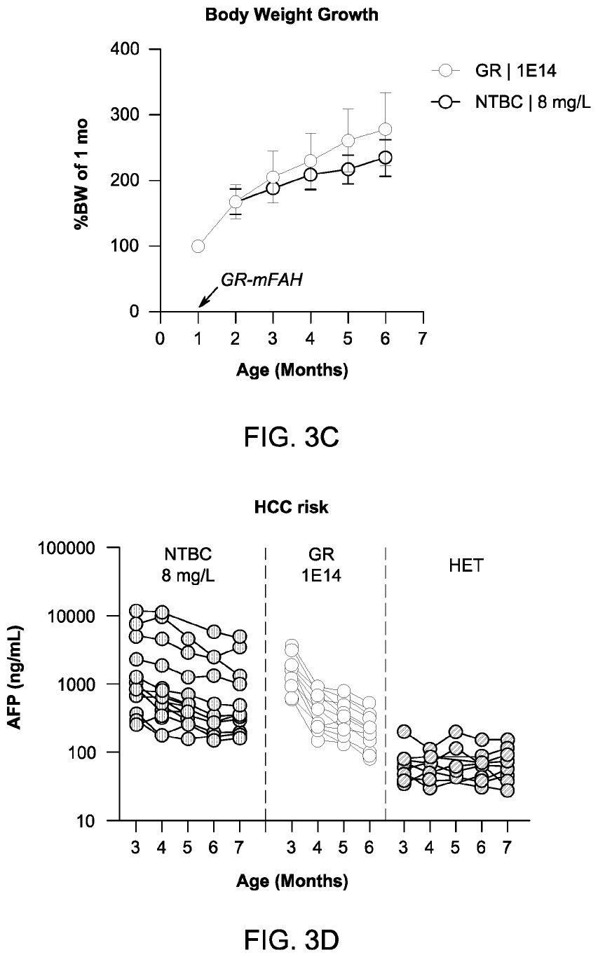

Viral vectors comprising an AAV-DJ viral capsid, mouse FAH (mFAH) transgene, P2A sequence, a flanking 5′ homology arm 1000 nucleotides (nt) in length, and a 3′ homology arm 1600 nt in length were constructed (FIG. 2A). Homology arms were designed for complementarity to a mouse genomic albumin target integration site. Vectors herein described were administered to Fah−/− mice via intravenous injection at 1 month of age (FIG. 3A). Mice were treated with 8 mg/L of NTBC from birth until administration of GENERIDE™ vectors. From 1 month of age until sacrifice (at least 12 month of age), NTBC administration was discontinued. Mice were assessed for circulating biomarkers (e.g., levels of ALB-2A) for at least 5 months of age (FIG. 3B) and body weight was monitored for at least 6 months post treatment (FIG. 3C). HCC risk (AFP level) was also assessed in these mice (at least 6 months of age) (FIG. 3D).

Among other things, the present disclosure demonstrates that treatment of a subject (e.g., a subject suffering from hereditary tyrosinemia type 1) with viral vectors of the present disclosure may provide a rapid selective advantage for cells (e.g. liver cells), leading to complete repopulation of the diseased liver within 4 weeks post-treatment. In some embodiments, treatment of a subject (e.g., a subject suffering from hereditary tyrosinemia) with viral vectors of the present disclosure may enable more than 50% (e.g., 60%, 70%, 80%, 90%, 95%, 99%, 100%, etc.) of cells (e.g., liver cells) within a particular tissue type (e.g. liver) to consist of cells that have successfully integrated a delivered transgene (e.g., FAH) within 4 weeks post-treatment. In some embodiments, treatment a subject (e.g., a subject suffering from hereditary tyrosinemia) with viral vectors of the present disclosure at certain doses (e.g., 3E13 vg/kg, 1E13 vg/kg, 1E14 vg/kg) may provide a selective advantage for cells (e.g. liver cells) within 4 weeks post-treatment.

Among other things, the present disclosure demonstrates that treatment with viral vectors comprising a sequence encoding mouse FAH (mFAH) may provide improved liver function in a subject suffering from hereditary tyrosinemia 1 (HT1) (e.g., in a FAH^{-/-} mouse model system) as compared to a reference (e.g., untreated). In some embodiments, treatment of a subject with viral vectors of the present disclosure may provide reduced levels of biomarkers associated with reduced liver function (e.g., ALT, bilirubin) as compared to a reference (e.g., untreated). In some embodiments, treatment of a subject (e.g., a subject suffering from hereditary tyrosinemia) with viral vectors of the present disclosure may provide reduced levels of a harmful metabolite (e.g., SUAC) relative to a reference (e.g., untreated or NTBC-treated).

Among other things, the present disclosure demonstrates that treatment of a subject (e.g., a subject suffering from hereditary tyrosinemia) with viral vectors of the present disclosure may lower HCC risk as compared to a reference (e.g., untreated or NTBC-treated subjects). In some embodiments, treatment of a subject (e.g., a subject suffering from hereditary tyrosinemia) with viral vectors of the present disclosure may provide reduced levels of a biomarker (e.g., AFP) associated with a disease (e.g., cancer, including HCC) at least 5 months post dosing (6 months of age), as compared to an age-matched reference (e.g., untreated or NTBC-treated subjects).

Among other things, the present disclosure demonstrates that treatment of a subject (e.g., a subject suffering from hereditary tyrosinemia) with viral vectors of the present disclosure may show continued transgene expression as adults. In some embodiments, administration of viral vectors of the present disclosure to a subject at least one month of age demonstrated continued expression of circulating biomarker (e.g., ALB-2A) at least 5 months post-dosing. In some embodiments, subjects treated with viral vectors of the present disclosure showed similar body weight as compared to a reference (e.g., NTBC-treated subjects). In some embodiments, treatment of a subject (e.g., a subject suffering from hereditary tyrosinemia) with viral vectors of the present disclosure may provide reduced levels of a biomarker (e.g., AFP) associated with a disease (e.g., cancer, including HCC) as compared to a reference (e.g., untreated or NTBC-treated subjects) at least 6 months of age.

### Example 4. Optimization of GENERIDE™ Combination Therapies

The present example demonstrates that, among other things, viral vectors comprising a sequence encoding fumarylacetoacetate hydrolase (FAH) may be administered to a subject (e.g., a subject suffering from hereditary tyrosinemia) in combination with certain dosages of one or more alternative therapies (e.g., NTBC-treatment) in order to optimize selective advantage for cells that have successfully integrated a FAH-encoding sequence.

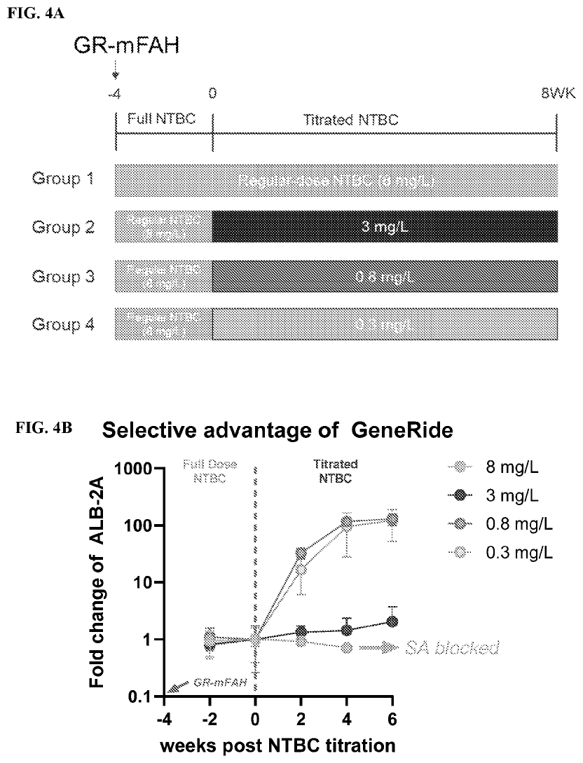

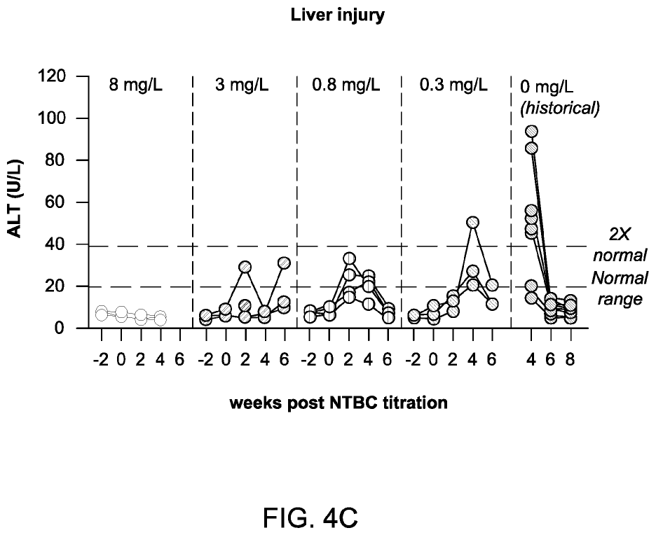

Viral vectors comprising an AAV-DJ viral capsid, mouse FAH (mFAH) transgene, P2A sequence, and flanking 5′ homology arm 1000 nucleotides (nt) in length and a 3′ homology arm 1600 nt in length homology arms were constructed. Homology arms were designed for complementarity to a mouse genomic albumin target integration site. Viral vectors herein described were administered at a dosage of 1e14 vg/kg to three groups (Groups 1, 2, 3, 4) of Fah−/− mice via intravenous injection at 4 weeks of age. Mice in all groups were maintained on NTBC drinking water (8 mg/L) for 4 weeks, followed by a titrated dose of NTBC (3 mg/L, 0.8 mg/L, and 0.3 mg/L, respectively), for 8 weeks. Mice in group 1 remained on the standard dose of NTBC for 8 weeks. Mice were assessed for circulating biomarkers (e.g., levels of ALB-2A) for up to 6 weeks post-NTBC titration (FIG. 4B).

Among other things, the present disclosure demonstrates that treatment of a subject (e.g., a subject suffering from hereditary tyrosinemia) with viral vectors of the present disclosure may comprise administration of viral vectors in combination with one or more alternative therapies (e.g. alternative HT1 therapy), in order to provide a selective advantage for cells (e.g. liver cells). In some embodiments, administration of viral vectors of the present disclosure in combination with one or more alternative therapies (e.g., NTBC) may be optimized (e.g., through optimization of dosage level and/or timing) to provide a selective advantage for cells (e.g., liver cells) while maintaining or improving liver function (e.g., reduced levels of biomarkers associated with reduced liver function (e.g., ALT, bilirubin) (FIG. 4C), reduced levels of harmful metabolites (e.g., SUAC), etc.). In some embodiments, dosage level of one or more alternative therapies (e.g., NTBC therapies) may be titrated to provide a selective advantage for cells (e.g., liver cells) while controlling disease severity (e.g., reducing symptoms and/or side effects of disease).

Among other things, the present disclosure demonstrates that treatment with viral vectors comprising a sequence encoding FAH (FAH) may provide improved integration with supplemented titrated lower doses of NTBC in a subject suffering from hereditary tyrosinemia 1 (e.g., in a Fah−/− mouse model system). In some embodiments, as demonstrated in FIG. 4B, treatment of a subject with viral vectors of the present disclosure may provide increased levels of circulating biomarkers (e.g., ALB-2A) in conjunction with sub-optimal NTBC dose, whereas high doses of NTBC may prevent selective expansion of GENERIDE™ edited cells (e.g., hepatocytes).

### Example 5. GENERIDE™ Treatment May Show Improved Liver Function Prior to Complete Selective Expansion of Edited Cells In Vivo

The present example demonstrates that, among other things, viral vectors comprising specific components along with a sequence encoding fumarylacetoacetate hydrolase (FAH) administered to a subject (e.g., a subject suffering from hereditary tyrosinemia 1) may improve liver function prior to complete selective expansion of cells that have successfully integrated a FAH-encoding sequence.

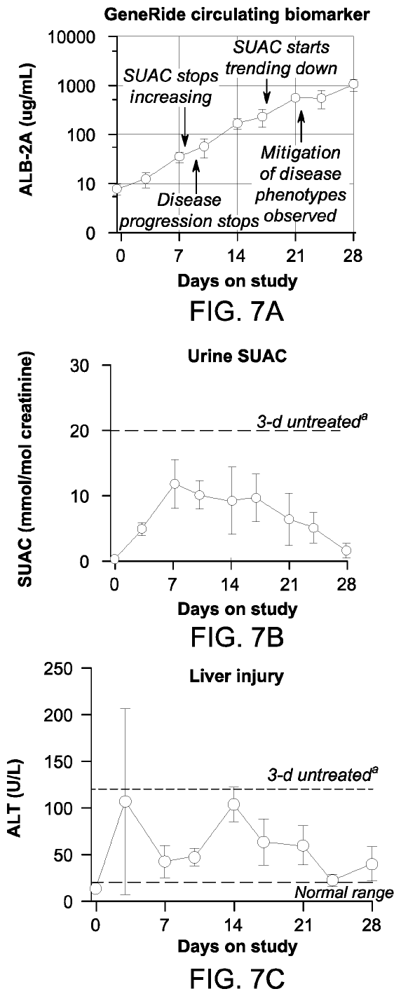

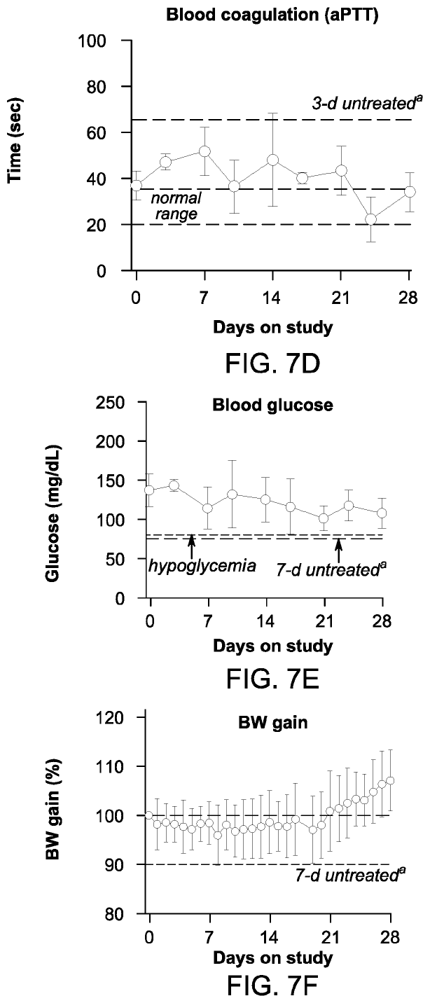

Viral vectors comprising an AAV-DJ viral capsid, mouse FAH (mFAH) transgene, P2A sequence, and flanking 5′ homology arm 1000 nucleotides (nt) in length and a 3′ homology arm 1600 nt in length homology arms were constructed. Homology arms were designed for complementarity to a mouse genomic albumin target integration site. Viral vectors herein described were administered to Fah−/− mice at a dosage of 1e14 vg/kg. All mice were kept on 8 mg/L of NTBC-drinking water prior to the initiation of the study and 3 weeks post dosing. Thereafter, animals were supplied regular water (NTBC off) until the conclusion of the study. Mice were assessed for circulating GENERIDE™ biomarkers (e.g., levels of ALB-2A) (FIG. 7A), presence of toxic metabolic (e.g., SUAC) (FIG. 7B), and markers of liver injury and synthetic function (e.g., ALT) (FIG. 7C), blood coagulation (e.g., activated partial thromboplastin time (aPTT)), (FIG. 7D), blood glucose (FIG. 7E), and/or body weight (FIG. 7F).

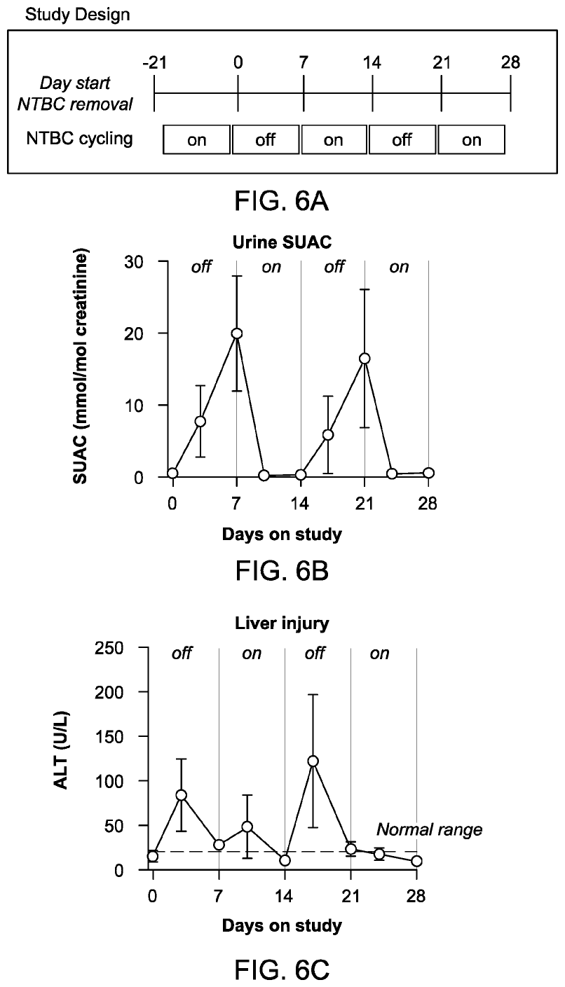

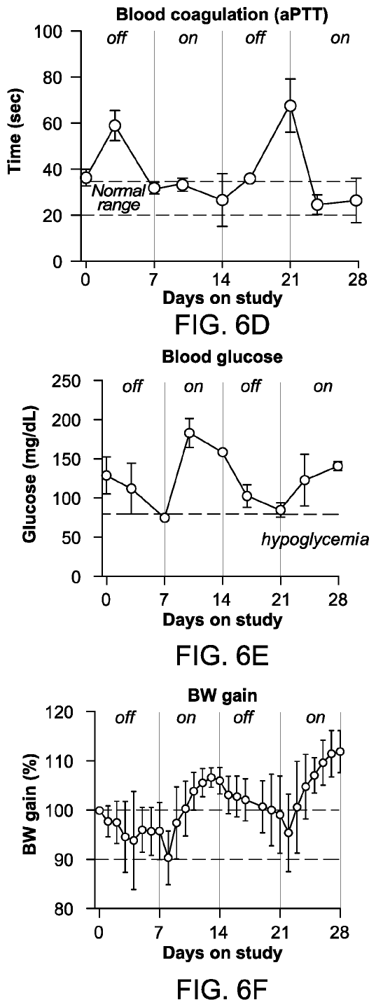

NTBC treatment, as demonstrated in FIG. 6A-6F, may quickly (e.g., less than 7 days) restore levels of hereditary tyrosinemia disease biomarkers (e.g., SUAC) and liver synthetic function (aPTT, ALT, blood glucose) and abnormal levels of hereditary tyrosinemia disease biomarkers (e.g., SUAC) and liver synthetic function (e.g., aPTT, ALT, blood glucose) may follow upon withdrawal (e.g., less than 3 days) of NTBC treatment.

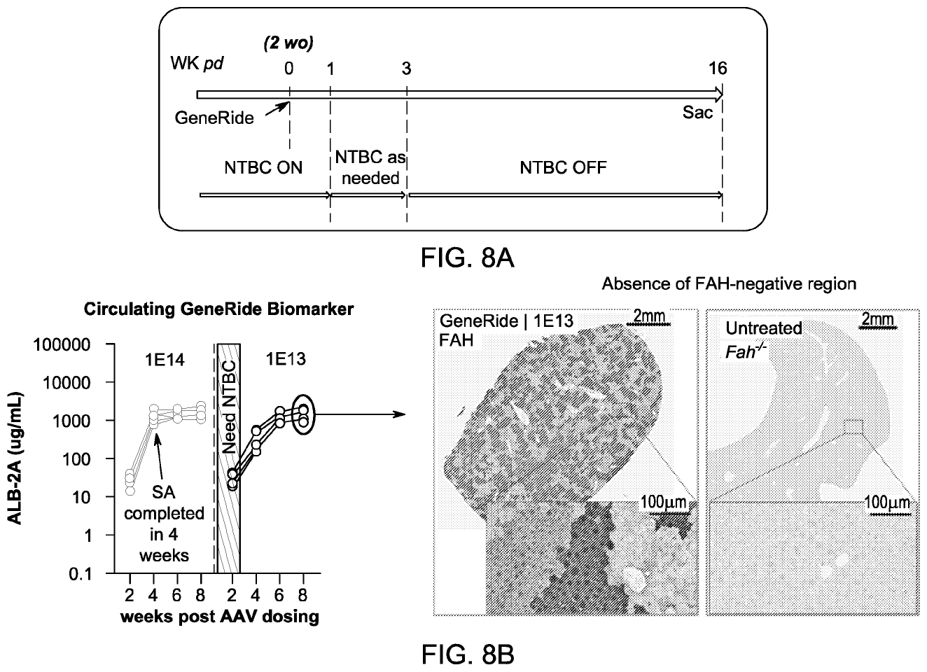

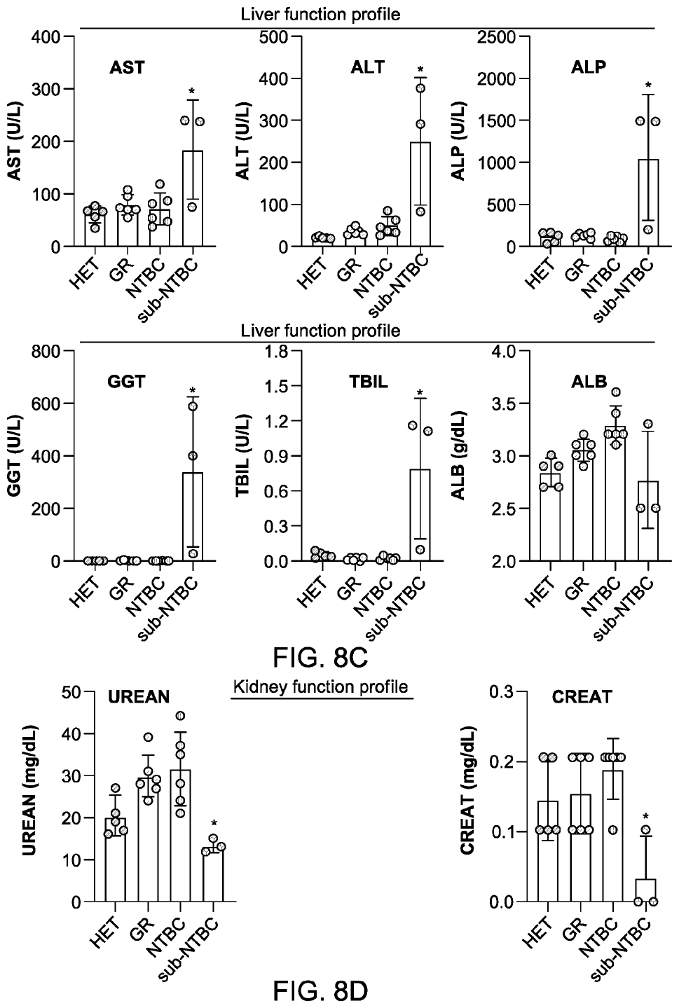

As demonstrated in FIG. 7A-7F, within 7 days of GENERIDE™ treatment replacement of NTBC in Fah−/− mice, the expansion of corrected cells may be sufficient to control SUAC without NTBC treatment. At day 7, only mild liver injury was observed and there was no severe metabolic dysfunction detected. From Day 17 to Day 28, SUAC levels trended down to the normal range, followed by body weight gain. Importantly, as demonstrated in FIG. 8B, by Day 28 (e.g., 4 weeks), treatment with GENERIDE™ fully repopulated the liver with GENERIDE™-edited cells that have successfully integrated the FAH transgene. As shown herein, GENERIDE™ treatment resulted in comparable liver and kidney biomarker profiles to full-dose NTBC treatment, and both GENERIDE™ and NTBC treatment showed similar levels as compared to healthy Fah+/− mice (FIGS. 8C and 8D.

### Example 6. Long-Term GENERIDE™ Treatment Allows Rapid Selective Expansion of Edited Cells in Liver with Low Hepatocellular Carcinoma (HCC) Risk

The present example demonstrates that, among other things, viral vectors comprising specific components along with a sequence encoding fumarylacetoacetate hydrolase (FAH) administered to a subject (e.g., a subject suffering from hereditary tyrosinemia 1) may provide a selective advantage for cells that have successfully integrated a FAH-encoding sequence, and that these edited hepatocytes may confer a lower risk of developing HCC than unedited hepatocytes.

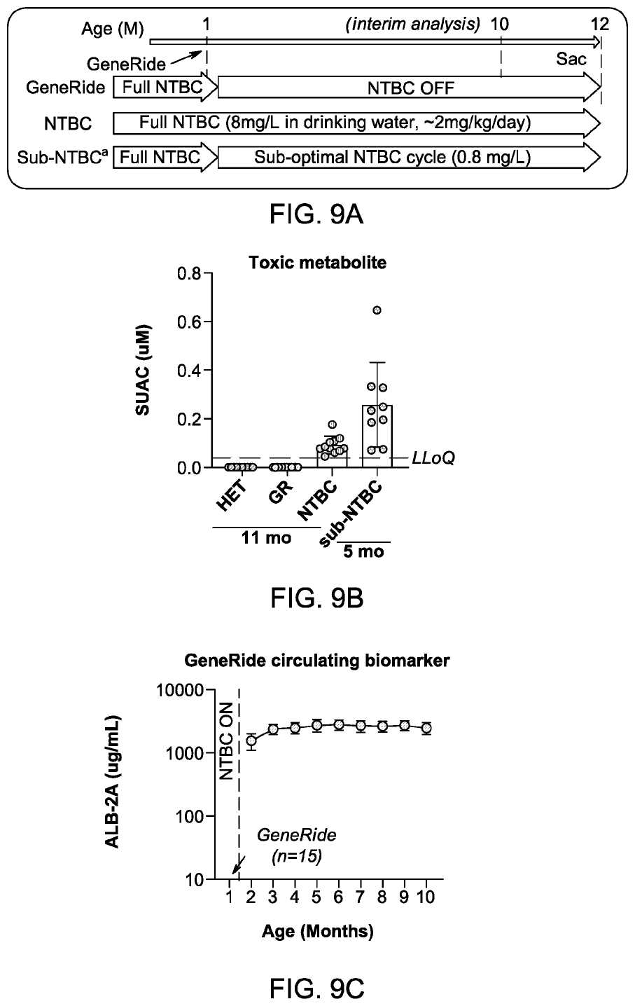

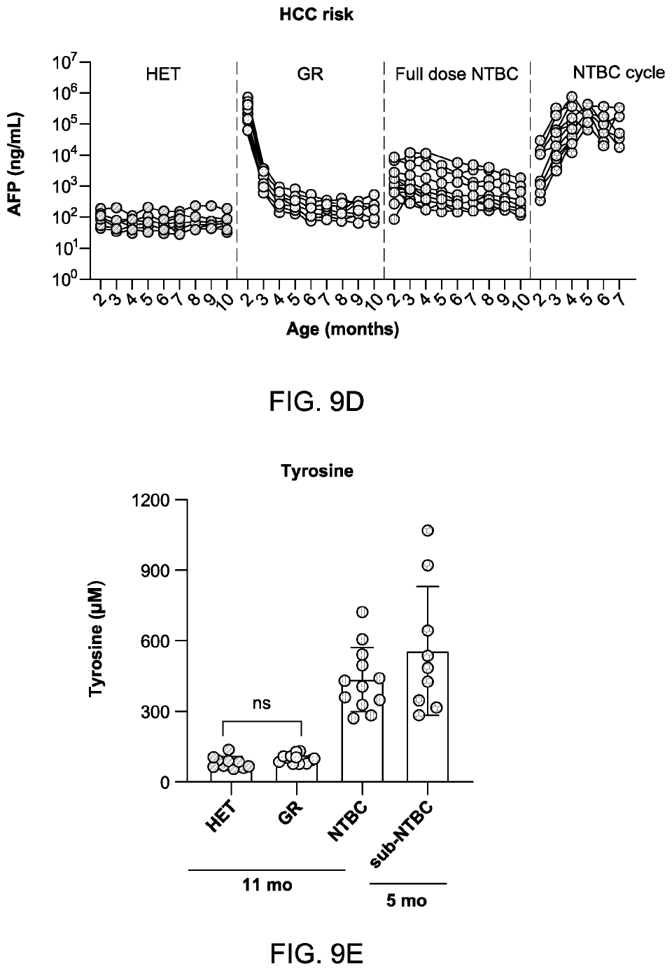

Viral vectors comprising an AAV-DJ viral capsid, mouse FAH (mFAH) transgene, P2A sequence, and flanking 5′ homology arm 1000 nucleotides (nt) in length and a 3′ homology arm 1600 nt in length homology arms were constructed. Homology arms were designed for complementarity to a mouse genomic albumin target integration site. Viral vectors herein described were administered to Fah−/− mice at a dosage of 1e14 vg/kg. GENERIDE™-treated mice were kept on 8 mg/L of NTBC-drinking water prior to the initiation of the study and 1 week post dosing. Thereafter, animals were supplied regular water (NTBC off) until the conclusion of study (FIG. 9A).

During the study, animals were monitored for body weight. For animals receiving sub-optimal NTBC dosage, body weights were monitored 2 times per week since the beginning of 0.8 mg/L NTBC treatment. The animal was subjected to 8 mg/L NTBC for 7 days if there was an observed 10% body weight drop from a previous week. Mice were assessed for circulating GENERIDE™ biomarkers (e.g., levels of ALB-2A) (FIG. 9B), presence of toxic metabolite (e.g., SUAC) (FIG. 9B), and levels of Tyrosine (FIG. 9E). HCC risk (AFP level) was also assessed in these mice (at least 7 months of age) (FIG. 9D).

As demonstrated in FIG. 9C, GENERIDE™ treated Fah−/− mice may show durable hepatocyte editing for at least 10 months. GENERIDE™ treatment may reduce the toxic metabolite (e.g., SUAC) (FIG. 9B) to non-detectable levels and restore tyrosine metabolism (FIG. 9E), demonstrating a long-term superiority to NTBC treatment. This superiority is clinically relevant, especially when diet restriction and NTBC-treatment are not strictly followed (e.g., comparable to a sub-optimal NTBC treatment group). GENERIDE™ treated Fah−/− mice may show a reduction in HCC biomarkers (e.g., AFP) as compared to a high dose of continuous NTBC (8 mg/L)(FIG. 9D), suggesting GENERIDE™ treatment may be associated with an equal or lower HCC risk than Standard of Care (e.g. NTBC).

### Example 7. Optimization of GENERIDE™ Therapies in Pediatric Mice

The present example demonstrates that viral vectors comprising a sequence encoding fumarylacetoacetate hydrolase (FAH) may be administered to a pediatric subject (e.g., a pediatric subject suffering from hereditary tyrosinemia) in order to optimize selective advantage for cells that have successfully integrated a FAH-encoding sequence.

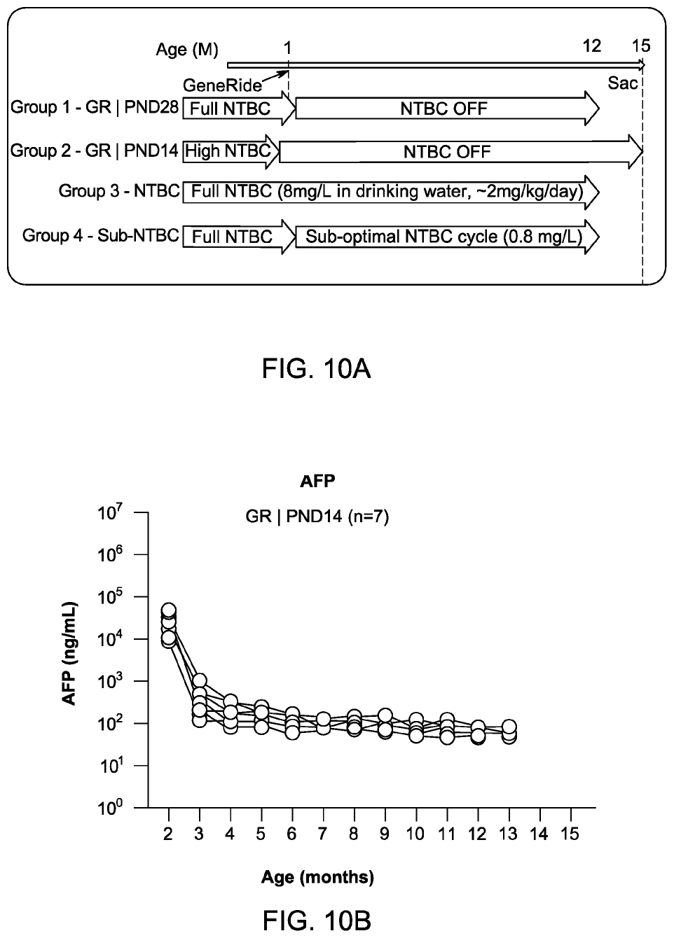

Viral vectors comprising an AAV-DJ viral capsid, mouse FAH (mFAH) transgene, P2A sequence, a flanking 5′ homology arm 1000 nucleotides (nt) in length and a flanking 3′ homology arm 1600 nt in length homology arms were constructed. Homology arms were designed for complementarity to a mouse genomic albumin target integration site. FAH^{-/-} mice were separated into 4 different groups (FIG. 10A). Mice in group 3 were maintained on NTBC drinking water (8 mg/L) for 4 weeks and remained on the standard dose of NTBC (8 mg/L) for 8 weeks. Mice in group 4 were maintained on NTBC drinking water (8 mg/L) for 4 weeks followed by a titrated 0.8 mg/L dose of NTBC for 8 weeks. Mice in group 1 were maintained on NTBC drinking water (8 mg/L) for 4 weeks and at 4 weeks of age were administered viral vectors described herein at a dose of 1E14 vg/kg. Mice in group 2 received milk supplied from a mother dosed with 24 mg/L of NTBC for 3 weeks. This ensured newborns received a dosage close to a standard dose of NTBC (e.g., 8 mg/L). At 2 weeks of age, mice in group 2, were administered viral vectors described herein at a dose of 1E14 vg/kg. HCC risk (e.g., AFP levels) was assessed in these mice

NTBC, which is current standard of care, reduces accumulation of toxic products in patients with hereditary tyrosinemia (see, e.g., Ginkel et al., Adv Exp Med Biol. 2017; 959:101-109). However, risk for developing liver cancer remains especially if NTBC treatment is initiated late (exhibited by a slow decrease of the tumor marker AFP) (see, e.g., Ginkel et al., Adv Exp Med Biol. 2017; 959:101-109).

As demonstrated in FIG. 10B, GENERIDE™ treatment may be effective in FAH^{-/-} mice as early as post-natal day 14 (e.g., mice in group 2) and FAH−/− mice may exhibit a reduction in AFP levels by 3 months of age after receiving treatment. AFP levels were at least about 10⁴ to 10⁵ ng/mL at 2 months of age for mice in group 2 and by 3 months of age AFP levels were reduced to at least about 10² to 10³ ng/mL. In addition, AFP levels remained at least about 10² to 10³ ng/mL for at least about 12 months post treatment.

Thus, the present Example demonstrates that early administration (e.g., by post-natal day 14) of viral vectors described herein may effectively reduce HCC risk in FAH^{-/-} mice characterized by a reduction in AFP levels post treatment.

### Example 8. GENERIDE™ Treatment in Pediatric Mice Allows Rapid Selective Expansion of Edited Cells In Vivo

The present example further confirms that viral vectors comprising a sequence encoding fumarylacetoacetate hydrolase (FAH) may be administered to a pediatric subject (e.g., a subject suffering from hereditary tyrosinemia type 1) and provide a selective advantage for cells that have successfully integrated a FAH-encoding sequence.

Viral vectors comprising an AAV-DJ viral capsid, mouse FAH (mFAH) transgene, P2A sequence, a flanking 5′ homology arm 1000 nucleotides (nt) in length, and a 3′ homology arm 1600 nt in length were constructed. Homology arms were designed for complementarity to a mouse genomic albumin target integration site. Fah−/− mice received milk supplied from a mother dosed with 24 mg/L of NTBC for 3 weeks. This ensured newborns received a dosage close to a standard dose of NTBC (e.g., 8 mg/L). Vectors described herein were administered to mice at a dose of 3E12 vg/kg or 1E13 vg/kg via intravenous injection at two weeks of age (i.e. post-natal day 14). Liver samples were harvested 16 weeks post dosing and analyzed through immunohistochemical staining with anti-FAH antibodies.

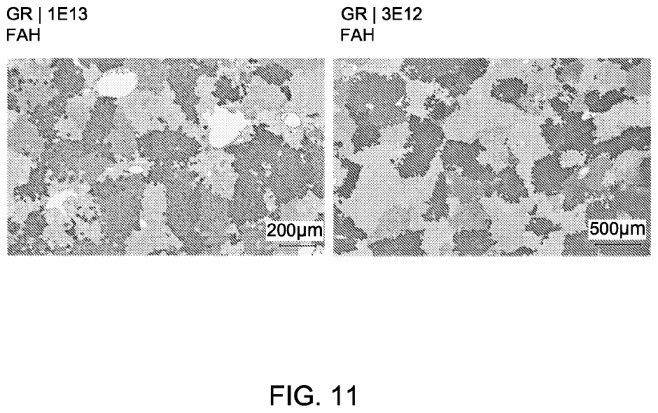

As demonstrated in FIG. 11, liver samples from Fah−/− mice administered a 3E12 vg/kg or 1E13 vg/kg dose of GENERIDE™ treatment, at post-natal day 14, exhibited robust staining for FAH protein at 16 weeks post treatment. Liver samples from Fah−/− mice administered a 3E12 vg/kg of GENERIDE™ treatment exhibited a higher intensity of staining for FAH protein as compared to Fah−/− mice administered a 1E13 vg/kg dose of GENERIDE™ treatment.

Thus, the present Example demonstrates that vectors described herein administered at additional doses (e.g., 3E12 vg/kg or 1E13 vg/kg) in pediatric mice (e.g., post-natal day 14) effectively provide a selective advantage for cells that have successfully integrated a FAH-encoding sequence characterized by robust staining for FAH protein in liver samples. Further, the present example demonstrates, Fah−/− mice administered a low dose of GENERIDE™ treatment (e.g., 3E12 vg/kg) can have improved disease outcomes characterized by robust liver repopulation of cells that have successfully integrated a FAH-encoding sequence.

### Example 9: Two-Plasmid and Three-Plasmid Systems May be Used to Produce Viral Vectors

The present example demonstrates that, among other things, a two-plasmid or three-plasmid system may be used to produce AAV vectors.

In some embodiments, HEK293F cells are expanded for use in vector production. Cells are split to 2e6 cells/mL in 200 mL of Expi293 media in a 500 mL flask. Plasmid mixes for various transfection conditions are made and filtered through a 0.22 μM filter unit. A transfection reagent mix (e.g., PEI or FectoVIR-AAV) is prepared according to manufacturer's protocol. Plasmid and transfection reagent mixes are combined to produce a single transfection mix. 20 mL of transfection mix is added to 100 mL of HEK293F cells in a 500 mL flask and allowed to incubate at 37° C. for 72 hours.

In some embodiments, plasmids used in a two-plasmid system comprise an AAV rep sequence and relevant sequences from a helper viruses (“Rep/Helper Plasmid”) or an AAV cap sequence and a payload (“Payload/Cap Plasmid”). In some embodiments, plasmids used in a three-plasmid system comprise separate plasmids, each encoding one of: 1) an AAV rep and AAV cap sequence, 2) relevant sequence from a helper virus, and 3) a payload. A human gene of interest sequence with flanking homology arms for mouse albumin (e.g., “mHA-FAH”), which is compatible with a GeneRide system, may be used as the payload for experiments in mice. A human gene of interest sequence with flanking homology arms for human albumin (“hHA-FAH”), which is compatible with a GeneRide system, may be used as the payload for experiments in humans or humanized mice. In some embodiments, a payload may comprise SEQ ID NO: 31. In some embodiments, a payload may consist of SEQ ID NO: 31. In some embodiments, a payload may comprise any payload described herein. A variety of AAV cap genes encoding different AAV capsids are assessed within the Payload/Cap plasmid. In some embodiments, the AAV cap gene may encode a AAV1, AAV2, AAV3, AAV4, AAV5, AAV6, AAV7, AAV8, AAV9, AAV10, AAV11, AAVC11.01, AAVC11.02, AAVC11.03, AAVC11.04, AAVC11.05, AAVC11.06, AAVC11.07, AAVC11.08, AAVC11.09, AAVC11.10, AAVC11.11, AAVC11.12, AAVC11.13, AAVC11.14, AAVC11.15, AAVC11.16, AAVC11.17, AAVC11.18, AAVC11.19, AAV-DJ, AAV-LK03, AAV-LK19, AAVrh.74, AAVrh.10, AAVhu.37, AAVrh.K, AAVrh.39, AAV12, AAV 13, AAVrh.8, avian AAV, bovine AAV, canine AAV, equine AAV, primate AAV, non-primate AAV, ovine AAV, a hybrid AAV (e.g., an AAV comprising one more sequences of one AAV subtype and one or more sequences of a second subtype). In some embodiments, a Payload/Cap plasmid may comprise SEQ ID NO: 32. In some embodiments, a Payload/Cap plasmid may consist of SEQ ID NO: 32. In some embodiments, a Payload/Cap plasmid may comprise SEQ ID NO: 33. In some embodiments, a Payload/Cap plasmid may consist of SEQ ID NO: 33. In some embodiments, a Payload/Cap plasmid may comprise any payload or capsid sequence disclosed herein.

TABLE 1A
Exemplary sequences for production of viral vectors.

| | | SEQ ID |
|---|---|---|
| Name | Sequence (5' to 3') | NO |
| Gene of | TTTCTAGATGTAAATAATTATTTTAAGTTTGCCCTAT | 31 |
| interest | GGTGGCCCCACACATGAGACAAACCCCCAAGATGTG | |
| (GOI) | ACTTTTGAGAATGAGACTTGGATAAAAAACATGTAG | |
| cassette | AAATGCAAGCCCTGAAGCTCAACTCCCTATTGCTATC | |
| | ACAGGGGTTATAATTGCATAAAATTTAGCTATAGAA | |
| | AGTTGCTGTCATCTCTTGTGGGCTGTAATCATCGTCT | |
| | AGGCTTAAGAGTAATATTGCAAAACCTGTCATGCCC | |
| | ACACAAATCTCTCCCTGGCATTGTTGTCTTTGCAGAT | |
| | GTCAGTGAAAGAGAACCAGCAGCTCCCATGAGTTTG | |
| | GATAGCCTTATTTTCTATAGCCTCCCCACTATTAGCTT | |
| | TGAAGGGAGCAAAGTTTAAGAACCAAATATAAAGTT | |
| | TCTCATCTTTATAGATGAGAAAAATTTTAAATAAAGT | |
| | CCAAGATAATTAAATTTTTAAGGATCATTTTTAGCTC | |
| | TTTAATAGCAATAAAACTCAATATGACATAATATGG | |
| | CACTTCCAAAATCTGAATAATATATAATTGCAATGAC | |
| | ATACTTCTTTTCAGAGATTTACTGAAAAGAAATTTGT | |
| | TGACACTACATAACGTGATGAGTGGTTTATACTGATT | |
| | GTTTCAGTTGGTCTTCCCACCAACTCCATGAAAGTGG | |
| | ATTTTATTATCCTCATCATGCAGATGAGAATATTGAG | |
| | ACTTATAGCGGTATGCCTGAGCCCCAAAGTACTCAG | |
| | AGTTGCCTGGCTCCAAGATTTATAATCTTAAATGATG | |
| | GGACTACCATCCTTACTCTCTCCATTTTTCTATACGTG | |
| | AGTAATGTTTTTTCTGTTTTTTTTTTTTCTTTTTCCATT | |
| | CAAACTCAGTGCACTTGTTGAGCTCGTGAAACACAA | |
| | GCCCAAGGCAACAAAAGAGCAACTGAAAGCTGTTAT | |
| | GGATGATTTCGCAGCTTTTGTAGAGAAGTGCTGCAA | |
| | GGCTGACGATAAGGAGACCTGCTTTGCCGAGGAGGT | |
| | ACTACAGTTCTCTTCATTTTAATATGTCCAGTATTCAT | |
| | TTTTGCATGTTTGGTTAGGCTAGGGCTTAGGGATTTA | |
| | TATATCAAAGGAGGCTTTGTACATGTGGGACAGGGA | |
| | TCTTATTTTACAAACAATTGTCTTACAAAATGAATAA | |
| | AACAGCACTTTGTTTTTATCTCCTGCTCTATTGTGCCA | |
| | TACTGTTAAATGTTTATAATGCCTGTTCTGTTTCCAA | |
| | ATTTGTGATGCTTATGAATATTAATAGGAATATTTGT | |
| | AAGGCCTGAAATATTTTGATCATGAAATCAAAACAT | |
| | TAATTTATTTAAACATTTACTTGAAATGTGGTGGTTT | |
| | GTGATTTAGTTGATTTTATAGGCTAGTGGGAGAATTT | |
| | ACATTCAAATGTCTAAATCACTTAAAATTGCCCTTTA | |
| | TGGCCTGACAGTAACTTTTTTTTATTCATTTGGGGAC | |
| | AACTATGTCCGTGAGCTTCCGTCCAGAGATTATAGTA | |
| | GTAAATTGTAATTAAAGGATATGATGCACGTGAAAT | |
| | CACTTTGCAATCATCAATAGCTTCATAAATGTTAATT | |
| | TTGTATCCTAATAGTAATGCTAATATTTTCCTAACAT | |
| | CTGTCATGTCTTTGTGTTCAGGGTAAAAAACTTGTTG | |
| | CTGCAAGTCAAGCTGCCTTAGGCTTAGGAAGCGGCG | |
| | CCACCAATTTCAGCCTGCTGAAACAGGCCGGCGACG | |
| | TGGAAGAGAACCCTGGCCCTTCCTTTATTCCTGTGGC | |
| | CGAAGATAGCGACTTCCCTATCCATAACCTCCCATAT | |
| | GGCGTCTTCTCAACCAGGGGCGACCCCAGGCCCAGG | |
| | ATTGGGGTGGCAATTGGAGACCAGATCCTGGACCTC | |
| | AGCATCATTAAGCACCTCTTTACAGGACCTGTGCTGA | |
| | GCAAGCACCAGGATGTGTTCAACCAGCCTACCCTGA | |
| | ATAGCTTTATGGGACTGGGCCAGGCAGCATGGAAAG | |
| | AAGCCAGAGTGTTCCTCCAGAATCTGCTGAGCGTGA | |
| | GCCAGGCCAGGCTCAGGGATGACACAGAACTGAGGA | |
| | AATGTGCCTTCATCTCACAGGCATCAGCCACAATGCA | |
| | CCTCCCTGCCACAATTGGGGACTACACTGACTTCTAC | |
| | AGCAGCAGACAGCATGCCACTAATGTGGGGATTATG | |
| | TTTAGAGACAAGGAAAATGCCCTCATGCCAAATTGG | |
| | CTGCACCTGCCTGTGGGCTACCACGGCAGAGCCTCAT | |
| | CAGTGGTGGTGTCAGGCACACCAATTAGGAGGCCAA | |
| | TGGGACAGATGAAGCCTGACGACTCAAAGCCACCAG | |
| | TCTATGGCGCCTGCAAACTGCTGGACATGGAGCTGG | |
| | AGATGGCTTTCTTTGTGGGGCCTGGCAACAGGCTGG | |
| | GAGAACCAATCCCTATTTCAAAGGCCCACGAGCACA | |
| | TTTTTGGCATGGTGCTCATGAATGACTGGTCTGCCAG | |
| | AGATATCCAGAAGTGGGAATACGTGCCCCTGGGCCC | |
| | TTTTCTGGGCAAGAGCTTTGGCACCACAGTGTCACCT | |
| | TGGGTGGTCCCAATGGATGCCCTGATGCCCTTTGCCG | |
| | TGCCCAACCCCAAGCAGGATCCCAGACCACTGCCCT | |
| | ACCTGTGCCACGATGAGCCCTATACCTTTGACATCAA | |
| | CCTGTCAGTCAACCTGAAGGGGGAGGGCATGAGCCA | |
| | GGCCGCCACTATTTGCAAGAGCAACTTTAAATATATG | |
| | TACTGGACTATGCTCCAACAGCTCACTCACCACTCAG | |
| | TGAATGGCTGCAATCTGAGGCCTGGCGACCTCCTGG | |
| | CTAGCGGCACTATCTCTGGCCCTGAACCTGAGAACTT | |
| | TGGGAGCATGCTGGAGCTGTCATGGAAAGGGACAAA | |
| | GCCTATTGACCTGGGGAATGGCCAGACAAGGAAGTT | |
| | TCTCCTGGATGGCGATGAGGTCATCATCACTGGGTAC | |
| | TGCCAGGGCGATGGCTACAGGATTGGATTTGGACAG | |
| | TGTGCTGGCAAAGTGCTCCCAGCTCTGCTGCCCTCAT | |
| | AACATCACATTTAAAAGCATCTCAGGTAACTATATTT | |
| | TGAATTTTTTAAAAAAGTAACTATAATAGTTATTATT | |
| | AAAATAGCAAAGATTGACCATTTCCAAGAGCCATAT | |
| | AGACCAGCACCGACCACTATTCTAAACTATTTATGTA | |
| | TGTAAATATTAGCTTTTAAAATTCTCAAAATAGTTGC | |
| | TGAGTTGGGAACCACTATTATTTCTATTTTGTAGATG | |
| | AGAAAATGAAGATAAACATCAAAGCATAGATTAAGT | |
| | AATTTTCCAAAGGGTCAAAATTCAAAATTGAAACCA | |
| | AAGTTTCAGTGTTGCCCATTGTCCTGTTCTGACTTAT | |
| | ATGATGCGGTACACAGAGCCATCCAAGTAAGTGATG | |
| | GCTCAGCAGTGGAATACTCTGGGAATTAGGCTGAAC | |
| | CACATGAAAGAGTGCTTTATAGGGCAAAAACAGTTG | |
| | AATATCAGTGATTTCACATGGTTCAACCTAATAGTTC | |
| | AACTCATCCTTTCCATTGGAGAATATGATGGATCTAC | |
| | CTTCTGTGAACTTTATAGTGAAGAATCTGCTATTACA | |
| | TTTCCAATTTGTCAACATGCTGAGCTTTAATAGGACT | |
| | TATCTTCTTATGACAACATTTATTGGTGTGTCCCCTTG | |
| | CCTAGCCCAACAGAAGAATTCAGCAGCCGTAAGTCT | |
| | AGGACAGGCTTAAATTGTTTTCACTGGTGTAAATTGC | |
| | AGAAAGATGATCTAAGTAATTTGGCATTTATTTTAAT | |
| | AGGTTTGAAAAACACATGCCATTTTACAAATAAGAC | |
| | TTATATTTGTCCTTTTGTTTTTCAGCCTACCATGAGAA | |
| | TAAGAGAAAGAAAATGAAGATCAAAAGCTTATTCAT | |
| | CTGTTTTTCTTTTTCGTTGGTGTAAAGCCAACACCCTG | |
| | TCTAAAAAACATAAATTTCTTTAATCATTTTGCCTCTT | |
| | TTCTCTGTGCTTCAATTAATAAAAAATGGAAAGAATC | |
| | TAATAGAGTGGTACAGCACTGTTATTTTTCAAAGATG | |
| | TGTTG | |
| LK03-GR | atggctgctgacggttatcttccagattggctcgaggacaacctttctgaaggcattcga | 32 |
| hFAH-col | gagtggtgggcgctgcaacctggagcccctaaacccaaggcaaatcaacaacatcag | |
| | gacaacgctcggggtcttgtgcttccgggttacaaatacctcggacccggcaacggact | |
| | cgacaagggggaacccgtcaacgcagcggacgcggcagccctcgagcacgacaag | |
| | gcctacgaccagcagctcaaggccggtgacaacccctacctcaagtacaaccacgcc | |
| | gacgccgagttccaggagcggctcaaagaagatacgtcttttgggggcaacctcggg | |
| | cgagcagtcttccaggccaaaaagaggcttcttgaacctcttggtctggttgaggaagc | |
| | ggctaagacggctcctggaaagaagaggcctgtagatcagtctcctcaggaaccgga | |
| | ctcatcatctggtgttggcaaatcgggcaaacagcctgccagaaaaagactaaatttcg | |
| | gtcagactggcgactcagagtcagtcccagaccctcaacctctcggagaaccaccagc | |
| | agcccccacaagtttgggatctaatacaatggcttcaggcggtggcgcaccaatggca | |
| | gacaataacgagggtgccgatggagtgggtaattcctcaggaaattggcattgcgattc | |
| | ccaatggctgggcgacagagtcatcaccaccagcaccagaacctgggccctgcccac | |
| | ttacaacaaccatctctacaagcaaatctccagccaatcaggagcttcaaacgacaacc | |
| | actactttggctacagcaccccttgggggtattttgactttaacagattccactgccacttct | |
| | caccacgtgactggcagcgactcattaacaacaactggggattccggcccaagaaact | |
| | cagcttcaagctcttcaacatccaagttaaagaggtcacgcagaacgatggcacgacg | |
| | actattgccaataaccttaccagcacggttcaagtgtttacggactcggagtatcagctcc | |
| | cgtacgtgctcgggtcggcgcaccaaggctgtctcccgccgtttccagcggacgtcttc | |
| | atggtccctcagtatggatacctcaccctgaacaacggaagtcaagcggtgggacgct | |
| | catccttttactgcctggagtacttcccttcgcagatgctaaggactggaaataacttccaa | |
| | ttcagctataccttcgaggatgtaccttttcacagcagctacgctcacagccagagtttgg | |
| | atcgcttgatgaatcctcttattgatcagtatctgtactacctgaacagaacgcaaggaac | |
| | aacctctggaacaaccaaccaatcacggctgctttttagccaggctgggcctcagtctat | |
| | gtctttgcaggccagaaattggctacctgggccctgctaccggcaacagagactttcaa | |
| | agactgctaacgacaacaacaacagtaactttccttggacagcggccagcaaatatcat | |
| | ctcaatggccgcgactcgctggtgaatccaggaccagctatggccagtcacaaggac | |
| | gatgaagaaaaatttttccctatgcacggcaatctaatatttggcaaagaagggacaacg | |
| | gcaagtaacgcagaattagataatgtaatgattacggatgaagaagagattcgtaccac | |
| | caatcctgtggcaacagagcagtatggaactgtggcaaataacttgcagagctcaaata | |
| | cagctcccacgactagaactgtcaatgatcagggggccttacctggcatggtgtggcaa | |
| | gatcgtgacgtgtaccttcaaggacctatctgggcaaagattcctcacacggatggaca | |
| | ctttcatccttctcctctgatgggaggctttggactgaaacatccgcctcctcaaatcatga | |
| | tcaaaaatactccggtaccggcaaatcctccgacgactttcagcccggccaagtttgctt | |
| | catttatcactcagtactccactggacaggtcagcgtggaaattgagtgggagctacag | |
| | aaagaaaacagcaaacgttggaatccagagattcagtacacttccaactacaacaagtc | |
| | tgttaatgtggactttactgtagacactaatggtgtttatagtgaacctcgccccattggca | |
| | cccgttaccttacccgtcccctgtaattgcttgttaatcaataaaccgtttaattcgtttcagtt | |
| | gaactttggtctctgcgtatttctttcttatctagtttccatatgcatgtagataagtagcatgg | |
| | cgggttaatcattaactaaccggtacctctagaactatagctagcgatgaccctgctgatt | |
| | ggttcgctgaccatttccgggtgcgggacggcgttaccagaaactcagaaggttcgtcc | |
| | aaccaaaccgactctgacggcagtttacgagagagatgatagggtctgcttcagtaagc | |
| | cagatgctacacaattaggcttgtacatattgtcgttagaacgcggctacaattaatacata | |
| | accttatgtatcatacacatacgatttaggtgacactatagaatacacggaattaattcttg | |
| | gccactccctctctgcgcgctcgctcgctcactgaggccgcccgggcaaagcccggg | |
| | cgtcgggcgacctttggtcgcccggcctcagtgagcgagcgagcgcgcagagaggg | |
| | agtggccaactccatcactaggggttcctTTTCTAGATGTAAATAATT | |
| | ATTTTAAGTTTGCCCTATGGTGGCCCCACACATGAGA | |
| | CAAACCCCCAAGATGTGACTTTTGAGAATGAGACTT | |
| | GGATAAAAAACATGTAGAAATGCAAGCCCTGAAGCT | |
| | CAACTCCCTATTGCTATCACAGGGGTTATAATTGCAT | |
| | AAAATTTAGCTATAGAAAGTTGCTGTCATCTCTTGTG | |
| | GGCTGTAATCATCGTCTAGGCTTAAGAGTAATATTGC | |
| | AAAACCTGTCATGCCCACACAAATCTCTCCCTGGCAT | |
| | TGTTGTCTTTGCAGATGTCAGTGAAAGAGAACCAGC | |
| | AGCTCCCATGAGTTTGGATAGCCTTATTTTCTATAGC | |
| | CTCCCCACTATTAGCTTTGAAGGGAGCAAAGTTTAAG | |
| | AACCAAATATAAAGTTTCTCATCTTTATAGATGAGAA | |
| | AAATTTTAAATAAAGTCCAAGATAATTAAATTTTTAA | |
| | GGATCATTTTTAGCTCTTTAATAGCAATAAAACTCAA | |
| | TATGACATAATATGGCACTTCCAAAATCTGAATAATA | |
| | TATAATTGCAATGACATACTTCTTTTCAGAGATTTAC | |
| | TGAAAAGAAATTTGTTGACACTACATAACGTGATGA | |
| | GTGGTTTATACTGATTGTTTCAGTTGGTCTTCCCACC | |
| | AACTCCATGAAAGTGGATTTTATTATCCTCATCATGC | |
| | AGATGAGAATATTGAGACTTATAGCGGTATGCCTGA | |
| | GCCCCAAAGTACTCAGAGTTGCCTGGCTCCAAGATTT | |
| | ATAATCTTAAATGATGGGACTACCATCCTTACTCTCT | |
| | CCATTTTTCTATACGTGAGTAATGTTTTTTCTGTTTTT | |
| | TTTTTTTCTTTTTCCATTCAAACTCAGTGCACTTGTTG | |
| | AGCTCGTGAAACACAAGCCCAAGGCAACAAAAGAG | |
| | CAACTGAAAGCTGTTATGGATGATTTCGCAGCTTTTG | |
| | TAGAGAAGTGCTGCAAGGCTGACGATAAGGAGACCT | |
| | GCTTTGCCGAGGAGGTACTACAGTTCTCTTCATTTTA | |
| | ATATGTCCAGTATTCATTTTTGCATGTTTGGTTAGGCT | |
| | AGGGCTTAGGGATTTATATATCAAAGGAGGCTTTGT | |
| | ACATGTGGGACAGGGATCTTATTTTACAAACAATTGT | |
| | CTTACAAAATGAATAAAACAGCACTTTGTTTTTATCT | |
| | CCTGCTCTATTGTGCCATACTGTTAAATGTTTATAAT | |
| | GCCTGTTCTGTTTCCAAATTTGTGATGCTTATGAATA | |
| | TTAATAGGAATATTTGTAAGGCCTGAAATATTTTGAT | |
| | CATGAAATCAAAACATTAATTTATTTAAACATTTACT | |
| | TGAAATGTGGTGGTTTGTGATTTAGTTGATTTTATAG | |
| | GCTAGTGGGAGAATTTACATTCAAATGTCTAAATCAC | |
| | TTAAAATTGCCCTTTATGGCCTGACAGTAACTTTTTTT | |
| | TATTCATTTGGGGACAACTATGTCCGTGAGCTTCCGT | |
| | CCAGAGATTATAGTAGTAAATTGTAATTAAAGGATA | |
| | TGATGCACGTGAAATCACTTTGCAATCATCAATAGCT | |
| | TCATAAATGTTAATTTTGTATCCTAATAGTAATGCTA | |
| | ATATTTTCCTAACATCTGTCATGTCTTTGTGTTCAGGG | |
| | TAAAAAACTTGTTGCTGCAAGTCAAGCTGCCTTAGGC | |
| | TTAGGAAGCGGCGCCACCAATTTCAGCCTGCTGAAA | |
| | CAGGCCGGCGACGTGGAAGAGAACCCTGGCCCTTCC | |
| | TTTATTCCTGTGGCCGAAGATAGCGACTTCCCTATCC | |
| | ATAACCTCCCATATGGCGTCTTCTCAACCAGGGGCGA | |
| | CCCCAGGCCCAGGATTGGGGTGGCAATTGGAGACCA | |
| | GATCCTGGACCTCAGCATCATTAAGCACCTCTTTACA | |
| | GGACCTGTGCTGAGCAAGCACCAGGATGTGTTCAAC | |
| | CAGCCTACCCTGAATAGCTTTATGGGACTGGGCCAG | |
| | GCAGCATGGAAAGAAGCCAGAGTGTTCCTCCAGAAT | |
| | CTGCTGAGCGTGAGCCAGGCCAGGCTCAGGGATGAC | |
| | ACAGAACTGAGGAAATGTGCCTTCATCTCACAGGCA | |
| | TCAGCCACAATGCACCTCCCTGCCACAATTGGGGACT | |
| | ACACTGACTTCTACAGCAGCAGACAGCATGCCACTA | |
| | ATGTGGGGATTATGTTTAGAGACAAGGAAAATGCCC | |
| | TCATGCCAAATTGGCTGCACCTGCCTGTGGGCTACCA | |
| | CGGCAGAGCCTCATCAGTGGTGGTGTCAGGCACACC | |
| | AATTAGGAGGCCAATGGGACAGATGAAGCCTGACGA | |
| | CTCAAAGCCACCAGTCTATGGCGCCTGCAAACTGCT | |
| | GGACATGGAGCTGGAGATGGCTTTCTTTGTGGGGCCT | |
| | GGCAACAGGCTGGGAGAACCAATCCCTATTTCAAAG | |
| | GCCCACGAGCACATTTTTGGCATGGTGCTCATGAATG | |
| | ACTGGTCTGCCAGAGATATCCAGAAGTGGGAATACG | |
| | TGCCCCTGGGCCCTTTTCTGGGCAAGAGCTTTGGCAC | |
| | CACAGTGTCACCTTGGGTGGTCCCAATGGATGCCCTG | |
| | ATGCCCTTTGCCGTGCCCAACCCCAAGCAGGATCCCA | |
| | GACCACTGCCCTACCTGTGCCACGATGAGCCCTATAC | |
| | CTTTGACATCAACCTGTCAGTCAACCTGAAGGGGGA | |
| | GGGCATGAGCCAGGCCGCCACTATTTGCAAGAGCAA | |
| | CTTTAAATATATGTACTGGACTATGCTCCAACAGCTC | |
| | ACTCACCACTCAGTGAATGGCTGCAATCTGAGGCCT | |
| | GGCGACCTCCTGGCTAGCGGCACTATCTCTGGCCCTG | |
| | AACCTGAGAACTTTGGGAGCATGCTGGAGCTGTCAT | |
| | GGAAAGGGACAAAGCCTATTGACCTGGGGAATGGCC | |
| | AGACAAGGAAGTTTCTCCTGGATGGCGATGAGGTCA | |
| | TCATCACTGGGTACTGCCAGGGCGATGGCTACAGGA | |
| | TTGGATTTGGACAGTGTGCTGGCAAAGTGCTCCCAGC | |
| | TCTGCTGCCCTCATAACATCACATTTAAAAGCATCTC | |
| | AGGTAACTATATTTTGAATTTTTTAAAAAAGTAACTA | |
| | TAATAGTTATTATTAAAATAGCAAAGATTGACCATTT | |
| | CCAAGAGCCATATAGACCAGCACCGACCACTATTCT | |
| | AAACTATTTATGTATGTAAATATTAGCTTTTAAAATT | |
| | CTCAAAATAGTTGCTGAGTTGGGAACCACTATTATTT | |
| | CTATTTTGTAGATGAGAAAATGAAGATAAACATCAA | |
| | AGCATAGATTAAGTAATTTTCCAAAGGGTCAAAATT | |
| | CAAAATTGAAACCAAAGTTTCAGTGTTGCCCATTGTC | |
| | CTGTTCTGACTTATATGATGCGGTACACAGAGCCATC | |
| | CAAGTAAGTGATGGCTCAGCAGTGGAATACTCTGGG | |
| | AATTAGGCTGAACCACATGAAAGAGTGCTTTATAGG | |
| | GCAAAAACAGTTGAATATCAGTGATTTCACATGGTTC | |
| | AACCTAATAGTTCAACTCATCCTTTCCATTGGAGAAT | |
| | ATGATGGATCTACCTTCTGTGAACTTTATAGTGAAGA | |
| | ATCTGCTATTACATTTCCAATTTGTCAACATGCTGAG | |
| | CTTTAATAGGACTTATCTTCTTATGACAACATTTATT | |
| | GGTGTGTCCCCTTGCCTAGCCCAACAGAAGAATTCA | |
| | GCAGCCGTAAGTCTAGGACAGGCTTAAATTGTTTTCA | |
| | CTGGTGTAAATTGCAGAAAGATGATCTAAGTAATTT | |
| | GGCATTTATTTTAATAGGTTTGAAAAACACATGCCAT | |
| | TTTACAAATAAGACTTATATTTGTCCTTTTGTTTTTCA | |
| | GCCTACCATGAGAATAAGAGAAAGAAAATGAAGATC | |
| | AAAAGCTTATTCATCTGTTTTTCTTTTTCGTTGGTGTA | |
| | AAGCCAACACCCTGTCTAAAAAACATAAATTTCTTTA | |
| | ATCATTTTGCCTCTTTTCTCTGTGCTTCAATTAATAAA | |
| | AAATGGAAAGAATCTAATAGAGTGGTACAGCACTGT | |
| | TATTTTTCAAAGATGTGTTGaggaacccctagtgatggagttggcca | |
| | ctccctctctgcgcgctcgctcgctcactgaggCCgcccgggcAAAgcccgggc | |
| | ggcctcagtgagcgagcgagcgcgcagagagggagtggccaactttttgcaaaagcc | |
| | taggcctccaaaaaagcctcctcactacttctggaatagctcagaggccgaggggcct | |
| | cggcctctgcataaataaaaaaaattagtcagccatggggcggagaatgggcggaact | |
| | gggcggagttaggggcgggatgggcggagttaggggcgggactatggttgctgacta | |
| | attgagatgcatgctttgcatacttctgcctgctggggagcctggggactttccacacctg | |
| | gttgctgactaattgagatgcatgctttgcatacttctgcctgctggggagcctggggact | |
| | ttccacaccctaactgacacacattccacagctgcattaatgaatcggccaacgcgcgg | |
| | ggagaggcggtttgcgtattgggcgctcttccgcttcctcgctcactgactcgctgcgct | |
| | cggtcgttcggctgcggcgagcggtatcagctcactcaaaggcggtaatacggttatcc | |
| | acagaatcaggggataacgcaggaaagaacatgtgagcaaaaggccagcaaaaggc | |
| | caggaaccgtaaaaaggccgcgttgctggcgtttttccataggctccgcccccctgacg | |
| | agcatcacaaaaatcgacgctcaagtcagaggtggcgaaacccgacaggactataaa | |
| | gataccaggcgtttccccctggaagctccctcgtgcgctctcctgttccgaccctgccgc | |
| | ttaccggatacctgtccgcctttctcccttcgggaagcgtggcgctttctcatagctcacg | |
| | ctgtaggtatctcagttcggtgtaggtcgttcgctccaagctgggctgtgtgcacgaacc | |
| | ccccgttcagcccgaccgctgcgccttatccggtaactatcgtcttgagtccaacccggt | |
| | aagacacgacttatcgccactggcagcagccactggtaacaggattagcagagcgag | |
| | gtatgtaggcggtgctacagagttcttgaagtggtggcctaactacggctacactagaa | |
| | gaacagtatttggtatctgcgctctgctgaagccagttaccttcggaaaaagagttggta | |
| | gctcttgatccggcaaacaaaccaccgctggtagcggtggtttttttgtttgcaagcagca | |
| | gattacgcgcagaaaaaaaggatctcaagaagatcctttgatcttttctacggggtctga | |
| | cgctcagtggaacgaaaactcacgttaagggattttggtcatgagattatcaaaaaggat | |
| | cttcacctagatccttttaaattaaaaatgaagttttaaatcaatctaaagtatatatgagtaa | |
| | acttggtctgacagttagaaaaactcatcgagcatcaaatgaaactgcaatttattcatatc | |
| | aggattatcaataccatatttttgaaaaagccgtttctgtaatgaaggagaaaactcaccg | |
| | aggcagttccataggatggcaagatcctggtatcggtctgcgattccgactcgtccaac | |
| | atcaatacaacctattaatttcccctcgtcaaaaataaggttatcaagtgagaaatcaccat | |
| | gagtgacgactgaatccggtgagaatggcaaaagtttatgcatttctttccagacttgttc | |
| | aacaggccagccattacgctcgtcatcaaaatcactcgcatcaaccaaaccgttattcatt | |
| | cgtgattgcgcctgagcgagacgaaatacgcgatcgctgttaaaaggacaattacaaa | |
| | caggaatcgaatgcaaccggcgcaggaacactgccagcgcatcaacaatattttcacc | |
| | tgaatcaggatattcttctaatacctggaatgctgtttttccggggatcgcagtggtgagta | |
| | accatgcatcatcaggagtacggataaaatgcttgatggtcggaagaggcataaattcc | |
| | gtcagccagtttagtctgaccatctcatctgtaacatcattggcaacgctacctttgccatg | |
| | tttcagaaacaactctggcgcatcgggcttcccatacaagcgatagattgtcgcacctga | |
| | ttgcccgacattatcgcgagcccatttatacccatataaatcagcatccatgttggaattta | |
| | atcgcggcctcgacgtttcccgttgaatatggctcatactcttcctttttcaatattattgaag | |
| | catttatcagggttattgtctcatgagcggatacatatttgaatgtatttagaaaaataaaca | |
| | aataggggttccgcgcacatttccccgaaaagtgccacctgacgtctaagaaaccattat | |
| | tatcatgacattaacctataaaaataggcgtatcacgaggccctttcgtctcgcgcgtttc | |
| | ggtgatgacggtgaaaacctctgacacatgcagctcccggagacggtcacagcttgtct | |
| | gtaagcggatgccgggagcagacaagcccgtcagggcgcgtcagcgggtgttggcg | |
| | ggtgtcggggctggcttaactatgcggcatcagagcagattgtactgagagtgcaccat | |
| | tcgacgctctcccttatgcgactcctgcattaggaagcagcccagtagtaggttgaggc | |
| | cgttgagcaccgccgccgcaaggaatggtgcatgcaaggagatggcgcccaacagt | |
| | cccccggccacggggcctgccaccatacccacgccgaaacaagcgctcatgagccc | |
| | gaagtggcgagcccgatcttccccatcggtgatgtcggcgatataggcgccagcaacc | |
| | gcacctgtggcgccggtgggtcaccaagcaggaagtcaaagactttttccggtgggca | |
| | aaggatcacgtggttgaggtggagcatgaattctacgtcaaaaagggtggagccaaga | |
| | aaagacccgcccccagtgacgcagatataagtgagcccaaacgggtgcgcgagtca | |
| | gttgcgcagccatcgacgtcagacgcggaagcttcgatcaactacgcagacaggtacc | |
| | aaaacaaatgttctcgtcacgtgggcatgaatctgatgctgtttccctgcagacaatgcg | |
| | agagaatgaatcagaattcaaatatctgcttcactcacggacagaaagactgtttagagt | |
| | gctttcccgtgtcagaatctcaacccgtttctgtcgtcaaaaaggcgtatcagaaactgtg | |
| | ctacattcatcatatcatgggaaaggtgccagacgcttgcactgcctgcgatctggtcaa | |
| | tgtggatttggatgactgcatctttgaacaataaatgatttaaatcaggt | |
| sL65-GR | CCCCTGTAAttgcttgttaatcaataaaccgtttaattcgtttcagttgaactttggtc | 33 |
| hFAH-col | tctgcgtatttctttcttatctagtttccatatgcatgtagataagtagcatgggggttaatc | |
| | attaactaaccggtacctctagaactatagctagcgatgaccctgctgattggttcgctga | |
| | ccatttccgggtgcgggacggcgttaccagaaactcagaaggttcgtccaaccaaacc | |
| | gactctgacggcagtttacgagagagatgatagggtctgcttcagtaagccagatgcta | |
| | cacaattaggcttgtacatattgtcgttagaacgcggctacaattaatacataaccttatgt | |
| | atcatacacatacgatttaggtgacactatagaatacacggaattaattcttggccactcc | |
| | ctctctgcgcgctcgctcgctcactgaggccgcccgggcaaagcccgggcgtcgggc | |
| | gacctttggtcgcccggcctcagtgagcgagcgagcgcgcagagagggagtggcca | |
| | actccatcactaggggttcctTTTCTAGATGTAAATAATTATTTTA | |
| | AGTTTGCCCTATGGTGGCCCCACACATGAGACAAAC | |
| | CCCCAAGATGTGACTTTTGAGAATGAGACTTGGATA | |
| | AAAAACATGTAGAAATGCAAGCCCTGAAGCTCAACT | |
| | CCCTATTGCTATCACAGGGGTTATAATTGCATAAAAT | |
| | TTAGCTATAGAAAGTTGCTGTCATCTCTTGTGGGCTG | |
| | TAATCATCGTCTAGGCTTAAGAGTAATATTGCAAAAC | |
| | CTGTCATGCCCACACAAATCTCTCCCTGGCATTGTTG | |
| | TCTTTGCAGATGTCAGTGAAAGAGAACCAGCAGCTC | |
| | CCATGAGTTTGGATAGCCTTATTTTCTATAGCCTCCC | |
| | CACTATTAGCTTTGAAGGGAGCAAAGTTTAAGAACC | |
| | AAATATAAAGTTTCTCATCTTTATAGATGAGAAAAAT | |
| | TTTAAATAAAGTCCAAGATAATTAAATTTTTAAGGAT | |
| | CATTTTTAGCTCTTTAATAGCAATAAAACTCAATATG | |
| | ACATAATATGGCACTTCCAAAATCTGAATAATATATA | |
| | ATTGCAATGACATACTTCTTTTCAGAGATTTACTGAA | |
| | AAGAAATTTGTTGACACTACATAACGTGATGAGTGG | |
| | TTTATACTGATTGTTTCAGTTGGTCTTCCCACCAACTC | |
| | CATGAAAGTGGATTTTATTATCCTCATCATGCAGATG | |
| | AGAATATTGAGACTTATAGCGGTATGCCTGAGCCCC | |
| | AAAGTACTCAGAGTTGCCTGGCTCCAAGATTTATAAT | |
| | CTTAAATGATGGGACTACCATCCTTACTCTCTCCATT | |
| | TTTCTATACGTGAGTAATGTTTTTTCTGTTTTTTTTTTT | |
| | TCTTTTTCCATTCAAACTCAGTGCACTTGTTGAGCTC | |
| | GTGAAACACAAGCCCAAGGCAACAAAAGAGCAACT | |
| | GAAAGCTGTTATGGATGATTTCGCAGCTTTTGTAGAG | |
| | AAGTGCTGCAAGGCTGACGATAAGGAGACCTGCTTT | |
| | GCCGAGGAGGTACTACAGTTCTCTTCATTTTAATATG | |
| | TCCAGTATTCATTTTTGCATGTTTGGTTAGGCTAGGG | |
| | CTTAGGGATTTATATATCAAAGGAGGCTTTGTACATG | |
| | TGGGACAGGGATCTTATTTTACAAACAATTGTCTTAC | |
| | AAAATGAATAAAACAGCACTTTGTTTTTATCTCCTGC | |
| | TCTATTGTGCCATACTGTTAAATGTTTATAATGCCTG | |
| | TTCTGTTTCCAAATTTGTGATGCTTATGAATATTAAT | |
| | AGGAATATTTGTAAGGCCTGAAATATTTTGATCATGA | |
| | AATCAAAACATTAATTTATTTAAACATTTACTTGAAA | |
| | TGTGGTGGTTTGTGATTTAGTTGATTTTATAGGCTAG | |
| | TGGGAGAATTTACATTCAAATGTCTAAATCACTTAAA | |
| | ATTGCCCTTTATGGCCTGACAGTAACTTTTTTTTATTC | |
| | ATTTGGGGACAACTATGTCCGTGAGCTTCCGTCCAGA | |
| | GATTATAGTAGTAAATTGTAATTAAAGGATATGATG | |
| | CACGTGAAATCACTTTGCAATCATCAATAGCTTCATA | |
| | AATGTTAATTTTGTATCCTAATAGTAATGCTAATATT | |
| | TTCCTAACATCTGTCATGTCTTTGTGTTCAGGGTAAA | |
| | AAACTTGTTGCTGCAAGTCAAGCTGCCTTAGGCTTAG | |
| | GAAGCGGCGCCACCAATTTCAGCCTGCTGAAACAGG | |
| | CCGGCGACGTGGAAGAGAACCCTGGCCCTTCCTTTAT | |
| | TCCTGTGGCCGAAGATAGCGACTTCCCTATCCATAAC | |
| | CTCCCATATGGCGTCTTCTCAACCAGGGGCGACCCCA | |
| | GGCCCAGGATTGGGGTGGCAATTGGAGACCAGATCC | |
| | TGGACCTCAGCATCATTAAGCACCTCTTTACAGGACC | |
| | TGTGCTGAGCAAGCACCAGGATGTGTTCAACCAGCC | |
| | TACCCTGAATAGCTTTATGGGACTGGGCCAGGCAGC | |
| | ATGGAAAGAAGCCAGAGTGTTCCTCCAGAATCTGCT | |
| | GAGCGTGAGCCAGGCCAGGCTCAGGGATGACACAGA | |
| | ACTGAGGAAATGTGCCTTCATCTCACAGGCATCAGC | |
| | CACAATGCACCTCCCTGCCACAATTGGGGACTACACT | |
| | GACTTCTACAGCAGCAGACAGCATGCCACTAATGTG | |
| | GGGATTATGTTTAGAGACAAGGAAAATGCCCTCATG | |
| | CCAAATTGGCTGCACCTGCCTGTGGGCTACCACGGC | |
| | AGAGCCTCATCAGTGGTGGTGTCAGGCACACCAATT | |
| | AGGAGGCCAATGGGACAGATGAAGCCTGACGACTCA | |
| | AAGCCACCAGTCTATGGCGCCTGCAAACTGCTGGAC | |
| | ATGGAGCTGGAGATGGCTTTCTTTGTGGGGCCTGGCA | |
| | ACAGGCTGGGAGAACCAATCCCTATTTCAAAGGCCC | |
| | ACGAGCACATTTTTGGCATGGTGCTCATGAATGACTG | |
| | GTCTGCCAGAGATATCCAGAAGTGGGAATACGTGCC | |
| | CCTGGGCCCTTTTCTGGGCAAGAGCTTTGGCACCACA | |
| | GTGTCACCTTGGGTGGTCCCAATGGATGCCCTGATGC | |
| | CCTTTGCCGTGCCCAACCCCAAGCAGGATCCCAGAC | |
| | CACTGCCCTACCTGTGCCACGATGAGCCCTATACCTT | |
| | TGACATCAACCTGTCAGTCAACCTGAAGGGGGAGGG | |
| | CATGAGCCAGGCCGCCACTATTTGCAAGAGCAACTT | |
| | TAAATATATGTACTGGACTATGCTCCAACAGCTCACT | |
| | CACCACTCAGTGAATGGCTGCAATCTGAGGCCTGGC | |
| | GACCTCCTGGCTAGCGGCACTATCTCTGGCCCTGAAC | |
| | CTGAGAACTTTGGGAGCATGCTGGAGCTGTCATGGA | |
| | AAGGGACAAAGCCTATTGACCTGGGGAATGGCCAGA | |
| | CAAGGAAGTTTCTCCTGGATGGCGATGAGGTCATCA | |
| | TCACTGGGTACTGCCAGGGCGATGGCTACAGGATTG | |
| | GATTTGGACAGTGTGCTGGCAAAGTGCTCCCAGCTCT | |
| | GCTGCCCTCATAACATCACATTTAAAAGCATCTCAGG | |
| | TAACTATATTTTGAATTTTTTAAAAAAGTAACTATAA | |
| | TAGTTATTATTAAAATAGCAAAGATTGACCATTTCCA | |
| | AGAGCCATATAGACCAGCACCGACCACTATTCTAAA | |
| | CTATTTATGTATGTAAATATTAGCTTTTAAAATTCTC | |
| | AAAATAGTTGCTGAGTTGGGAACCACTATTATTTCTA | |
| | TTTTGTAGATGAGAAAATGAAGATAAACATCAAAGC | |
| | ATAGATTAAGTAATTTTCCAAAGGGTCAAAATTCAA | |
| | AATTGAAACCAAAGTTTCAGTGTTGCCCATTGTCCTG | |
| | TTCTGACTTATATGATGCGGTACACAGAGCCATCCAA | |
| | GTAAGTGATGGCTCAGCAGTGGAATACTCTGGGAAT | |
| | TAGGCTGAACCACATGAAAGAGTGCTTTATAGGGCA | |
| | AAAACAGTTGAATATCAGTGATTTCACATGGTTCAAC | |
| | CTAATAGTTCAACTCATCCTTTCCATTGGAGAATATG | |
| | ATGGATCTACCTTCTGTGAACTTTATAGTGAAGAATC | |
| | TGCTATTACATTTCCAATTTGTCAACATGCTGAGCTT | |
| | TAATAGGACTTATCTTCTTATGACAACATTTATTGGT | |
| | GTGTCCCCTTGCCTAGCCCAACAGAAGAATTCAGCA | |
| | GCCGTAAGTCTAGGACAGGCTTAAATTGTTTTCACTG | |
| | GTGTAAATTGCAGAAAGATGATCTAAGTAATTTGGC | |
| | ATTTATTTTAATAGGTTTGAAAAACACATGCCATTTT | |
| | ACAAATAAGACTTATATTTGTCCTTTTGTTTTTCAGCC | |
| | TACCATGAGAATAAGAGAAAGAAAATGAAGATCAA | |
| | AAGCTTATTCATCTGTTTTTCTTTTTCGTTGGTGTAAA | |
| | GCCAACACCCTGTCTAAAAAACATAAATTTCTTTAAT | |
| | CATTTTGCCTCTTTTCTCTGTGCTTCAATTAATAAAAA | |
| | ATGGAAAGAATCTAATAGAGTGGTACAGCACTGTTA | |
| | TTTTTCAAAGATGTGTTGaggaacccctagtgatggagttggccactc | |
| | cctctctgcgcgctcgctcgctcactgaggCCgcccgggcAAAgcccgggcggc | |
| | ctcagtgagcgagcgagcgcgcagagagggagtggccaactttttgcaaaagcctag | |
| | gcctccaaaaaagcctcctcactacttctggaatagctcagaggccgaggcggcctcg | |
| | gcctctgcataaataaaaaaaattagtcagccatggggcggagaatgggcggaactgg | |
| | gcggagttaggggcgggatgggcggagttaggggcgggactatggttgctgactaatt | |
| | gagatgcatgctttgcatacttctgcctgctggggagcctggggactttccacacctggtt | |
| | gctgactaattgagatgcatgctttgcatacttctgcctgctggggagcctggggactttc | |
| | cacaccctaactgacacacattccacagctgcattaatgaatcggccaacgcgcgggg | |
| | agaggcggtttgcgtattgggcgctcttccgcttcctcgctcactgactcgctgcgctcg | |
| | gtcgttcggctgcggcgagcggtatcagctcactcaaaggcggtaatacggttatccac | |
| | agaatcaggggataacgcaggaaagaacatgtgagcaaaaggccagcaaaaggcca | |
| | ggaaccgtaaaaaggccgcgttgctggcgtttttccataggctccgcccccctgacgag | |
| | catcacaaaaatcgacgctcaagtcagaggtggcgaaacccgacaggactataaagat | |
| | accaggcgtttccccctggaagctccctcgtgcgctctcctgttccgaccctgccgctta | |
| | ccggatacctgtccgcctttctcccttcgggaagcgtggcgctttctcatagctcacgctg | |
| | taggtatctcagttcggtgtaggtcgttcgctccaagctgggctgtgtgcacgaaccccc | |
| | cgttcagcccgaccgctgcgccttatccggtaactatcgtcttgagtccaacccggtaag | |
| | acacgacttatcgccactggcagcagccactggtaacaggattagcagagcgaggtat | |
| | gtaggcggtgctacagagttcttgaagtggtggcctaactacggctacactagaagaac | |
| | agtatttggtatctgcgctctgctgaagccagttaccttcggaaaaagagttggtagctctt | |
| | gatccggcaaacaaaccaccgctggtagcggtggtttttttgtttgcaagcagcagatta | |
| | cgcgcagaaaaaaaggatctcaagaagatcctttgatcttttctacggggtctgacgctc | |
| | agtggaacgaaaactcacgttaagggattttggtcatgagattatcaaaaaggatcttca | |
| | cctagatccttttaaattaaaaatgaagttttaaatcaatctaaagtatatatgagtaaacttg | |
| | gtctgacagttagaaaaactcatcgagcatcaaatgaaactgcaatttattcatatcagga | |
| | ttatcaataccatatttttgaaaaagccgtttctgtaatgaaggagaaaactcaccgaggc | |
| | agttccataggatggcaagatcctggtatcggtctgcgattccgactcgtccaacatcaa | |
| | tacaacctattaatttcccctcgtcaaaaataaggttatcaagtgagaaatcaccatgagt | |
| | gacgactgaatccggtgagaatggcaaaagtttatgcatttctttccagacttgttcaaca | |
| | ggccagccattacgctcgtcatcaaaatcactcgcatcaaccaaaccgttattcattcgtg | |
| | attgcgcctgagcgagacgaaatacgcgatcgctgttaaaaggacaattacaaacagg | |
| | aatcgaatgcaaccggcgcaggaacactgccagcgcatcaacaatattttcacctgaat | |
| | caggatattcttctaatacctggaatgctgtttttccggggatcgcagtggtgagtaaccat | |
| | gcatcatcaggagtacggataaaatgcttgatggtcggaagaggcataaattccgtcag | |
| | ccagtttagtctgaccatctcatctgtaacatcattggcaacgctacctttgccatgtttcag | |
| | aaacaactctggcgcatcgggcttcccatacaagcgatagattgtcgcacctgattgcc | |
| | cgacattatcgcgagcccatttatacccatataaatcagcatccatgttggaatttaatcgc | |
| | ggcctcgacgtttcccgttgaatatggctcatactcttcctttttcaatattattgaagcattta | |
| | tcagggttattgtctcatgagcggatacatatttgaatgtatttagaaaaataaacaaatag | |
| | gggttccgcgcacatttccccgaaaagtgccacctgacgtctaagaaaccattattatca | |
| | tgacattaacctataaaaataggcgtatcacgaggccctttcgtctcgcgcgtttcggtga | |
| | tgacggtgaaaacctctgacacatgcagctcccggagacggtcacagcttgtctgtaag | |
| | cggatgccgggagcagacaagcccgtcagggcgcgtcagcgggtgttggcgggtgt | |
| | cggggctggcttaactatgcggcatcagagcagattgtactgagagtgcaccattcgac | |
| | gctctcccttatgcgactcctgcattaggaagcagcccagtagtaggttgaggccgttga | |
| | gcaccgccgccgcaaggaatggtgcatgcaaggagatggcgcccaacagtcccccg | |
| | gccacggggcctgccaccatacccacgccgaaacaagcgctcatgagcccgaagtg | |
| | gcgagcccgatcttccccatcggtgatgtcggcgatataggcgccagcaaccgcacct | |
| | gtggcgccggtgggtcaccaagcaggaagtcaaagactttttccggtgggcaaaggat | |
| | cacgtggttgaggtggagcatgaattctacgtcaaaaagggggagccaagaaaagac | |
| | ccgcccccagtgacgcagatataagtgagcccaaacgggtgcgcgagtcagttgcgc | |
| | agccatcgacgtcagacgcggaagcttcgatcaactacgcagacaggtaccaaaaca | |
| | aatgttctcgtcacgtgggcatgaatctgatgctgtttccctgcagacaatgcgagagaat | |
| | gaatcagaattcaaatatctgcttcactcacggacagaaagactgtttagagtgctttccc | |
| | gtgtcagaatctcaacccgtttctgtcgtcaaaaaggcgtatcagaaactgtgctacattc | |
| | atcatatcatgggaaaggtgccagacgcttgcactgcctgcgatctggtcaatgtggatt | |
| | tggatgactgcatctttgaacaataaatgatttaaatcaggtATGGCTGCtGAcG | |
| | GTTATCTTCCAGATTGGCTCGAGGACACTCTCTCTGA | |
| | AGGCATTCGCGAGTGGTGGGCGCTGAAACCTGGAGC | |
| | TCCACAACCCAAGGCCAACCAACAGCATCAGGACAA | |
| | CGGCAGGGGTCTTGTGCTTCCTGGGTACAAGTACCTC | |
| | GGACCCTTCAACGGACTCGACAAGGGAGAGCCGGTC | |
| | AACGAGGCAGACGCCGCGGCCCTCGAGCACGACAAG | |
| | GCCTACGACAAGCAGCTCGAGCAGGGGGACAACCCG | |
| | TACCTCAAGTACAACCACGCCGACGCCGAGTTTCAG | |
| | GAGCGTCTGCAAGAAGATACGTCTTTTGGGGGCAAC | |
| | CTCGGGCGAGCAGTCTTCCAGGCCAAGAAGCGGGTT | |
| | CTCGAACCTCTCGGTCTGGTTGAGGAAGGCGCTAAG | |
| | ACGGCTCCTGGAAAGAAGAGACCGGTAGAGCCGTCA | |
| | CCTCAGCGTTCCCCCGACTCCTCCACGGGCATCGGCA | |
| | AGAAAGGCCAGCAGCCCGCCAGAAAGAGACTCAATT | |
| | TCGGTCAGACTGGCGACTCAGAGTCAGTCCCCGACC | |
| | CTCAACCTCTCGGAGAACCTCCAGCAGCGCCCTCTAG | |
| | TGTGGGATCTGGTACAGTGGCTGCAGGCGGTGGCGC | |
| | ACCAATGGCAGACAATAACGAAGGTGCCGACGGAGT | |
| | GGGTAATGCCTCAGGAAATTGGCATTGCGATTCCAC | |
| | ATGGCTGGGCGACAGAGTCATTACCACCAGCACCCG | |
| | AACCTGGGCCCTGCCCACCTACAACAACCACCTCTAC | |
| | AAGCAAATCTCCAGCCAATCAGGAGCTTCAAACGAC | |
| | AACCACTACTTTGGCTACAGCACCCCTTGGGGGTATT | |
| | TTGACTTTAACAGATTCCACTGCCACTTCTCACCACG | |
| | TGACTGGCAGCGACTCATTAACAACAACTGGGGATT | |
| | CCGGCCCAAGAGACTCAACTTCAAGCTCTTCAACATC | |
| | CAAGTCAAGGAGGTCACGACGAATGATGGCGTCACG | |
| | ACCATCGCTAATAACCTTACCAGCACGGTTCAAGTCT | |
| | TCTCGGACTCGGAGTACCAGTTGCCGTACGTCCTCGG | |
| | CTCTGCGCACCAGGGCTGCCTCCCTCCGTTCCCGGCG | |
| | GACGTGTTCATGATTCCCCAGTACGGCTACCTAACAC | |
| | TCAACAACGGTAGTCAGGCCGTGGGACGCTCCTCCTT | |
| | TTACTGCCTGGAATATTTCCCATCGCAGATGCTGAGA | |
| | ACGGGCAATAACTTTGAGTTCAGCTACAGCTTCGAG | |
| | GACGTGCCTTTCCACAGCAGCTACGCACACAGCCAG | |
| | AGCTTGGACCGACTGATGAATCCTCTCATTGACCAGT | |
| | ACCTGTACTACTTATCCAGAACTCAGTCCACAGGAG | |
| | GAACTCAAGGTACCCAGCAATTGTTATTTTCTCAAGC | |
| | TGGGCCTGCAAACATGTCGGCTCAGGCCAAGAACTG | |
| | GCTGCCTGGACCTTGCTACCGGCAGCAGCGAGTCTCC | |
| | ACGACACTGTCGCAAAACAACAACAGCAACTTTGCT | |
| | TGGACTGGTGCCACCAAATATCACCTGAACGGCAGA | |
| | AACTCGTTGGTTAATCCCGGCGTCGCCATGGCAACTC | |
| | ACAAGGACGACGAGGACCGCTTTTTCCCATCCAGCG | |
| | GAGTCCTGATTTTTGGAAAAACTGGAGCAACTAACA | |
| | AAACTACATTGGAAAATGTGTTAATGACAAATGAAG | |
| | AAGAAATTCGTCCTACTAATCCTGTAGCCACGGAAG | |
| | AATACGGGATAGTCAGCAGCAACTTACAAGCGGCTA | |
| | ATACTGCAGCCCAGACACAAGTTGTCAACAACCAGG | |
| | GAGCCTTACCTGGCATGGTCTGGCAGAACCGGGACG | |
| | TGTACCTGCAGGGTCCCATTTGGGCCAAAATTCCTCA | |
| | CACAGATGGACACTTTCACCCGTCTCCTCTTATGGGC | |
| | GGCTTTGGACTCAAGAACCCGCCTCCTCAGATCCTCA | |
| | TCAAAAACACGCCTGTTCCTGCGAATCCTCCGGCGG | |
| | AGTTTTCAGCTACAAAGTTTGCTTCATTCATCACCCA | |
| | GTATTCCACAGGACAAGTGAGCGTGGAGATTGAATG | |
| | GGAGCTGCAGAAAGAAAACAGCAAACGCTGGAATC | |
| | CCGAAGTGCAGTATACATCTAACTATGCAAAATCTG | |
| | CCAACGTTGATTTCACTGTGGACAACAATGGACTTTA | |
| | TACTGAGCCTCGCCCCATTGGCACCCGTTACCTTACC | |
| | CGT | |

### EXEMPLARY EMBODIMENTS

1. A composition comprising:
   a polynucleotide cassette comprising:
      an expression cassette comprising a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a cell;
   a third nucleic acid sequence positioned 5′ to the expression cassette and comprising a sequence that is substantially homologous to a genomic sequence 5′ of a target integration site in a genome of a cell; and
   a fourth nucleic acid sequence positioned 3′ to expression cassette and comprising a sequence that is substantially homologous to a genomic sequence 3′ of a target integration site in the genome of the cell.
2. The composition of embodiment 1, wherein the composition further comprises a delivery vehicle.
3. The composition of embodiment 2, wherein the delivery vehicle comprises a lipid nanoparticle.
4. The composition of embodiment 3, wherein the delivery vehicle comprises a recombinant viral vector.
5. The composition of embodiment 4, wherein the recombinant viral vector is a recombinant adeno-associated (AAV) viral vector.
6. The composition of embodiment 5, wherein the recombinant viral vector is or comprises a capsid protein comprising an amino acid sequence having at least 95% sequence identity with the amino acid sequence of, AAV8, AAV-DJ; AAV-LK03; sL65; or AAVNP59.
7. The composition of any one of the above embodiments, wherein the transgene is or comprises a fumarylacetoacetate hydrolase (FAH) transgene.
8. The composition of embodiment 7, wherein the FAH transgene is a wt human FAH; a codon optimized FAH; a synthetic FAH; a FAH variant; a FAH mutant, or a FAH fragment.
9. The composition of embodiment 7, wherein the FAH transgene has 80% sequence identity to SEQ ID NO. 18, 19, 20, 21, or 22.
10. The composition of any one of the above embodiments, wherein the composition further comprises AAV2 ITR sequences and/or ITR sequences selected from SEQ ID NO: 27-30.
11. The composition of any one of the above embodiments, wherein the polynucleotide cassette does not comprise a promoter sequence.
12. The composition of any one of the above embodiments, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and C-terminal intein splicing region; or
   d) a nucleic acid sequence encoding a splice donor and a splice acceptor.
13. The composition of any one of the above embodiments, wherein the third and fourth nucleic acid sequences are homology arms that integrate the expression cassette at a target integration site comprising an endogenous promoter and an endogenous gene.
14. The composition of embodiment 13, wherein the target integration site is the endogenous albumin gene locus.
15. A method of integrating a transgene into the genome of at least a population of cells in a tissue in a subject, said method comprising
   administering to a subject a composition comprising:
      a polynucleotide cassette comprising:
         an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell;
      a third nucleic acid sequence positioned 5′ to the expression cassette and comprising a sequence that is substantially homologous to a genomic sequence 5′ of the target integration site in the genome of the cell; and
      a fourth nucleic acid sequence positioned 3′ to the expression and comprising a sequence that is substantially homologous to a genomic sequence 3′ of the target integration site in the genome of the cell;
   wherein, after administering the composition, the transgene is integrated into the genome of the population of cells.
16. The method of embodiment 15, wherein the integration does not comprise nuclease activity.
17. The method of embodiment 15, wherein the composition further comprises a delivery vehicle.
18. The method of embodiment 17, wherein the delivery vehicle comprises a lipid nanoparticle.
19. The method of embodiment 17, wherein the delivery vehicle comprises a recombinant viral vector.
20. The method of embodiment 19, wherein the recombinant viral vector is a recombinant adeno-associated (AAV) viral vector.
21. The method of embodiment 19, wherein the recombinant viral vector is or comprises a capsid protein comprising an amino acid sequence having at least 95% sequence identity with the amino acid sequence of AAV8, AAV-DJ; AAV-LK03; sL65; or AAVNP59.
22. The method of any one of embodiments 15-21, wherein the transgene is or comprises a fumarylacetoacetate hydrolase (FAH) transgene.
23. The method of embodiment 22, wherein wherein the FAH transgene is a wt human FAH; a codon optimized FAH; a synthetic FAH; a FAH variant; a FAH mutant, or a FAH fragment.
24. The method of embodiment 22, wherein the FAH transgene has 80% sequence identity to SEQ ID NO. 18, 19, 20, 21, or 22.
25. The method of any one of embodiments 15-24, wherein the composition further comprises AAV2 ITR sequences and/or ITR sequences selected from SEQ ID NO: 27-30.
26. The method of any one of embodiments 15-25, wherein the polynucleotide cassette does not comprise a promoter sequence.
27. The method of any one of embodiments 15-26, wherein upon integration of the expression cassette into the target integration site in the genome of the cell, the transgene is expressed under control of an endogenous promoter at the target integration site.
28. The method of embodiment 27, wherein the target integration site is the albumin locus comprising an endogenous albumin promoter and an endogenous albumin gene.
29. The method of embodiment 28, wherein upon integration of the expression cassette into the target integration site in the genome of a cell, the transgene is expressed under control of the endogenous albumin promoter without disruption of the endogenous albumin gene expression.
30. The method of any one of the above embodiments, wherein the tissue is the liver.
31. The method of any one of the above embodiments, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and C-terminal intein splicing region; or
   d) a nucleic acid sequence encoding a splice donor and a splice acceptor.
32. The method of any one of the above embodiments, wherein the third and fourth nucleic acid sequences are homology arms that integrate the expression cassette at a target integration site comprising an endogenous promoter and an endogenous gene.
33. The method of embodiment 32, wherein the target integration site is the endogenous albumin gene locus.
34. A method of increasing a level of expression of a transgene in a tissue over a period of time, said method comprising
   administering to a subject in need thereof a composition that delivers a transgene that integrates into the genome of at least a population of cells in the tissue of the subject, wherein the composition comprises:
      a polynucleotide cassette comprising
         an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell;
      a third nucleic acid sequence positioned 5′ to the expression cassette and comprising a sequence that is substantially homologous to a genomic sequence 5′ of the target integration site in the genome of the cell; and
      a fourth nucleic acid sequence positioned 3′ to the expression cassette and comprising a sequence that is substantially homologous to a genomic sequence 3′ of the target integration site in the genome of the cell;
   wherein, after administering the composition, the transgene is integrated into the genome of the population of cells and the level of expression of the transgene in the tissue increases over a period of time.
35. The method of embodiment 34, wherein the integration of the transgene does not comprise nuclease activity.
36. The method of embodiment 34 or 35, wherein the increased level of expression comprises an increased percent of cells in the tissue expressing the transgene.
37. The method of embodiment 34, wherein the composition further comprises a delivery vehicle.
38. The method of embodiment 37, wherein the delivery vehicle comprises a lipid nanoparticle.
39. The method of embodiment 37, wherein the delivery vehicle comprises a recombinant viral vector.
40. The method of embodiment 39, wherein the recombinant viral vector is a recombinant AAV vector.
41. The method of embodiment 40, wherein the recombinant viral vector is or comprises a capsid protein comprising an amino acid sequence having at least 95% sequence identity with the amino acid sequence of, AAV8, AAV-DJ; AAV-LK03; sL65; or AAVNP59.
42. The method of any one of embodiments 34-41, wherein the transgene is or comprises a fumarylacetoacetate hydrolase (FAH) transgene.
43. The method of embodiment 42, wherein the FAH transgene is a wt human FAH; a codon optimized FAH; a synthetic FAH; a FAH variant; a FAH mutant, or a FAH fragment.
44. The method of embodiment 42, wherein the FAH transgene has 80% sequence identity to SEQ ID NO. 18, 19, 20, 21, or 22.
45. The method of any one of embodiments 34-44, wherein the composition further comprises AAV2 ITR sequences and/or ITR sequences selected from SEQ ID NO: 27-30.
46. The method of any one of embodiments 34-45, wherein the polynucleotide cassette does not comprise a promoter sequence.
47. The method of any one of embodiments 34-46, wherein upon integration of the expression cassette into the target integration site in the genome of the cell, the transgene is expressed under control of an endogenous promoter at the target integration site.
48. The method of embodiment 47, wherein the target integration site is the albumin locus comprising an endogenous albumin promoter and an endogenous albumin gene.
49. The method of embodiment 48, wherein upon integration of the expression cassette into the target integration site in the genome of a cell, the transgene is expressed under control of the endogenous albumin promoter without disruption of the endogenous albumin gene expression.
50. The method of any one of embodiments 34-49, wherein the tissue is the liver.
51. The method of any one of embodiments 34-50, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and C-terminal intein splicing region; or
   d) a nucleic acid sequence encoding a splice donor and a splice acceptor.
52. The method of any one of embodiments 34-51, wherein the third and fourth nucleic acid sequences are homology arms that integrate the expression cassette at a target integration site comprising an endogenous promoter and an endogenous gene.
53. The method of embodiment 52, wherein the target integration site is the endogenous albumin gene locus.
54. A recombinant viral vector for integrating a transgene into a target integration site in the genome of a cell, comprising a polynucleotide cassette comprising:
   (i) an expression cassette comprising a first nucleic acid sequence and a second nucleic acid sequence, wherein the first nucleic acid sequence comprises a FAH transgene; and the second nucleic acid sequence is positioned 5′ or 3′ to the first nucleic acid sequence and promotes the production of two independent gene products upon integration into the target integration site in the genome of the cell;
   (ii) a third nucleic acid sequence positioned 5′ to the expression cassette and comprising a sequence that is substantially homologous to a genomic sequence 5′ of the target integration site in the genome of the cell; and
   (iii) a fourth nucleic acid sequence positioned 3′ of the expression cassette and comprising a sequence that is substantially homologous to a genomic sequence 3′ of the target integration site in the genome of the cell.
55. The recombinant viral vector of embodiment 54, wherein the third nucleic acid is between 900-1150 nucleotides.
56. The recombinant viral vector of embodiment 54 or embodiment 55, wherein the fourth nucleic acid is between 1500-1750 nucleotides.
57. The recombinant viral vector of any one of embodiments 54-56, wherein the recombinant viral vector is a recombinant AAV vector.
58 The recombinant viral vector of embodiment 57, wherein the recombinant viral vector is or comprises a capsid protein comprising an amino acid sequence having at least 95% sequence identity with the amino acid sequence of AAV8, AAV-DJ; AAV-LK03; sL65; or AAV-NP59.
59. The recombinant viral vector of any one of embodiments 54-58, further comprising AAV2 ITR sequences and/or ITR sequences selected from SEQ ID NO: 27-30.
60. The recombinant viral vector of any one of embodiments 54-59, wherein the polynucleotide cassette does not comprise a promoter sequence.
61. The recombinant viral vector of any one of embodiments 54-60, wherein upon integration of the expression cassette into the target integration site in the genome of a cell, the FAH transgene is expressed under control of an endogenous promoter at the target integration site.
62. The recombinant viral vector of any one of embodiments 61, wherein the target integration site is the albumin locus comprising an endogenous albumin promoter and an endogenous albumin gene.
63. The recombinant viral vector of embodiment 61, wherein upon integration of the expression cassette into the target integration site in the genome of a cell, the FAH transgene is expressed under control of the endogenous albumin promoter without disruption of the endogenous albumin gene expression.
64. The recombinant viral vector of any one of embodiments 61, wherein the two independent gene products are a FAH protein expressed from the FAH transgene and a peptide comprising an endogenous protein expressed from an endogenous gene at the integration site.
65. The recombinant viral vector of any one of embodiments 54-64, wherein the cell is a liver cell.
66. The recombinant viral vector of any one of embodiments 54-65, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and a C-terminal intein splicing region; or
   d) a nucleic acid sequence encoding a splice donor and a splice acceptor.
67. The recombinant viral vector of any of embodiments 54-66, wherein the third and fourth nucleic acid sequences are homology arms that integrate the FAH transgene and the second nucleic acid sequence into an endogenous albumin gene locus comprising an endogenous albumin promoter and an endogenous albumin gene.
68. The recombinant viral vector of embodiment 67, wherein the third and fourth nucleic acid sequences are homology arms that integrate the FAH transgene and the second nucleic acid sequence into an endogenous albumin gene locus in frame with the endogenous albumin promoter and the endogenous albumin gene.
69. The recombinant viral vector of embodiment 67 or embodiment 68, wherein the homology arms direct integration of the polynucleotide cassette immediately 3′ of the start codon of the endogenous albumin gene or immediately 5′ of the stop codon of the endogenous albumin gene.
70. The recombinant viral vector of any one of embodiments 54-69, wherein the FAH transgene is a wt human FAH; a codon optimized FAH; a synthetic FAH; a FAH variant; a FAH mutant, or a FAH fragment.
71. A method comprising a step of
   administering to a subject a dose of a composition that delivers to cells in a tissue of the subject a transgene, wherein the transgene (i) encodes FAH; (ii) integrates at a target integration site in the genome of a plurality of the cells; (iii) functionally expresses FAH once integrated; and (iv) confers a selective advantage to the plurality of cells relative to other cells in the tissue, so that, over time, the tissue achieves a level of functional expression of FAH that is greater than cells that did not integrate the transgene wherein the composition comprises:
   a polynucleotide cassette comprising:
      an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products when the transgene is integrated at the target integration site;
   a third nucleic acid sequence positioned 5′ to the expression cassette and comprising a sequence that is substantially homologous to a genomic sequence 5′ of the target integration site; and
   a fourth nucleic acid sequence positioned 3′ to the expression cassette and comprising a sequence that is substantially homologous to a genomic sequence 3′ of the target integration site.
72. The method of embodiment 71, wherein the integration of the transgene does not comprise nuclease activity.
73. The method of embodiment 71 or 72, wherein the selective advantage comprises an increased percent of cells in the tissue expressing the transgene.
74. The method of any one of embodiments 71-73, wherein the composition further comprises a delivery vehicle.
75. The method of embodiment 74, wherein the delivery vehicle comprises a lipid nanoparticle.
76. The method of embodiment 74, wherein the composition comprises a recombinant viral vector.
77. The method of embodiment 76, wherein the recombinant viral vector is a recombinant AAV vector.
78. The method of embodiment 77, wherein the recombinant viral vector is or comprises a capsid protein comprising an amino acid sequence having at least 95% sequence identity with the amino acid sequence of AAV8, AAV-DJ; AAV-LK03; sL65 or AAVNP59.
79. The method of embodiment 71, wherein the FAH transgene is a wt human FAH; a codon optimized FAH; a synthetic FAH; a FAH variant; a FAH mutant, or a FAH fragment.
80. The method of any one of embodiments 71-79, wherein the composition further comprises AAV2 ITR sequences and/or ITR sequences selected from SEQ ID NO: 27-30.
81. The method of any one of embodiments 71-80, wherein the polynucleotide cassette does not comprise a promoter sequence.
82. The method of any one of embodiments 71-81, wherein upon integration of the expression cassette into the target integration site in the genome of the cell, the transgene is expressed under control of an endogenous promoter at the target integration site.
83. The method of embodiment 82, wherein the target integration site is the albumin locus comprising an endogenous albumin promoter and an endogenous albumin gene.
84. The method of embodiment 83, wherein upon integration of the polynucleotide cassette into the target integration site in the genome of a cell, the transgene is expressed under control of the endogenous albumin promoter without disruption of the endogenous albumin gene expression.
85. The method of any one of embodiments 71-84, wherein the tissue is the liver.
86. The method of any one of embodiments 71-85, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and C-terminal intein splicing region; or
   d) a nucleic acid sequence encoding a splice donor and a splice acceptor.
87. The method of any one of embodiments 71-86, wherein the third and fourth nucleic acid sequences are homology arms that integrate the expression cassette at a target integration site comprising an endogenous promoter and an endogenous gene.
88. The method of embodiment 87, wherein the target integration site is the endogenous albumin locus.
89. A method of treating hereditary tyrosinemia type 1 (HT1), the method comprising administering to a subject a dose of a composition comprising:
   a polynucleotide cassette comprising
      an expression cassette comprising a first nucleic acid sequence and a second nucleic acid sequence, wherein the first nucleic acid sequence encodes a FAH transgene; and the second nucleic acid sequence is positioned 5′ or 3′ to the first nucleic acid sequence and promotes the production of two independent gene products when the transgene is integrated at the target integration site;
   a third nucleic acid sequence positioned 5′ to the first nucleic acid sequence and comprising a sequence that is substantially homologous to a genomic sequence 5′ of the target integration site; and
   a fourth nucleic acid sequence positioned 3′ to the second nucleic acid sequence and comprising a sequence that is substantially homologous to a genomic sequence 3′ of the target integration site.
      wherein, after administering the composition, the transgene is integrated into the genome of the population of cells.
90. The method of embodiment 89, wherein the integration does not comprise nuclease activity.
91. The method of embodiment 89, wherein the composition further comprises a delivery vehicle.
92. The method of embodiment 91, wherein the delivery vehicle comprises a lipid nanoparticle.
93. The method of embodiment 91, wherein the delivery vehicle comprises a recombinant viral vector.
94. The method of embodiment 93, wherein the recombinant viral vector is a recombinant adeno-associated (AAV) viral vector.
95. The method of embodiment 93, wherein the recombinant viral vector is or comprises a capsid protein comprising an amino acid sequence having at least 95% sequence identity with the amino acid sequence of AAV8, AAV-DJ; AAV-LK03; sL65; or AAVNP59.
96. The method of embodiment 95, wherein the FAH transgene is a wt human FAH; a codon optimized FAH; a synthetic FAH; a FAH variant; a FAH mutant, or a FAH fragment.
97. The method of embodiment 95, wherein the FAH transgene has 80% sequence identity to SEQ ID NO. 18, 19, 20, 21, or 22.
98. The method of any one of embodiments 89-97, wherein the composition further comprises AAV2 ITR sequences and/or ITR sequences selected from SEQ ID NO: 27-30.
99. The method of any one of embodiments 89-98, wherein the polynucleotide cassette does not comprise a promoter sequence.
100. The method of any one of embodiments 89-99, wherein upon integration of the expression cassette into the target integration site in the genome of the cell, the transgene is expressed under control of an endogenous promoter at the target integration site.
101. The method of embodiment 100, wherein the target integration site is the albumin locus comprising an endogenous albumin promoter and an endogenous albumin gene.
102. The method of embodiment 101, wherein upon integration of the polynucleotide cassette into the target integration site in the genome of a cell, the transgene is expressed under control of the endogenous albumin promoter without disruption of the endogenous albumin gene expression.
103. The method of any one of embodiments 89-102, wherein the tissue is the liver.
104. The method of any one of embodiments 89-103, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and C-terminal intein splicing region; or
   d) a nucleic acid sequence encoding a splice donor and a splice acceptor.
105. The method of any one of embodiments 89-104, wherein the third and fourth nucleic acid sequences are homology arms that integrate expression cassette into at a target integration site comprising an an endogenous promoter and an endogenous gene.
106. The method of embodiment 105, wherein the target integration site is the endogenous albumin gene locus.
107. The method of embodiment 89, wherein the subject has received or is receiving treatment for HT1.
108. The method of embodiment 107, wherein the subject has received or is receiving NTBC ((2-(2-nitro-4-trifluoromethylbenzoyl)-1,3cyclohexanedione)).
109. The method of embodiment 107 or 108, wherein NTBC and the composition are administered in combination.
110. The method of embodiment 109, wherein NTBC and the composition are administered simultaneously, or sequentially.
111. The method of embodiment 89, wherein the subject receives a lower or reduced dose of the treatment the subject is receiving after treatment with the composition.
112. The method of embodiment 89, wherein the subject receives the same dose of the treatment the subject has received or is receiving after treatment with the composition.
113. The method of embodiment 89, wherein the subject stops receiving the treatment the subject has received or is receiving after treatment with the composition.
114. The method of embodiment 89, wherein the subject is a neonate.
115. The method of embodiment 89, wherein the subject is one week, two weeks, three weeks, four weeks, or five weeks old.
116. A method of monitoring gene therapy, the method comprising a step of:
   detecting, in a biological sample from a subject who has received gene therapy treatment comprising the composition of embodiment 1, a level or activity of a biomarker generated by integration of the integrating gene therapy treatment, as a surrogate for one or more characteristics of the status of the gene therapy treatment, wherein the one or more characteristics of the status of the gene therapy treatment is selected from the group consisting of level of a payload, activity of a payload, level of integration of the gene therapy treatment in a population of cells, and combinations thereof.
117. The method of embodiment 116, wherein the payload is or comprises a peptide expressed intracellularly.
118. The method of embodiment 116, wherein the payload is or comprises a peptide that is secreted extracellularly.
119. The method of any one of embodiments 116-118, wherein the payload is encoded by the polynucleotide cassette
120. The method of any one embodiments 116-119, wherein the biological sample is or comprises hair, skin, feces, blood, plasma, serum, cerebrospinal fluid, urine, saliva, tears, vitreous humor, liver biopsy or mucus.
121. The method of any one of embodiments 116-120, wherein the step of detecting comprises an immunological assay or a nucleic acid amplification assay.
122. The method of any embodiments 116-121, wherein the biomarker comprises a detectable moiety that, after translation of the polypeptide encoded by the target site, becomes fused to the polypeptide encoded by the target site.
123. The method of any one of embodiments 116-123, wherein the biomarker comprises a detectable moiety that, after translation of the polypeptide encoded by the target site, becomes fused to the polypeptide encoded by the payload.
124. The method of any one of embodiments 116-123, wherein the biomarker comprises a detectable moiety that is a 2A peptide
125. The method of embodiment 124, wherein the 2A peptide is selected from the group consisting of P2A, T2A, E2A and F2A.
126. The method of any one of embodiments 116-125, wherein the subject receives a single dose of the gene therapy treatment or gene-integrating composition.
127. The method of any one of embodiments 116-126, wherein the detecting step is performed 1, 2, 3, 4, 5, 6, 7, 8 or more weeks after the subject has received the gene therapy treatment or gene-integrating composition.
128. The method of any one of embodiments 116-127, wherein the detecting step is performed at multiple time points after the subject has received the gene therapy treatment or gene-integrating composition.
129. The method of any one of embodiments 116-128, wherein the detecting step is performed over a period of at least 3 months after the subject has received the gene therapy treatment or gene-integrating composition.
130. The method of any one of embodiments 116-129, wherein the method further comprises monitoring the subject for autoimmune response to the gene therapy.
131. A composition comprising a recombinant AAV construct comprising:
   a polynucleotide cassette comprising:
      an expression cassette comprising a first nucleic acid sequence and a second nucleic acid sequence, wherein the first nucleic acid sequence has 80% sequence identity to SEQ ID NO. 18, 19, 20, 21, or 22; and the second nucleic acid sequence
      (i) is positioned 5′ or 3′ to the first nucleic acid sequence; and
      (ii) promotes the production of two independent gene products upon integration into a target integration site in the genome of a cell;
   a third nucleic acid sequence positioned 5′ to the expression cassette and comprising a sequence that is substantially homologous to a genomic sequence 5′ of a target integration site in a genome of a cell; and
   a fourth nucleic acid sequence positioned 3′ to expression cassette and comprising a sequence that is substantially homologous to a genomic sequence 3′ of a target integration site in the genome of the cell.
132. The composition of embodiment 131, wherein the recombinant viral vector is or comprises a capsid protein comprising an amino acid sequence having at least 95% sequence identity with the amino acid sequence of, AAV8, AAV-DJ; AAV-LK03; sL65; or AAVNP59.
133. The composition of embodiments 131 or 132, wherein the composition further comprises AAV2 ITR sequences and/or ITR sequences selected from SEQ ID NO: 27-30.
134. The composition of embodiments 131-133, where the second nucleic acid sequence has 80% sequence identity to SEQ ID NO. 6.
135. The composition of embodiments 131-133, where the second nucleic acid sequence encodes a P2A peptide having 90% sequence identity to SEQ ID NO. 7.
136. A recombinant AAV construct comprising a nucleic acid sequence comprising SEQ ID NO: 23.
137. A recombinant AAV construct comprising a nucleic acid sequence comprising SEQ ID NO: 24.
138. A recombinant AAV construct comprising a nucleic acid sequence comprising SEQ ID NO: 25.
139. A recombinant AAV construct comprising a nucleic acid sequence comprising SEQ ID NO: 26.
140. A composition comprising the recombinant AAV construct of any one of embodiments 136-139.
141. A method of integrating a transgene into the genome of at least a population of cells in a tissue in a subject, said method comprising administering to a subject a composition comprising the recombinant AAV construct of any one of embodiments 136-139.
142. A composition comprising the recombinant viral vector of embodiments 54-70.
143. A method of treatment comprising administering a composition of any one of embodiments 1, 131, or 142, wherein the composition is administered to a subject in dosages between 1E12 and 1E14 vg/kg.
144. The method of embodiment 143, wherein the composition is administered to a subject in dosages between 3E12 and 1E13 vg/kg.
145. The method of embodiment 143, wherein the composition is administered to a subject in dosages between 3E12 and 3E13 vg/kg.
146. The method of embodiment 143, wherein the composition is administered to a subject in dosages of no more than 3E13 vg/kg.
147. The method of embodiment 143, wherein the composition is administered to a subject in dosages of no more than 3E12 vg/kg.
148. The method of any one of embodiments 142-147, wherein the composition is administered only once.
149. The method of any one of embodiments 142-147, wherein the composition is administered more than once.
150. The method of any one of embodiments 143-149, wherein the subject is a newborn.
151. The method of any one of embodiments 143-149, wherein the subject is between 0 days and 1 month of age.
152. The method of any one of embodiments 143-149, wherein the subject is between 3 months of age and 1 year of age.
153. The method of any one of embodiments 143-149, wherein the subject is between 1 year of age and 5 years of age.
154. The method of any one of embodiments 143-149, wherein the subject is 5 years of age or older.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A composition comprising:
a closed circular cDNA integrating gene therapy construct comprising, from 5′ to 3′, a polynucleotide sequence encoding (a) a 5′ homology arm between 1 kb and 1.6 kb in length, (b) a P2A coding sequence encoding a P2A peptide, (c) a therapeutic payload, and (d) a 3′ homology arm between 1 kb and 1.6 kb in length, wherein:
the therapeutic payload comprises a transgene sequence encoding fumarylacetoacetate hydrolase (FAH); and
the homology arm sequences promote integration of the construct at an endogenous albumin target site such that the albumin locus can result in the simultaneous production of albumin-2A and the transgene as separate proteins.

2. The composition of claim 1, wherein the closed circular cDNA integrating gene therapy construct comprises the nucleotide sequence of SEQ ID NO: 23, SEQ ID NO: 24, SEQ ID NO: 25, or SEQ ID NO: 26.

3. (canceled)

4. The composition of claim 1, wherein;
(a) the 5′ homology arm sequence comprises the nucleotide sequence of SEQ ID NO: 1 or SEQ ID NO: 3; and/or
(b) the 3′ homology arm sequence comprises SEQ ID NO: 2.

5. (canceled)

6. The composition of claim 1, wherein;
(a) the P2A coding sequence comprises the nucleotide sequence of SEQ ID NO: 6; and/or
(b) the P2A coding sequence encodes a peptide comprising the amino acid sequence of SEQ ID NO: 7.

7. (canceled)

8. The composition of claim 1, wherein the transgene sequence encoding FAH comprises the nucleotide sequence of SEQ ID NO: 20, SEQ ID NO: 21, or SEQ ID NO: 22.

9. (canceled)

10. The composition of claim 1, wherein the composition further comprises an adeno-associated viral (AAV) capsid protein.

11. The composition of claim 10, wherein the AAV capsid protein comprises an amino acid sequence having at least 95% sequence identity to the amino acid sequence of AAV8, AAV-DJ, AAV-LK03, sL65, or AAVNP59.

12. A method of treating hereditary tyrosinemia type 1 (HT1) comprising administering to a subject a dose of the composition of claim 1.

13. The method of claim 12, wherein the closed circular cDNA integrating gene therapy construct comprises the nucleotide sequence of SEQ ID NO: 23, SEQ ID NO: 24, SEQ ID NO: 25, or SEQ ID NO: 26.

14. (canceled)

15. The method of claim 12, wherein;
(a) the 5′ homology arm comprises the nucleotide sequence of SEQ ID NO: 1 or SEQ ID NO: 3; and/or
(b) the 3′ homology arm comprises SEQ ID NO: 2.

16. (canceled)

17. The method of claim 12, wherein:
(a) the P2A coding sequence comprises the nucleotide sequence of SEQ ID NO: 6; and/or the P2A coding sequence encodes a peptide comprising the amino acid sequence of SEQ ID NO: 7.

18. (canceled)

19. The method of claim 12, wherein the transgene sequence encoding FAH comprises the nucleotide sequence of SEQ ID NO: 20, SEQ ID NO: 21, or SEQ ID NO: 22.

20. (canceled)

21. The method of claim 12, wherein the composition further comprises an AAV capsid protein.

22. The method of claim 21, wherein the AAV capsid protein comprises an amino acid sequence having at least 95% sequence identity to the amino acid sequence of AAV8, AAV-DJ; AAV-LK03; sL65; or AAVNP59.

23. The method of claim 12, wherein:
(a) the composition is administered to the subject in dosages between 1E12 and 1E14 vg/kg;
(b) the composition is administered to the subject in dosages between 3E12 and 1E13 vg/kg;
(c) the composition is administered to the subject in dosages between 3E12 and 3E13 vg/kg;
(d) the composition is administered to the subject in dosages of no more than 3E13 vg/kg; or
(e) the composition is administered to the subject in dosages of no more than 3E12 vg/kg.

24-27. (canceled)

28. The method of claim 12, wherein:
(a) the composition is administered to the subject only once;
(b) the composition is administered more than once;
(c) the subject is a newborn;
(d) the subject is between 0 days and 1 month of age;
(e) the subject is between 3 months of age and 1 year of age;
(f) the subject is between 1 year of age and 5 years of age; or
(g) the subject is 5 years of age or older.

29.-34. (canceled)

35. A closed circular DNA consisting of the polynucleotide sequence of SEQ ID NO: 23, the polynucleotide sequence of SEQ ID NO: 24, the polynucleotide sequence of SEQ ID NO: 25, or the polynucleotide sequence of SEQ ID NO: 26.

36-38. (canceled)

39. A liver-targeted, recombinant AAV vector for treating HT1 and encoding a human FAH, the liver-targeted recombinant AAV vector comprising a closed, circular cDNA polynucleotide sequence comprising:
a human FAH polynucleotide sequence encoding a functional FAH transgene comprising the nucleotide sequence of SEQ ID NO: 20, SEQ ID NO: 21, or SEQ ID NO: 22, preceded by a 2A-peptide sequence encoding a 2A-peptide comprising the amino acid sequence of SEQ ID NO: 7;
the human FAH polynucleotide sequence and 2A-peptide sequence together flanked by a 3′ homology arm comprising the nucleotide sequence of SEQ ID NO: 2; and a 5′ homology arm comprising the nucleotide sequence of SEQ ID NO: 1 or SEQ ID NO: 3.

40-43. (canceled)

44. The method of claim 12, wherein the closed circular cDNA integrating gene therapy construct is a liver-targeted, recombinant AAV.

45-51. (canceled)

52. A method of treating HT1 comprising administering to a subject in need thereof a therapeutically effective dose of a liver-targeted, recombinant AAV vector encoding human FAH, the liver-targeted, recombinant AAV vector comprising a cDNA polynucleotide sequence comprising
a FAH polynucleotide sequence encoding a functional human FAH, the FAH polynucleotide sequence comprising the nucleotide sequence of SEQ ID NO: 20, SEQ ID NO: 21, or SEQ ID NO: 22, preceded by a 2A-peptide sequence encoding a 2A-peptide comprising the amino acid sequence of SEQ ID NO: 7;
the FAH polynucleotide sequence and 2A-peptide sequence together flanked by a 3′ homology arm comprising the nucleotide sequence of SEQ ID NO: 2; and a 5′ homology arm comprising the nucleotide sequence of SEQ ID NO: 1, or SEQ ID NO: 3.


SEQUENCE LISTING (41 sequences)

SEQ ID NO: 1 - DNA, 1645 bp, Homo sapiens
  misc_feature 1..1645: note = Human albumin 1.6 kb 5 homology arm
  source 1..1645: mol_type = genomic DNA
tttctagatgtaaataattattttaagtttgccctatggtggccccacacatgagacaaacccccaagatgtgacttttgagaatgagacttggataaaaaacatgtagaaatgcaagccctgaagctcaactccctattgctatcacaggggttataattgcataaaatttagctatagaaagttgctgtcatctcttgtgggctgtaatcatcgtctaggcttaagagtaatattgcaaaacctgtcatgcccacacaaatctctccctggcattgttgtctttgcagatgtcagtgaaagagaaccagcagctcccatgagtttggatagccttattttctatagcctccccactattagctttgaagggagcaaagtttaagaaccaaatataaagtttctcatctttatagatgagaaaaattttaaataaagtccaagataattaaatttttaaggatcatttttagctctttaatagcaataaaactcaatatgacataatatggcacttccaaaatctgaataatatataattgcaatgacatacttcttttcagagatttactgaaaagaaatttgttgacactacataacgtgatgagtggtttatactgattgtttcagttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggctta

SEQ ID NO: 2 - DNA, 1000 bp, Homo sapiens
  misc_feature 1..1000: note = Human albumin 1kb 3 homology arm
  source 1..1000: mol_type = genomic DNA
taacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttg

SEQ ID NO: 3 - DNA, 1000 bp, Homo sapiens
  misc_feature 1..1000: note = Human albumin 1kb 5 homology arm
  source 1..1000: mol_type = genomic DNA
actccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggctta

SEQ ID NO: 4 - DNA, 1000 bp, Mus musculus
  misc_feature 1..1000: note = Mouse albumin 1kb 5 homology arm
  source 1..1000: mol_type = genomic DNA
ctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagcc

SEQ ID NO: 5 - DNA, 1648 bp, Mus musculus
  misc_feature 1..1648: note = Mouse albumin 1.6kb 3 homology arm
  source 1..1648: mol_type = genomic DNA
taaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaacagatgtcagctaagactaggtcatgtgtaggctgtctacaccagtgaaaatcgcaaaaagaatctaagaaattccacatttctagaaaataggtttggaaaccgtattccattttacaaaggacacttacatttctctttttgttttccaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctactctgtggttcagaactctatcttccaaaggcgcgcttcaccctagcagcctctttggctcagaggaatccctgcctttcctcccttcatctcagcagagaatgtagttccacatgggcaacacaatgaaaataaacgttaatactctcccatcttatgggtggtgaccctagaaaccaatacttcaacattacgagaattctgaatgagagactaaaagcttatgaactgtggctttcctttgtcagtgggactctaagaatgagttggggacaaaagagataggaatggctttaaaggtgactagttgaactgataaagtaaatgaactgaggaaaaaaaatatcactcaa

SEQ ID NO: 6 - DNA, 66 bp, Porcine teschovirus-1
  misc_feature 1..66: note = P2A Nucleotide Sequence
  source 1..66: mol_type = genomic DNA
ggaagcggcgccaccaatttcagcctgctgaaacaggccggcgacgtggaagagaaccctggccct

SEQ ID NO: 7 - protein, 22 aa, Porcine teschovirus-1
  REGION 1..22: note = misc_feature - P2A Peptide Sequence
GSGATNFSLLKQAGDVEENPGP

SEQ ID NO: 8 - DNA, 2211 bp, synthetic construct
  misc_feature 1..2211: note = Synthetic: AAV LK03 (nucleotide)
  source 1..2211: mol_type = other DNA
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaa

SEQ ID NO: 9 - protein, 736 aa, synthetic construct
  REGION 1..736: note = Synthetic: AAV-LK03 (protein)
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 10 - DNA, 2217 bp, synthetic construct
  misc_feature 1..2217: note = Synthetic: AAV8 (nucleotide)
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 11 - protein, 738 aa, synthetic construct
  REGION 1..738: note = Synthetic: AAV8 (protein)
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 12 - DNA, 2214 bp, synthetic construct
  misc_feature 1..2214: note = Synthetic: AAV-DJ (nucleotide)
  source 1..2214: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 13 - protein, 737 aa, synthetic construct
  REGION 1..737: note = Synthetic: AAV-DJ (protein)
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 14 - DNA, 2208 bp, synthetic construct
  misc_feature 1..2208: note = Synthetic: sL65 (nucleotide)
  source 1..2208: mol_type = other DNA
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccccagtacggctacctaacactcaacaacggtagtcaggccgtgggacgctcctccttttactgcctggaatatttcccatcgcagatgctgagaacgggcaataactttgagttcagctacagcttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttaccttacccgtcccctgtaa

SEQ ID NO: 15 - protein, 735 aa, synthetic construct
  REGION 1..735: note = Synthetic: sL65 (protein)
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYSFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 16 - DNA, 2208 bp, synthetic construct
  misc_feature 1..2208: note = Synthetic: AAV-NP59 (nucleotide)
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggcaagacaggccagcagcccgctaaaaagagactcaattttggtcagactggcgactcagagtcagtcccagaccctcaacctctcggagaaccaccagcagccccctctggtctgggaactaatacgatggctacaggcagtggcgcaccaatggcagacaataacgagggtgccgatggagtgggtaattcctcaggaaattggcattgcgattcccaatggctgggcgacagagtcatcaccaccagcacccgaacctgggccctgcccacctacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtgggctggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcgacacacaaggcgttcttccaggcatggtatggcaggacagagatgtgtaccttcagggacccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactcaacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 17 - protein, 735 aa, synthetic construct
  REGION 1..735: note = Synthetic: AAV-NP59 (protein)
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKTGQQPAKKRLNFGQTGDSESVPDPQPLGEPPAAPSGLGTNTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWAGATKYHLNGRDSLVNPGPAMASHKDDEEKFFPQSGVLIFGKQGSEKTNVDIEKVMITDEEEIRTTNPVATEQYGSVSTNLQRGNRQAATADVDTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 18 - DNA, 1254 bp, Mus musculus
  misc_feature 1..1254: note = Mouse FAH (mFAH)
  source 1..1254: mol_type = genomic DNA
tcctttattccagtggccgaggactccgactttcccatccaaaacctgccctatggtgttttctccactcaaagcaacccaaagccacggattggtgtagccatcggtgaccagatcttggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcgcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggggcaggggcagaccaggaccttcctgctggacggcgatgaagtcatcataacaggtcactgccagggggacggctaccgtgttggctttggccagtgtgctgggaaagtgctgcctgccctttcaccagcc

SEQ ID NO: 19 - DNA, 1254 bp, Homo sapiens
  misc_feature 1..1254: note = Human FAH (hFAH)
  source 1..1254: mol_type = genomic DNA
tccttcatcccggtggccgaggattccgacttccccatccacaacctgccctacggcgtcttctcgaccagaggcgacccaagaccgaggataggtgtggccattggcgaccagatcctggacctcagcatcatcaagcacctctttactggtcctgtcctctccaaacaccaggatgtcttcaatcagcctacactcaacagcttcatgggcctgggtcaggctgcctggaaggaggcgagagtgttcttgcagaacttgctgtctgtgagccaagccaggctcagagatgacaccgaacttcggaagtgtgcattcatctcccaggcttctgccacgatgcaccttccagccaccataggagactacacagacttctattcctctcggcagcatgctaccaacgtcggaatcatgttcagggacaaggagaatgcgttgatgccaaattggctgcacttaccagtgggctaccatggccgtgcctcctctgtcgtggtgtctggcaccccaatccgaaggcccatgggacagatgaaacctgatgactctaagcctcccgtatatggtgcctgcaagctcttggacatggagctggaaatggctttttttgtaggccctggaaacagattgggagagccgatccccatttccaaggcccatgagcacatttttggaatggtccttatgaacgactggagtgcacgagacattcagaagtgggagtatgtccctctcgggccattccttgggaagagttttgggaccactgtctctccgtgggtggtgcccatggatgctctcatgccctttgctgtgcccaacccgaagcaggaccccaggcccctgccgtatctgtgccatgacgagccctacacatttgacatcaacctctctgttaacctgaaaggagaaggaatgagccaggcggctaccatatgcaagtccaattttaagtacatgtactggacgatgctgcagcagctcactcaccactctgtcaacggctgcaacctgcggccgggggacctcctggcttctgggaccatcagcgggccggagccagaaaacttcggctccatgttggaactgtcgtggaagggaacgaagcccatagacctggggaatggtcagaccaggaagtttctgctggacggggatgaagtcatcataacagggtactgccagggggatggttaccgcatcggctttggccagtgtgctggaaaagtgctgcctgctctcctgccatca

SEQ ID NO: 20 - DNA, 1254 bp, synthetic construct
  misc_feature 1..1254: note = Synthetic: Human FAH Codon Optimized Variant 3 (hFAH-co3)
  source 1..1254: mol_type = other DNA
tccttcattcctgtggccgaggattctgactttcccattcacaacctgccctatggcgtcttttcaaccaggggagatcccaggcccagaatcggggtggccattggagatcagatcctggacctgtcaatcatcaagcacctgtttacaggccctgtgctgagcaagcaccaggatgtgttcaaccagccaactctgaacagcttcatggggctggggcaggctgcctggaaggaggcaagggtgtttctgcagaatctgctgagcgtgtcacaggctaggctgagggatgataccgagctgaggaagtgtgcatttatctcacaggcatcagccacaatgcatctgccagctacaatcggcgactataccgacttttactcaagcaggcagcatgccaccaatgtgggcatcatgttcagggacaaagagaatgccctgatgccaaattggctgcacctgcctgtgggctatcatggcagagccagctcagtggtggtgtcagggacaccaattaggaggcctatgggccagatgaagcctgatgacagcaaaccacctgtctacggcgcctgcaagctcctggatatggaactggagatggctttttttgtgggcccaggcaatagactgggagagcccattccaatctcaaaggcccatgaacatatctttggcatggtcctgatgaatgactggtcagccagagacattcagaagtgggagtacgtgccactgggaccatttctgggaaagagctttggcaccacagtgtcaccatgggtggtgcccatggatgccctgatgccctttgctgtgccaaatccaaaacaagaccccaggcccctcccctatctctgtcatgatgaaccatatacttttgacattaacctgagcgtgaacctgaaaggagaaggcatgagccaagccgccactatctgtaagagcaacttcaaatatatgtactggacaatgctgcagcagctcacccaccatagcgtgaatgggtgtaacctgaggccaggcgacctgctggcatcaggcactatttcagggcctgagcctgaaaattttggatcaatgctggagctgtcatggaagggaactaaacccatcgacctggggaatggccagaccagaaagtttctcctggatggcgatgaggtgatcattacaggctactgtcagggggatggctatagaattggatttggccaatgcgccggcaaagtcctccctgccctgctgcccagc

SEQ ID NO: 21 - DNA, 1254 bp, synthetic construct
  misc_feature 1..1254: note = Synthetic: Human FAH Codon Optimized Variant 2 (hFAH-co2)
  source 1..1254: mol_type = other DNA
tccttcatcccagtggctgaagattctgacttccccattcacaacctcccttatggagtctttagcacaagaggagacccaagacctagaattggcgtggctattggggatcagatcctggacctgagcattatcaaacatctctttacaggcccagtcctcagcaagcaccaagatgtgttcaatcaacccacactgaactcatttatggggctgggccaagctgcctggaaggaggcaagagtgtttctccagaacctcctgtcagtgagccaggccaggctgagagatgacacagagctgaggaagtgtgcctttatctcacaagccagcgccacaatgcacctgccagcaaccatcggggactacacagacttttacagcagcaggcagcacgccactaatgtgggcatcatgtttagagacaaagagaatgccctcatgcctaactggctgcatctgccagtgggctaccatggcagagccagcagcgtggtggtgtctggcacccctatcaggaggcccatgggccagatgaagcctgatgacagcaagccacctgtgtatggggcatgtaagctgctggacatggagctggaaatggctttctttgtggggcctgggaacaggctgggcgaaccaattcccatcagcaaagctcatgaacacatttttgggatggtgctcatgaatgactggtctgccagagacatccagaagtgggagtatgtccccctgggaccattcctgggcaagagctttgggaccactgtgtcaccatgggtggtgcccatggatgccctgatgccatttgctgtgcctaatcccaaacaagatccaaggccactgccctacctctgccacgatgagccctacacatttgatatcaacctctctgtgaatctgaagggagaaggaatgagccaagctgctaccatttgcaaaagcaactttaagtatatgtattggaccatgctgcagcaactcacccaccacagcgtgaatggctgtaacctgaggcctggcgacctgctggcctctggcaccatcagcggccctgaacctgagaactttggcagcatgctggagctgagctggaagggaaccaagcccattgacctgggcaatggacagaccaggaaattcctcctggacggggacgaggtgatcatcacaggctattgccagggggatgggtataggattggctttggccaatgtgccggcaaagtgctgcctgccctgctccctagc

SEQ ID NO: 22 - DNA, 1254 bp, synthetic construct
  misc_feature 1..1254: note = Synthetic: Human FAH Codon Optimized Variant 1 (hFAH-co1)
  source 1..1254: mol_type = other DNA
tcctttattcctgtggccgaagatagcgacttccctatccataacctcccatatggcgtcttctcaaccaggggcgaccccaggcccaggattggggtggcaattggagaccagatcctggacctcagcatcattaagcacctctttacaggacctgtgctgagcaagcaccaggatgtgttcaaccagcctaccctgaatagctttatgggactgggccaggcagcatggaaagaagccagagtgttcctccagaatctgctgagcgtgagccaggccaggctcagggatgacacagaactgaggaaatgtgccttcatctcacaggcatcagccacaatgcacctccctgccacaattggggactacactgacttctacagcagcagacagcatgccactaatgtggggattatgtttagagacaaggaaaatgccctcatgccaaattggctgcacctgcctgtgggctaccacggcagagcctcatcagtggtggtgtcaggcacaccaattaggaggccaatgggacagatgaagcctgacgactcaaagccaccagtctatggcgcctgcaaactgctggacatggagctggagatggctttctttgtggggcctggcaacaggctgggagaaccaatccctatttcaaaggcccacgagcacatttttggcatggtgctcatgaatgactggtctgccagagatatccagaagtgggaatacgtgcccctgggcccttttctgggcaagagctttggcaccacagtgtcaccttgggtggtcccaatggatgccctgatgccctttgccgtgcccaaccccaagcaggatcccagaccactgccctacctgtgccacgatgagccctatacctttgacatcaacctgtcagtcaacctgaagggggagggcatgagccaggccgccactatttgcaagagcaactttaaatatatgtactggactatgctccaacagctcactcaccactcagtgaatggctgcaatctgaggcctggcgacctcctggctagcggcactatctctggccctgaacctgagaactttgggagcatgctggagctgtcatggaaagggacaaagcctattgacctggggaatggccagacaaggaagtttctcctggatggcgatgaggtcatcatcactgggtactgccagggcgatggctacaggattggatttggacagtgtgctggcaaagtgctcccagctctgctgccctca

SEQ ID NO: 23 - DNA, 10558 bp, synthetic construct
  misc_feature 1..10558: note = Synthetic: GR-FAH-co3_1.6/1.0_kb
  source 1..10558: mol_type = other DNA
cgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtatttctagatgtaaataattattttaagtttgccctatggtggccccacacatgagacaaacccccaagatgtgacttttgagaatgagacttggataaaaaacatgtagaaatgcaagccctgaagctcaactccctattgctatcacaggggttataattgcataaaatttagctatagaaagttgctgtcatctcttgtgggctgtaatcatcgtctaggcttaagagtaatattgcaaaacctgtcatgcccacacaaatctctccctggcattgttgtctttgcagatgtcagtgaaagagaaccagcagctcccatgagtttggatagccttattttctatagcctccccactattagctttgaagggagcaaagtttaagaaccaaatataaagtttctcatctttatagatgagaaaaattttaaataaagtccaagataattaaatttttaaggatcatttttagctctttaatagcaataaaactcaatatgacataatatggcacttccaaaatctgaataatatataattgcaatgacatacttcttttcagagatttactgaaaagaaatttgttgacactacataacgtgatgagtggtttatactgattgtttcagttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggaagcggcgccaccaatttcagcctgctgaaacaggccggcgacgtggaagagaaccctggcccttccttcattcctgtggccgaggattctgactttcccattcacaacctgccctatggcgtcttttcaaccaggggagatcccaggcccagaatcggggtggccattggagatcagatcctggacctgtcaatcatcaagcacctgtttacaggccctgtgctgagcaagcaccaggatgtgttcaaccagccaactctgaacagcttcatggggctggggcaggctgcctggaaggaggcaagggtgtttctgcagaatctgctgagcgtgtcacaggctaggctgagggatgataccgagctgaggaagtgtgcatttatctcacaggcatcagccacaatgcatctgccagctacaatcggcgactataccgacttttactcaagcaggcagcatgccaccaatgtgggcatcatgttcagggacaaagagaatgccctgatgccaaattggctgcacctgcctgtgggctatcatggcagagccagctcagtggtggtgtcagggacaccaattaggaggcctatgggccagatgaagcctgatgacagcaaaccacctgtctacggcgcctgcaagctcctggatatggaactggagatggctttttttgtgggcccaggcaatagactgggagagcccattccaatctcaaaggcccatgaacatatctttggcatggtcctgatgaatgactggtcagccagagacattcagaagtgggagtacgtgccactgggaccatttctgggaaagagctttggcaccacagtgtcaccatgggtggtgcccatggatgccctgatgccctttgctgtgccaaatccaaaacaagaccccaggcccctcccctatctctgtcatgatgaaccatatacttttgacattaacctgagcgtgaacctgaaaggagaaggcatgagccaagccgccactatctgtaagagcaacttcaaatatatgtactggacaatgctgcagcagctcacccaccatagcgtgaatgggtgtaacctgaggccaggcgacctgctggcatcaggcactatttcagggcctgagcctgaaaattttggatcaatgctggagctgtcatggaagggaactaaacccatcgacctggggaatggccagaccagaaagtttctcctggatggcgatgaggtgatcattacaggctactgtcagggggatggctatagaattggatttggccaatgcgccggcaaagtcctccctgccctgctgcccagctaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaattgcttgttaatcaataaaccgtttaattcgtttcagttgaactttggtctctgcgtatttctttcttatctagtttccatatgcatgtagataagtagcatggcgggttaatcattaactaaccggtacctctagaactatagctagcatgcgcaaatttaaagcgctgatatcgatcgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccaacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggc

SEQ ID NO: 24 - DNA, 10558 bp, synthetic construct
  misc_feature 1..10558: note = Synthetic: GR-FAH-co2_1.6/1.0_kb
  source 1..10558: mol_type = other DNA
cgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtatttctagatgtaaataattattttaagtttgccctatggtggccccacacatgagacaaacccccaagatgtgacttttgagaatgagacttggataaaaaacatgtagaaatgcaagccctgaagctcaactccctattgctatcacaggggttataattgcataaaatttagctatagaaagttgctgtcatctcttgtgggctgtaatcatcgtctaggcttaagagtaatattgcaaaacctgtcatgcccacacaaatctctccctggcattgttgtctttgcagatgtcagtgaaagagaaccagcagctcccatgagtttggatagccttattttctatagcctccccactattagctttgaagggagcaaagtttaagaaccaaatataaagtttctcatctttatagatgagaaaaattttaaataaagtccaagataattaaatttttaaggatcatttttagctctttaatagcaataaaactcaatatgacataatatggcacttccaaaatctgaataatatataattgcaatgacatacttcttttcagagatttactgaaaagaaatttgttgacactacataacgtgatgagtggtttatactgattgtttcagttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggaagcggcgccaccaatttcagcctgctgaaacaggccggcgacgtggaagagaaccctggcccttccttcatcccagtggctgaagattctgacttccccattcacaacctcccttatggagtctttagcacaagaggagacccaagacctagaattggcgtggctattggggatcagatcctggacctgagcattatcaaacatctctttacaggcccagtcctcagcaagcaccaagatgtgttcaatcaacccacactgaactcatttatggggctgggccaagctgcctggaaggaggcaagagtgtttctccagaacctcctgtcagtgagccaggccaggctgagagatgacacagagctgaggaagtgtgcctttatctcacaagccagcgccacaatgcacctgccagcaaccatcggggactacacagacttttacagcagcaggcagcacgccactaatgtgggcatcatgtttagagacaaagagaatgccctcatgcctaactggctgcatctgccagtgggctaccatggcagagccagcagcgtggtggtgtctggcacccctatcaggaggcccatgggccagatgaagcctgatgacagcaagccacctgtgtatggggcatgtaagctgctggacatggagctggaaatggctttctttgtggggcctgggaacaggctgggcgaaccaattcccatcagcaaagctcatgaacacatttttgggatggtgctcatgaatgactggtctgccagagacatccagaagtgggagtatgtccccctgggaccattcctgggcaagagctttgggaccactgtgtcaccatgggtggtgcccatggatgccctgatgccatttgctgtgcctaatcccaaacaagatccaaggccactgccctacctctgccacgatgagccctacacatttgatatcaacctctctgtgaatctgaagggagaaggaatgagccaagctgctaccatttgcaaaagcaactttaagtatatgtattggaccatgctgcagcaactcacccaccacagcgtgaatggctgtaacctgaggcctggcgacctgctggcctctggcaccatcagcggccctgaacctgagaactttggcagcatgctggagctgagctggaagggaaccaagcccattgacctgggcaatggacagaccaggaaattcctcctggacggggacgaggtgatcatcacaggctattgccagggggatgggtataggattggctttggccaatgtgccggcaaagtgctgcctgccctgctccctagctaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaattgcttgttaatcaataaaccgtttaattcgtttcagttgaactttggtctctgcgtatttctttcttatctagtttccatatgcatgtagataagtagcatggcgggttaatcattaactaaccggtacctctagaactatagctagcatgcgcaaatttaaagcgctgatatcgatcgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccaacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggc

SEQ ID NO: 25 - DNA, 10558 bp, synthetic construct
  misc_feature 1..10558: note = Synthetic: GR-FAH-co1_1.6/1.0_kb
  source 1..10558: mol_type = other DNA
cgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtatttctagatgtaaataattattttaagtttgccctatggtggccccacacatgagacaaacccccaagatgtgacttttgagaatgagacttggataaaaaacatgtagaaatgcaagccctgaagctcaactccctattgctatcacaggggttataattgcataaaatttagctatagaaagttgctgtcatctcttgtgggctgtaatcatcgtctaggcttaagagtaatattgcaaaacctgtcatgcccacacaaatctctccctggcattgttgtctttgcagatgtcagtgaaagagaaccagcagctcccatgagtttggatagccttattttctatagcctccccactattagctttgaagggagcaaagtttaagaaccaaatataaagtttctcatctttatagatgagaaaaattttaaataaagtccaagataattaaatttttaaggatcatttttagctctttaatagcaataaaactcaatatgacataatatggcacttccaaaatctgaataatatataattgcaatgacatacttcttttcagagatttactgaaaagaaatttgttgacactacataacgtgatgagtggtttatactgattgtttcagttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggaagcggcgccaccaatttcagcctgctgaaacaggccggcgacgtggaagagaaccctggcccttcctttattcctgtggccgaagatagcgacttccctatccataacctcccatatggcgtcttctcaaccaggggcgaccccaggcccaggattggggtggcaattggagaccagatcctggacctcagcatcattaagcacctctttacaggacctgtgctgagcaagcaccaggatgtgttcaaccagcctaccctgaatagctttatgggactgggccaggcagcatggaaagaagccagagtgttcctccagaatctgctgagcgtgagccaggccaggctcagggatgacacagaactgaggaaatgtgccttcatctcacaggcatcagccacaatgcacctccctgccacaattggggactacactgacttctacagcagcagacagcatgccactaatgtggggattatgtttagagacaaggaaaatgccctcatgccaaattggctgcacctgcctgtgggctaccacggcagagcctcatcagtggtggtgtcaggcacaccaattaggaggccaatgggacagatgaagcctgacgactcaaagccaccagtctatggcgcctgcaaactgctggacatggagctggagatggctttctttgtggggcctggcaacaggctgggagaaccaatccctatttcaaaggcccacgagcacatttttggcatggtgctcatgaatgactggtctgccagagatatccagaagtgggaatacgtgcccctgggcccttttctgggcaagagctttggcaccacagtgtcaccttgggtggtcccaatggatgccctgatgccctttgccgtgcccaaccccaagcaggatcccagaccactgccctacctgtgccacgatgagccctatacctttgacatcaacctgtcagtcaacctgaagggggagggcatgagccaggccgccactatttgcaagagcaactttaaatatatgtactggactatgctccaacagctcactcaccactcagtgaatggctgcaatctgaggcctggcgacctcctggctagcggcactatctctggccctgaacctgagaactttgggagcatgctggagctgtcatggaaagggacaaagcctattgacctggggaatggccagacaaggaagtttctcctggatggcgatgaggtcatcatcactgggtactgccagggcgatggctacaggattggatttggacagtgtgctggcaaagtgctcccagctctgctgccctcataacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaattgcttgttaatcaataaaccgtttaattcgtttcagttgaactttggtctctgcgtatttctttcttatctagtttccatatgcatgtagataagtagcatggcgggttaatcattaactaaccggtacctctagaactatagctagcatgcgcaaatttaaagcgctgatatcgatcgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccaacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggc

SEQ ID NO: 26 - DNA, 9913 bp, synthetic construct
  misc_feature 1..9913: note = Synthetic: GR-FAH-co1_1kb
  source 1..9913: mol_type = other DNA
cgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggaagcggcgccaccaatttcagcctgctgaaacaggccggcgacgtggaagagaaccctggcccttcctttattcctgtggccgaagatagcgacttccctatccataacctcccatatggcgtcttctcaaccaggggcgaccccaggcccaggattggggtggcaattggagaccagatcctggacctcagcatcattaagcacctctttacaggacctgtgctgagcaagcaccaggatgtgttcaaccagcctaccctgaatagctttatgggactgggccaggcagcatggaaagaagccagagtgttcctccagaatctgctgagcgtgagccaggccaggctcagggatgacacagaactgaggaaatgtgccttcatctcacaggcatcagccacaatgcacctccctgccacaattggggactacactgacttctacagcagcagacagcatgccactaatgtggggattatgtttagagacaaggaaaatgccctcatgccaaattggctgcacctgcctgtgggctaccacggcagagcctcatcagtggtggtgtcaggcacaccaattaggaggccaatgggacagatgaagcctgacgactcaaagccaccagtctatggcgcctgcaaactgctggacatggagctggagatggctttctttgtggggcctggcaacaggctgggagaaccaatccctatttcaaaggcccacgagcacatttttggcatggtgctcatgaatgactggtctgccagagatatccagaagtgggaatacgtgcccctgggcccttttctgggcaagagctttggcaccacagtgtcaccttgggtggtcccaatggatgccctgatgccctttgccgtgcccaaccccaagcaggatcccagaccactgccctacctgtgccacgatgagccctatacctttgacatcaacctgtcagtcaacctgaagggggagggcatgagccaggccgccactatttgcaagagcaactttaaatatatgtactggactatgctccaacagctcactcaccactcagtgaatggctgcaatctgaggcctggcgacctcctggctagcggcactatctctggccctgaacctgagaactttgggagcatgctggagctgtcatggaaagggacaaagcctattgacctggggaatggccagacaaggaagtttctcctggatggcgatgaggtcatcatcactgggtactgccagggcgatggctacaggattggatttggacagtgtgctggcaaagtgctcccagctctgctgccctcataacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaattgcttgttaatcaataaaccgtttaattcgtttcagttgaactttggtctctgcgtatttctttcttatctagtttccatatgcatgtagataagtagcatggcgggttaatcattaactaaccggtacctctagaactatagctagcatgcgcaaatttaaagcgctgatatcgatcgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccaacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggc

SEQ ID NO: 27 - DNA, 145 bp, synthetic construct
  misc_feature 1..145: note = Synthetic: 145bp ITR
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 28 - DNA, 130 bp, synthetic construct
  misc_feature 1..130: note = Synthetic: 130bp ITR
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 29 - DNA, 123 bp, synthetic construct
  misc_feature 1..123: note = Synthetic: B loop deletion ITR
  source 1..123: mol_type = other DNA
aggaacccctagtgatggagttggccactccctctctgcgcgctcgctcgctcactgaggccgcccgggctttgcccgggcggcctcagtgagcgagcgagcgcgcagagagggagtggccaa

SEQ ID NO: 30 - DNA, 123 bp, synthetic construct
  misc_feature 1..123: note = Synthetic: C loop deletion ITR
  source 1..123: mol_type = other DNA
aggaacccctagtgatggagttggccactccctctctgcgcgctcgctcgctcactgaggccgggcgaccaaaggtcgcccggcctcagtgagcgagcgagcgcgcagagagggagtggccaa

SEQ ID NO: 31 - DNA, 3965 bp, synthetic construct
  misc_feature 1..3965: note = Synthetic: Gene of interest (GOI) cassette
  source 1..3965: mol_type = other DNA
tttctagatgtaaataattattttaagtttgccctatggtggccccacacatgagacaaacccccaagatgtgacttttgagaatgagacttggataaaaaacatgtagaaatgcaagccctgaagctcaactccctattgctatcacaggggttataattgcataaaatttagctatagaaagttgctgtcatctcttgtgggctgtaatcatcgtctaggcttaagagtaatattgcaaaacctgtcatgcccacacaaatctctccctggcattgttgtctttgcagatgtcagtgaaagagaaccagcagctcccatgagtttggatagccttattttctatagcctccccactattagctttgaagggagcaaagtttaagaaccaaatataaagtttctcatctttatagatgagaaaaattttaaataaagtccaagataattaaatttttaaggatcatttttagctctttaatagcaataaaactcaatatgacataatatggcacttccaaaatctgaataatatataattgcaatgacatacttcttttcagagatttactgaaaagaaatttgttgacactacataacgtgatgagtggtttatactgattgtttcagttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggaagcggcgccaccaatttcagcctgctgaaacaggccggcgacgtggaagagaaccctggcccttcctttattcctgtggccgaagatagcgacttccctatccataacctcccatatggcgtcttctcaaccaggggcgaccccaggcccaggattggggtggcaattggagaccagatcctggacctcagcatcattaagcacctctttacaggacctgtgctgagcaagcaccaggatgtgttcaaccagcctaccctgaatagctttatgggactgggccaggcagcatggaaagaagccagagtgttcctccagaatctgctgagcgtgagccaggccaggctcagggatgacacagaactgaggaaatgtgccttcatctcacaggcatcagccacaatgcacctccctgccacaattggggactacactgacttctacagcagcagacagcatgccactaatgtggggattatgtttagagacaaggaaaatgccctcatgccaaattggctgcacctgcctgtgggctaccacggcagagcctcatcagtggtggtgtcaggcacaccaattaggaggccaatgggacagatgaagcctgacgactcaaagccaccagtctatggcgcctgcaaactgctggacatggagctggagatggctttctttgtggggcctggcaacaggctgggagaaccaatccctatttcaaaggcccacgagcacatttttggcatggtgctcatgaatgactggtctgccagagatatccagaagtgggaatacgtgcccctgggcccttttctgggcaagagctttggcaccacagtgtcaccttgggtggtcccaatggatgccctgatgccctttgccgtgcccaaccccaagcaggatcccagaccactgccctacctgtgccacgatgagccctatacctttgacatcaacctgtcagtcaacctgaagggggagggcatgagccaggccgccactatttgcaagagcaactttaaatatatgtactggactatgctccaacagctcactcaccactcagtgaatggctgcaatctgaggcctggcgacctcctggctagcggcactatctctggccctgaacctgagaactttgggagcatgctggagctgtcatggaaagggacaaagcctattgacctggggaatggccagacaaggaagtttctcctggatggcgatgaggtcatcatcactgggtactgccagggcgatggctacaggattggatttggacagtgtgctggcaaagtgctcccagctctgctgccctcataacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttg

SEQ ID NO: 32 - DNA, 10130 bp, synthetic construct
  misc_feature 1..10130: note = Synthetic: LK03-GR hFAH-co1
  source 1..10130: mol_type = other DNA
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaattgcttgttaatcaataaaccgtttaattcgtttcagttgaactttggtctctgcgtatttctttcttatctagtttccatatgcatgtagataagtagcatggcgggttaatcattaactaaccggtacctctagaactatagctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttttctagatgtaaataattattttaagtttgccctatggtggccccacacatgagacaaacccccaagatgtgacttttgagaatgagacttggataaaaaacatgtagaaatgcaagccctgaagctcaactccctattgctatcacaggggttataattgcataaaatttagctatagaaagttgctgtcatctcttgtgggctgtaatcatcgtctaggcttaagagtaatattgcaaaacctgtcatgcccacacaaatctctccctggcattgttgtctttgcagatgtcagtgaaagagaaccagcagctcccatgagtttggatagccttattttctatagcctccccactattagctttgaagggagcaaagtttaagaaccaaatataaagtttctcatctttatagatgagaaaaattttaaataaagtccaagataattaaatttttaaggatcatttttagctctttaatagcaataaaactcaatatgacataatatggcacttccaaaatctgaataatatataattgcaatgacatacttcttttcagagatttactgaaaagaaatttgttgacactacataacgtgatgagtggtttatactgattgtttcagttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggaagcggcgccaccaatttcagcctgctgaaacaggccggcgacgtggaagagaaccctggcccttcctttattcctgtggccgaagatagcgacttccctatccataacctcccatatggcgtcttctcaaccaggggcgaccccaggcccaggattggggtggcaattggagaccagatcctggacctcagcatcattaagcacctctttacaggacctgtgctgagcaagcaccaggatgtgttcaaccagcctaccctgaatagctttatgggactgggccaggcagcatggaaagaagccagagtgttcctccagaatctgctgagcgtgagccaggccaggctcagggatgacacagaactgaggaaatgtgccttcatctcacaggcatcagccacaatgcacctccctgccacaattggggactacactgacttctacagcagcagacagcatgccactaatgtggggattatgtttagagacaaggaaaatgccctcatgccaaattggctgcacctgcctgtgggctaccacggcagagcctcatcagtggtggtgtcaggcacaccaattaggaggccaatgggacagatgaagcctgacgactcaaagccaccagtctatggcgcctgcaaactgctggacatggagctggagatggctttctttgtggggcctggcaacaggctgggagaaccaatccctatttcaaaggcccacgagcacatttttggcatggtgctcatgaatgactggtctgccagagatatccagaagtgggaatacgtgcccctgggcccttttctgggcaagagctttggcaccacagtgtcaccttgggtggtcccaatggatgccctgatgccctttgccgtgcccaaccccaagcaggatcccagaccactgccctacctgtgccacgatgagccctatacctttgacatcaacctgtcagtcaacctgaagggggagggcatgagccaggccgccactatttgcaagagcaactttaaatatatgtactggactatgctccaacagctcactcaccactcagtgaatggctgcaatctgaggcctggcgacctcctggctagcggcactatctctggccctgaacctgagaactttgggagcatgctggagctgtcatggaaagggacaaagcctattgacctggggaatggccagacaaggaagtttctcctggatggcgatgaggtcatcatcactgggtactgccagggcgatggctacaggattggatttggacagtgtgctggcaaagtgctcccagctctgctgccctcataacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgaggaacccctagtgatggagttggccactccctctctgcgcgctcgctcgctcactgaggccgcccgggcaaagcccgggcggcctcagtgagcgagcgagcgcgcagagagggagtggccaactttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggt

SEQ ID NO: 33 - DNA, 10127 bp, synthetic construct
  misc_feature 1..10127: note = Synthetic: sL65-GR hFAH-co1
  source 1..10127: mol_type = other DNA
cccctgtaattgcttgttaatcaataaaccgtttaattcgtttcagttgaactttggtctctgcgtatttctttcttatctagtttccatatgcatgtagataagtagcatggcgggttaatcattaactaaccggtacctctagaactatagctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttttctagatgtaaataattattttaagtttgccctatggtggccccacacatgagacaaacccccaagatgtgacttttgagaatgagacttggataaaaaacatgtagaaatgcaagccctgaagctcaactccctattgctatcacaggggttataattgcataaaatttagctatagaaagttgctgtcatctcttgtgggctgtaatcatcgtctaggcttaagagtaatattgcaaaacctgtcatgcccacacaaatctctccctggcattgttgtctttgcagatgtcagtgaaagagaaccagcagctcccatgagtttggatagccttattttctatagcctccccactattagctttgaagggagcaaagtttaagaaccaaatataaagtttctcatctttatagatgagaaaaattttaaataaagtccaagataattaaatttttaaggatcatttttagctctttaatagcaataaaactcaatatgacataatatggcacttccaaaatctgaataatatataattgcaatgacatacttcttttcagagatttactgaaaagaaatttgttgacactacataacgtgatgagtggtttatactgattgtttcagttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggaagcggcgccaccaatttcagcctgctgaaacaggccggcgacgtggaagagaaccctggcccttcctttattcctgtggccgaagatagcgacttccctatccataacctcccatatggcgtcttctcaaccaggggcgaccccaggcccaggattggggtggcaattggagaccagatcctggacctcagcatcattaagcacctctttacaggacctgtgctgagcaagcaccaggatgtgttcaaccagcctaccctgaatagctttatgggactgggccaggcagcatggaaagaagccagagtgttcctccagaatctgctgagcgtgagccaggccaggctcagggatgacacagaactgaggaaatgtgccttcatctcacaggcatcagccacaatgcacctccctgccacaattggggactacactgacttctacagcagcagacagcatgccactaatgtggggattatgtttagagacaaggaaaatgccctcatgccaaattggctgcacctgcctgtgggctaccacggcagagcctcatcagtggtggtgtcaggcacaccaattaggaggccaatgggacagatgaagcctgacgactcaaagccaccagtctatggcgcctgcaaactgctggacatggagctggagatggctttctttgtggggcctggcaacaggctgggagaaccaatccctatttcaaaggcccacgagcacatttttggcatggtgctcatgaatgactggtctgccagagatatccagaagtgggaatacgtgcccctgggcccttttctgggcaagagctttggcaccacagtgtcaccttgggtggtcccaatggatgccctgatgccctttgccgtgcccaaccccaagcaggatcccagaccactgccctacctgtgccacgatgagccctatacctttgacatcaacctgtcagtcaacctgaagggggagggcatgagccaggccgccactatttgcaagagcaactttaaatatatgtactggactatgctccaacagctcactcaccactcagtgaatggctgcaatctgaggcctggcgacctcctggctagcggcactatctctggccctgaacctgagaactttgggagcatgctggagctgtcatggaaagggacaaagcctattgacctggggaatggccagacaaggaagtttctcctggatggcgatgaggtcatcatcactgggtactgccagggcgatggctacaggattggatttggacagtgtgctggcaaagtgctcccagctctgctgccctcataacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgaggaacccctagtgatggagttggccactccctctctgcgcgctcgctcgctcactgaggccgcccgggcaaagcccgggcggcctcagtgagcgagcgagcgcgcagagagggagtggccaactttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccccagtacggctacctaacactcaacaacggtagtcaggccgtgggacgctcctccttttactgcctggaatatttcccatcgcagatgctgagaacgggcaataactttgagttcagctacagcttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttaccttacccgt

SEQ ID NO: 34 - DNA, 7393 bp, synthetic construct
  misc_feature 1..7393: note = Synthetic: AAV-DJ-mha-mFAH (PM-0550)
  source 1..7393: mol_type = other DNA
cagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaatgcatggatcccctagggcggccgcctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggcccttcctttattccagtggccgaggactccgactttcccatccaaaacctgccctatggtgttttctccactcaaagcaacccaaagccacggattggtgtagccatcggtgaccagatcttggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcgcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggggcaggggcagaccaggaccttcctgctggacggcgatgaagtcatcataacaggtcactgccagggggacggctaccgtgttggctttggccagtgtgctgggaaagtgctgcctgccctttcaccagcctaaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaacagatgtcagctaagactaggtcatgtgtaggctgtctacaccagtgaaaatcgcaaaaagaatctaagaaattccacatttctagaaaataggtttggaaaccgtattccattttacaaaggacacttacatttctctttttgttttccaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctactctgtggttcagaactctatcttccaaaggcgcgcttcaccctagcagcctctttggctcagaggaatccctgcctttcctcccttcatctcagcagagaatgtagttccacatgggcaacacaatgaaaataaacgttaatactctcccatcttatgggtggtgaccctagaaaccaatacttcaacattacgagaattctgaatgagagactaaaagcttatgaactgtggctttcctttgtcagtgggactctaagaatgagttggggacaaaagagataggaatggctttaaaggtgactagttgaactgataaagtaaatgaactgaggaaaaaaaatatcactcaacctgcaggggacgtcctacgtaatgcataggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagc

SEQ ID NO: 35 - DNA, 7393 bp, synthetic construct
  misc_feature 1..7393: note = Synthetic: AAV-DJ-mha-hFAH (PM-0549)
  source 1..7393: mol_type = other DNA
cagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaatgcatggatcccctagggcggccgcctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggcccttccttcatcccggtggccgaggattccgacttccccatccacaacctgccctacggcgtcttctcgaccagaggcgacccaagaccgaggataggtgtggccattggcgaccagatcctggacctcagcatcatcaagcacctctttactggtcctgtcctctccaaacaccaggatgtcttcaatcagcctacactcaacagcttcatgggcctgggtcaggctgcctggaaggaggcgagagtgttcttgcagaacttgctgtctgtgagccaagccaggctcagagatgacaccgaacttcggaagtgtgcattcatctcccaggcttctgccacgatgcaccttccagccaccataggagactacacagacttctattcctctcggcagcatgctaccaacgtcggaatcatgttcagggacaaggagaatgcgttgatgccaaattggctgcacttaccagtgggctaccatggccgtgcctcctctgtcgtggtgtctggcaccccaatccgaaggcccatgggacagatgaaacctgatgactctaagcctcccgtatatggtgcctgcaagctcttggacatggagctggaaatggctttttttgtaggccctggaaacagattgggagagccgatccccatttccaaggcccatgagcacatttttggaatggtccttatgaacgactggagtgcacgagacattcagaagtgggagtatgtccctctcgggccattccttgggaagagttttgggaccactgtctctccgtgggtggtgcccatggatgctctcatgccctttgctgtgcccaacccgaagcaggaccccaggcccctgccgtatctgtgccatgacgagccctacacatttgacatcaacctctctgttaacctgaaaggagaaggaatgagccaggcggctaccatatgcaagtccaattttaagtacatgtactggacgatgctgcagcagctcactcaccactctgtcaacggctgcaacctgcggccgggggacctcctggcttctgggaccatcagcgggccggagccagaaaacttcggctccatgttggaactgtcgtggaagggaacgaagcccatagacctggggaatggtcagaccaggaagtttctgctggacggggatgaagtcatcataacagggtactgccagggggatggttaccgcatcggctttggccagtgtgctggaaaagtgctgcctgctctcctgccatcataaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaacagatgtcagctaagactaggtcatgtgtaggctgtctacaccagtgaaaatcgcaaaaagaatctaagaaattccacatttctagaaaataggtttggaaaccgtattccattttacaaaggacacttacatttctctttttgttttccaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctactctgtggttcagaactctatcttccaaaggcgcgcttcaccctagcagcctctttggctcagaggaatccctgcctttcctcccttcatctcagcagagaatgtagttccacatgggcaacacaatgaaaataaacgttaatactctcccatcttatgggtggtgaccctagaaaccaatacttcaacattacgagaattctgaatgagagactaaaagcttatgaactgtggctttcctttgtcagtgggactctaagaatgagttggggacaaaagagataggaatggctttaaaggtgactagttgaactgataaagtaaatgaactgaggaaaaaaaatatcactcaacctgcaggggacgtcctacgtaatgcataggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagc

SEQ ID NO: 36 - DNA, 10221 bp, synthetic construct
  misc_feature 1..10221: note = Synthetic: AAV-DJ-mha-mFAH (PM-0549)
  source 1..10221: mol_type = other DNA
gtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcttgttaatcaataaaccgtttaattcgtttcagttgaactttggtctctgcgtatttctttcttatctagtttccatatgcatgtagataagtagcatggcgggttaatcattaactaaccggtacctctagaactatagctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttaccgactgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggcccttcctttattccagtggccgaggactccgactttcccatccaaaacctgccctatggtgttttctccactcaaagcaacccaaagccacggattggtgtagccatcggtgaccagatcttggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcgcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggggcaggggcagaccaggaccttcctgctggacggcgatgaagtcatcataacaggtcactgccagggggacggctaccgtgttggctttggccagtgtgctgggaaagtgctgcctgccctttcaccagcctaaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaacagatgtcagctaagactaggtcatgtgtaggctgtctacaccagtgaaaatcgcaaaaagaatctaagaaattccacatttctagaaaataggtttggaaaccgtattccattttacaaaggacacttacatttctctttttgttttccaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctactctgtggttcagaactctatcttccaaaggcgcgcttcaccctagcagcctctttggctcagaggaatccctgcctttcctcccttcatctcagcagagaatgtagttccacatgggcaacacaatgaaaataaacgttaatactctcccatcttatgggtggtgaccctagaaaccaatacttcaacattacgagaattctgaatgagagactaaaagcttatgaactgtggctttcctttgtcagtgggactctaagaatgagttggggacaaaagagataggaatggctttaaaggtgactagttgaactgataaagtaaatgaactgaggaaaaaaaatatcactcaatcggtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaa

SEQ ID NO: 37 - DNA, 19 bp, synthetic construct
  misc_feature 1..19: note = Synthetic: F1
  source 1..19: mol_type = other DNA
atgttccacgaagaagcca

SEQ ID NO: 38 - DNA, 19 bp, synthetic construct
  misc_feature 1..19: note = Synthetic: R1
  source 1..19: mol_type = other DNA
tcagcaggctgaaattggt

SEQ ID NO: 39 - DNA, 23 bp, synthetic construct
  misc_feature 1..23: note = Synthetic: R2
  source 1..23: mol_type = other DNA
agctgtttcttactccattctca

SEQ ID NO: 40 - DNA, 24 bp, synthetic construct
  misc_feature 1..24: note = Synthetic: P1
  source 1..24: mol_type = other DNA
aggcaacgtcatgggtgtgacttt

SEQ ID NO: 41 - protein, 8 aa, synthetic construct
  REGION 1..8: note = Synthetic: consensus motif
DVEXNPGP